(19) United States
(12) Patent Application Publication
(10) Pub. No.: US 2025/0066341 A1
(43) Pub. Date: Feb. 27, 2025

(54) CYCLOHEXANE ACID DERIVATIVES AS LPA RECEPTOR INHIBITORS

(71) Applicant: CHIESI FARMACEUTICI S.P.A., Parma (IT)

(72) Inventors: Elisabetta ARMANI, Parma (IT); Gabriele AMARI, Parma (IT); Mafalda PAGANO, Parma (IT); Marta GIULIANI, Parma (IT)

(73) Assignee: CHIESI FARMACEUTICI S.P.A., Parma (IT)

(21) Appl. No.: 18/723,049

(22) PCT Filed: Dec. 21, 2022
(86) PCT No.: PCT/EP2022/087156

§ 371 Date: Jun. 21, 2024

(30) Foreign Application Priority Data
Dec. 23, 2021 (EP) 21217271.2

(51) Int. Cl. C07D 413/14; A61K 31/415; A61K 31/4439; A61K 31/497; A61K 31/506; C07D 261/04; C07D 401/04; C07D 401/14; C07D 403/12; C07D 413/04; C07D 413/12

(52) U.S. Cl. CPC C07D 413/14; A61K 31/415; A61K 31/4439; A61K 31/497; A61K 31/506; C07D 261/04; C07D 401/04; C07D 401/14; C07D 403/12; C07D 413/04; C07D 413/12

(57) ABSTRACT

The present invention relates to a compounds of general formula (I) inhibiting lysophosphatidic acid receptor 1 (LPA1), particularly the invention relates to compounds that are cyclohexane acid derivatives, methods of preparing such compounds, pharmaceutical compositions containing them and therapeutic use thereof. The compounds of the invention may be useful in the treatment of diseases or conditions associated with a dysregulation of LPA receptors, in particular fibrosis.

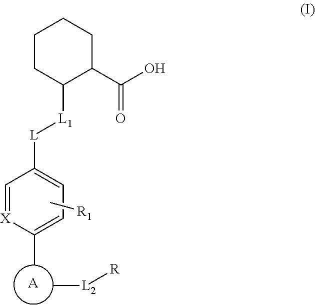

### FIELD OF THE INVENTION

The present invention generally relates to compounds inhibiting lysophosphatidic acid receptors (hereinafter LPA inhibitors); the invention relates to compounds that are amido cyclohexane acid derivatives, methods of preparing such compounds, pharmaceutical compositions containing them and therapeutic use thereof.

The compounds of the invention may be useful for instance in the treatment of many disorders associated with LPA receptors mechanisms.

### BACKGROUND OF THE INVENTION

Lysophosphatidic acid (LPA) is a phospholipid mediator concentrated in serum that acts as a potent extracellular signalling molecule through at least six cognate G protein-coupled receptors (GPCRs) in numerous developmental and adult processes including cell survival, proliferation, migration, differentiation, vascular regulation, and cytokine release.

These LPA-mediated processes involve nervous system function, vascular development, immune system function, cancer, reproduction, fibrosis, and obesity (see e.g. Yung et al., J Lipid Res. 2014 July; 55(7):1192-214). The formation of an LPA species depends on its precursor phospholipid, which can vary typically by acyl chain length and degree of saturation. The term LPA generally refers to 18:1 oleoyl-LPA (1-acyl-2-hydroxy-sn-glycero3-phosphate), that is the most quantitatively abundant forms of LPA in human plasma with 16:0-, 18:2-, and 18:1-LPA (see e.g. Sano et al., J Biol Chem. 2002 Dec. 13; 277(50):21197-206). All LPA species are produced from membrane phospholipids via two major metabolic routes. Depending upon the site of synthesis, membrane phospholipids get converted to the corresponding lysophospholipids by the action of phospholipase A1 (PLA1), phospholipase A2 (PLA2), or PLA1 and lecithin-cholesterol acyltransferase (LCAT). Autotaxin (ATX) then acts on the lysophospholipids and converts them into LPA species. The second pathway first converts the phospholipids into phosphatidic acid by the action of phospholipase D. Then PLA1 or PLA2 metabolize phosphatidic acid to the lysophosphatidic acids (see e.g. Riaz et al., Int J Mol Sci. 2016 February; 17(2): 215).

ATX activity is the major source of plasma extracellular LPA but the source of tissue LPA that contributes to signalling pools likely involves not only ATX but other enzymes as well. The biological functions of LPA are mediated by at least six recognized cell-surface receptors.

All LPA receptors are rhodopsin-like 7-TM proteins that signal through at least two of the four Ga subunit families (Gα12/13, Gαq/11, Gαi/o and GαS). LPA receptors usually trigger response from multiple heterotrimeric G-proteins, resulting in diverse outcomes in a context and cell type dependent manner. Gα12/13-mediated LPA signalling regulates cell migration, invasion and cytoskeletal re-adjustments through activation of RHO pathway proteins. RAC activation downstream of Gαi/o-PI3K also regulates similar processes, but the most notable function of LPA-induced Gαi/o is mitogenic signalling through the RAF-MEK-MAPK cascade and survival signalling through the PI3K-AKT pathway. The LPA-coupled Gαq/11 protein primarily regulates Ca2+ homeostasis through PLC and the second messengers IP3 and DAG. Lastly, GαS can activate adenylyl cyclase and increase cAMP concentration upon LPA stimulation (see e.g. Riaz et al., Int J Mol Sci. 2016 February; 17(2): 215).

LPA, especially LPA1, LPA2 and LPA3, have been implicated in migration, invasion, metastasis, proliferation and survival and differ in their tissue distribution and downstream signalling pathways.

LPA1 is a 41-kD protein that is widely expressed, albeit at different levels, in all human adult tissues examined and the importance of LPA1 signalling during development and adult life has been demonstrated through numerous approaches (see e.g. Ye at al., 2002, Neuroreport. December 3; 13(17):2169-75). Wide expression of LPA1 is observed in adult mice, with clear presence in at least brain, uterus, testis, lung, small intestine, heart, stomach, kidney, spleen, thymus, placenta, and skeletal muscle. LPA1 is also widely expressed in humans where the expression is more spatially restricted during embryonic development. LPA1 couples with and activates three types of G proteins: Gαi/o, Gαq/11, and Gα12/13. LPA1 activation induces a range of cellular responses: cell proliferation and survival, cell migration, cytoskeletal changes, Ca2+ mobilization, adenylyl cyclase inhibition and activation of mitogen-activated protein kinase, phospholipase C, Akt, and Rho pathways (see e.g. Choi et al., Annu Rev Pharmacol Toxicol. 2010; 50:157-86).

LPA2 in humans is a 39-kD protein and shares ˜55% amino acid sequence homology with LPA1 (see e.g. Yung et al., J Lipid Res. 2014 July; 55(7):1192-214). In mouse, LPA2 is highly expressed in kidney, uterus, and testis and moderately expressed in lung; in human tissues, high expression of LPA2 is detected in testis and leukocytes, with moderate expression found in prostate, spleen, thymus, and pancreas.

In terms of signalling activity, LPA2 mostly activates the same pathways as triggered by LPA1 with some exceptions that regards its unique cross-talk behaviour. For example, LPA2 promotes cell migration through interactions with focal adhesion molecule TRIP6 (see e.g. Lai Y J, 2005, Mol. Cell. Biol. 25:5859-68), and several PDZ proteins and zinc finger proteins are also reported to interact directly with the carboxyl-terminal tail of LPA2 (see e.g. Lin F T, 2008, Biochim. Biophys. Acta 1781:558-62).

Human LPA3 is a 40-kD protein and shares sequence homology with LPA1 (˜54%) and LPA2 (˜49%). In adult humans LPA3 is highly expressed in heart, pancreas, prostate and testis. Moderate levels of expression are also found in brain, lungs and ovary. Like LPA1 and LPA2 the signalling activity of LPA3 results from its coupling to Gαi/o and Gαq/11 (see e.g Ishii et al., Mol Pharmacol 58:895-902, 2000). Each LPA has multiple important regulatory functions throughout the body.

As LPA signalling has been strongly implicated in many disease states, great interest has been expressed in developing specific LPA inhibitors (see e.g. Stoddard et el., Biomol Ther (Seoul) 2015 January; 23(1):1-11). Different studies have demonstrated a positive role for LPA in the pathogenesis of pulmonary fibrosis (PF), a devastating disease characterized by alveolar epithelial cell injury, accumulation of myofibroblasts and deposition of extracellular matrix proteins leading to a loss of lung function and death (see e.g. Wilson M S, Wynn T A (2009), Mucosal Immunol 2; 103-121).

Evidences showed that lysophosphatidic acid levels dramatically increase in bronchoalveolar lavage fluid of PF patients where it mediates fibroblast migration in the injured lung acting through LPA1 (see e.g. Tager et al., Nat Med. 2008 January; 14(1):45-54). In addition, mice lacking LPA1 or LPA2 are markedly protected from fibrosis and mortality in a mouse model of the bleomycin induced pulmonary fibrosis (see e.g. Huang et al., Am J Respir Cell Mol Biol. 2013 December; 49(6): 912-922 and Tager et al., Nat Med. 2008 January; 14(1):45-54).

In vitro, LPA1 is known to induce the proliferation and differentiation of lung fibroblasts (see e.g. Shiomi et al., Wound Repair Regen. 2011 March-April; 19(2): 229-240), and to augment the fibroblast-mediated contraction of released collagen gels (see e.g. Mio et al., Journal of Laboratory and Clinical Medicine, Volume 139, Issue 1, January 2002, Pages 20-27). In human lung fibroblasts, the knockdown of LPA2 attenuated the LPA-induced expression of TGF-β1 and the differentiation of lung fibroblasts to myofibroblasts, resulting in the decreased expression of different profibrotic markers such as FN, α-SMA, and collagen, as well as decreased activation of extracellular regulated kinase 1/2, Akt, Smad3, and p38 mitogen-activated protein kinase (see e.g. Huang et al., Am J Respir Cell Mol Biol. 2013 December; 49(6): 912-922). Moreover Xu et al., confirmed that the expression of LPA2 was also up-regulated in lungs from bleomycin-challenged mice where it is able to induce the activation of TGF-β pathway, a key cytokine that play an essential role during the development of the disease, via a RhoA and Rho kinase pathway (see e.g. Xu et al., Am J Pathol. 2009 April; 174(4):1264-79). In in vivo preclinical model, the oral administration of an LPA1 antagonist significantly reduced bleomycin-induced pulmonary fibrosis in mice (Tager et al., Nat Med. 2008 January; 14(1):45-54; Swaney et al., Br J Pharmacol. 2010 August; 160(7): 1699-1713), and the intraperitoneal injection of an LPA1/3 antagonist ameliorated irradiation-induced lung fibrosis (see e.g. Gan et al., 2011, Biochem Biophys Res Commun 409; 7-13). In a renal fibrosis model, LPA1 administration of an LPA1 antagonist suppressed renal interstitial fibrosis (see e.g Pradere et al., J Am Soc Nephrol 2007; 18:3110-3118).

Various compounds have been described in the literature as LPA1 or LPA2 antagonist.

WO2019126086 and WO2019126087 (Bristol-Myers Squibb) disclose cyclohexyl acid isoxazole azines as LPA1 antagonists, useful for the treatment of disorder or condition associated with dysregulation of lysophosphatidic acid receptor 1.

WO2019126099 (Bristol-Myers Squibb) discloses isoxazole N-linked carbamoyl cyclohexyl acid as LPA1 antagonist for the treatment of disorder or condition associated with dysregulation of lysophosphatidic acid receptor 1.

WO2019126090 (Bristol-Myers Squibb) discloses triazole N-linked carbamoyl cyclohexyl acids as LPA1 antagonists. The compounds are selective LPA1 receptor inhibitors and are useful for the treatment of disorder or condition associated with dysregulation of lysophosphatidic acid receptor 1.

WO2017223016 (Bristol-Myers Squibb) discloses carbamoyloxymethyl triazole cyclohexyl acids as LPA1 antagonists for the treatment of fibrosis including idiopathic pulmonary fibrosis.

WO2012028243 (Merck) discloses pyrazolopyridinone derivatives according to formula (I) and a process of manufacturing thereof as LPA2 receptor antagonists for the treatment of various diseases.

WO2012100436 (Curegenix) discloses phenyl isoxazole carbamate derivatives as LPA1 antagonists for the treatment of LPA mediated disorder, such as fibrosis.

Amgen Inc. discloses in “Discovery of potent LPA2 (EDG4) antagonists as potential anticancer agents” Bioorg Med Chem Lett. 2008 Feb. 1; 18(3):1037-41, LPA2 antagonists. Key compounds were evaluated in vitro for inhibition of LPA2 mediated Erk activation and proliferation of HCT-116 cells. These compounds could be used as tool compounds to evaluate the anticancer effects of blocking LPA2 signaling.

Of note, antagonizing the LPA receptors may be useful for the treatment of fibrosis and disease, disorder and conditions that result from fibrosis, and antagonizing receptors LPA1 may be efficacious in the treatment of the above-mentioned diseases, disorders and conditions.

Despite the above cited prior art, there remains a potential for developing inhibitors of receptors LPA1 with a proper antagonist activity useful for the treatment of diseases or conditions associated with a dysregulation of LPA receptors, in particular fibrosis.

In this respect, the state of the art does not describe or suggest cyclohexane acid derivatives of general formula (I) of the present invention having a good antagonist activity on receptors LPA1.

### SUMMARY OF THE INVENTION

In a first aspect the invention refers to a compound of formula (I)

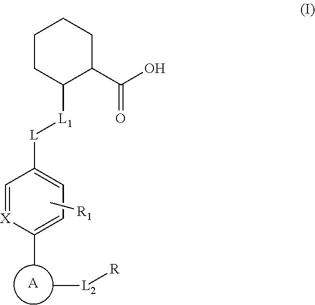

wherein X is —CH— or N;
R₁ is H or —(C₁-C₄)alkyl,
L is —O— or —NH—;
L₁ is (—CH₂—)ₙ, or —C(O)—;
n is an integer between 0 and 2;
A is selected from the group consisting of

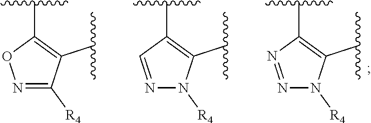

L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, —(C₁-C₄)alkyl-R₂, —NR₆R₇, —OC(O)NR₆R₇, —NHC(O)O—(C₁-C₄)alkyl-R₂, heteroaryl, wherein any of such heteroaryl may be optionally substituted by one or more groups selected from —OR₃, —(C₁-C₄)alkyl, —(C₁-C₄)haloalkyl, —(C₃-C₆)cycloalkyl, aryl, heteroaryl, —NR₆R₇;
R₂ is H or selected from the group consisting of —(C₁-C₄)alkyl, aryl, heteroaryl wherein any of such aryl, heteroaryl and alkyl are optionally substituted by one or more groups selected from —(C₁-C₄)alkyl, halo, —OR₅, —R₅;
R₃ is H or selected from the group consisting of —(C₁-C₄)alkyl, —(C₁-C₄)haloalkyl, —(C₃-C₆)cycloalkyl optionally substituted by one or more halo;
R₄ is H or —(C₁-C₄)alkyl;
R₅ is aryl optionally substituted by one or more groups selected from —(C₁-C₄)alkyl and halo;
R₆ and R₇ are at each occurrence independently H or selected from the group consisting of —(C₁-C₆)alkyl, —(C₃-C₆)cycloalkyl, heteroaryl wherein any of such heteroaryl may be optionally substituted by one or more groups selected from —OR₄, aryl, —(C₃-C₆)cycloalkyl, heteroaryl
   with the proviso that when A is

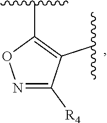

L is —NH—, L₁ is —C(O)—, X is —CH— and L₂ is NH, R is not —C(O)O(C₁-C₅)alkyl-R₂.

In a second aspect, the invention refers to pharmaceutical composition comprising a compound of formula (I) in admixture with one or more pharmaceutically acceptable carrier or excipient.

In a third aspect, the invention refers to a compound of formula (I) for the use as a medicament.

In a further aspect, the invention refers to a compound of formula (I) for use in treating disease, disorder, or condition associated with dysregulation of lysophosphatidic acid receptor 1 (LPA1).

In a further aspect, the invention refers to a compound of formula (I) for use in the prevention and/or treatment of fibrosis and/or diseases, disorders, or conditions that involve fibrosis.

In a further aspect, the invention refers to a compound of formula (I) for use in the prevention and/or treatment idiopathic pulmonary fibrosis (IPF)

### DETAILED DESCRIPTION OF THE INVENTION

Unless otherwise provided, the term compound of formula (I) comprises in its meaning stereoisomer, tautomer or pharmaceutically acceptable salt or solvate.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, iodic acid, formic acid, benzoic acid, sulfuric acid, phosphoric acid, methane sulfonic acid, camphor sulfonic acid, nitric acid, acetic acid, oxalic acid, maleic acid, fumaric acid, succinic acid and p-toluenesulfonic acid, trifluoroacetic acid, 2-naphthoic acid, tartaric acid, 1-hydroxy-2-naphthoic acid, naphthalene-2,7-disulfonic acid and citric acid. The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “5-membered heterocyclyl” refers to a mono saturated or unsaturated group containing one or more heteroatoms selected from N and O.

The term “(Cₓ-C_{y}) alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y}) alkylene” wherein x and y are integers, refers to a Cₓ-C_{y}alkyl radical having in total two unsatisfied valencies, such as a divalent methylene radical.

The expressions “(Cₓ-C_{y}) haloalkyl” wherein x and y are integers, refer to the above defined “(Cₓ-C_{y}) alkyl” groups wherein one or more hydrogen atoms are replaced by one or more halogen atoms, which can be the same or different.

Examples of said “(Cₓ-C_{y}) haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y}) cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono- or bi-cyclic carbon ring systems wherein the ring is aromatic. Examples of suitable aryl ring systems include, for instance, phenyl or naphthyl. The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond. A group may be optionally substituted, wherein the term “optionally substituted” refers to being substituted or unsubstituted. When the term “one or more” refers to any atoms or groups as substituents of the groups of the compound of formula (I), it is intended that from 1 to 3, preferably 1 to 2, more preferably 1 of such substituents may replace hydrogens on such variables.

A bond pointing to a wavy or squiggly line, such as

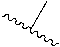

as used in structural formulas herein, depicts the bond that is the point of attachment of the moiety or substituent to the core or backbone structure.

A dash (“-”) that is not between two letters or symbols is meant to represent the point of attachment for a substituent.

The term “IC₅₀” refers to the half maximal inhibitory concentration as a measure of the potency of a substance in inhibiting a specific biological or biochemical function.

Whenever basic amino or quaternary ammonium groups are present in the compounds of formula I, physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate, emipamoate, xinafoate and naphthalene disulfonate. Likewise, in the presence of acidic groups such as COOH groups, corresponding physiological cation salts may be present as well, for instance including alkaline or alkaline earth metal ions.

As above indicated, the present invention refers to a series of compounds represented by the general formula (I) as herein below described in details, which are endowed with an antagonist property versus receptor LPA1.

Differently from similar compounds of the prior art, the compounds of formula (I) of the present invention are able to act as antagonist LPA1 in a substantive and effective way, particularly appreciated by the skilled person when looking at a suitable and efficacious compounds useful for the treatment of fibrosis, in particular idiopatic pulmonary fibrosis.

As it can be appreciated in the experimental part, in particular in Table 5, the compounds of formula (I) of the present invention show a notable potency with respect to their inhibitory activity on receptor LPA1 below about 600 nM, preferably below 250 nM and more preferably below 50 nM, confirming that they are able to antagonize the isoform of LPA1 receptor involved in fibrosis and diseases, disorders and conditions that involve fibrosis.

Advantageously, the compounds of the present invention characterized by a very high potency, could be administered in human at a lower dosage, thus reducing the adverse events that typically occur administering higher dosages of drug.

Therefore, the compounds of the present invention are particularly appreciated by the skilled person when looking at a suitable and efficacious compounds useful for the treatment of fibrosis, in particular idiopatic pulmonary fibrosis.

Thus, in one aspect the present invention relates to a compound of general formula (I) as LPA1 antagonist

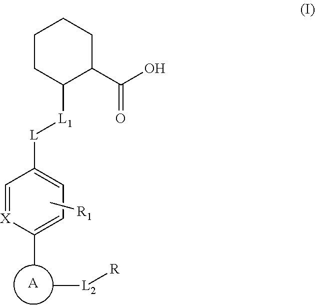

wherein X is —CH— or N;
R₁ is H or (C₁-C₄)alkyl;
L is —O— or —NH—;
L₁ is (—CH₂—)ₙ, or —C(O)—;
n is an integer between 0 and 2;
A is selected from the group consisting of

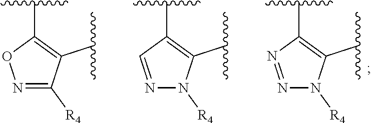

L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, —(C₁-C₄)alkyl-R₂, —NR₆R₇, —OC(O)NR₆R₇, —NHC(O)O—(C₁-C₄)alkyl-R₂, heteroaryl, wherein any of such heteroaryl may be optionally substituted by one or more groups selected from —OR₃, —(C₁-C₄)alkyl, —(C₁-C₄)haloalkyl, —(C₃-C₆)cycloalkyl, aryl, heteroaryl, —NR₆R₇;
R₂ is H or selected from the group consisting of —(C₁-C₄)alkyl, aryl, heteroaryl wherein any of such aryl, heteroaryl and alkyl are optionally substituted by one or more groups selected from —(C₁-C₄)alkyl, halo; —OR₅, —R₅;
R₃ is H or selected from the group consisting of —(C₁-C₄)alkyl, —(C₁-C₄)haloalkyl, —(C₃-C₆)cycloalkyl optionally substituted by one or more halo;
R₄ is H or (C₁-C₄)alkyl;
R₅ is aryl optionally substituted by one or more groups selected from —(C₁-C₄)alkyl and halo;
R₆ and R₇ are at each occurrence independently H or selected from the group consisting of —(C₁-C₆)alkyl, —(C₃-C₆)cycloalkyl, heteroaryl wherein any of such heteroaryl may be optionally substituted by one or more groups selected from —OR₄, aryl, —(C₃-C₆)cycloalkyl, heteroaryl
   with the proviso that when A is

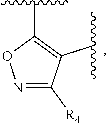

L is —NH—, L₁ is —C(O)—, X is —CH— and L₂ is NH, R is not —C(O)O(C₁-C₅)alkyl-R₂.
The invention further concerns the corresponding deuterated derivatives of compounds of formula (I).

In a preferred embodiment, the invention refers to at least one of the compounds listed in the Table 1 below and pharmaceutical acceptable salts thereof.

TABLE 1
List of preferred compounds of Formula (I)

| Compound | | |
|---|---|---|
| Nº | Structure | Chemical Name |
| Compound 1 | 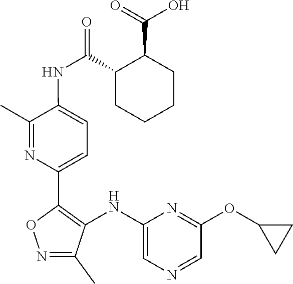 [SMILES: Cc1nc(-c2onc(C)c2Nc2cncc(OC3CC3)n2)ccc1NC(=O)[C@H]1CCCC[C@@H]1C(=O)O] | (1S,2S)-2-((6-(4-((6- cyclopropoxypyrazin-2- yl)amino)-3-methylisoxazol-5- yl)-2-methylpyridin-3- yl)carbamoyl)cyclohexane-1- carboxylic acid |
| Compound 2 | 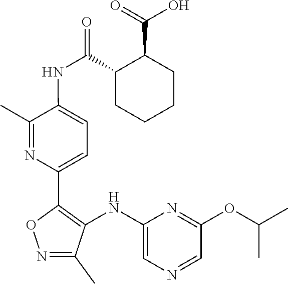 [SMILES: Cc1nc(-c2onc(C)c2Nc2cncc(OC(C)C)n2)ccc1NC(=O)[C@H]1CCCC[C@@H]1C(=O)O] | (1S,2S)-2-((6-(4-((6- isopropoxypyrazin-2-yl)amino)- 3-methylisoxazol-5-yl)-2- methylpyridin-3- yl)carbamoyl)cyclohexane-1- carboxylic acid |
| Compound 3 | 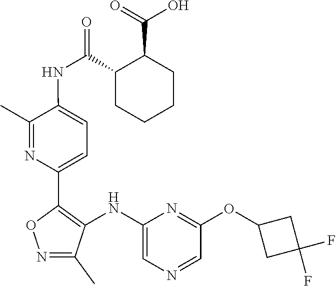 [SMILES: Cc1nc(-c2onc(C)c2Nc2cncc(OC3CC(F)(F)C3)n2)ccc1NC(=O)[C@H]1CCCC[C@@H]1C(=O)O] | (1S,2S)-2-((6-(4-((6-(3,3- difluorocyclobutoxy)pyrazin-2- yl)amino)-3-methylisoxazol-5- yl)-2-methylpyridin-3- yl)carbamoyl)cyclohexane-1- carboxylic acid |
| Compound 4 | 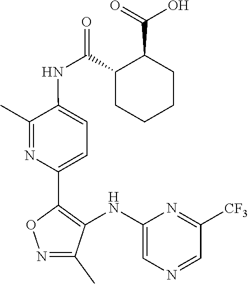 [SMILES: Cc1nc(-c2onc(C)c2Nc2cncc(C(F)(F)F)n2)ccc1NC(=O)[C@H]1CCCC[C@@H]1C(=O)O] | (1S,2S)-2-((2-methyl-6-(3- methyl-4-((6- (trifluoromethyl)pyrazin-2- yl)amino)isoxazol-5-yl)pyridin-3- yl)carbamoyl)cyclohexane-1- carboxylic acid |
| Compound 5 | 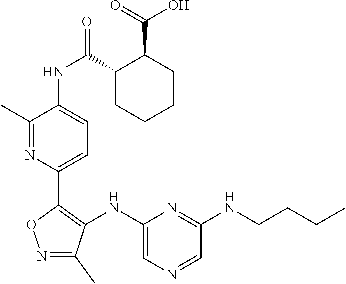 [SMILES: CCCCNc1cncc(Nc2c(C)noc2-c2ccc(NC(=O)[C@H]3CCCC[C@@H]3C(=O)O)c(C)n2)n1] | (1S,2S)-2-((6-(4-((6- (butylamino)pyrazin-2-yl)amino)- 3-methylisoxazol-5-yl)-2- methylpyridin-3- yl)carbamoyl)cyclohexane-1- carboxylic acid |
| Compound 6 | 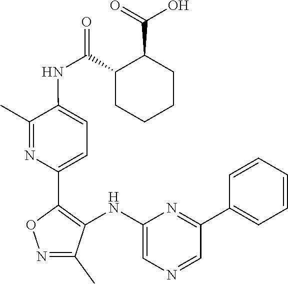 [SMILES: Cc1nc(-c2onc(C)c2Nc2cncc(-c3ccccc3)n2)ccc1NC(=O)[C@H]1CCCC[C@@H]1C(=O)O] | (1S,2S)-2-((2-methyl-6-(3- methyl-4-((6-phenylpyrazin-2- yl)amino)isoxazol-5-yl)pyridin-3- yl)carbamoyl)cyclohexane-1- carboxylic acid |
| Compound 7 | 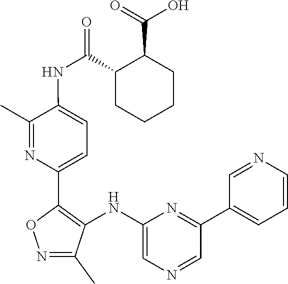 [SMILES: Cc1nc(-c2onc(C)c2Nc2cncc(-c3cccnc3)n2)ccc1NC(=O)[C@H]1CCCC[C@@H]1C(=O)O] | (1S,2S)-2-((2-methyl-6-(3- methyl-4-((6-(pyridin-3- yl)pyrazin-2-yl)amino)isoxazol-5- yl)pyridin-3- yl)carbamoyl)cyclohexane-1- carboxylic acid |
| Compound 8 | 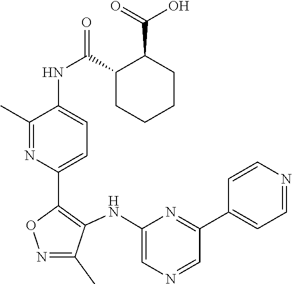 [SMILES: Cc1nc(-c2onc(C)c2Nc2cncc(-c3ccncc3)n2)ccc1NC(=O)[C@H]1CCCC[C@@H]1C(=O)O] | (1S,2S)-2-((2-methyl-6-(3- methyl-4-((6-(pyridin-4- yl)pyrazin-2-yl)amino)isoxazol-5- yl)pyridin-3- yl)carbamoyl)cyclohexane-1- carboxylic acid |
| Compound 9 | 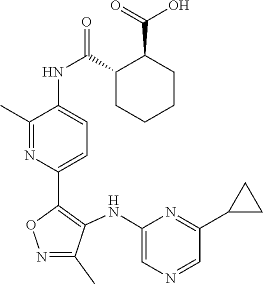 [SMILES: Cc1nc(-c2onc(C)c2Nc2cncc(C3CC3)n2)ccc1NC(=O)[C@H]1CCCC[C@@H]1C(=O)O] | (1S,2S)-2-((6-(4-((6- cyclopropylpyrazin-2-yl)amino)- 3-methylisoxazol-5-yl)-2- methylpyridin-3- yl)carbamoyl)cyclohexane-1- carboxylic acid |
| Compound 10 | 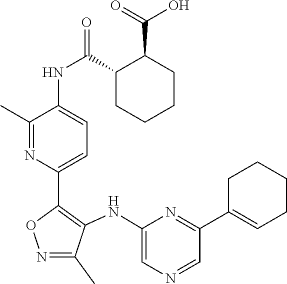 [SMILES: Cc1nc(-c2onc(C)c2Nc2cncc(C3=CCCCC3)n2)ccc1NC(=O)[C@H]1CCCC[C@@H]1C(=O)O] | (1S,2S)-2-((6-(4-((6-(cyclohex-1- en-1-yl)pyrazin-2-yl)amino)-3- methylisoxazol-5-yl)-2- methylpyridin-3- yl)carbamoyl)cyclohexane-1- carboxylic acid |
| Compound 11 | 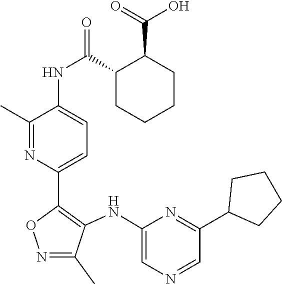 [SMILES: Cc1nc(-c2onc(C)c2Nc2cncc(C3CCCC3)n2)ccc1NC(=O)[C@H]1CCCC[C@@H]1C(=O)O] | (1S,2S)-2-((6-(4-((6- cyclopentylpyrazin-2-yl)amino)- 3-methylisoxazol-5-yl)-2- methylpyridin-3- yl)carbamoyl)cyclohexane-1- carboxylic acid |
| Compound 12 | 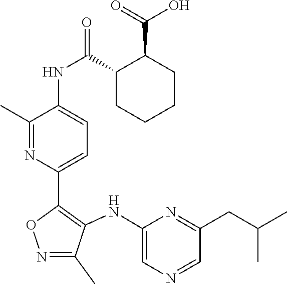 [SMILES: Cc1nc(-c2onc(C)c2Nc2cncc(CC(C)C)n2)ccc1NC(=O)[C@H]1CCCC[C@@H]1C(=O)O] | (1S,2S)-2-((6-(4-((6- isobutylpyrazin-2-yl)amino)-3- methylisoxazol-5-yl)-2- methylpyridin-3- yl)carbamoyl)cyclohexane-1- carboxylic acid |
| Compound 13 | 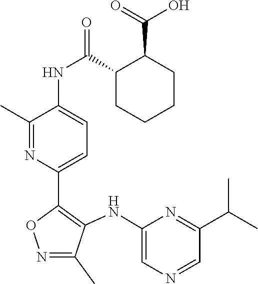 [SMILES: Cc1nc(-c2onc(C)c2Nc2cncc(C(C)C)n2)ccc1NC(=O)[C@H]1CCCC[C@@H]1C(=O)O] | (1S,2S)-2-((6-(4-((6- isopropylpyrazin-2-yl)amino)-3- methylisoxazol-5-yl)-2- methylpyridin-3- yl)carbamoyl)cyclohexane-1- carboxylic acid |
| Compound 14 | 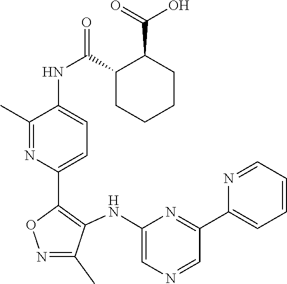 [SMILES: Cc1nc(-c2onc(C)c2Nc2cncc(-c3ccccn3)n2)ccc1NC(=O)[C@H]1CCCC[C@@H]1C(=O)O] | (1S,2S)-2-((2-methyl-6-(3- methyl-4-((6-(pyridin-2- yl)pyrazin-2-yl)amino)isoxazol-5- yl)pyridin-3- yl)carbamoyl)cyclohexane-1- carboxylic acid |
| Compound 15 | 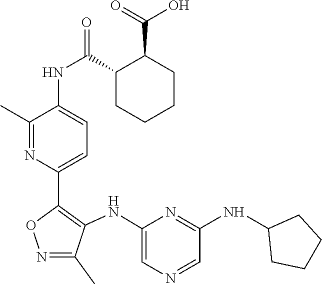 [SMILES: Cc1nc(-c2onc(C)c2Nc2cncc(NC3CCCC3)n2)ccc1NC(=O)[C@H]1CCCC[C@@H]1C(=O)O] | (1S,2S)-2-((6-(4-((6- (cyclopentylamino)pyrazin-2- yl)amino)-3-methylisoxazol-5- yl)-2-methylpyridin-3- yl)carbamoyl)cyclohexane-1- carboxylic acid |
| Compound 16 | 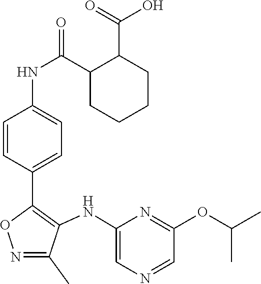 [SMILES: Cc1noc(-c2ccc(NC(=O)C3CCCCC3C(=O)O)cc2)c1Nc1cncc(OC(C)C)n1] | 2-((4-(4-((6-isopropoxypyrazin-2- yl)amino)-3-methylisoxazol-5- yl)phenyl)carbamoyl)cyclohexane- 1-carboxylic acid (trans racemate) |
| Compound 17 | 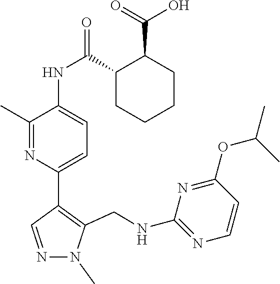 [SMILES: Cc1nc(-c2cnn(C)c2CNc2nccc(OC(C)C)n2)ccc1NC(=O)[C@H]1CCCC[C@@H]1C(=O)O] | (1S,2S)-2-((6-(5-(((4- isopropoxypyrimidin-2- yl)amino)methyl)-1-methyl-1H- pyrazol-4-yl)-2-methylpyridin-3- yl)carbamoyl)cyclohexane-1- carboxylic acid |
| Compound 18 | 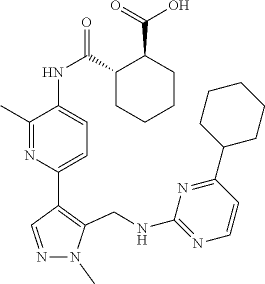 [SMILES: Cc1nc(-c2cnn(C)c2CNc2nccc(C3CCCCC3)n2)ccc1NC(=O)[C@H]1CCCC[C@@H]1C(=O)O] | ((1S,2S)-2-((6-(5-(((4- cyclohexylpyrimidin-2- yl)amino)methyl)-1-methyl-1H- pyrazol-4-yl)-2-methylpyridin-3- yl)carbamoyl)cyclohexane-1- carboxylic acid |
| Compound 19 | 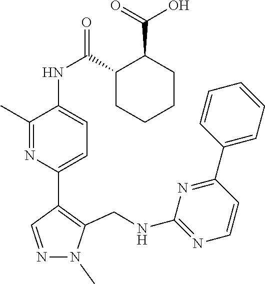 [SMILES: Cc1nc(-c2cnn(C)c2CNc2nccc(-c3ccccc3)n2)ccc1NC(=O)[C@H]1CCCC[C@@H]1C(=O)O] | (1S,2S)-2-((2-methyl-6-(1- methyl-5-(((4-phenylpyrimidin-2- yl)amino)methyl)-1H-pyrazol-4- yl)pyridin-3- yl)carbamoyl)cyclohexane-1- carboxylic acid |
| Compound 20 | 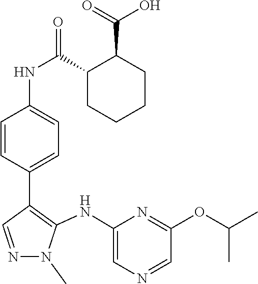 [SMILES: CC(C)Oc1cncc(Nc2c(-c3ccc(NC(=O)[C@H]4CCCC[C@@H]4C(=O)O)cc3)cnn2C)n1] | (1S,2S)-2-((4-(5-((6- isopropoxypyrazin-2-yl)amino)- 1-methyl-1H-pyrazol-4- yl)phenyl)carbamoyl)cyclohexane- 1-carboxylic acid |
| Compound 21 | 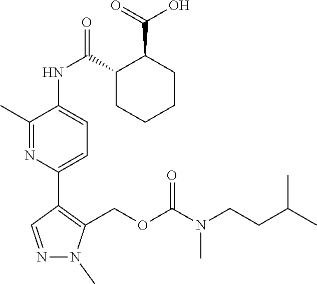 [SMILES: Cc1nc(-c2cnn(C)c2COC(=O)N(C)CCC(C)C)ccc1NC(=O)[C@H]1CCCC[C@@H]1C(=O)O] | (1S,2S)-2-((6-(5- (((isopentyl(methyl)carbamoyl) oxy)methyl)-1-methyl-1H-pyrazol- 4-yl)-2-methylpyridin-3- yl)carbamoyl)cyclohexane-1- carboxylic acid |
| Compound 22 | 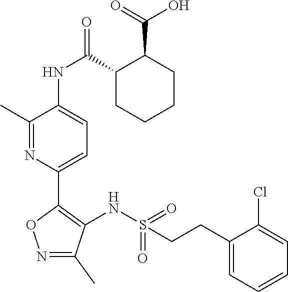 [SMILES: Cc1nc(-c2onc(C)c2NS(=O)(=O)CCc2ccccc2Cl)ccc1NC(=O)[C@H]1CCCC[C@@H]1C(=O)O] | (1S,2S)-2-((6-(4-((2-(2- chlorophenyl)ethyl)sulfonamido)- 3-methylisoxazol-5-yl)-2- methylpyridin-3- yl)carbamoyl)cyclohexane-1- carboxylic acid |
| Compound 23 | 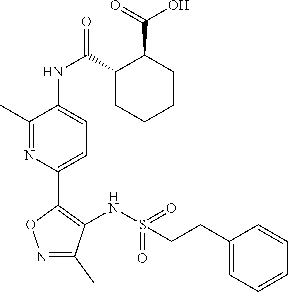 [SMILES: Cc1nc(-c2onc(C)c2NS(=O)(=O)CCc2ccccc2)ccc1NC(=O)[C@H]1CCCC[C@@H]1C(=O)O] | (1S,2S)-2-((2-methyl-6-(3- methyl-4-((2- phenylethyl)sulfonamido)isoxazol- 5-yl)pyridin-3- yl)carbamoyl)cyclohexane-1- carboxylic acid |
| Compound 24 | 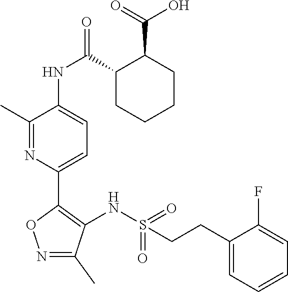 [SMILES: Cc1nc(-c2onc(C)c2NS(=O)(=O)CCc2ccccc2F)ccc1NC(=O)[C@H]1CCCC[C@@H]1C(=O)O] | (1S,2S)-2-((6-(4-((2-(2- fluorophenyl)ethyl)sulfonamido)- 3-methylisoxazol-5-yl)-2- methylpyridin-3- yl)carbamoyl)cyclohexane-1- carboxylic acid |
| Compound 25 | 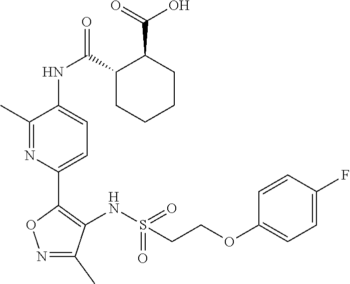 [SMILES: Cc1nc(-c2onc(C)c2NS(=O)(=O)CCOc2ccc(F)cc2)ccc1NC(=O)[C@H]1CCCC[C@@H]1C(=O)O] | (1S,2S)-2-((6-(4-((2-(4- fluorophenoxy)ethyl)sulfonamido)- 3-methylisoxazol-5-yl)-2- methylpyridin-3- yl)carbamoyl)cyclohexane-1- carboxylic acid |
| Compound 26 | 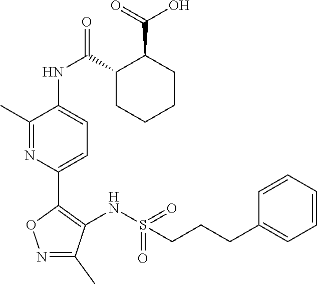 [SMILES: Cc1nc(-c2onc(C)c2NS(=O)(=O)CCCc2ccccc2)ccc1NC(=O)[C@H]1CCCC[C@@H]1C(=O)O] | (1S,2S)-2-((2-methyl-6-(3- methyl-4-((3- phenylpropyl)sulfonamido) isoxazol-5-yl)pyridin-3- yl)carbamoyl)cyclohexane-1- carboxylic acid |
| Compound 27 | 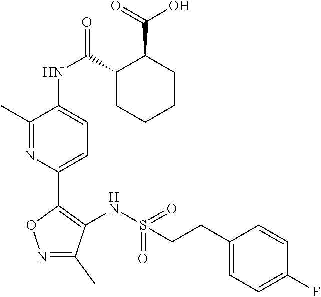 [SMILES: Cc1nc(-c2onc(C)c2NS(=O)(=O)CCc2ccc(F)cc2)ccc1NC(=O)[C@H]1CCCC[C@@H]1C(=O)O] | (1S,2S)-2-((6-(4-((2-(4- fluorophenyl)ethyl)sulfonamido)- 3-methylisoxazol-5-yl)-2- methylpyridin-3- yl)carbamoyl)cyclohexane-1- carboxylic acid |
| Compound 28 | 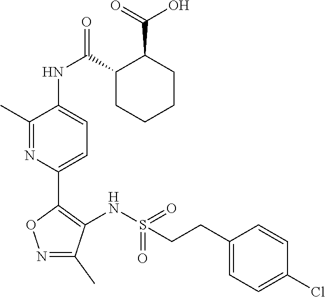 [SMILES: Cc1nc(-c2onc(C)c2NS(=O)(=O)CCc2ccc(Cl)cc2)ccc1NC(=O)[C@H]1CCCC[C@@H]1C(=O)O] | (1S,2S)-2-((6-(4-((2-(4- chlorophenyl)ethyl)sulfonamido)- 3-methylisoxazol-5-yl)-2- methylpyridin-3- yl)carbamoyl)cyclohexane-1- carboxylic acid |
| Compound 29 | 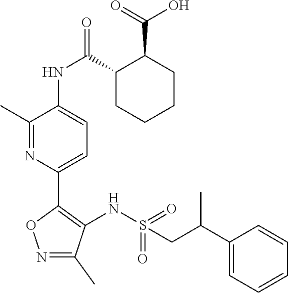 [SMILES: Cc1nc(-c2onc(C)c2NS(=O)(=O)CC(C)c2ccccc2)ccc1NC(=O)[C@H]1CCCC[C@@H]1C(=O)O] | (1S,2S)-2-((2-methyl-6-(3- methyl-4-((2- phenylpropyl)sulfonamido) isoxazol-5-yl)pyridin-3- yl)carbamoyl)cyclohexane-1- carboxylic acid |
| Compound 30 | 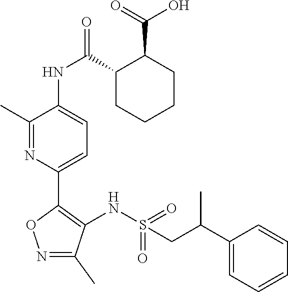 [SMILES: Cc1nc(-c2onc(C)c2NS(=O)(=O)CC(C)c2ccccc2)ccc1NC(=O)[C@H]1CCCC[C@@H]1C(=O)O] | Single Diastereomer 1 of (1S,2S)- 2-((2-methyl-6-(3-methyl-4-((2- phenylpropyl)sulfonamido) isoxazol-5-yl)pyridin-3- yl)carbamoyl)cyclohexane-1- carboxylic acid |
| Compound 31 | 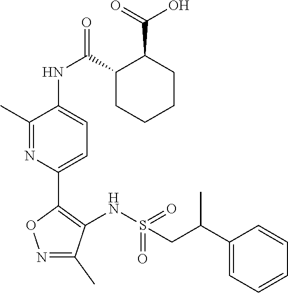 [SMILES: Cc1nc(-c2onc(C)c2NS(=O)(=O)CC(C)c2ccccc2)ccc1NC(=O)[C@H]1CCCC[C@@H]1C(=O)O] | Single Diastereomer 2 of (1S,2S)- 2-((2-methyl-6-(3-methyl-4-((2- phenylpropyl)sulfonamido) isoxazol-5-yl)pyridin-3- yl)carbamoyl)cyclohexane-1- carboxylic acid |
| Compound 32 | 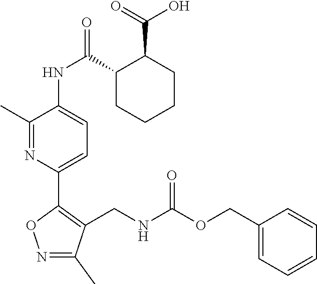 [SMILES: Cc1nc(-c2onc(C)c2CNC(=O)OCc2ccccc2)ccc1NC(=O)[C@H]1CCCC[C@@H]1C(=O)O] | (1S,2S)-2-((6-(4- ((((benzyloxy)carbonyl)amino) methyl)-3-methylisoxazol-5-yl)-2- methylpyridin-3- yl)carbamoyl)cyclohexane-1- carboxylic acid |
| Compound 33 | 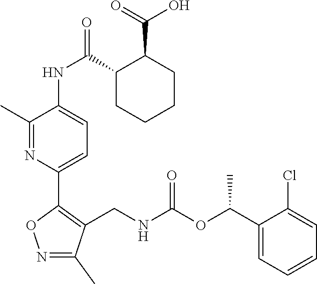 [SMILES: Cc1nc(-c2onc(C)c2CNC(=O)O[C@H](C)c2ccccc2Cl)ccc1NC(=O)[C@H]1CCCC[C@@H]1C(=O)O] | (1S,2S)-2-((6-(4-(((((R)-1-(2- chlorophenyl)ethoxy)carbonyl) amino)methyl)-3-methylisoxazol-5- yl)-2-methylpyridin-3- yl)carbamoyl)cyclohexane-1- carboxylic acid |
| Compound 34 | 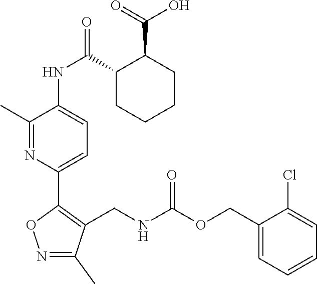 [SMILES: Cc1nc(-c2onc(C)c2CNC(=O)OCc2ccccc2Cl)ccc1NC(=O)[C@H]1CCCC[C@@H]1C(=O)O] | (1S,2S)-2-((6-(4-(((((2- chlorobenzyl)oxy)carbonyl)amino) methyl)-3-methylisoxazol-5-yl)- 2-methylpyridin-3- yl)carbamoyl)cyclohexane-1- carboxylic acid |
| Compound 35 | 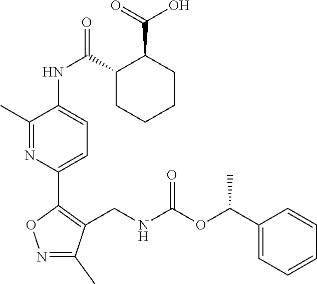 [SMILES: Cc1nc(-c2onc(C)c2CNC(=O)O[C@H](C)c2ccccc2)ccc1NC(=O)[C@H]1CCCC[C@@H]1C(=O)O] | (1S,2S)-2-((2-methyl-6-(3- methyl-4-(((((R)-1- phenylethoxy)carbonyl)amino) methyl)isoxazol-5-yl)pyridin-3- yl)carbamoyl)cyclohexane-1- carboxylic acid |
| Compound 36 | 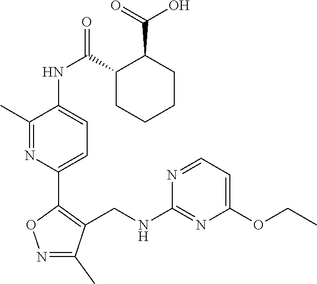 [SMILES: CCOc1ccnc(NCc2c(C)noc2-c2ccc(NC(=O)[C@H]3CCCC[C@@H]3C(=O)O)c(C)n2)n1] | (1S,2S)-2-((6-(4-(((4- ethoxypyrimidin-2- yl)amino)methyl)-3- methylisoxazol-5-yl)-2- methylpyridin-3- yl)carbamoyl)cyclohexane-1- carboxylic acid |
| Compound 37 | 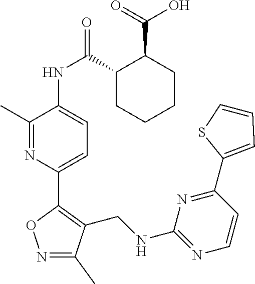 [SMILES: Cc1nc(-c2onc(C)c2CNc2nccc(-c3cccs3)n2)ccc1NC(=O)[C@H]1CCCC[C@@H]1C(=O)O] | (1S,2S)-2-((2-methyl-6-(3- methyl-4-(((4-(thiophen-2- yl)pyrimidin-2- yl)amino)methyl)isoxazol-5- yl)pyridin-3- yl)carbamoyl)cyclohexane-1- carboxylic acid |
| Compound 38 | 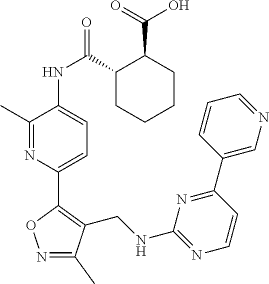 [SMILES: Cc1nc(-c2onc(C)c2CNc2nccc(-c3cccnc3)n2)ccc1NC(=O)[C@H]1CCCC[C@@H]1C(=O)O] | (1S,2S)-2-((2-methyl-6-(3- methyl-4-(((4-(pyridin-3- yl)pyrimidin-2- yl)amino)methyl)isoxazol-5- yl)pyridin-3- yl)carbamoyl)cyclohexane-1- carboxylic acid |
| Compound 39 | 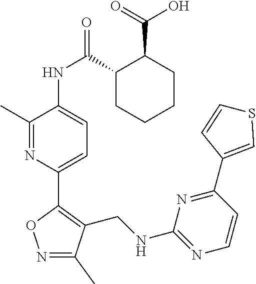 [SMILES: Cc1nc(-c2onc(C)c2CNc2nccc(-c3ccsc3)n2)ccc1NC(=O)[C@H]1CCCC[C@@H]1C(=O)O] | (1S,2S)-2-((2-methyl-6-(3- methyl-4-(((4-(thiophen-3- yl)pyrimidin-2- yl)amino)methyl)isoxazol-5- yl)pyridin-3- yl)carbamoyl)cyclohexane-1- carboxylic acid |
| Compound 40 | 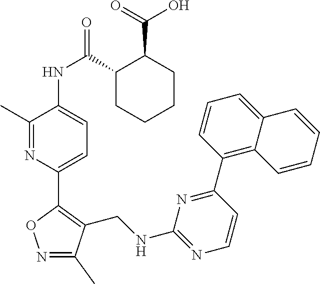 [SMILES: Cc1nc(-c2onc(C)c2CNc2nccc(-c3cccc4ccccc34)n2)ccc1NC(=O)[C@H]1CCCC[C@@H]1C(=O)O] | (1S,2S)-2-((2-methyl-6-(3- methyl-4-(((4-(naphthalen-1- yl)pyrimidin-2- yl)amino)methyl)isoxazol-5- yl)pyridin-3- yl)carbamoyl)cyclohexane-1- carboxylic acid |
| Compound 41 | 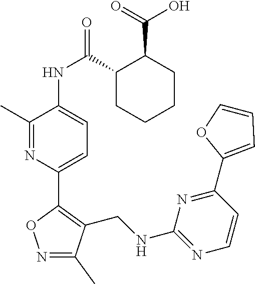 [SMILES: Cc1nc(-c2onc(C)c2CNc2nccc(-c3ccco3)n2)ccc1NC(=O)[C@H]1CCCC[C@@H]1C(=O)O] | (1S,2S)-2-((6-(4-(((4-(furan-2- yl)pyrimidin-2-yl)amino)methyl)- 3-methylisoxazol-5-yl)-2- methylpyridin-3- yl)carbamoyl)cyclohexane-1- carboxylic acid |
| Compound 42 | 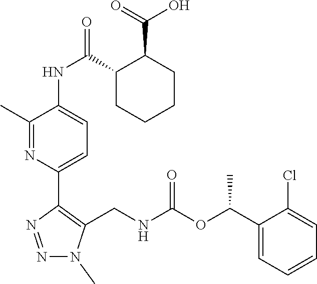 [SMILES: Cc1nc(-c2nnn(C)c2CNC(=O)O[C@H](C)c2ccccc2Cl)ccc1NC(=O)[C@H]1CCCC[C@@H]1C(=O)O] | (1S,2S)-2-((6-(5-(((((R)-1-(2- chlorophenyl)ethoxy)carbonyl) amino)methyl)-1-methyl-1H-1,2,3- triazol-4-yl)-2-methylpyridin-3- yl)carbamoyl)cyclohexane-1- carboxylic acid |
| Compound 43 | 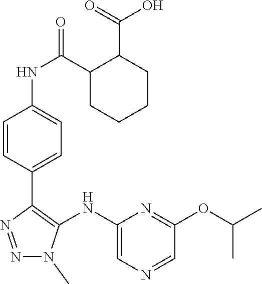 [SMILES: CC(C)Oc1cncc(Nc2c(-c3ccc(NC(=O)C4CCCCC4C(=O)O)cc3)nnn2C)n1] | 2-((4-(5-((6-isopropoxypyrazin-2- yl)amino)-1-methyl-1H-1,2,3- triazol-4- yl)phenyl)carbamoyl)cyclohexane- 1-carboxylic acid (cis racemate) |
| Compound 44 | 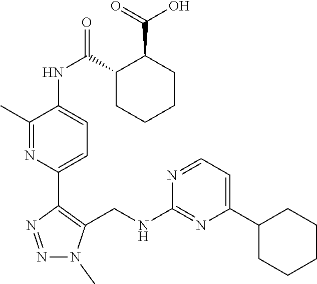 [SMILES: Cc1nc(-c2nnn(C)c2CNc2nccc(C3CCCCC3)n2)ccc1NC(=O)[C@H]1CCCC[C@@H]1C(=O)O] | (1S,2S)-2-((6-(5-(((4- cyclohexylpyrimidin-2- yl)amino)methyl)-1-methyl-1H- 1,2,3-triazol-4-yl)-2- methylpyridin-3- yl)carbamoyl)cyclohexane-1- carboxylic acid |
| Compound 45 | 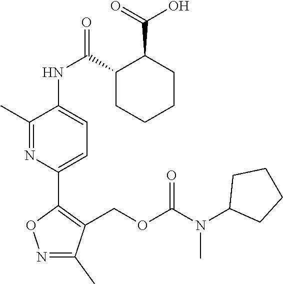 [SMILES: Cc1nc(-c2onc(C)c2COC(=O)N(C)C2CCCC2)ccc1NC(=O)[C@H]1CCCC[C@@H]1C(=O)O] | (1S,2S)-2-((6-(4- (((cyclopentyl(methyl)carbamoyl) oxy)methyl)-3-methylisoxazol-5- yl)-2-methylpyridin-3- yl)carbamoyl)cyclohexane-1- carboxylic acid |
| Compound 46 | 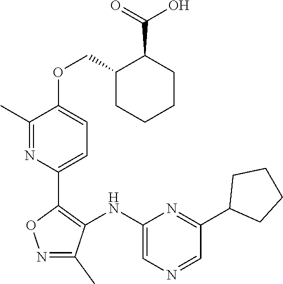 [SMILES: Cc1nc(-c2onc(C)c2Nc2cncc(C3CCCC3)n2)ccc1OC[C@H]1CCCC[C@@H]1C(=O)O] | (1S,2S)-2-(((6-(4-((6- cyclopentylpyrazin-2-yl)amino)- 3-methylisoxazol-5-yl)-2- methylpyridin-3- yl)oxy)methyl)cyclohexane-1- carboxylic acid |
| Compound 47 | 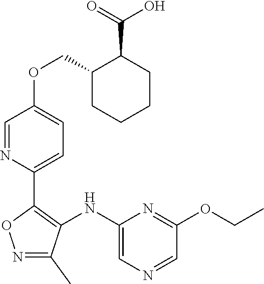 [SMILES: CCOc1cncc(Nc2c(C)noc2-c2ccc(OC[C@H]3CCCC[C@@H]3C(=O)O)cn2)n1] | (1S,2S)-2-(((6-(4-((6- ethoxypyrazin-2-yl)amino)-3- methylisoxazol-5-yl)pyridin-3- yl)oxy)methyl)cyclohexane-1- carboxylic acid |
| Compound 48 | 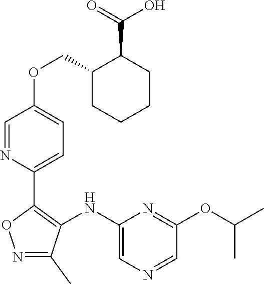 [SMILES: Cc1noc(-c2ccc(OC[C@H]3CCCC[C@@H]3C(=O)O)cn2)c1Nc1cncc(OC(C)C)n1] | (1S,2S)-2-(((6-(4-((6- isopropoxypyrazin-2-yl)amino)- 3-methylisoxazol-5-yl)pyridin-3- yl)oxy)methyl)cyclohexane-1- carboxylic acid |
| Compound 49 | 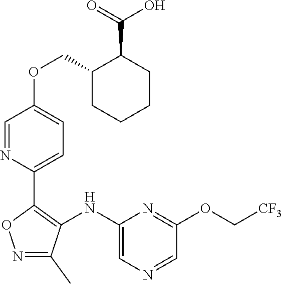 [SMILES: Cc1noc(-c2ccc(OC[C@H]3CCCC[C@@H]3C(=O)O)cn2)c1Nc1cncc(OCC(F)(F)F)n1] | (1S,2S)-2-(((6-(3-methyl-4-((6- (2,2,2-trifluoroethoxy)pyrazin-2- yl)amino)isoxazol-5-yl)pyridin-3- yl)oxy)methyl)cyclohexane-1- carboxylic acid |
| Compound 50 | 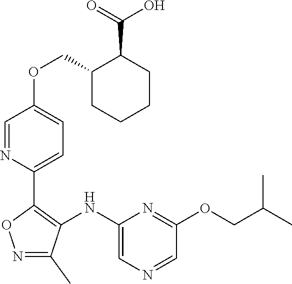 [SMILES: Cc1noc(-c2ccc(OC[C@H]3CCCC[C@@H]3C(=O)O)cn2)c1Nc1cncc(OCC(C)C)n1] | (1S,2S)-2 -(((6-(4-((6- isobutoxypyrazin-2-yl)amino)-3- methylisoxazol-5-yl)pyridin-3- yl)oxy)methyl)cyclohexane-1- carboxylic acid |
| Compound 51 | 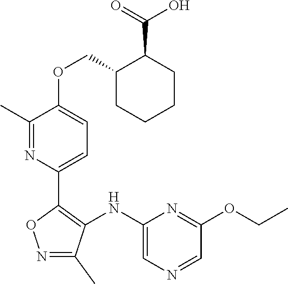 [SMILES: CCOc1cncc(Nc2c(C)noc2-c2ccc(OC[C@H]3CCCC[C@@H]3C(=O)O)c(C)n2)n1] | (1S,2S)- 2-(((6-(4-((6- ethoxypyrazin-2-yl)amino)-3- methylisoxazol-5-yl)-2- methylpyridin-3- yl)oxy)methyl)cyclohexane-1- carboxylic acid |
| Compound 52 | 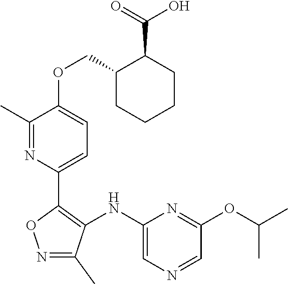 [SMILES: Cc1nc(-c2onc(C)c2Nc2cncc(OC(C)C)n2)ccc1OC[C@H]1CCCC[C@@H]1C(=O)O] | (1S,2S)-2-(((6-(4-((6- isopropoxypyrazin-2-yl)amino)- 3-methylisoxazol-5-yl)-2- methylpyridin-3- yl)oxy)methyl)cyclohexane-1- carboxylic acid |
| Compound 53 | 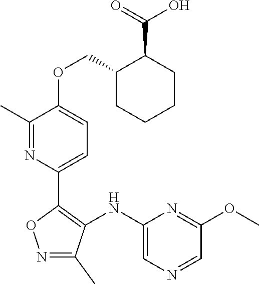 | (1S,2S)-2-(((6- (4-((6- methoxypyrazin-2-yl)amino)-3- methylisoxazol-5-yl)-2- methylpyridin-3- yl)oxy)methyl)cyclohexane-1- carboxylic acid |
| Compound 54 | 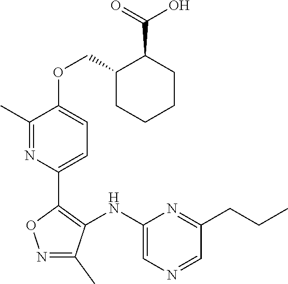 [SMILES: CCCc1cncc(Nc2c(C)noc2-c2ccc(OC[C@H]3CCCC[C@@H]3C(=O)O)c(C)n2)n1] | (1S,2S)-2-(((2-methyl-6-(3- methyl-4-((6-propylpyrazin-2- yl)amino)isoxazol-5-yl)pyridin-3- yl)oxy)methyl)cyclohexane-1- carboxylic acid |
| Compound 55 | 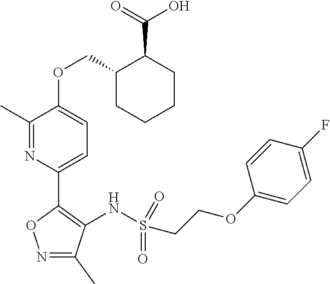 [SMILES: Cc1nc(-c2onc(C)c2NS(=O)(=O)CCOc2ccc(F)cc2)ccc1OC[C@H]1CCCC[C@@H]1C(=O)O] | (1S,2S)-2-(((6-(4-((2-(4- fluorophenoxy)ethyl)sulfonamido)- 3-methylisoxazol-5-yl)-2- methylpyridin-3- yl)oxy)methyl)cyclohexane-1- carboxylic acid |
| Compound 56 | 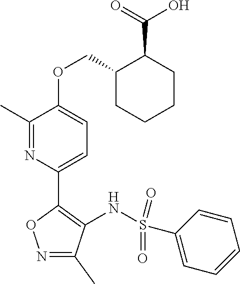 [SMILES: Cc1nc(-c2onc(C)c2NS(=O)(=O)c2ccccc2)ccc1OC[C@H]1CCCC[C@@H]1C(=O)O] | (1S,2S)-2-(((2-methyl-6-(3- methyl-4- (phenylsulfonamido)isoxazol-5- yl)pyridin-3- yl)oxy)methyl)cyclohexane-1- carboxylic acid |
| Compound 57 | 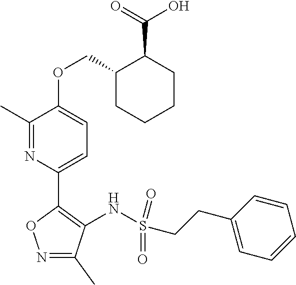 [SMILES: Cc1nc(-c2onc(C)c2NS(=O)(=O)CCc2ccccc2)ccc1OC[C@H]1CCCC[C@@H]1C(=O)O] | (1S,2S)-2-(((2-methyl-6-(3- methyl-4-((2- phenylethyl)sulfonamido)isoxazol- 5-yl)pyridin-3- yl)oxy)methyl)cyclohexane-1- carboxylic acid |
| Compound 58 | 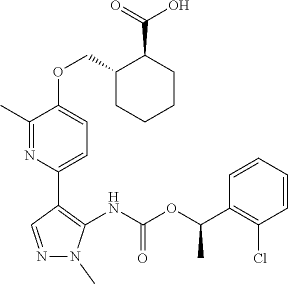 [SMILES: Cc1nc(-c2cnn(C)c2NC(=O)O[C@H](C)c2ccccc2Cl)ccc1OC[C@H]1CCCC[C@@H]1C(=O)O] | (1S,2S)-2-(((6-(5-((((R)-1-(2- chlorophenyl)ethoxy)carbonyl) amino)-1-methyl-1H-pyrazol-4-yl)- 2-methylpyridin-3- yl)oxy)methyl)cyclohexane-1- carboxylic acid |
| Compound 59 | 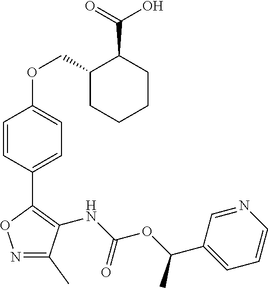 [SMILES: Cc1noc(-c2ccc(OC[C@H]3CCCC[C@@H]3C(=O)O)cc2)c1NC(=O)O[C@H](C)c1cccnc1] | (1S,2S)-2-((4-(3-methyl-4-((((R)- 1-(pyridin-3- yl)ethoxy)carbonyl)amino) isoxazol-5- yl)phenoxy)methyl)cyclohexane- 1-carboxylic acid |
| Compound 60 | 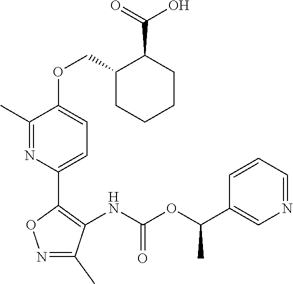 [SMILES: Cc1nc(-c2onc(C)c2NC(=O)O[C@H](C)c2cccnc2)ccc1OC[C@H]1CCCC[C@@H]1C(=O)O] | methyl (1S,2S)-2-(((2-methyl-6- (3-methyl-4-((((R)-1-(pyridin-3- yl)ethoxy)carbonyl)amino) isoxazol-5-yl)pyridin-3- yl)oxy)methyl)cyclohexane-1- carboxylic acid |
| Compound 61 | 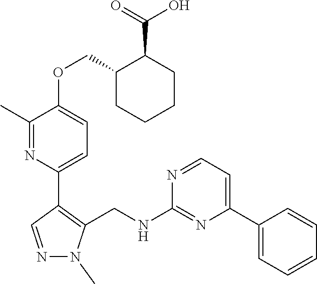 [SMILES: Cc1nc(-c2cnn(C)c2CNc2nccc(-c3ccccc3)n2)ccc1OC[C@H]1CCCC[C@@H]1C(=O)O] | (1S,2S)-2-(((2-methyl-6-(1- methyl-5-(((4-phenylpyrimidin-2- yl)amino)methyl)-1H-pyrazol-4- yl)pyridin-3- yl)oxy)methyl)cyclohexane-1- carboxylic acid |
| Compound 62 | 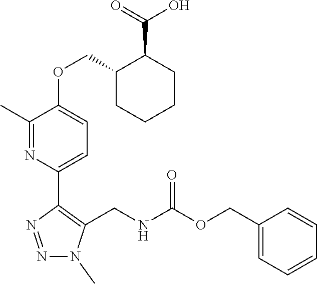 [SMILES: Cc1nc(-c2nnn(C)c2CNC(=O)OCc2ccccc2)ccc1OC[C@H]1CCCC[C@@H]1C(=O)O] | (1S,2S)-2-(((6-(5- ((((benzyloxy)carbonyl)amino) methyl)-1-methyl-1H-1,2,3-triazol- 4-yl)-2-methylpyridin-3- yl)oxy)methyl)cyclohexane-1- carboxylic acid |
| Compound 63 | 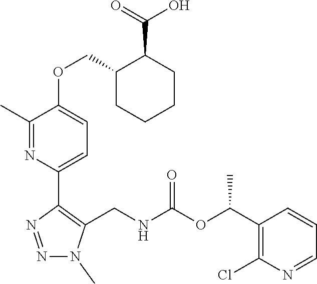 [SMILES: Cc1nc(-c2nnn(C)c2CNC(=O)O[C@H](C)c2cccnc2Cl)ccc1OC[C@H]1CCCC[C@@H]1C(=O)O] | (1S,2S)-2-(((6-(5-(((((R)-1-(2- chloropyridin-3- yl)ethoxy)carbonyl)amino)methyl)- 1-methyl-1H-1,2,3-triazol-4-yl)- 2-methylpyridin-3- yl)oxy)methyl)cyclohexane-1- carboxylic acid |
| Compound 64 | 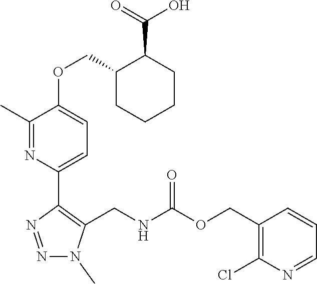 [SMILES: Cc1nc(-c2nnn(C)c2CNC(=O)OCc2cccnc2Cl)ccc1OC[C@H]1CCCC[C@@H]1C(=O)O] | (1S,2S)-2-(((6-(5-(((((2- chloropyridin-3- yl)methoxy)carbonyl)amino)methyl)- 1-methyl-1H-1,2,3-triazol-4- yl)-2-methylpyridin-3- yl)oxy)methyl)cyclohexane-1- carboxylic acid |
| Compound 65 | 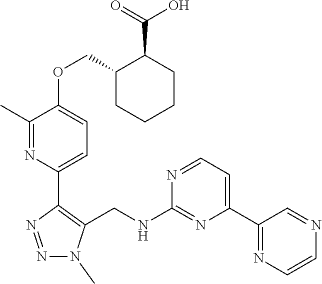 [SMILES: Cc1nc(-c2nnn(C)c2CNc2nccc(-c3cnccn3)n2)ccc1OC[C@H]1CCCC[C@@H]1C(=O)O] | (1S,2S)-2-(((2-methyl-6-(1- methyl-5-(((4-(pyrazin-2- yl)pyrimidin-2-yl)amino)methyl)- 1H-1,2,3-triazol-4-yl)pyridin-3- yl)oxy)methyl)cyclohexane-1- carboxylic acid |
| Compound 66 | 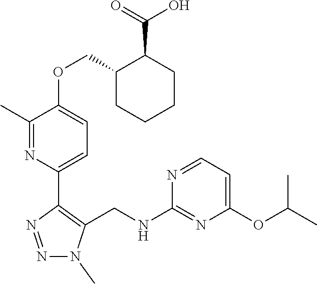 [SMILES: Cc1nc(-c2nnn(C)c2CNc2nccc(OC(C)C)n2)ccc1OC[C@H]1CCCC[C@@H]1C(=O)O] | (1S,2S)-2-(((6-(5-(((4- isopropoxypyrimidin-2- yl)amino)methyl)-1-methyl-1H- 1,2,3-triazol-4-yl)-2- methylpyridin-3- yl)oxy)methyl)cyclohexane-1- carboxylic acid |
| Compound 67 | 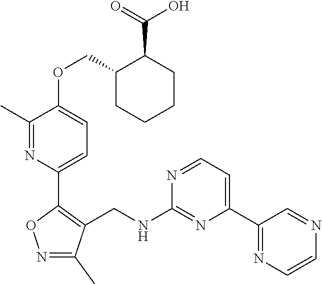 [SMILES: Cc1nc(-c2onc(C)c2CNc2nccc(-c3cnccn3)n2)ccc1OC[C@H]1CCCC[C@@H]1C(=O)O] | (1S,2S)-2-(((2-methyl-6-(3- methyl-4-(((4-(pyrazin-2- yl)pyrimidin-2- yl)amino)methyl)isoxazol-5- yl)pyridin-3- yl)oxy)methyl)cyclohexane-1- carboxylic acid |
| Compound 68 | 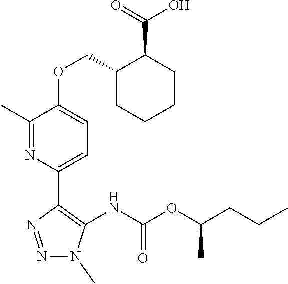 [SMILES: CCC[C@@H](C)OC(=O)Nc1c(-c2ccc(OC[C@H]3CCCC[C@@H]3C(=O)O)c(C)n2)nnn1C] | (1S,2S)-2-(((2-methyl-6-(1- methyl-5-(((((R)-pentan-2- yl)oxy)carbonyl)amino)-1H- 1,2,3-triazol-4-yl)pyridin-3- yl)oxy)methyl)cyclohexane-1- carboxylic acid |
| Compound 69 | 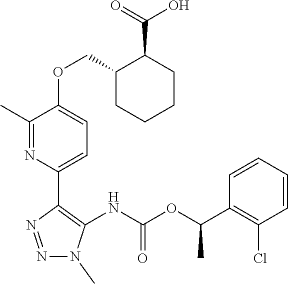 [SMILES: Cc1nc(-c2nnn(C)c2NC(=O)O[C@H](C)c2ccccc2Cl)ccc1OC[C@H]1CCCC[C@@H]1C(=O)O] | (1S,2S)-2-(((6-(5-((((R)-1-(2- chlorophenyl)ethoxy)carbonyl) amino)-1-methyl-1H-1,2,3-triazol-4- yl)-2-methylpyridin-3- yl)oxy)methyl)cyclohexane-1- carboxylic acid |
| Compound 70 | 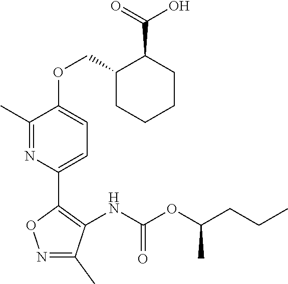 [SMILES: CCC[C@@H](C)OC(=O)Nc1c(C)noc1-c1ccc(OC[C@H]2CCCC[C@@H]2C(=O)O)c(C)n1] | (1S,2S)-2-(((2-methyl-6-(1- methyl-5-(((((R)-pentan-2- yl)oxy)carbonyl)amino)-1H- 1,2,3-triazol-4-yl)pyridin-3- yl)oxy)methyl)cyclohexane-1- carboxylic acid |
| Compound 71 | 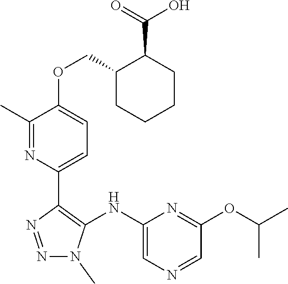 [SMILES: Cc1nc(-c2nnn(C)c2Nc2cncc(OC(C)C)n2)ccc1OC[C@H]1CCCC[C@@H]1C(=O)O] | (1S,2S)-2-(((6-(5-((6- isopropoxypyrazin-2-yl)amino)- 1-methyl-1H-1,2,3-triazol-4-yl)- 2-methylpyridin-3- yl)oxy)methyl)cyclohexane-1- carboxylic acid |
| Compound 72 | 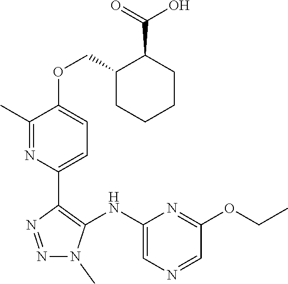 [SMILES: CCOc1cncc(Nc2c(-c3ccc(OC[C@H]4CCCC[C@@H]4C(=O)O)c(C)n3)nnn2C)n1] | (1S,2S)-2-(((6-(5-((6- ethoxypyrazin-2-yl)amino)-1- methyl-1H-1,2,3-triazol-4-yl)-2- methylpyridin-3- yl)oxy)methyl)cyclohexane-1- carboxylic acid |
| Compound 73 | 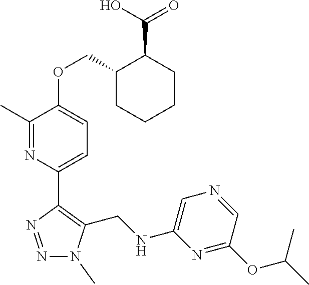 [SMILES: Cc1nc(-c2nnn(C)c2CNc2cncc(OC(C)C)n2)ccc1OC[C@H]1CCCC[C@@H]1C(=O)O] | (1S,2S)-2-(((6-(5-(((6- isopropoxypyrazin-2- yl)amino)methyl)-1-methyl-1H- 1,2,3-triazol-4-yl)-2- methylpyridin-3- yl)oxy)methyl)cyclohexane-1- carboxylic acid |
| Compound 74 | 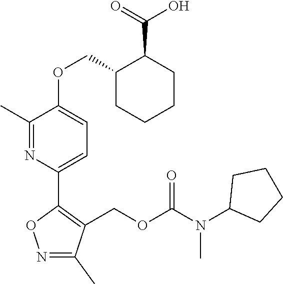 [SMILES: Cc1nc(-c2onc(C)c2COC(=O)N(C)C2CCCC2)ccc1OC[C@H]1CCCC[C@@H]1C(=O)O] | (1S,2S)-2-(((6-(4- (((cyclopentyl(methyl)carbamoyl) oxy)methyl)-3-methylisoxazol-5- yl)-2-methylpyridin-3- yl)oxy)methyl)cyclohexane-1- carboxylic acid |
| Compound 75 | 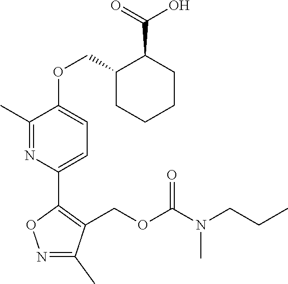 [SMILES: CCCN(C)C(=O)OCc1c(C)noc1-c1ccc(OC[C@H]2CCCC[C@@H]2C(=O)O)c(C)n1] | (1S,2S)-2-(((2-methyl-6-(3- methyl-4- (((methyl(propyl)carbamoyl)oxy) methyl)isoxazol-5-yl)pyridin-3- yl)oxy)methyl)cyclohexane-1- carboxylic acid |
| Compound 76 | 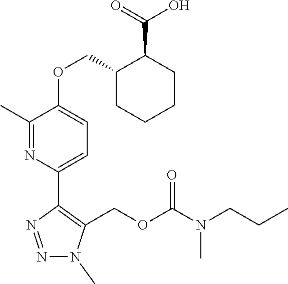 [SMILES: CCCN(C)C(=O)OCc1c(-c2ccc(OC[C@H]3CCCC[C@@H]3C(=O)O)c(C)n2)nnn1C] | (1S,2S)-2-(((2-methyl-6-(1- methyl-5- (((methyl(propyl)carbamoyl)oxy) methyl)-1H-1,2,3-triazol-4- yl)pyridin-3- yl)oxy)methyl)cyclohexane-1- carboxylic acid |
| Compound 77 | 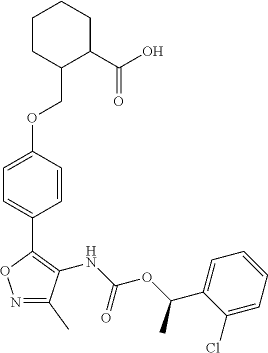 [SMILES: Cc1noc(-c2ccc(OCC3CCCCC3C(=O)O)cc2)c1NC(=O)O[C@H](C)c1ccccc1Cl] | 2-((4-(4-((((R)-1-(2-chloro phenyl)ethoxy)carbonyl)amino)- 3-methylisoxazol-5- yl)phenoxy)methyl)cyclohexane- 1-carboxylic acid (trans mixture) |
| Compound 78 | 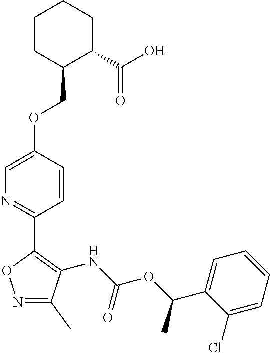 [SMILES: Cc1noc(-c2ccc(OC[C@H]3CCCC[C@@H]3C(=O)O)cn2)c1NC(=O)O[C@H](C)c1ccccc1Cl] | (1S,2S)-2-(((6-(4-((((R)-1-(2- chlorophenyl)ethoxy)carbonyl) amino)-3-methylisoxazol-5- yl)pyridin-3- yl)oxy)methyl)cyclohexane-1- carboxylic acid |
| Compound 79 | 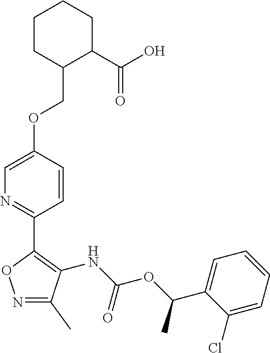 [SMILES: Cc1noc(-c2ccc(OCC3CCCCC3C(=O)O)cn2)c1NC(=O)O[C@H](C)c1ccccc1Cl] | 2-(((6-(4-((((R)-1-(2- chlorophenyl)ethoxy)carbonyl) amino)-3-methylisoxazol-5- yl)pyridin-3- yl)oxy)methyl)cyclohexane-1- carboxylic acid (cis mixture) |
| Compound 80 | 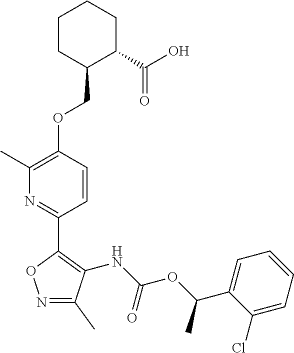 [SMILES: Cc1nc(-c2onc(C)c2NC(=O)O[C@H](C)c2ccccc2Cl)ccc1OC[C@H]1CCCC[C@@H]1C(=O)O] | (1S,2S)-2-(((6-(4-((((R)-1-(2- chlorophenyl)ethoxy)carbonyl) amino)-3-methylisoxazol-5-yl)-2- methylpyridin-3- yl)oxy)methyl)cyclohexane-1- carboxylic acid |
| Compound 81 | 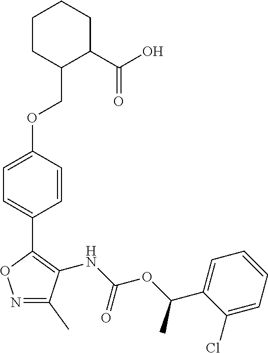 [SMILES: Cc1noc(-c2ccc(OCC3CCCCC3C(=O)O)cc2)c1NC(=O)O[C@H](C)c1ccccc1Cl] | Single Diastereomer 1 of cis-2- (((6-(4-((((R)-1-(2- chlorophenyl)ethoxy)carbonyl) amino)-3-methylisoxazol-5- yl)pyridin-3- yl)oxy)methyl)cyclohexane-1- carboxylic acid |
| Compound 82 | 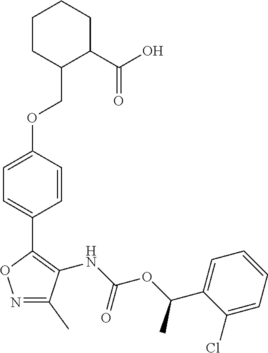 [SMILES: Cc1noc(-c2ccc(OCC3CCCCC3C(=O)O)cc2)c1NC(=O)O[C@H](C)c1ccccc1Cl] | Single Diastereomer 2 of cis-2- (((6-(4-((((R)-1-(2- chlorophenyl)ethoxy)carbonyl) amino)-3-methylisoxazol-5- yl)pyridin-3- yl)oxy)methyl)cyclohexane-1- carboxylic acid |

It is to be understood that all the single enantiomers, diastereoisomers and mixtures thereof, in any proportion, of the compounds of formula (I) of the invention are encompassed within the scope of the present invention.

In a preferred embodiment, the invention refers to a compound of formula (I) as LPA1 antagonist, wherein L is —O— and L₁ is —CH₂—, represented by the general formula (Ia)

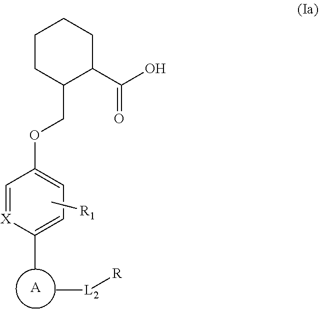

wherein
X is —CH— or N;
R₁ is H or (C₁-C₄)alkyl;
A is selected from the group consisting of

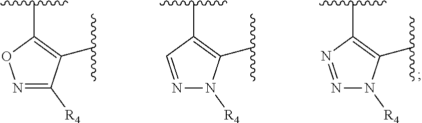

L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, heteroaryl, —(C₁-C₄)alkyl-R₂, —NR₆R₇, —OC(O)NR₆R₇, —NH—C(O)O—(C₁-C₄)alkyl-R₂, wherein any of such heteroaryl may be optionally substituted by one or more groups selected from —OR₃, —(C₁-C₄)alkyl, —(C₁-C₄)haloalkyl, —(C₃-C₆)cycloalkyl, aryl, heteroaryl, —NR₆R₇;
R₂ is H or selected from the group consisting of —(C₁-C₄)alkyl, aryl, heteroaryl wherein any of such aryl, heteroaryl and alkyl may be optionally substituted by one or more groups selected from —(C₁-C₄)alkyl, halo, —OR₅, —R₅;
R₃ is H or selected from the group consisting of —(C₁-C₄)alkyl, —(C₁-C₄)haloalkyl, —(C₃-C₆)cycloalkyl optionally substituted by one or more halo;
R₄ is H or (C₁-C₄)alkyl;
R₅ is aryl optionally substituted by one or more groups selected from (C₁-C₄)alkyl, halo;
R₆ and R₇ are at each occurrence independently H or selected from the group consisting of —(C₁-C₆)alkyl, —(C₃-C₆)cycloalkyl, heteroaryl wherein any of such heteroaryl may be optionally substituted by one or more groups selected from —OR₄, aryl, C₃-C₆)cycloalkylheteroaryl.

In a preferred embodiment, the invention refers to at least one of the compounds listed in the Table 2 below and pharmaceutical acceptable salts thereof.

TABLE 2
List of preferred compounds of Formula (Ia)

| Compound No | Structure | Chemical Name |
|---|---|---|
| Compound 77 | 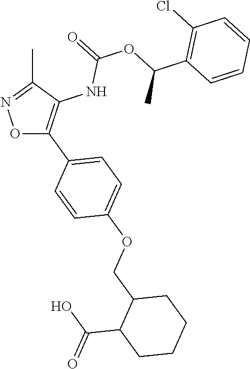 [SMILES: Cc1noc(-c2ccc(OCC3CCCCC3C(=O)O)cc2)c1NC(=O)O[C@H](C)c1ccccc1Cl] | 2-((4-(4-((((R)-1-(2- chlorophenyl)ethoxy)carbonyl) amino)-3-methylisoxazol-5- yl)phenoxy)methyl)cyclohexane- 1-carboxylic acid (trans mixture) |
| Compound 56 | 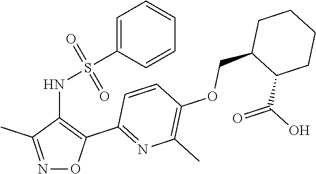 [SMILES: Cc1nc(-c2onc(C)c2NS(=O)(=O)c2ccccc2)ccc1OC[C@H]1CCCC[C@@H]1C(=O)O] | (1S,2S)-2-(((2-methyl-6-(3- methyl-4- (phenylsulfonamido)isoxazol-5- yl)pyridin-3- yl)oxy)methyl)cyclohexane-1- carboxylic acid |
| Compound 78 | 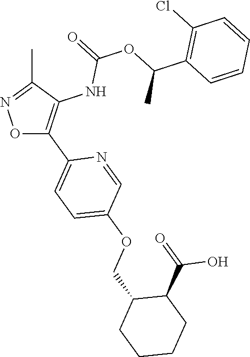 [SMILES: Cc1noc(-c2ccc(OC[C@H]3CCCC[C@@H]3C(=O)O)cn2)c1NC(=O)O[C@H](C)c1ccccc1Cl] | (1S,2S)-2-(((6-(4-((((R)-1-(2- chlorophenyl)ethoxy)carbonyl) amino)-3-methylisoxazol-5-yl)pyridin- 3-yl)oxy)methyl)cyclohexane-1- carboxylic acid |
| Compound 79 | 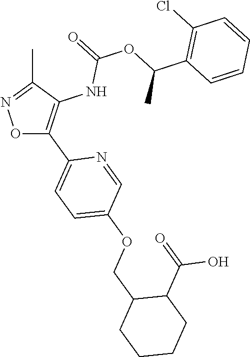 [SMILES: Cc1noc(-c2ccc(OCC3CCCCC3C(=O)O)cn2)c1NC(=O)O[C@H](C)c1ccccc1Cl] | 2-(((6-(4-((((R)-1-(2- chlorophenyl)ethoxy)carbonyl) amino)-3-methylisoxazol-5-yl)pyridin- 3-yl)oxy)methyl)cyclohexane-1- carboxylic acid |
| Compound 47 | 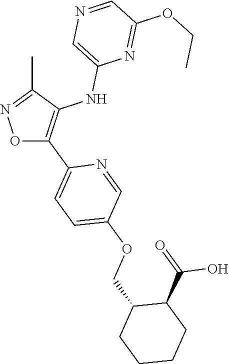 [SMILES: CCOc1cncc(Nc2c(C)noc2-c2ccc(OC[C@H]3CCCC[C@@H]3C(=O)O)cn2)n1] | (1S,2S)-2-(((6-(4-((6- ethoxypyrazin-2-yl)amino)-3- methylisoxazol-5-yl)pyridin-3- yl)oxy)methyl)cyclohexane-1- carboxylic acid |
| Compound 80 | 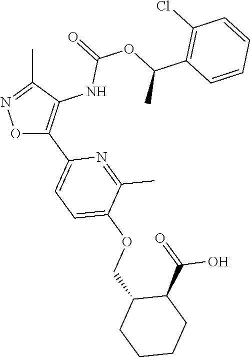 [SMILES: Cc1nc(-c2onc(C)c2NC(=O)O[C@H](C)c2ccccc2Cl)ccc1OC[C@H]1CCCC[C@@H]1C(=O)O] | (1S,2S)-2-(((6-(4-((((R)-1-(2- chlorophenyl)ethoxy)carbonyl) amino)-3-methylisoxazol-5-yl)-2- methylpyridin-3- yl)oxy)methyl)cyclohexane-1- carboxylic acid |
| Compound 48 | 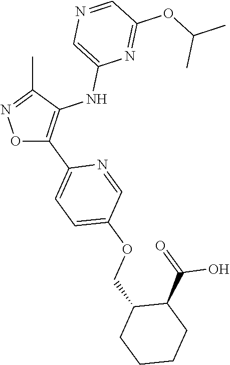 [SMILES: Cc1noc(-c2ccc(OC[C@H]3CCCC[C@@H]3C(=O)O)cn2)c1Nc1cncc(OC(C)C)n1] | (1S,2S)-2-(((6-(4-((6- isopropoxypyrazin-2-yl)amino)-3- methylisoxazol-5-yl)pyridin-3- yl)oxy)methyl)cyclohexane-1- carboxylic acid |
| Compound 69 | 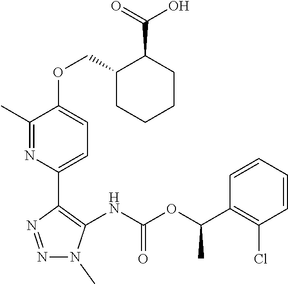 [SMILES: Cc1nc(-c2nnn(C)c2NC(=O)O[C@H](C)c2ccccc2Cl)ccc1OC[C@H]1CCCC[C@@H]1C(=O)O] | (1S,2S)-2-(((6-(5-((((R)-1-(2- chlorophenyl)ethoxy)carbonyl) amino)-1-methyl-1H-1,2,3-triazol-4- yl)-2-methylpyridin-3- yl)oxy)methyl)cyclohexane-1- carboxylic acid |
| Compound 49 | 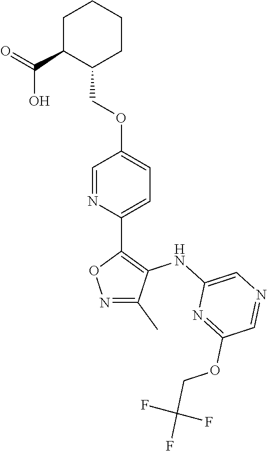 [SMILES: Cc1noc(-c2ccc(OC[C@H]3CCCC[C@@H]3C(=O)O)cn2)c1Nc1cncc(OCC(F)(F)F)n1] | (1S,2S)-2-(((6-(3-methyl-4-((6- (2,2,2-trifluoroethoxy)pyrazin-2- yl)amino)isoxazol-5-yl)pyridin-3- yl)oxy)methyl)cyclohexane-1- carboxylic acid |
| Compound 50 | 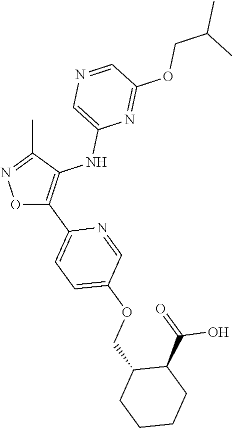 [SMILES: Cc1noc(-c2ccc(OC[C@H]3CCCC[C@@H]3C(=O)O)cn2)c1Nc1cncc(OCC(C)C)n1] | (1S,2S)-2-(((6-(4-((6- isobutoxypyrazin-2-yl)amino)-3- methylisoxazol-5-yl)pyridin-3- yl)oxy)methyl)cyclohexane-1- carboxylic acid |
| Compound 81 | 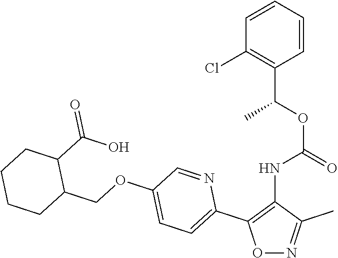 [SMILES: Cc1noc(-c2ccc(OCC3CCCCC3C(=O)O)cn2)c1NC(=O)O[C@H](C)c1ccccc1Cl] | Single Diastereoisomer 1 of cis-2- (((6-(4-((((R)-1-(2- chlorophenyl)ethoxy)carbonyl) amino)-3-methylisoxazol-5-yl)pyridin- 3-yl)oxy)methyl)cyclohexane-1- carboxylic acid |
| Compound 82 | 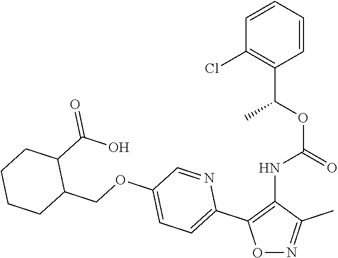 [SMILES: Cc1noc(-c2ccc(OCC3CCCCC3C(=O)O)cn2)c1NC(=O)O[C@H](C)c1ccccc1Cl] | Single Diastereomer 2 of cis-2- (((6-(4-((((R)-1-(2- chlorophenyl)ethoxy)carbonyl) amino)-3-methylisoxazol-5-yl)pyridin- 3-yl)oxy)methyl)cyclohexane-1- carboxylic acid |
| Compound 74 | 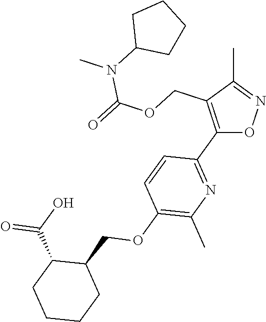 [SMILES: Cc1nc(-c2onc(C)c2COC(=O)N(C)C2CCCC2)ccc1OC[C@H]1CCCC[C@@H]1C(=O)O] | (1S,2S)-2-(((6-(4- (((cyclopentyl(methyl)carbamoyl) oxy)methyl)-3-methylisoxazol-5- yl)-2-methylpyridin-3- yl)oxy)methyl)cyclohexane-1- carboxylic acid |
| Compound 76 | 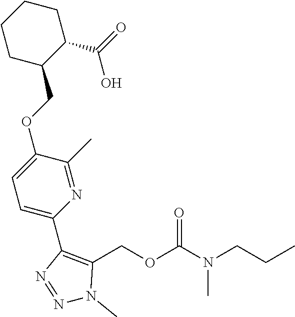 [SMILES: CCCN(C)C(=O)OCc1c(-c2ccc(OC[C@H]3CCCC[C@@H]3C(=O)O)c(C)n2)nnn1C] | (1S,2S)-2-(((2-methyl-6-(1- methyl-5- (((methyl(propyl)carbamoyl)oxy) methyl)-1H-1,2,3-triazol-4- yl)pyridin-3- yl)oxy)methyl)cyclohexane-1- carboxylic acid |
| Compound 51 | 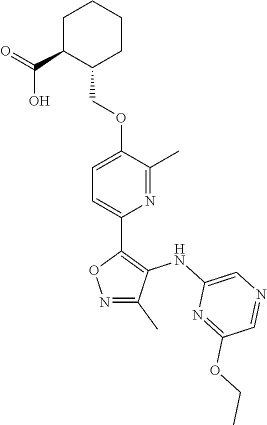 [SMILES: CCOc1cncc(Nc2c(C)noc2-c2ccc(OC[C@H]3CCCC[C@@H]3C(=O)O)c(C)n2)n1] | (1S,2S)-2-(((6-(4-((6- ethoxypyrazin-2-yl)amino)-3- methylisoxazol-5-yl)-2- methylpyridin-3- yl)oxy)methyl)cyclohexane-1- carboxylic acid |
| Compound 52 | 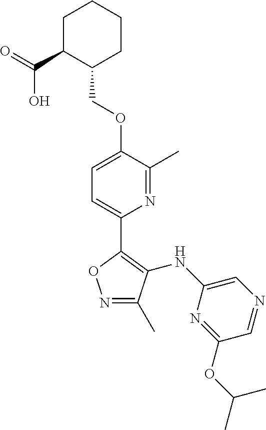 [SMILES: Cc1nc(-c2onc(C)c2Nc2cncc(OC(C)C)n2)ccc1OC[C@H]1CCCC[C@@H]1C(=O)O] | (1S,2S)-2-(((6-(4-((6- isopropoxypyrazin-2-yl)amino)-3- methylisoxazol-5-yl)-2- methylpyridin-3- yl)oxy)methyl)cyclohexane-1- carboxylic acid |
| Compound 58 | 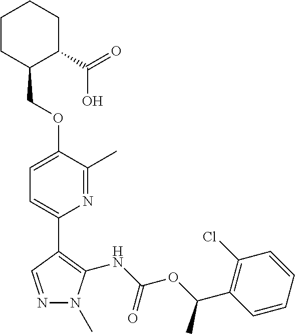 [SMILES: Cc1nc(-c2cnn(C)c2NC(=O)O[C@H](C)c2ccccc2Cl)ccc1OC[C@H]1CCCC[C@@H]1C(=O)O] | (1S,2S)-2-(((6-(5-((((R)-1-(2- chlorophenyl)ethoxy)carbonyl) amino)-1-methyl-1H-pyrazol-4-yl)-2- methylpyridin-3- yl)oxy)methyl)cyclohexane-1- carboxylic acid |
| Compound 53 | 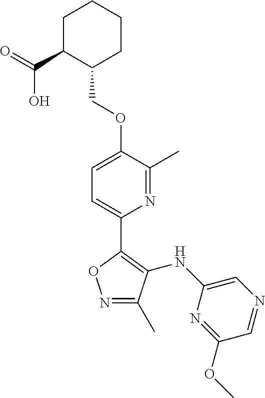 [SMILES: COc1cncc(Nc2c(C)noc2-c2ccc(OC[C@H]3CCCC[C@@H]3C(=O)O)c(C)n2)n1] | (1S,2S)-2-(((6-(4-((6- methoxypyrazin-2-yl)amino)-3- methylisoxazol-5-yl)-2- methylpyridin-3- yl)oxy)methyl)cyclohexane-1- carboxylic acid |
| Compound 70 | 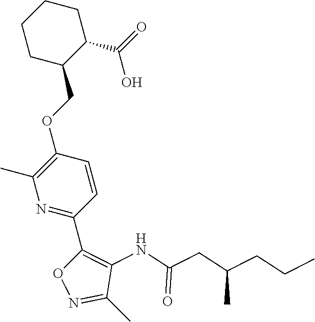 [SMILES: CCC[C@@H](C)CC(=O)Nc1c(C)noc1-c1ccc(OC[C@H]2CCCC[C@@H]2C(=O)O)c(C)n1] | (1S,2S)-2-(((2-methyl-6-(3- methyl-4-(((((R)-pentan-2- yl)oxy)carbonyl)amino)isoxazol-5- yl)pyridin-3- yl)oxy)methyl)cyclohexane-1- carboxylic acid |
| Compound 62 | 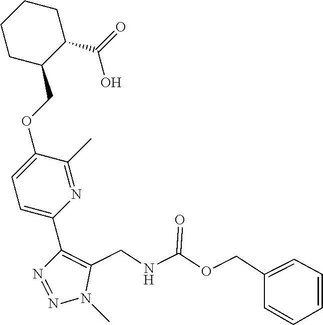 [SMILES: Cc1nc(-c2nnn(C)c2CNC(=O)OCc2ccccc2)ccc1OC[C@H]1CCCC[C@@H]1C(=O)O] | (1S,2S)-2-(((6-(5- ((((benzyloxy)carbonyl)amino) methyl)-1-methyl-1H-1,2,3-triazol-4- yl)-2-methylpyridin-3- yl)oxy)methyl)cyclohexane-1- carboxylic acid |
| Compound 46 | 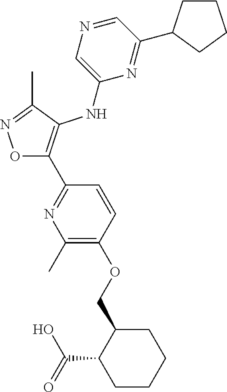 [SMILES: Cc1nc(-c2onc(C)c2Nc2cncc(C3CCCC3)n2)ccc1OC[C@H]1CCCC[C@@H]1C(=O)O] | (1S,2S)-2-(((6-(4-((6- cyclopentylpyrazin-2-yl)amino)-3- methylisoxazol-5-yl)-2- methylpyridin-3- yl)oxy)methyl)cyclohexane-1- carboxylic acid |
| Compound 61 | 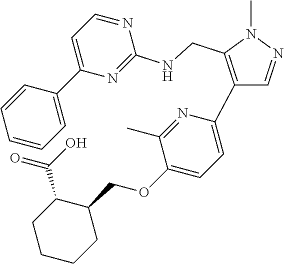 [SMILES: Cc1nc(-c2cnn(C)c2CNc2nccc(-c3ccccc3)n2)ccc1OC[C@H]1CCCC[C@@H]1C(=O)O] | (1S,2S)-2-(((2-methyl-6-(1- methyl-5-(((4-phenylpyrimidin-2- yl)amino)methyl)-1H-pyrazol-4- yl)pyridin-3- yl)oxy)methyl)cyclohexane-1- carboxylic acid |
| Compound 55 | 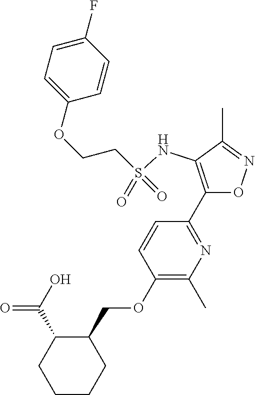 [SMILES: Cc1nc(-c2onc(C)c2NS(=O)(=O)CCOc2ccc(F)cc2)ccc1OC[C@H]1CCCC[C@@H]1C(=O)O] | (1S,2S)-2-(((6-(4-((2-(4- fluorophenoxy)ethyl)sulfonamido)- 3-methylisoxazol-5-yl)-2- methylpyridin-3- yl)oxy)methyl)cyclohexane-1- carboxylic acid |
| Compound 75 | 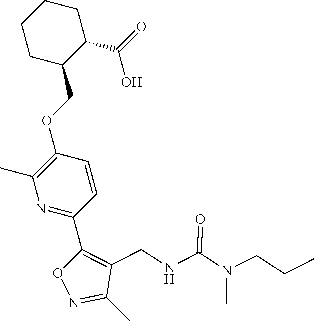 [SMILES: CCCN(C)C(=O)NCc1c(C)noc1-c1ccc(OC[C@H]2CCCC[C@@H]2C(=O)O)c(C)n1] | (1S,2S)-2-(((2-methyl-6-(3- methyl-4- (((methyl(propyl)carbamoyl)oxy) methyl)isoxazol-5-yl)pyridin-3- yl)oxy)methyl)cyclohexane-1- carboxylic acid |
| Compound 54 | 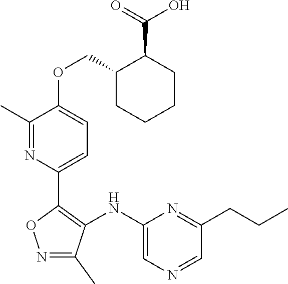 [SMILES: CCCc1cncc(Nc2c(C)noc2-c2ccc(OC[C@H]3CCCC[C@@H]3C(=O)O)c(C)n2)n1] | (1S,2S)-2-(((2-methyl-6-(3- methyl-4-((6-propylpyrazin-2- yl)amino)isoxazol-5-yl)pyridin-3- yl)oxy)methyl)cyclohexane-1- carboxylic acid |
| Compound 59 | 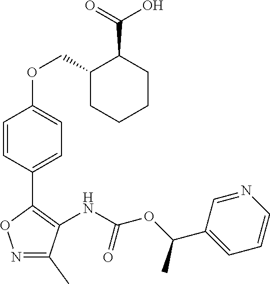 [SMILES: Cc1noc(-c2ccc(OC[C@H]3CCCC[C@@H]3C(=O)O)cc2)c1NC(=O)O[C@H](C)c1cccnc1] | (1S,2S)-2-((4-(3-methyl-4-((((R)- 1-(pyridin-3- yl)ethoxy)carbonyl)amino)isoxazol- 5- yl)phenoxy)methyl)cyclohexane- 1-carboxylic acid |
| Compound 60 | 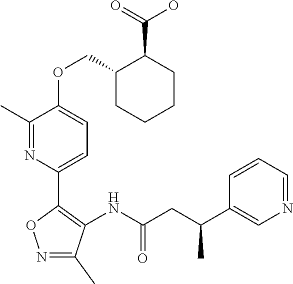 [SMILES: Cc1nc(-c2onc(C)c2NC(=O)C[C@H](C)c2cccnc2)ccc1OC[C@H]1CCCC[C@@H]1C(=O)O] | methyl (1S,2S)-2-(((2-methyl-6- (3-methyl-4-((((R)-1-(pyridin-3- yl)ethoxy)carbonyl)amino)isoxazol- 5-yl)pyridin-3- yl)oxy)methyl)cyclohexane-1- carboxylic acid |
| Compound 57 | 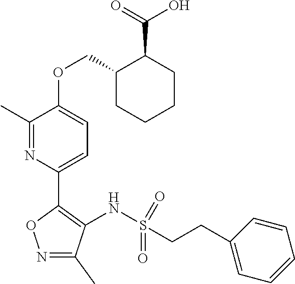 [SMILES: Cc1nc(-c2onc(C)c2NS(=O)(=O)CCc2ccccc2)ccc1OC[C@H]1CCCC[C@@H]1C(=O)O] | (1S,2S)-2-(((2-methyl-6-(3-methyl- 4-((2- phenylethyl)sulfonamido)isoxazol- 5-yl)pyridin-3- yl)oxy)methyl)cyclohexane-1- carboxylic acid |
| Compound 63 | 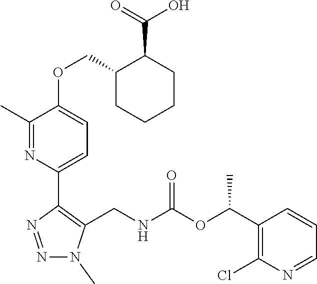 [SMILES: Cc1nc(-c2nnn(C)c2CNC(=O)O[C@H](C)c2cccnc2Cl)ccc1OC[C@H]1CCCC[C@@H]1C(=O)O] | (1S,2S)-2-(((6-(5-(((((R)-1-(2- chloropyridin-3- yl)ethoxy)carbonyl)amino)methyl)- 1-methyl-1H-1,2,3-triazol-4-yl)-2- methylpyridin-3- yl)oxy)methyl)cyclohexane-1- carboxylic acid |
| Compound 64 | 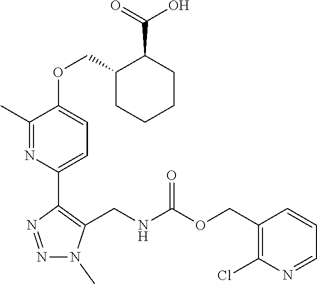 [SMILES: Cc1nc(-c2nnn(C)c2CNC(=O)OCc2cccnc2Cl)ccc1OC[C@H]1CCCC[C@@H]1C(=O)O] | (1S,2S)-2-(((6-(5-(((((2- chloropyridin-3- yl)methoxy)carbonyl)amino)methyl)- 1-methyl-1H-1,2,3-triazol-4-yl)- 2-methylpyridin-3- yl)oxy)methyl)cyclohexane-1- carboxylic acid |
| Compound 65 | 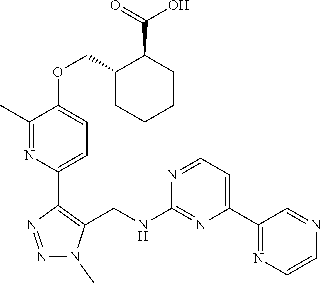 [SMILES: Cc1nc(-c2nnn(C)c2CNc2nccc(-c3cnccn3)n2)ccc1OC[C@H]1CCCC[C@@H]1C(=O)O] | (1S,2S)-2-(((2-methyl-6-(1-methyl- 5-(((4-(pyrazin-2-yl)pyrimidin-2- yl)amino)methyl)-1H-1,2,3-triazol- 4-yl)pyridin-3- yl)oxy)methyl)cyclohexane-1- carboxylic acid |
| Compound 66 | 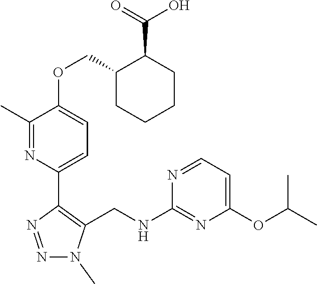 [SMILES: Cc1nc(-c2nnn(C)c2CNc2nccc(OC(C)C)n2)ccc1OC[C@H]1CCCC[C@@H]1C(=O)O] | (1S,2S)-2-(((6-(5-(((4- isopropoxypyrimidin-2- yl)amino)methyl)-1-methyl-1H- 1,2,3-triazol-4-yl)-2-methylpyridin- 3-yl)oxy)methyl)cyclohexane-1- carboxylic acid |
| Compound 67 | 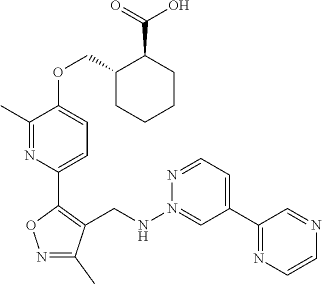 | (1S,2S)-2-(((2-methyl-6-(3-methyl- 4-(((4-(pyrazin-2-yl)pyrimidin-2- yl)amino)methyl)isoxazol-5- yl)pyridin-3- yl)oxy)methyl)cyclohexane-1- carboxylic acid |
| Compound 68 | 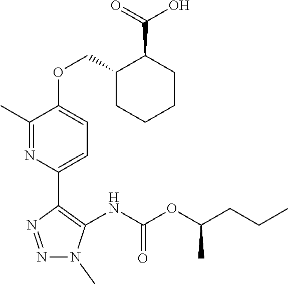 [SMILES: CCC[C@@H](C)OC(=O)Nc1c(-c2ccc(OC[C@H]3CCCC[C@@H]3C(=O)O)c(C)n2)nnn1C] | (1S,2S)-2-(((2-methyl-6-(1-methyl- 5-(((((R)-pentan-2- yl)oxy)carbonyl)amino)-1H-1,2,3- triazol-4-yl)pyridin-3- yl)oxy)methyl)cyclohexane-1- carboxylic acid |
| Compound 71 | 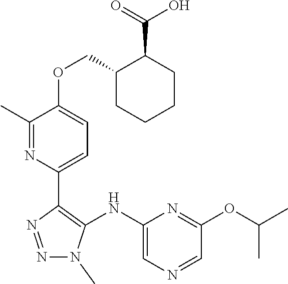 [SMILES: Cc1nc(-c2nnn(C)c2Nc2cncc(OC(C)C)n2)ccc1OC[C@H]1CCCC[C@@H]1C(=O)O] | (1S,2S)-2-(((6-(5-((6- isopropoxypyrazin-2-yl)amino)-1- methyl-1H-1,2,3-triazol-4-yl)-2- methylpyridin-3- yl)oxy)methyl)cyclohexane-1- carboxylic acid |
| Compound 72 | 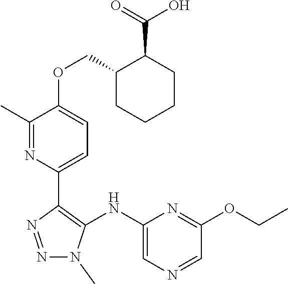 [SMILES: CCOc1cncc(Nc2c(-c3ccc(OC[C@H]4CCCC[C@@H]4C(=O)O)c(C)n3)nnn2C)n1] | (1S,2S)-2-(((6-(5-((6- ethoxypyrazin-2-yl)amino)-1- methyl-1H-1,2,3-triazol-4-yl)-2- methylpyridin-3- yl)oxy)methyl)cyclohexane-1- carboxylic acid |
| Compound 73 | 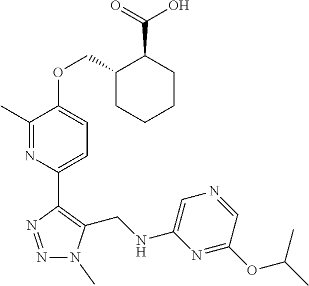 [SMILES: Cc1nc(-c2nnn(C)c2CNc2cncc(OC(C)C)n2)ccc1OC[C@H]1CCCC[C@@H]1C(=O)O] | (1S,2S)-2-(((6-(5-(((6- isopropoxypyrazin-2- yl)amino)methyl)-1-methyl-1H- 1,2,3-triazol-4-yl)-2-methylpyridin- 3-yl)oxy)methyl)cyclohexane-1- carboxylic acid |

In a further preferred embodiment, the invention refers to a compound of formula (I) as LPA1 antagonist, wherein X is N, L is —NH— and L₁ is —C(O)—, represented by the general formula (Ib)

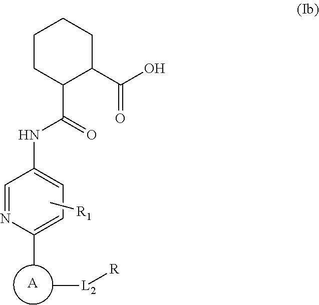

wherein
R₁ is H or —(C₁-C₄)alkyl;
A is selected from the group consisting of

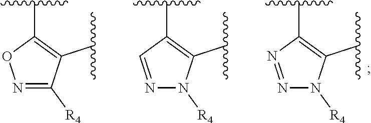

L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, heteroaryl, —NR₆R₇, —(C₁-C₄)alkyl-R₂, —OC(O)NR₆R₇, —NH—C(O)O—(C₁-C₄)alkyl-R₂, wherein any of such heteroaryl may be optionally substituted by one or more groups selected from —OR₃, —(C₁-C₄)alkyl, —(C₁-C₄)haloalkyl, —(C₃-C₆)cycloalkyl, aryl, heteroaryl, —NR₆R₇;
R₂ is H or selected from the group consisting of —(C₁-C₄)alkyl, aryl, heteroaryl wherein any of such aryl, heteroaryl and alkyl are optionally substituted by one or more groups selected from —(C₁-C₄)alkyl, halo; —OR₅ and —R₅;
R₃ is H or selected from the group consisting of —(C₁-C₄)alkyl, —(C₁-C₄)haloalkyl, —(C₃-C₆)cycloalkyl optionally substituted by one or more halo;
R₄ is H or (C₁-C₄)alkyl;
R₅ is aryl optionally substituted by one or more groups selected from —(C₁-C₄)alkyl, halo;
R₆ and R₇ are at each occurrence independently H or selected from the group consisting of —(C₁-C₆)alkyl, —(C₃-C₆)cycloalkyl, heteroaryl wherein any of such heteroaryl may be optionally substituted by one or more groups selected from —OR₄, aryl, —(C₃-C₆)cycloalkyl, heteroaryl.

In a still preferred embodiment, the invention refers to at least one of the compounds listed in the Table 3 below and pharmaceutical acceptable salts thereof.

TABLE 3
List of preferred compounds of Formula (Ib)

| Compound No. | Structure | Chemical Name |
|---|---|---|
| Compound 2 | 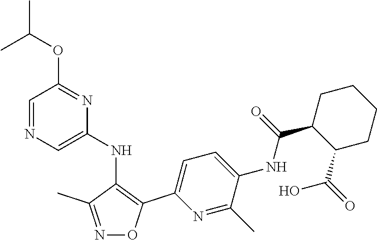 [SMILES: Cc1nc(-c2onc(C)c2Nc2cncc(OC(C)C)n2)ccc1NC(=O)[C@H]1CCCC[C@@H]1C(=O)O] | (1S,2S)-2-((6-(4-((6- isopropoxypyrazin-2-yl)amino)-3- methylisoxazol-5-yl)-2- methylpyridin-3- yl)carbamoyl)cyclohexane-1- carboxylic acid |
| Compound 23 | 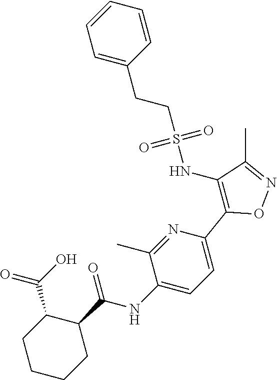 [SMILES: Cc1nc(-c2onc(C)c2NS(=O)(=O)CCc2ccccc2)ccc1NC(=O)[C@H]1CCCC[C@@H]1C(=O)O] | (1S,2S)-2-((2-methyl-6-(3-methyl- 4-((2- phenylethyl)sulfonamido)isoxazol- 5-yl)pyridin-3- yl)carbamoyl)cyclohexane-1- carboxylic acid |
| Compound 1 | 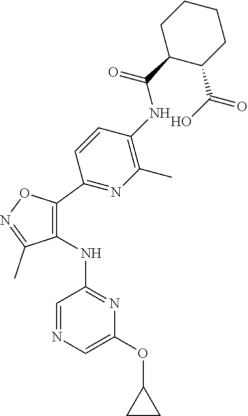 [SMILES: Cc1nc(-c2onc(C)c2Nc2cncc(OC3CC3)n2)ccc1NC(=O)[C@H]1CCCC[C@@H]1C(=O)O] | (1S,2S)-2-((6-(4-((6- cyclopropoxypyrazin-2-yl)amino)- 3-methylisoxazol-5-yl)-2- methylpyridin-3- yl)carbamoyl)cyclohexane-1- carboxylic acid |
| Compound 4 | 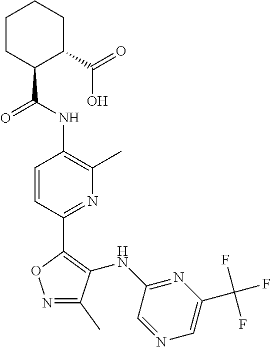 [SMILES: Cc1nc(-c2onc(C)c2Nc2cncc(C(F)(F)F)n2)ccc1NC(=O)[C@H]1CCCC[C@@H]1C(=O)O] | (1S,2S)-2-((2-methyl-6-(3-methyl- 4-((6-(trifluoromethyl)pyrazin-2- yl)amino)isoxazol-5-yl)pyridin-3- yl)carbamoyl)cyclohexane-1- carboxylic acid |
| Compound 5 | 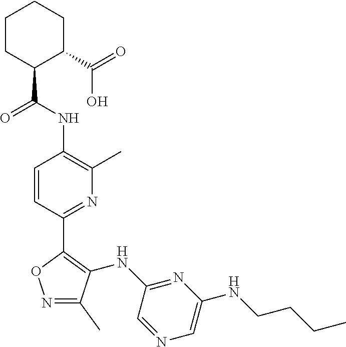 [SMILES: CCCCNc1cncc(Nc2c(C)noc2-c2ccc(NC(=O)[C@H]3CCCC[C@@H]3C(=O)O)c(C)n2)n1] | (1S,2S)-2-((6-(4-((6- (butylamino)pyrazin-2-yl)amino)- 3-methylisoxazol-5-yl)-2- methylpyridin-3- yl)carbamoyl)cyclohexane-1- carboxylic acid |
| Compound 3 | 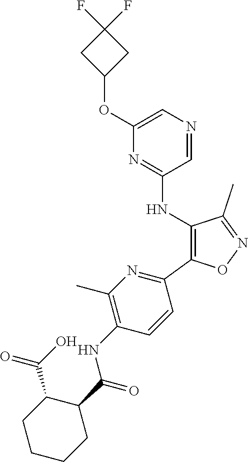 [SMILES: Cc1nc(-c2onc(C)c2Nc2cncc(OC3CC(F)(F)C3)n2)ccc1NC(=O)[C@H]1CCCC[C@@H]1C(=O)O] | (1S,2S)-2-((6-(4-((6-(3,3- difluorocyclobutoxy)pyrazin-2- yl)amino)-3-methylisoxazol-5-yl)- 2-methylpyridin-3- yl)carbamoyl)cyclohexane-1- carboxylic acid |
| Compound 6 | 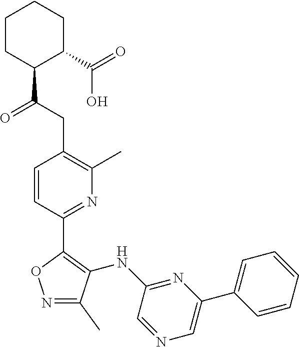 [SMILES: Cc1nc(-c2onc(C)c2Nc2cncc(-c3ccccc3)n2)ccc1CC(=O)[C@H]1CCCC[C@@H]1C(=O)O] | (1S,2S)-2-((2-methyl-6-(3-methyl- 4-((6-phenylpyrazin-2- yl)amino)isoxazol-5-yl)pyridin-3- yl)carbamoyl)cyclohexane-1- carboxylic acid |
| Compound 32 | 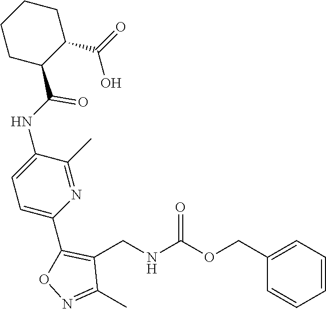 [SMILES: Cc1nc(-c2onc(C)c2CNC(=O)OCc2ccccc2)ccc1NC(=O)[C@H]1CCCC[C@@H]1C(=O)O] | (1S,2S)-2-((6-(4- ((((benzyloxy)carbonyl)amino) methyl)-3-methylisoxazol-5-yl)-2- methylpyridin-3- yl)carbamoyl)cyclohexane-1- carboxylic acid |
| Compound 7 | 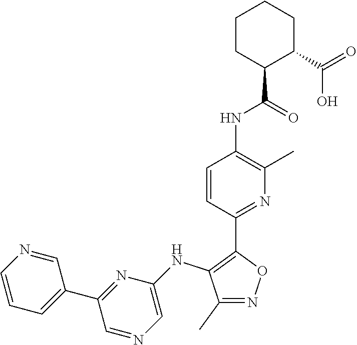 [SMILES: Cc1nc(-c2onc(C)c2Nc2cncc(-c3cccnc3)n2)ccc1NC(=O)[C@H]1CCCC[C@@H]1C(=O)O] | (1S,2S)-2-((2-methyl-6-(3-methyl- 4-((6-(pyridin-3-yl)pyrazin-2- yl)amino)isoxazol-5-yl)pyridin-3- yl)carbamoyl)cyclohexane-1- carboxylic acid |
| Compound 33 | 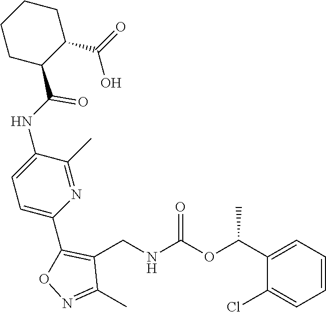 [SMILES: Cc1nc(-c2onc(C)c2CNC(=O)O[C@H](C)c2ccccc2Cl)ccc1NC(=O)[C@H]1CCCC[C@@H]1C(=O)O] | (1S,2S)-2-((6-(4-(((((R)-1-(2- chlorophenyl)ethoxy)carbonyl) amino)methyl)-3-methylisoxazol- 5-yl)- 2-methylpyridin-3- yl)carbamoyl)cyclohexane-1- carboxylic acid |
| Compound 34 | 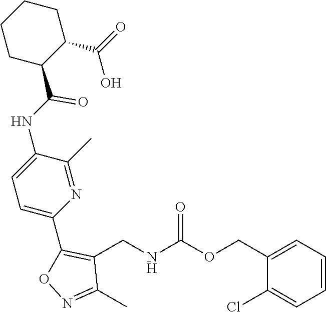 [SMILES: Cc1nc(-c2onc(C)c2CNC(=O)OCc2ccccc2Cl)ccc1NC(=O)[C@H]1CCCC[C@@H]1C(=O)O] | (1S,2S)-2-((6-(4-(((((R)-1-(2- chlorophenyl)ethoxy)carbonyl) amino)methyl)-3-methylisoxazol- 5-yl)- 2-methylpyridin-3- yl)carbamoyl)cyclohexane-1- carboxylic acid |
| Compound 36 | 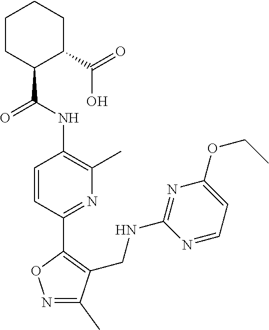 [SMILES: CCOc1ccnc(NCc2c(C)noc2-c2ccc(NC(=O)[C@H]3CCCC[C@@H]3C(=O)O)c(C)n2)n1] | (1S,2S)-2-((6-(4-(((4- ethoxypyrimidin-2- yl)amino)methyl)-3- methylisoxazol-5-yl)-2- methylpyridin-3- yl)carbamoyl)cyclohexane-1- carboxylic acid |
| Compound 22 | 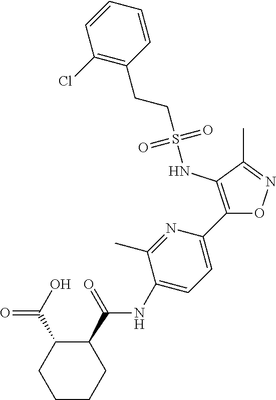 [SMILES: Cc1nc(-c2onc(C)c2NS(=O)(=O)CCc2ccccc2Cl)ccc1NC(=O)[C@H]1CCCC[C@@H]1C(=O)O] | (1S,2S)-2-((6-(4-((2-(2- chlorophenyl)ethyl)sulfonamido)-3- methylisoxazol-5-yl)-2- methylpyridin-3- yl)carbamoyl)cyclohexane-1- carboxylic acid |
| Compound 24 | 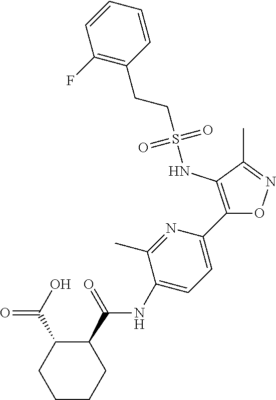 [SMILES: Cc1nc(-c2onc(C)c2NS(=O)(=O)CCc2ccccc2F)ccc1NC(=O)[C@H]1CCCC[C@@H]1C(=O)O] | (1S,2S)-2-((6-(4-((2-(2- fluorophenyl)ethyl)sulfonamido)-3- methylisoxazol-5-yl)-2- methylpyridin-3- yl)carbamoyl)cyclohexane-1- carboxylic acid |
| Compound 25 | 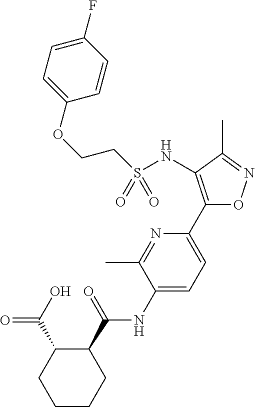 [SMILES: Cc1nc(-c2onc(C)c2NS(=O)(=O)CCOc2ccc(F)cc2)ccc1NC(=O)[C@H]1CCCC[C@@H]1C(=O)O] | (1S,2S)-2-((6-(4-((2-(4- fluorophenoxy)ethyl)sulfonamido)- 3-methylisoxazol-5-yl)-2- methylpyridin-3- yl)carbamoyl)cyclohexane-1- carboxylic acid |
| Compound 26 | 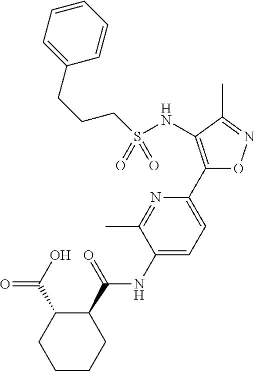 [SMILES: Cc1nc(-c2onc(C)c2NS(=O)(=O)CCCc2ccccc2)ccc1NC(=O)[C@H]1CCCC[C@@H]1C(=O)O] | (1S,2S)-2-((2-methyl-6-(3-methyl- 4-((3- phenylpropyl)sulfonamido)isoxazol- 5-yl)pyridin-3- yl)carbamoyl)cyclohexane-1- carboxylic acid |
| Compound 27 | 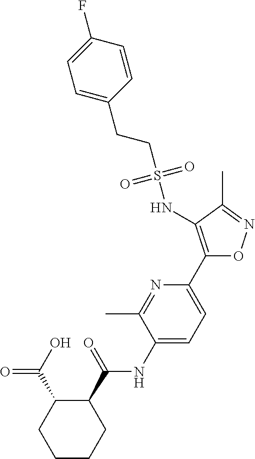 [SMILES: Cc1nc(-c2onc(C)c2NS(=O)(=O)CCc2ccc(F)cc2)ccc1NC(=O)[C@H]1CCCC[C@@H]1C(=O)O] | (1S,2S)-2-((6-(4-((2-(4- fluorophenyl)ethyl)sulfonamido)-3- methylisoxazol-5-yl)-2- methylpyridin-3- yl)carbamoyl)cyclohexane-1- carboxylic acid |
| Compound 42 | 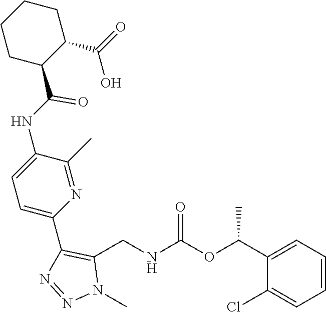 [SMILES: Cc1nc(-c2nnn(C)c2CNC(=O)O[C@H](C)c2ccccc2Cl)ccc1NC(=O)[C@H]1CCCC[C@@H]1C(=O)O] | (1S,2S)-2-((6-(5-(((((R)-1-(2- chlorophenyl)ethoxy)carbonyl) amino)methyl)-1-methyl-1H-1,2,3- triazol-4-yl)-2-methylpyridin-3- yl)carbamoyl)cyclohexane-1- carboxylic acid |
| Compound 29 | 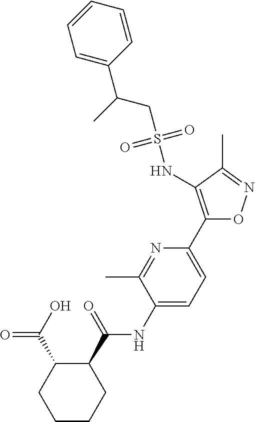 [SMILES: Cc1nc(-c2onc(C)c2NS(=O)(=O)CC(C)c2ccccc2)ccc1NC(=O)[C@H]1CCCC[C@@H]1C(=O)O] | (1S,2S)-2-((2-methyl-6-(3-methyl- 4-((2- phenylpropyl)sulfonamido) isoxazol-5-yl)pyridin-3- yl)carbamoyl)cyclohexane-1- carboxylic acid |
| Compound 28 | 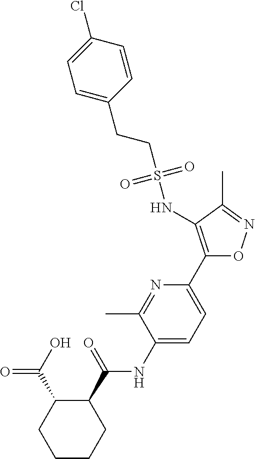 [SMILES: Cc1nc(-c2onc(C)c2NS(=O)(=O)CCc2ccc(Cl)cc2)ccc1NC(=O)[C@H]1CCCC[C@@H]1C(=O)O] | (1S,2S)-2-((6-(4-((2-(4- chlorophenyl)ethyl)sulfonamido)-3- methylisoxazol-5-yl)-2- methylpyridin-3- yl)carbamoyl)cyclohexane-1- carboxylic acid |
| Compound 35 | 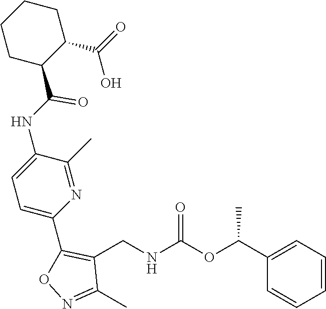 [SMILES: Cc1nc(-c2onc(C)c2CNC(=O)O[C@H](C)c2ccccc2)ccc1NC(=O)[C@H]1CCCC[C@@H]1C(=O)O] | (1S,2S)-2-((2-methyl-6-(3-methyl- 4-(((( ®-1- phenylethoxy)carbonyl)amino) methyl)isoxazol-5-yl)pyridin-3- yl)carbamoyl)cyclohexane-1- carboxylic acid |
| Compound 8 | 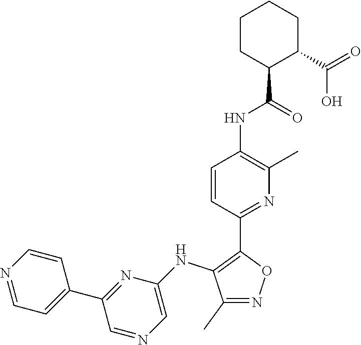 [SMILES: Cc1nc(-c2onc(C)c2Nc2cncc(-c3ccncc3)n2)ccc1NC(=O)[C@H]1CCCC[C@@H]1C(=O)O] | (1S,2S)-2-((2-methyl-6-(3-methyl- 4-((6-(pyridin-4-yl)pyrazin-2- yl)amino)isoxazol-5-yl)pyridin-3- yl)carbamoyl)cyclohexane-1- carboxylic acid |
| Compound 12 | 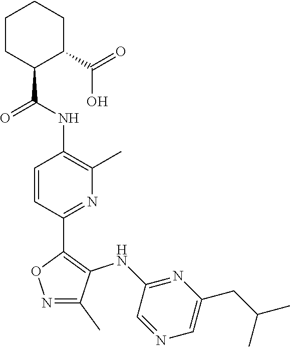 [SMILES: Cc1nc(-c2onc(C)c2Nc2cncc(CC(C)C)n2)ccc1NC(=O)[C@H]1CCCC[C@@H]1C(=O)O] | (1S,2S)-2-((6-(4-((6- isobutylpyrazin-2-yl)amino)-3- methylisoxazol-5-yl)-2- methylpyridin-3- yl)carbamoyl)cyclohexane-1- carboxylic acid |
| Compound 11 | 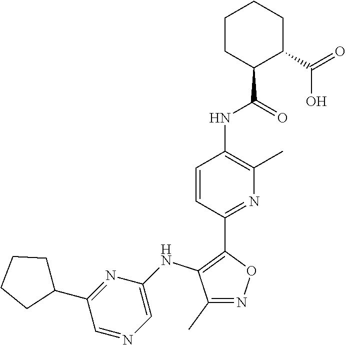 [SMILES: Cc1nc(-c2onc(C)c2Nc2cncc(C3CCCC3)n2)ccc1NC(=O)[C@H]1CCCC[C@@H]1C(=O)O] | (1S,2S)-2-((6-(4-((6- cyclopentylpyrazin-2-yl)amino)-3- methylisoxazol-5-yl)-2- methylpyridin-3- yl)carbamoyl)cyclohexane-1- carboxylic acid |
| Compound 14 | 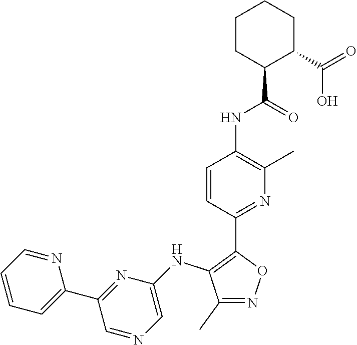 [SMILES: Cc1nc(-c2onc(C)c2Nc2cncc(-c3ccccn3)n2)ccc1NC(=O)[C@H]1CCCC[C@@H]1C(=O)O] | (1S,2S)-2-((2-methyl-6-(3-methyl- 4-((6-(pyridin-2-yl)pyrazin-2- yl)amino)isoxazol-5-yl)pyridin-3- yl)carbamoyl)cyclohexane-1- carboxylic acid |
| Compound 19 | 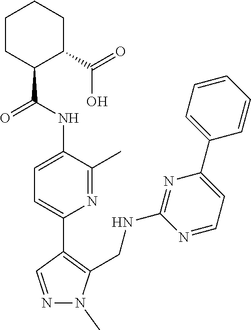 [SMILES: Cc1nc(-c2cnn(C)c2CNc2nccc(-c3ccccc3)n2)ccc1NC(=O)[C@H]1CCCC[C@@H]1C(=O)O] | (1S,2S)-2-((2-methyl-6-(1-methyl- 5-(((4-phenylpyrimidin-2- yl)amino)methyl)-1H-pyrazol-4- yl)pyridin-3- yl)carbamoyl)cyclohexane-1- carboxylic acid |
| Compound 30 | 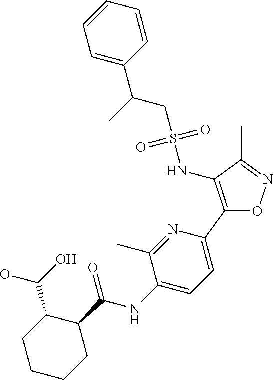 [SMILES: Cc1nc(-c2onc(C)c2NS(=O)(=O)CC(C)c2ccccc2)ccc1NC(=O)[C@H]1CCCC[C@@H]1C(O)O] | Single Diasteroisomer 1 of (1S,2S)- 2-((2-methyl-6-(3-methyl-4-((2- phenylpropyl)sulfonamido) isoxazol-5-yl)pyridin-3- yl)carbamoyl)cyclohexane-1- carboxylic acid |
| Compound 31 | 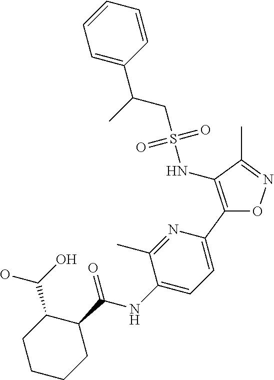 [SMILES: Cc1nc(-c2onc(C)c2NS(=O)(=O)CC(C)c2ccccc2)ccc1NC(=O)[C@H]1CCCC[C@@H]1C(O)O] | Single Diastereoisomer 1 of (1S,2S)-2-((2-methyl-6-(3-methyl- 4-((2- phenylpropyl)sulfonamido) isoxazol-5-yl)pyridin-3- yl)carbamoyl)cyclohexane-1- carboxylic acid |
| Compound 45 | 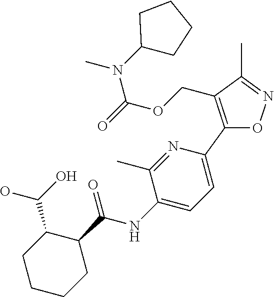 [SMILES: Cc1nc(-c2onc(C)c2COC(=O)N(C)C2CCCC2)ccc1NC(=O)[C@H]1CCCC[C@@H]1C(O)O] | (1S,2S)-2-((6-(4- (((cyclopentyl(methyl)carbamoyl) oxy)methyl)-3-methylisoxazol-5- yl)-2-methylpyridin-3- yl)carbamoyl)cyclohexane-1- carboxylic acid |
| Compound 21 | 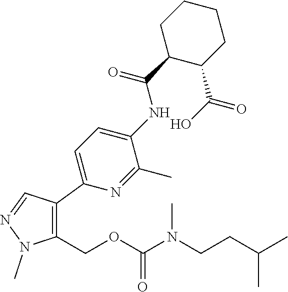 [SMILES: Cc1nc(-c2cnn(C)c2COC(=O)N(C)CCC(C)C)ccc1NC(=O)[C@H]1CCCC[C@@H]1C(=O)O] | (1S,2S)-2-((6-(5- (((isopentyl(methyl)carbamoyl)oxy) methyl)-1-methyl-1H-pyrazol-4- yl)-2-methylpyridin-3- yl)carbamoyl)cyclohexane-1- carboxylic acid |
| Compound 9 | 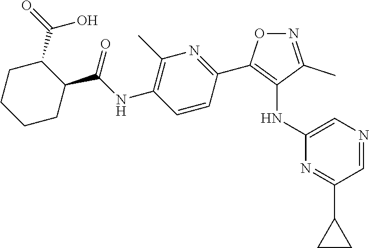 [SMILES: Cc1nc(-c2onc(C)c2Nc2cncc(C3CC3)n2)ccc1NC(=O)[C@H]1CCCC[C@@H]1C(=O)O] | (1S,2S)-2-((6-(4-((6- cyclopropylpyrazin-2-yl)amino)-3- methylisoxazol-5-yl)-2- methylpyridin-3- yl)carbamoyl)cyclohexane-1- carboxylic acid |
| Compound 13 | 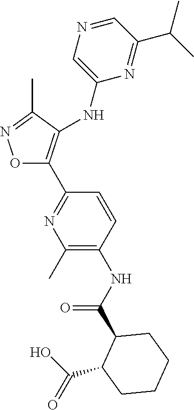 [SMILES: Cc1nc(-c2onc(C)c2Nc2cncc(C(C)C)n2)ccc1NC(=O)[C@H]1CCCC[C@@H]1C(=O)O] | (1S,2S)-2-((6-(4-((6- isopropylpyrazin-2-yl)amino)-3- methylisoxazol-5-yl)-2- methylpyridin-3- yl)carbamoyl)cyclohexane-1- carboxylic acid |
| Compound 18 | 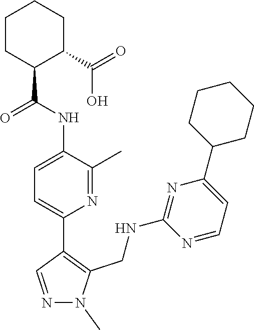 [SMILES: Cc1nc(-c2cnn(C)c2CNc2nccc(C3CCCCC3)n2)ccc1NC(=O)[C@H]1CCCC[C@@H]1C(=O)O] | (1S,2S)-2-((6-(5-(((4- cyclohexylpyrimidin-2- yl)amino)methyl)-1-methyl-1H- pyrazol-4-yl)-2-methylpyridin-3- yl)carbamoyl)cyclohexane-1- carboxylic acid |
| Compound 15 | 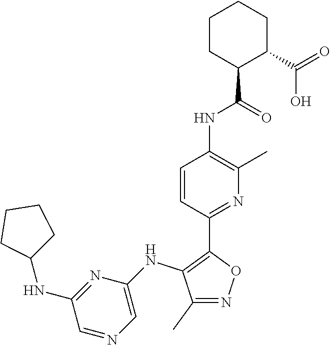 [SMILES: Cc1nc(-c2onc(C)c2Nc2cncc(NC3CCCC3)n2)ccc1NC(=O)[C@H]1CCCC[C@@H]1C(=O)O] | (1S,2S)-2-((6-(4-((6- (cyclopentylamino)pyrazin-2- yl)amino)-3-methylisoxazol-5-yl)- 2-methylpyridin-3- yl)carbamoyl)cyclohexane-1- carboxylic acid |
| Compound 10 | 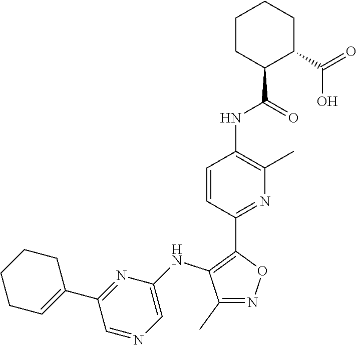 [SMILES: Cc1nc(-c2onc(C)c2Nc2cncc(C3=CCCCC3)n2)ccc1NC(=O)[C@H]1CCCC[C@@H]1C(=O)O] | (1S,2S)-2-((6-(4-((6-(cyclohex-1- en-1-yl)pyrazin-2-yl)amino)-3- methylisoxazol-5-yl)-2- methylpyridin-3- yl)carbamoyl)cyclohexane-1- carboxylic acid |
| Compound 17 | 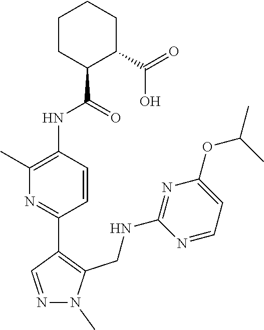 [SMILES: Cc1nc(-c2cnn(C)c2CNc2nccc(OC(C)C)n2)ccc1NC(=O)[C@H]1CCCC[C@@H]1C(=O)O] | (1S,2S)-2-((6-(5-(((4- isopropoxypyrimidin-2- yl)amino)methyl)-1-methyl-1H- pyrazol-4-yl)-2-methylpyridin-3- yl)carbamoyl)cyclohexane-1- carboxylic acid |
| Compound 44 | 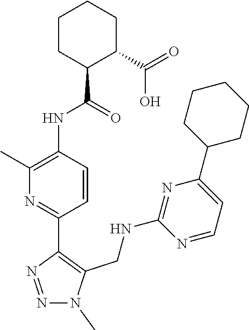 [SMILES: Cc1nc(-c2nnn(C)c2CNc2nccc(C3CCCCC3)n2)ccc1NC(=O)[C@H]1CCCC[C@@H]1C(=O)O] | (1S,2S)-2-((6-(5-(((4- cyclohexylpyrimidin-2- yl)amino)methyl)-1-methyl-1H- 1,2,3-triazol-4-yl)-2-methylpyridin- 3-yl)carbamoyl)cyclohexane-1- carboxylic acid |
| Compound 37 | 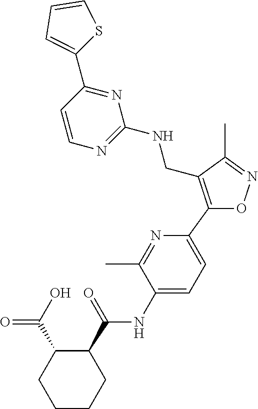 [SMILES: Cc1nc(-c2onc(C)c2CNc2nccc(-c3cccs3)n2)ccc1NC(=O)[C@H]1CCCC[C@@H]1C(=O)O] | (1S,2S)-2-((2-methyl-6-(3-methyl- 4-(((4-(thiophen-2-yl)pyrimidin-2- yl)amino)methyl)isoxazol-5- yl)pyridin-3- yl)carbamoyl)cyclohexane-1- carboxylic acid |
| Compound 38 | 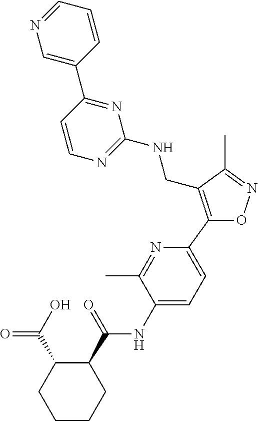 [SMILES: Cc1nc(-c2onc(C)c2CNc2nccc(-c3cccnc3)n2)ccc1NC(=O)[C@H]1CCCC[C@@H]1C(=O)O] | (1S,2S)-2-((2-methyl-6-(3-methyl- 4-(((4-(pyridin-3-yl)pyrimidin-2- yl)amino)methyl)isoxazol-5- yl)pyridin-3- yl)carbamoyl)cyclohexane-1- carboxylic acid |
| Compound 39 | 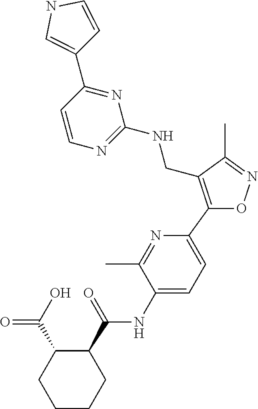 [SMILES: Cc1nc(-c2onc(C)c2CNc2nccc(-c3cc[nH]c3)n2)ccc1NC(=O)[C@H]1CCCC[C@@H]1C(=O)O] | (1S,2S)-2-((2-methyl-6-(3-methyl- 4-(((4-(thiophen-3-yl)pyrimidin-2- yl)amino)methyl)isoxazol-5- yl)pyridin-3- yl)carbamoyl)cyclohexane-1- carboxylic acid |
| Compound 40 | 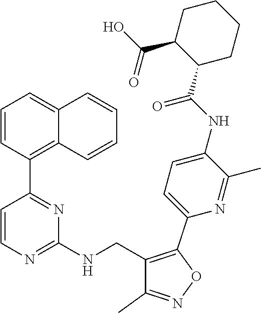 [SMILES: Cc1nc(-c2onc(C)c2CNc2nccc(-c3cccc4ccccc34)n2)ccc1NC(=O)[C@H]1CCCC[C@@H]1C(=O)O] | (1S,2S)-2-((2-methyl-6-(3-methyl- 4-(((4-(naphthalen-1-yl)pyrimidin- 2-yl)amino)methyl)isoxazol-5- yl)pyridin-3- yl)carbamoyl)cyclohexane-1- carboxylic acid |
| Compound 41 | 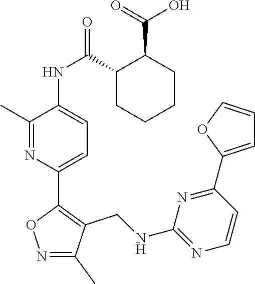 [SMILES: Cc1nc(-c2onc(C)c2CNc2nccc(-c3ccco3)n2)ccc1NC(=O)[C@H]1CCCC[C@@H]1C(=O)O] | (1S,2S)-2-((6-(4-(((4-(furan-2- yl)pyrimidin-2-yl)amino)methyl)-3- methylisoxazol-5-yl)-2- methylpyridin-3- yl)carbamoyl)cyclohexane-1- carboxylic acid |

In a further preferred embodiment, the invention refers to a compound of formula (I) as LPA1 antagonist, wherein X is —CH—, L is —NH— and L₁ is —C(O)—, L2 is —NH—, represented by the general formula (Ic)

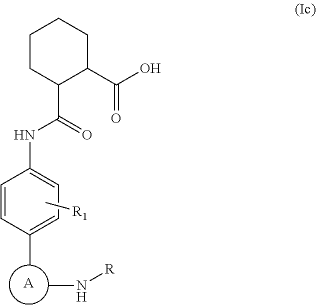

wherein
R₁ is H or —(C₁-C₄)alkyl;
A is selected from the group consisting of

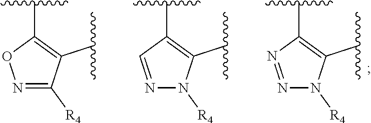

R is selected from the group consisting of —SO₂R₂, —SO₂(C₁-C₄)alkyl-OR₅, heteroaryl, —(C₁-C₄)alkyl-R₂, —NR₆R₇, —OC(O)NR₆R₇, —NH—C(O)O—(C₁-C₄)alkyl-R₂, wherein any of such heteroaryl may be optionally substituted by one or more groups selected from —OR₃, —(C₁-C₄)alkyl, —(C₁-C₄)haloalkyl, —(C₃-C₆)cycloalkyl, aryl, heteroaryl, —NR₆R₇;
R₂ is H or selected from the group consisting of —(C₁-C₄)alkyl, aryl, heteroaryl wherein any of such aryl, heteroaryl and alkyl may be optionally substituted by one or more groups selected from —(C₁-C₄)alkyl, halo; —OR₅, —R₅;
R₃ is H or selected from the group consisting of —(C₁-C₄)alkyl, —(C₁-C₄)haloalkyl, —(C₃-C₆)cycloalkyl optionally substituted by one or more halo,
R₄ is H or —(C₁-C₄)alkyl;
R₅ is aryl optionally substituted by one or more groups selected from —(C₁-C₄)alkyl, halo;
R₆ and R₇ are at each occurrence independently H or selected from the group consisting of —(C₁-C₆)alkyl, —(C₃-C₆)cycloalkyl, heteroaryl wherein any of such heteroaryl may be optionally substituted by one or more groups selected from —OR₄, aryl, —(C₃-C₆)cycloalkyl, heteroaryl.

In a still preferred embodiment, the invention refers to at least one of the compounds listed in the Table 4 below and pharmaceutical acceptable salts thereof.

TABLE 4
List of preferred compounds of Formula (Ic)

| Compound No. | Structure | Chemical Name |
|---|---|---|
| Compound 16 | 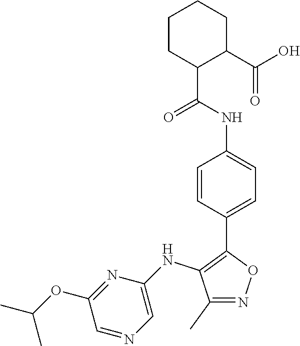 [SMILES: Cc1noc(-c2ccc(NC(=O)C3CCCCC3C(=O)O)cc2)c1Nc1cncc(OC(C)C)n1] | 2-((4-(4-((6-isopropoxypyrazin-2- yl)amino)-3-methylisoxazol-5- yl)phenyl)carbamoyl)cyclohexane carboxylic acid (trans racemate) |
| Compound 20 | 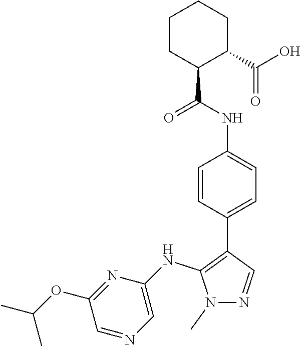 [SMILES: CC(C)Oc1cncc(Nc2c(-c3ccc(NC(=O)[C@H]4CCCC[C@@H]4C(=O)O)cc3)cnn2C)n1] | (1S,2S)-2-((4-(5-((6- isopropoxypyrazin-2-yl)amino)-1- methyl-1H-pyrazol-4- yl)phenyl)carbamoyl)cyclohexane- 1-carboxylic acid |
| Compound 43 | 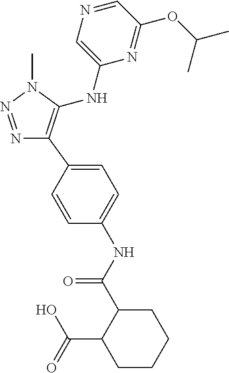 [SMILES: CC(C)Oc1cncc(Nc2c(-c3ccc(NC(=O)C4CCCCC4C(=O)O)cc3)nnn2C)n1] | yl)amino)-1-methyl-1H-1,2,3- triazol-4- yl)phenyl)carbamoyl)cyclohexane- 1-carboxylic acid (cis racemate) |

In an even more preferred embodiment, the invention refers to a compound of formula (I) as LPA1 antagonist, wherein L is —NH— and L₁ is —C(O)—, A is isoxazole, L2 is —NH—, R is pyrazine, represented by the general formula (Id)

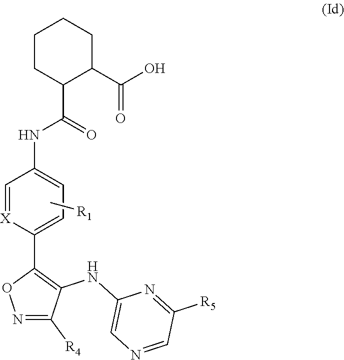

wherein X is —CH— or N;
R₁ is H or —(C₁-C₄)alkyl;
R₄ is H or —(C₁-C₄)alkyl; and
R₅ is aryl optionally substituted by one or more groups selected from —(C₁-C₄)alkyl, halo.

In another more preferred embodiment, the invention refers to a compound of formula (I) as LPA1 antagonist, wherein L is —O— and L₁ is —CH2-, A is isoxazole, L₂ is —NH—, R is pyrazine, represented by the general formula (Ie)

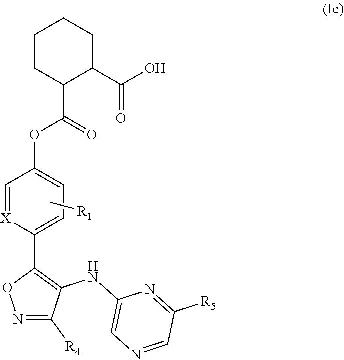

wherein X is —CH— or N;
R₁ is H or —(C₁-C₄)alkyl;
R₄ is H or —(C₁-C₄)alkyl; and
R₅ is aryl optionally substituted by one or more groups selected from —(C₁-C₄)alkyl, halo.

In another preferred embodiment, the invention refers to a compound of formula (I) as LPA1 antagonist, wherein L is —O— and L₁ is —CH2-, A is triazole, L₂ is —NH—, R₄ is methyl, R is pyrazine, represented by the general formula (If)

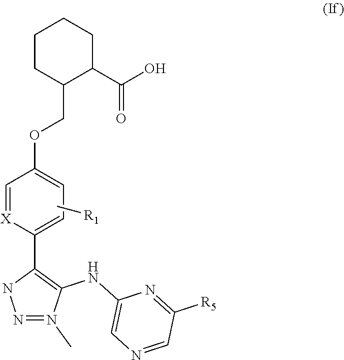

wherein X is —CH— or N;
R₁ is H or —(C₁-C₄)alkyl;
R₅ is aryl optionally substituted by one or more groups selected from —(C₁-C₄)alkyl, halo.

In another more preferred embodiment, the invention refers to a compound of formula (I) as LPA1 antagonist, wherein L is —O— and L₁ is —CH2-, A is isoxazole, L₂ is —NH—, R is —C(O)O(C₁-C₅)alkyl-R₃R₂, represented by the general formula (Ig)

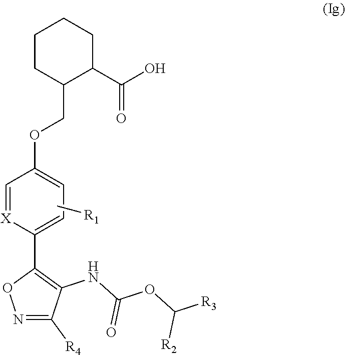

wherein X is —CH— or N;
R₁ is H or —(C₁-C₄)alkyl;
R₂ is H or selected from the group consisting of —(C₁-C₄)alkyl, aryl, heteroaryl wherein any of such aryl, heteroaryl and alkyl are optionally substituted by one or more groups selected from —(C₁-C₄)alkyl, halo, —OR₅, —R₅;
R₃ is H or selected from the group consisting of —(C₁-C₄)alkyl, —(C₁-C₄)haloalkyl, —(C₃-C₆)cycloalkyl optionally substituted by one or more halo;
R₄ is H or —(C₁-C₄)alkyl; and
R₅ is aryl optionally substituted by one or more groups selected from —(C₁-C₄)alkyl, halo.

It has been surprisingly found that the above indicated compounds are particularly effective as antagonists of LPA1 receptor, as e.g. indicated to the Table 5 of the herein below experimental part.

In this respect, it has now been found that the compounds of formula (I) of the present invention have an antagonist drug potency expressed as half maximal inhibitory concentration (IC₅₀) on LPA1 lesser than 600 nM.

Preferably, the compounds of the present invention have an IC₅₀ on LPA1 lesser or equal than 250 nM.

More preferably, the compounds of the present invention have an IC₅₀ on LPA1 lesser or equal than 50 nM.

In one aspect, the present invention refers to a compound of formula (I) for use as a medicament. Thus, the invention refers to a compound of formula (I) in the preparation of a medicament, preferably for use in the treatment of disorders associated with LPA receptors mechanism.

In a preferred embodiment, the invention refers to a compound of formula (I) for use in the treatment of disorders associated with LPA receptors mechanism.

In a further embodiment, the present invention refers to a compound of formula (I) for use in the treatment of a disease, disorder or condition associated with dysregulation of lysophosphatidic acid receptor 1 (LPA1).

In one embodiment, the present invention refers to a compound of formula (I) useful for the prevention and/or treatment of fibrosis and/or diseases, disorders, or conditions that involve fibrosis.

The terms “fibrosis” or “fibrotic disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention are useful for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CIP), kidney or renal fibrosis, ocular fibrosis, cardiac fibrosis, arterial fibrosis and systemic sclerosis.

More preferably, the compounds of formula (I) of the present invention are useful for the treatment of idiopathic pulmonary fibrosis (IPF).

In one aspect, the invention also refers to a method for the prevention and/or treatment of disorders associated with LPA receptors mechanisms, said method comprises administering to a patient in need of such treatment a therapeutically effective amount of a compound of formula (I).

In a further aspect, the invention refers to a method for the prevention and/or treatment of disorder or condition associated with dysregulation of lysophosphatidic acid receptor 1 (LPA1) administering a patient in need of such treatment a therapeutically effective amount of a compound of formula (I).

In a further aspect, the invention refers to a method for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

In a further aspect, the invention refers to the use of a compound of formula (I) according to the invention, for the treatment of disorders associated with LPA receptors mechanism.

In a further aspect, the invention refers to the use of the compound of formula (I) for the preparation of a medicament for the treatment of disorders associated with LPA receptors mechanism.

In a further aspect, the invention refers to the use of the compound of formula (I) for the preparation of a medicament for the treatment and/or prevention of fibrosis such as pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

In a further aspect, the present invention refers to the use of a compound of formula (I) for the treatment of a disease, disorder or condition associated with dysregulation of lysophosphatidic acid receptor 1 (LPA1).

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

More preferably, the compounds of the present invention are administered orally.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula I may be combined among each other and apply as well mutatis mutandis.

The various aspects of the invention described in this application are illustrated by the following examples which are not meant to limit the invention in any way.

The compounds of the present invention can be prepared in several ways known to one skilled in the art of organic synthesis. It will be understood by those skilled in the art of organic synthesis that the functionality present on the molecule should be consistent with the transformation proposed. This will sometimes require a modification of the order of synthetic steps in order to obtain a desired compound of the invention.

Thus, processes described below should not be viewed as limiting the scope of the synthetic methods available for the preparation of the compounds of the invention.

In some cases, generally known protective groups (PG) may be employed when needed to mask or protect sensitive or reactive moieties, in accordance with general principles of chemistry (Protective group in organic syntheses, 3rd ed. T. W. Greene, P. G. M. Wuts).

In the procedures that follow, some of the starting materials are identified through an “Intermediate” or “Compound” number with indications on step number. This is provided merely for assistance to the skilled chemist.

When reference is made to the use of a “similar” or “analogous” procedure, as it will be appreciated by those skilled in the art, such a procedure may involve minor variations, for example reaction temperature, reagent/solvent amount, reaction time, work-up conditions or chromatographic purification conditions, that will be appreciated by those skilled in the art.

In one embodiment of the present invention, some compounds of the invention may be prepared according to Scheme 1.

Scheme 1 describes the synthesis of isoxazole amido cyclohexane acid derivatives of formula (Id). Commercially available bromo-substituted carboxylic acid (VI) is converted to the corresponding acid chloride using a chlorinating agent, like for instance SOCl₂ or Oxalyl chloride/catalytic DMF. This acid chloride is then reacted with a suitable β-enamino-ester (VII) followed by condensation with hydroxylamine to provide isoxazole (VIII). Reaction of isoxazole (VIII) with tert-Butyl carbamate (IX) under Buchwald reaction conditions (e.g. Buchwald, S. L. et al, Chem. Rev. 2016, 116, 12564-12649) provides intermediate (X). Deprotection of the ester and subsequent Curtius rearrangement in the presence of the commercially available alcohols (XI) provide the isoxazole carbamate (XII). Cleavage of carbamate using hydrogenolysis, followed by Buchwald reaction with intermediate (XIII) afford compound (XIV). Boc removal under suitable conditions (i.e. TFA/DCM), followed by nucleophilic addition of the commercially available anhydride (XV), give the final compound (Id).

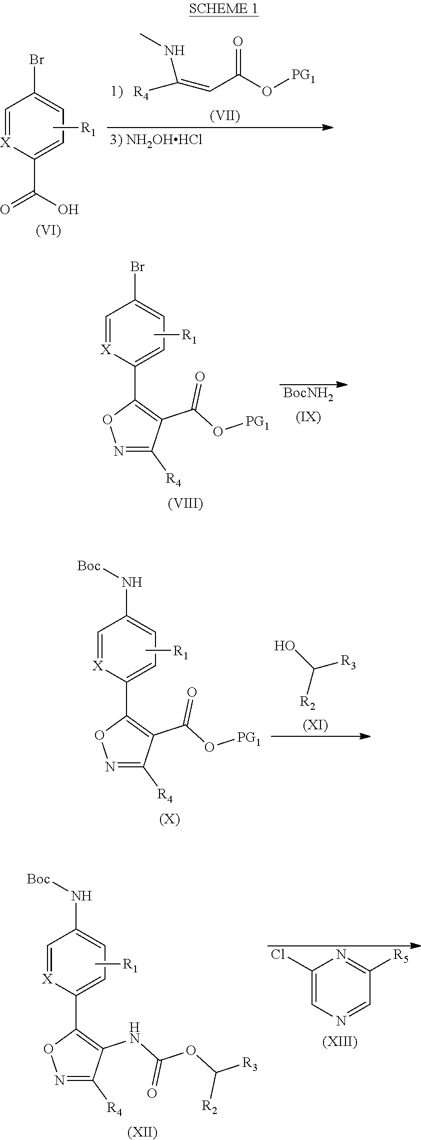

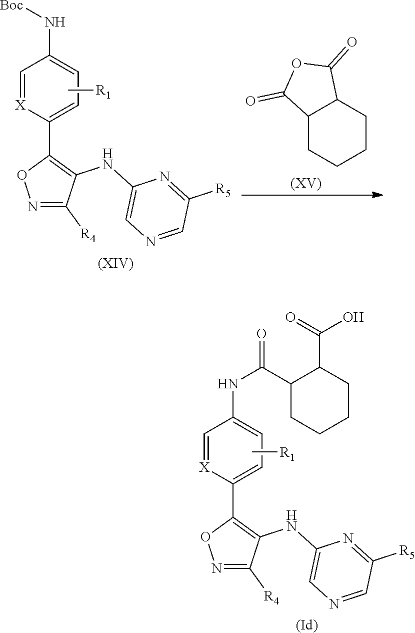

In another embodiment of the present invention, compounds of the invention of formula (Id) may be prepared according to Scheme 2.

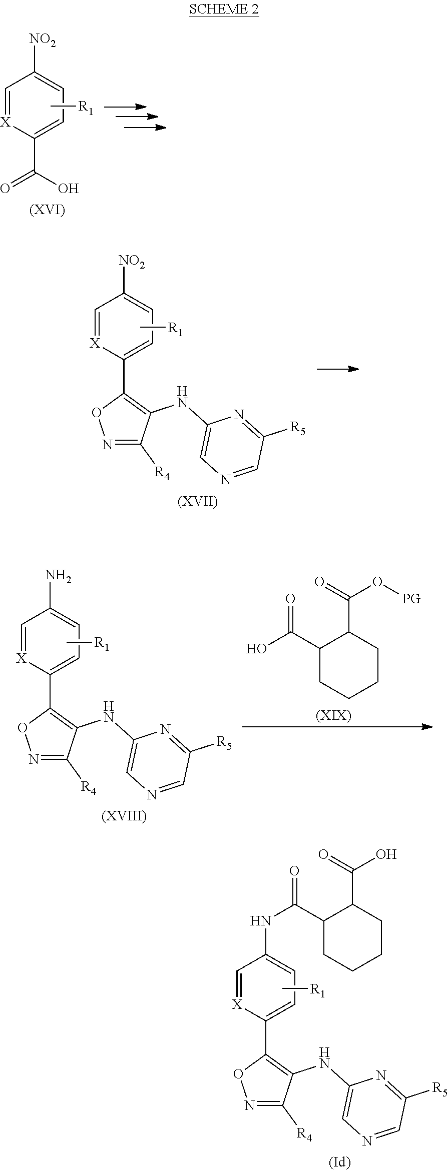

Scheme 2 describes an alternative synthetic route to isoxazole amido cyclohexane acid derivatives (Id). Commercially available nitro-substituted carboxylic acid (XVI) undergoes the same synthetic sequence previously outlined in Scheme 1 to provide intermediate (XVII). Reduction of the nitro group under suitable conditions, like for instance Iron (Fe) in acidic conditions (non-limiting example is HCl) leads to the amino intermediate (XVIII). Final compound (Id) can be obtained through amide coupling of intermediate (XVIII) with the commercially available ester carboxylic acid (XIX), followed by deprotection.

In another embodiment of the present invention, compounds of the invention of formula (Ie) may be prepared according to Scheme 3.

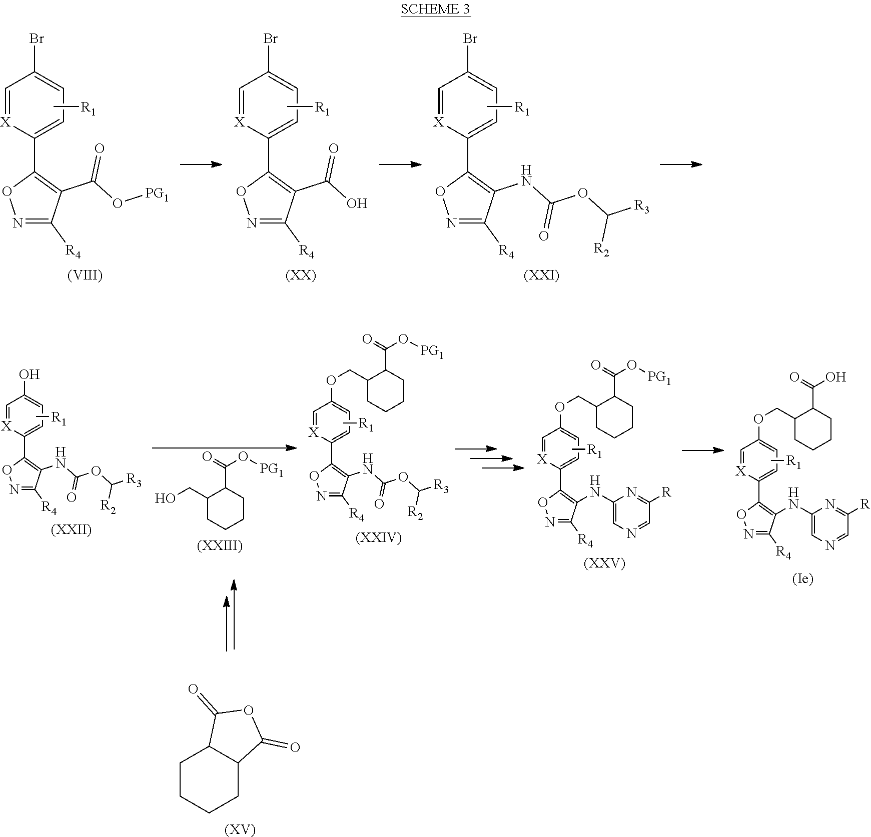

Intermediate (VIII) was deprotected to give carboxylic acid intermediate (XX) that undergoes Curtius rearrangement to provide the corresponding carbamate (XXI). Reaction of intermediate (XXI) under Miyaura Borylation conditions, followed by oxidation, allow the formation of the hydroxyl-derivative (XXII). Mitsunobu reaction of intermediate (XXII) and alcohol (XXIII), prepared from commercially available anhydride (XV), provide compound (XXIV). Derivative (XXIV) is then submitted to the synthetic sequence previously outlined in Scheme 1 to give the intermediate (XXV). Final deprotection under well-known procedures provides the final compound (Ie).

In a further embodiment of the present invention, some compounds of the invention of formula (If) may be prepared according to Scheme 4. A 4-nitro substituted halide (XXVI) undergoes Sonogashira coupling with propargyl alcohol (XXVII) in the presence of a suitable palladium catalyst such as for example Bis(triphenylphosphine)palladium(II) dichloride to give the corresponding intermediate (XXVIII). Trimethylsilyl azide (XXIX) can be used for the cycloaddition to the hydroxyl alkyl alkyne (XXVIII) to afford, after desilylation of (XXX), the desired methyl triazole (XXXI). Oxidation of the alcohol provides the corresponding triazole carboxylic acid (XXXII). Curtius rearrangement of intermediate (XXXII) and subsequent reduction of the nitro group provide compound (XXXIV). Sandmeyer reaction using NaNO₂ and H₂SO₂ provides intermediate (XXXV) that undergoes the same synthetic sequence previously outlined in Scheme 3 to provide final compound (If).

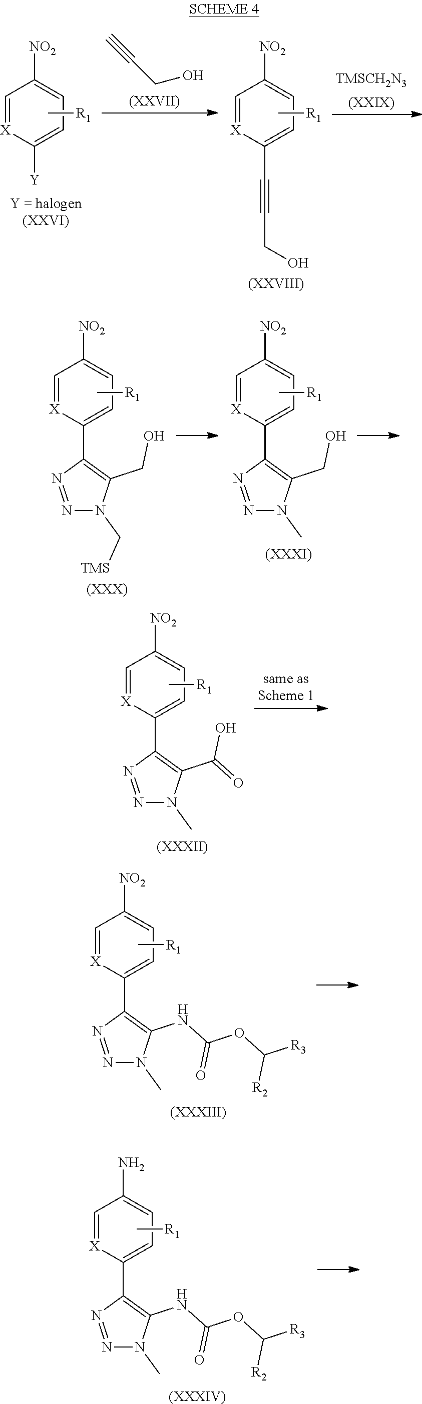

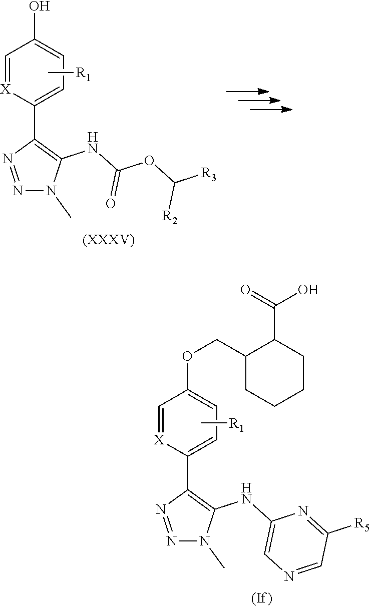

In a more embodiment of the present invention, some compounds of the invention of formula (Ig) may be prepared according to Scheme 5.

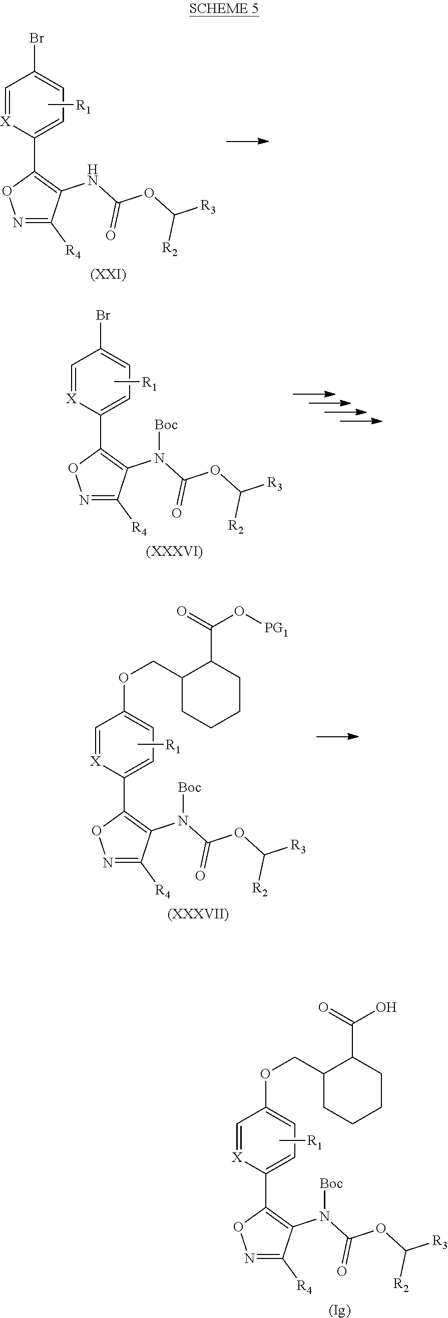

Intermediate (XXI) was submitted to Boc protection under classical conditions (i.e. (Boc)₂O, DIPEA, DMAP, DCM) to provide intermediate (XXXVI), which undergoes the same synthetic sequence previously outlined in Scheme 3 to provide intermediate (XXXVII). Final compound (Ig) is then obtained by Boc removal and ester hydrolysis.

The various aspects of the invention described in this application are illustrated by the following examples which are not meant to limit the invention in any way.

Accordingly, the present invention provides intermediate compounds (XIV), as defined above, and their use in the preparation of compounds of formula (I). In a preferred embodiment the present invention provides intermediate compounds (XIV), as defined above, and their use in the preparation of compounds of formula (Id).

In another embodiment, the present invention provides intermediate compounds (XVIII), as defined above, and their use in the preparation of compounds of formula (I). In a preferred embodiment, the present invention provides intermediate compounds (XVIII), as defined above, and their use in the preparation of compounds of formula (Id). In another embodiment, the present invention provides intermediate compounds (XXV), as defined above, and their use in the preparation of compounds of formula (I). In another preferred embodiment, the present invention provides intermediate compounds (XXV), as defined above, and their use in the preparation of compounds of formula (Ie).

In another embodiment, the present invention provides intermediate compounds (XXXV), as defined above, and their use in the preparation of compounds of formula (I). In another preferred embodiment, the present invention provides intermediate compounds (XXXV), as defined above, and their use in the preparation of compounds of formula (If). In another embodiment, the present invention provides intermediate compounds (XXXVII), as defined above, and their use in the preparation of compounds of formula (I). In another preferred embodiment, the present invention provides intermediate compounds (XXXVII), as defined above, and their use in the preparation of compounds of formula (Ig).

In another aspect, the invention provides the use of an intermediate compound selected from the group consisting of compound (XIV) or (XVIII), as defined above, in particular in Scheme 1 or 2, for the preparation of the compounds of formula (I), as defined above.

In a preferred embodiment, the invention provides the use of an intermediate compound selected from the group consisting of compound (XIV) or (XVIII), as defined above, in particular in Scheme 1 or 2, for the preparation of the compounds of formula (Id), as defined above.

In another embodiment, the invention provides the use of an intermediate compound (XXV), as defined above, in particular in Scheme 3, for the preparation of the compounds of formula (I), as defined above.

In another preferred embodiment, the invention provides the use of an intermediate compound (XXV), as defined above, in particular in Scheme 3, for the preparation of the compounds of formula (Ie), as defined above.

In another embodiment, the invention provides the use of an intermediate compound (XXXV), as defined above, in particular in Scheme 4, for the preparation of the compounds of formula (I), as defined above.

In another preferred embodiment, the invention provides the use of an intermediate compound (XXXV), as defined above, in particular in Scheme 4, for the preparation of the compounds of formula (If), as defined above.

In another embodiment, the invention provides the use of an intermediate compound (XXXVII), as defined above, in particular in Scheme 5, for the preparation of the compounds of formula (I), as defined above.

In another preferred embodiment, the invention provides the use of an intermediate compound (XXXVII), as defined above, in particular in Scheme 5, for the preparation of the compounds of formula (Ig), as defined above.

Exemplified preparation processes are given in the following experimental part.

### PREPARATIONS OF INTERMEDIATES AND EXAMPLES

All reagents, for which the synthesis is not described in the experimental part, are either commercially available, or are known compounds or may be formed from known compounds by known methods by a person skilled in the art.

The chemical names are assigned accordingly to Chem Draw Professional Version 20.1.0.110.

### Abbreviation—Meaning

B₂pin₂=Bis(pinacolato)diboron;
(Boc)₂O=Di-tert-butyl dicarbonate; CDI=1,1′-Carbonyldiimidazole; Cs₂CO₃=Cesium carbonate
Cp*RuCl(PPh₃)₂=Pentamethylcyclopentadienylbis(triphenylphosphine)ruthenium(II) chloride;
CuI=copper iodide;
CyHex=Cyclohexane;
DavePhos=2-(2-dicyclohexylphosphinophenyl)-N,N-dimethylaniline;
DBAD=di-tert-butyl azodicarboxylate;
DCE=1,2-dichloroethane;
DCM=Dichloromethane
DMAP=4-Dimethylaminopyridine
DMF=Dimethylformamide
DMSO=Dimethylsulfoxide
DPPA=Diphenylphosphoryl azide
EtOAc=Ethyl acetate
Fe⁰=iron
HCl=Hydrochloric acid
HCOOH=Formic acid
H₂O=Water
H₂SO₄=sulfuric acid
K₂CO₃=Potassium carbonate
KF=Potassium fluoride
K₂HPO₄=Potassium phosphate dibasic
KHSO₄=Potassium bisulfate
KOAc=Potassium acetate
KMnO₄=Potassium permanganate
K₃PO₄=Potassium phosphate tribasic
LC-MS=liquid chromatography/mass spectrometry
LiOH=Litium hydroxyde
MeCN=Acetonitrile
MeOH=Methanol
N₂=Nitrogen
NaBH₄=Sodium borohydride
NaOH═Sodium hydroxide
Na₂CO₃=Sodium carbonate
NaHCO₃=Sodium bicarbonate
Na₂SO₄=Sodium sulfate
NH₄Cl=Ammonium chloride
PdCl₂(PPh₃)₂=Bis(triphenylphosphine)palladium(II) dichloride
Pd(dppf)Cl₂=[1,1′-Bis(diphenylphosphino)ferrocene]dichloropalladium(II)
Pd(PPh₃)₄=Tetrakis(triphenylphosphine)palladium(0)
PTSA=p-Toluenesulfonic acid
RT=room temperature
SnCl₂·2H₂O=Tin(II) chloride dihydrate
TFA=Trifluoroacetic acid
TMSN3=trymethylsilylazide
TBAF=tetrabutylammonium fluoride
THF=Tetrahydrofuran
XantPhos=4,5-Bis(diphenylphosphino)-9,9-dimethylxanthene
XPhos Pd G2=Chloro(2-dicyclohexylphosphino-2′,4′,6′-triisopropyl-1,1′-biphenyl)[2-(2′-amino-1,1′-biphenyl)]palladium(II)

### GENERAL EXPERIMENTAL DETAILS AND METHODS

### Analytical Method

### Instruments, Materials and Methods Employed for Analyses

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift, or on AgilentVNMRS-500/600 or on a Bruker Avance 300/400 spectrometers or on a bruker Fourier 300. Chemical shift are reported as 6 values in ppm relative to trimethylsilane (TMS) as an internal standard. Coupling constants (J values) are given in hertz (Hz) and multiplicities are reported using the following abbreviation (s=singlet, d=doublet, t=triplet, q=quartet, m=multiplet, br. s=broad singlet, nd=not determined).

### LC/UV/MS Analytical Methods

LC/MS retention times are estimated to be affected by an experimental error of +0.5 min. LCMS may be recorded under the following conditions: diode array DAD chromatographic traces, mass chromatograms and mass spectra may be taken on UPLC/PDA/MS Acquity™ system coupled with Micromass ZQ™ or Waters SQD single quadrupole mass spectrometer or Waters Alliance e2695 HPLC with Photodiode Detector 2998 coupled with Column Oven and Mass Spectrometer ZQ operated in positive and/or negative electron spray ES ionization mode and/or Fractionlynx system used in analytical mode coupled with ZQ™ single quadrupole operated in positive and/or negative ES ionisation mode or on a Shimadzu LCMS-2020 Single Quadrupole Liquid Chromatograph Mass Spectrometer and LCMS spectra were measured on Dionex UHPLC Ultimate 3000 with DAD detector/Thermo Scientific MSQ Plus.

Quality Control methods used operated under low pH conditions or under high pH conditions:

Method 1, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min.

The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 2, low pH conditions: column Acquity UPLC 1.7 μm BEH-C18 (2.1×100 mm) 130A, the column temperature was 40° C.; mobile phase solvent A was water+0.1% HCOOH, mobile phase solvent B MeCN+0.1% HCOOH. The flow rate was 0.5 mL/min. The gradient table was t=0.10 min 80% A 20% B, t=4.00 min 5% A, 95% B, t=5.00 min 5% A, 95% B, t=5.20 min 80% A, 20% B, t=6.00 min 80% A, 20% B, UV detection was 220 and 254 nm and ES+/ES− range was 100 to 1000 AMU.

Method 3, low pH conditions: column Kinetex®2.6 μm XB-C18 (4.6×50 mm), 110A, the column temperature was 25° C.; mobile phase solvent A was pQ-water for LCMS+0.1% HCOOH, mobile phase solvent B MeCN+0.1% HCOOH. The flow rate was 1 mL/min. The gradient table was t=0.00 min 50% A 50% B, t=3.35 min 20% A, 80% B, t=3.75 min 20% A, 80% B, t=3.90 min 5% A, 95% B, t=4.75 min 5% A, 95% B, t=5.00 min 50% A, 50% B, t=6.00 min 50% A, 50% B, UV detection range was 190-340 nm and ES+/ES− range was 100 to 1000 AMU.

Method 4, low pH conditions: column Acquity UPLC 1.7 μm BEH-C18 (2.1×100 mm) 130A, the column temperature was 40° C.; mobile phase solvent A was water+0.1% HCOOH, mobile phase solvent B MeCN+0.1% HCOOH. The flow rate was 0.5 mL/min. The gradient table was t=0.00 min 80% A 20% B, t=0.10 min 80% A 20% B, t=1.10 min 0% A, 100% B, t=2.00 min 0% A, 100% B, t=2.50 min 80% A, 20% B, t=3.00 min 80% A, 20% B, UV detection range was 200-400 nm and ES+/ES− range was 100 to 1000 AMU.

Method 5, high pH conditions: column Acquity UPLC 1.7 μm BEH-C18 (2.1×100 mm) 130A, the column temperature was 40° C.; mobile phase solvent A was 0.05% pQ water solution of ammonium hydroxide (28.0-30.0% NH₃ basis), mobile phase solvent B MeCN. The flow rate was 0.5 mL/min. The gradient table was t=0.00 min 80% A 20% B, t=0.10 min 80% A, 20% B, t=1.10 min 0% A, 100% B, t=2.00 min 0% A, 100% B, t=2.50 min 80% A, 20% B, t=3.00 min 80% A, 20% B, UV detection range was 200-400 nm and ES+/ES− range was 100 to 1000 AMU.

Method 6, low pH conditions: column Kinetex®2.6 μm XB-C18 (4.6×50 mm), 110A, the column temperature was 25° C.; mobile phase solvent A was pQ-water for LCMS+0.1% HCOOH, mobile phase solvent B MeCN+0.1% HCOOH. The flow rate was 1 mL/min. The gradient table was t=0.00 min 80% A 20% B, t=3.35 min 20% A, 80% B, t=3.75 min 20% A, 80% B, t=3.90 min 5% A, 95% B, t=4.75 min 5% A, 95% B, t=5.00 min 80% A, 20% B, t=6.00 min 80% A, 20% B, UV detection range was 190-340 nm and ES+/ES− range was 100 to 1000 AMU.

Method 7, high pH conditions: column Kinetex®2.6 μm XB-C18 (4.6×50 mm), 110A, the column temperature was 40° C.; mobile phase solvent A was 0.05% pQ water solution of ammonium hydroxide (28.0-30.0% NH₃ basis), mobile phase solvent B MeCN. The flow rate was 1 mL/min. The gradient table was t=0.00 min 70% A 30% B, t=3.35 min 30% A, 70% B, t=3.75 min 30% A, 70% B, t=3.90 min 5% A, 95% B, t=4.75 min 5% A, 95% B, t=5.00 min 70% A, 30% B, t=6.00 min 70% A, 30% B, UV detection range was 190-350 nm and ES+/ES− range was 100 to 1000 AMU.

Method 8, low pH conditions: column Kinetex®2.6 μm XB-C18 (4.6×50 mm), 110A, the column temperature was 25° C.; mobile phase solvent A was pQ-water for LCMS+0.1% HCOOH, mobile phase solvent B MeCN+0.1% HCOOH. The flow rate was 1 mL/min. The gradient table was t=0.00 min 60% A 40% B, t=3.35 min 20% A, 80% B, t=3.75 min 20% A, 80% B, t=3.90 min 5% A, 95% B, t=4.75 min 5% A, 95% B, t=5.00 min 60% A, 40% B, t=6.00 min 60% A, 40% B, UV detection range was 190-340 nm and ES+/ES− range was 100 to 1000 AMU.

Method 9, low pH conditions: column Kinetex®2.6 μm XB-C18 (4.6×50 mm), 110A, the column temperature was 40° C.; mobile phase solvent A was 0.05% μQ-water for LCMS+0.1% HCOOH, mobile phase solvent B MeCN+0.1% HCOOH. The flow rate was 1 mL/min. The gradient table was t=0.00 min 70% A 30% B, t=3.35 min 30% A, 70% B, t=3.75 min 30% A, 70% B, t=3.90 min 5% A, 95% B, t=4.75 min 5% A, 95% B, t=5.00 min 70% A, 30% B, t=6.00 min 70% A, 30% B, UV detection range was 190-340 nm and ES+/ES− range was 100 to 1000 AMU.

Method 10, low pH conditions: column Kinetex®2.6 μm XB-C18 (4.6×50 mm), 110A, the column temperature was 40° C.; mobile phase solvent A was 0.05% pQ-water for LCMS+0.1% HCOOH, mobile phase solvent B MeCN+0.1% HCOOH. The flow rate was 1 mL/min. The gradient table was t=0.00 min 70% A 30% B, t=3.35 min 20% A, 80% B, t=3.75 min 20% A, 80% B, t=3.90 min 5% A, 95% B, t=4.75 min 5% A, 95% B, t=5.00 min 70% A, 30% B, t=6.00 min 70% A, 30% B, UV detection range was 190-340 nm and ES+/ES− range was 100 to 1000 AMU.

Method 11, low pH conditions: column Kinetex®2.6 μm XB-C18 (4.6×50 mm), 110A, the column temperature was 25° C.; mobile phase solvent A was pQ-water for LCMS+0.1% HCOOH, mobile phase solvent B MeCN+0.1% HCOOH. The flow rate was 1 mL/min. The gradient table was t=0.00 min 30% A 70% B, t=3.35 min 20% A, 80% B, t=3.75 min 20% A, 80% B, t=3.90 min 5% A, 95% B, t=4.75 min 5% A, 95% B, t=5.00 min 30% A, 70% B, t=6.00 min 30% A, 70% B, UV detection range was 190-340 nm and ES+/ES− range was 100 to 1000 AMU.

Method 12, low pH conditions: column: Phenomenex Gemini-NX C18, 150×2.0 mm, 3 μm with security guard Gemini-NX C18, 4×2.0 mm, 3 μm, the column temperature was 25° C.; mobile phase solvent A was water+0.1% Formic acid filtered with 0.22 μm nylon filter, mobile phase solvent B Acetonitrile+0.1% Formic acid filtered with 0.22 μm nylon filter. The flow rate was 0.2 mL/min. The gradient table was t=0 min 95% A 5% B, t=12 min 20% A 80% B, t=35 min 20% A 80% B. The UV detection λ was 214 nm and ES+ range was 50 to 900 Da.

Method 13, low pH conditions: column: Phenomenex Gemini-NX C18, 150×2.0 mm, 3 μm with security guard Gemini-NX C18, 4×2.0 mm, 3 μm, the column temperature was 25° C.; mobile phase solvent A was water+0.1% Formic acid filtered with 0.22 μm nylon filter, mobile phase solvent B Acetonitrile+0.1% Formic acid filtered with 0.22 μm nylon filter. The flow rate was 0.2 mL/min. The gradient table was t=0 min 95% A 5% B, t=15 min 15% A 85% B, t=35 min 15% A 85% B. The UV detection λ was 215 nm and ES+/ES− range was 50 to 900 Da.

Method 14, low pH conditions: column: Phenomenex Gemini-NX C18, 150×2.0 mm, 3 μm with security guard Gemini-NX C18, 4×2.0 mm, 3 μm, the column temperature was 25° C.; mobile phase solvent A was water+0.1% Formic acid filtered with 0.22 μm nylon filter, mobile phase solvent B Acetonitrile+0.1% Formic acid filtered with 0.22 μm nylon filter. The flow rate was 0.2 mL/min. The gradient table was t=0 min 90% A 10% B, t=12 min 10% A 90% B, t=35 min 10% A 90% B. The UV detection X was 214 nm and ES+ range was 50 to 900 Da.

Method 15: low pH conditions: column: Phenomenex Gemini-NX C18, 150×2.0 mm, 3 μm with security guard Gemini-NX C18, 4×2.0 mm, 3 μm, the column temperature was 25° C.; mobile phase solvent A was water+0.1% Formic acid filtered with 0.22 μm nylon filter, mobile phase solvent B Acetonitrile+0.1% Formic acid filtered with 0.22 μm nylon filter. The flow rate was 0.2 mL/min. The gradient table was t=0 min 95% A 5% B, t=10 min 5% A 95% B, t=35 min 5% A 95% B. The UV detection λ was 215 nm and ES+ range was 50 to 900 Da.

### Chiral Preparative HPLC for Chiral Compounds

Chiral resolutions were performed using a Semipreparative Waters 600 system or a Semipreparative Agilent 1100 system. The conditions are reported in the Examples.

Where the preparation of starting materials is not described, these are commercially available, known in the literature, or readily obtainable by those skilled in the art using standard procedures.

Flash chromatography is carried out using an Isolera MPLC system (manufactured by Biotage) using pre-packed silica gel or reverse-phase cartridges (supplied by Biotage or Sepachrom).

Many of the compounds described in the following Examples have been prepared from stereochemically pure starting materials, for example 95% ee.

The stereochemistry of the compounds in the Examples, where indicated, has been assigned on the assumption that absolute configuration at resolved stereogenic centers of staring materials is maintained throughout any subsequent reaction conditions.

In the procedures that follow, after each starting material, reference to a compound number is sometimes provided. This is provided merely for assistance to the skilled chemist. The starting material may not necessarily have been prepared from the batch referred to.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

### Example 1

### Process of Synthesis of Compound 1

### (1S,2S)-2-((6-(4-((6-cyclopropoxypyrazin-2-yl)amino)-3-methylisoxazol-5-yl)-2-methylpyridin-3-yl)carbamoyl)cyclohexane-1-carboxylic acid

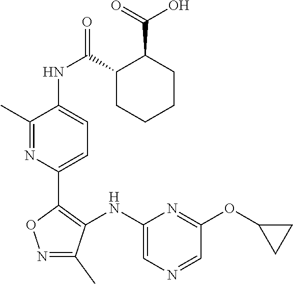 [SMILES: Cc1nc(-c2onc(C)c2Nc2cncc(OC3CC3)n2)ccc1NC(=O)[C@H]1CCCC[C@@H]1C(=O)O]

### Step 1: 5-bromo-6-methylpicolinoyl chloride (Intermediate A1)

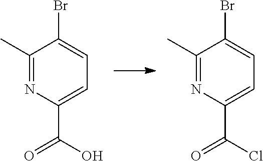

Oxalyl dichloride (19.85 mL, 231.45 mmol) was added at 0° C. to a suspension of 5-bromo-6-methylpicolinic acid (25.0 g, 115.72 mmol) in dry DCM (270 mL) and dry DMF (0.27 mL, 3.47 mmol). The mixture was then stirred at RT for 2 h and the solvent was removed under reduced pressure to give the desired compound (27 g, crude) as a brown solid.

¹H NMR (400 MHz, DMSO-d₆) δ ppm 8.19 (d, J 8.22 Hz, 1H), 7.78 (d, J 8.22 Hz, 1H), 2.64 (s, 3H)

### Step 2: methyl (Z)-2-(5-bromo-6-methylpicolinoyl)-3-(methylimino)butanoate (Intermediate A2)

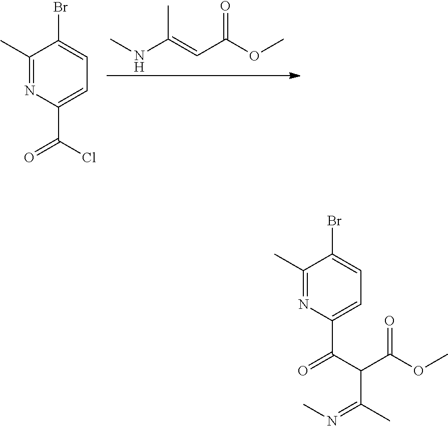

To a solution of methyl (E)-3-(methylamino)but-2-enoate (14.87 g, 115.15 mmol), prepared according to the procedure reported in J. Org. Chem., 1965, 30, 3033-3037, in dry THE (100 mL), Pyridine (13.91 mL, 172.72 mmol) was added dropwise at RT. The mixture was cooled to 0 C and a solution of 5-bromo-6-methylpyridine-2-carbonyl chloride (Intermediate A1, 27.0 g, crude) in dry THE (250 mL) was slowly added. The mixture was then allowed to warm up to RT and stirred overnight. THE was partially removed then H₂O was added and the mixture was extracted with EtOAc for 3 times. The combined organic phases were washed with water and brine, dried over Na2SO4, filtered and concentrated in vacuo to give the title product (29 g, 88.64 mmol, 76.98% yield) as a brown oil that was used in the next step without further purification.

LC-MS (ESI): m/z (M+1): 329.1 (Method 1)

### Step 3: methyl 5-(5-bromo-6-methylpyridin-2-yl)-3-methylisoxazole-4-carboxylate (Intermediate A3)

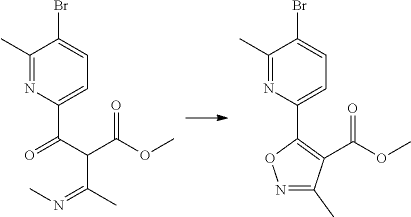

Methyl (Z)-2-(5-bromo-6-methylpicolinoyl)-3-(methylimino)butanoate (Intermediate A2, 29 g, 88.64 mmol,) was dissolved in Acetic acid (186.38 mL) and hydroxylamine hydrochloride (5.97 g, 85.98 mmol) was added and the mixture was heated at 80° C. for 15 min. The mixture was evaporated in vacuum and NaHCO₃saturated aqueous solution (400 mL) was added and the mixture was extracted with EtOAc (500 mL) for 3 times. The combined organic layer was further washed with water and brine, dried over Na2SO4, and solved removed under reduced pressure. The crude was purified by flash chromatography eluting with a gradient of DCM/EtOAc from 100/0 to 90/10 and then the solid residue was triturated with EtOAc/cyclohexane to provide title compound (14.6 g, 46.93 mmol, 52.94% yield) as a white solid.

LC-MS (ESI): m/z (M+1): 313 (Method 1)

¹H NMR (400 MHz, Chloroform-d) δ 7.97 (d, J=8.19 Hz, 1H), 7.65 (d, J=8.19 Hz, 1H), 3.87 (s, 3H), 2.76 (s, 3H), 2.52 (s, 3H)

The Intermediate in the following table was prepared from reagents reported below by using methods analogous to Intermediate A3.

| Intermediate | Structure & Name | Reagents | Analytical data |
|---|---|---|---|
| A4 | 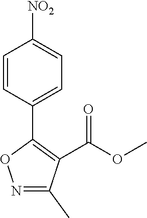 [SMILES: COC(=O)c1c(C)noc1-c1ccc([N+](=O)[O-])cc1] | 4- nitrobenzoyl chloride | LC-MS (ESI): m/z (M + 1): 263.1 (Method 1) ¹H NMR (400 MHz, Chloroform-d) δ 8.44-8.30 (m, 2H). 8.23- 8.07 (m, 2H), 3.90 (s, 3H), 2.56 (s, 3H) |
| | methyl 3-methyl-5-(4- | | |
| | nitrophenyl)isoxazole-4- | | |
| | carboxylate | | |
| A5 | 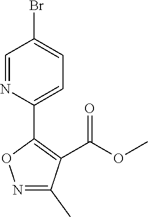 [SMILES: COC(=O)c1c(C)noc1-c1ccc(Br)cn1] | 5- bromopicolinic acid | LC-MS (ESI): m/z (M + 1): 296.0 (Method 12) |
| | methyl 5-(5- | | |
| | bromopyridin-2-yl)-3- | | |
| | methylisoxazole-4- | | |
| | carboxylate | | |
| A6 | 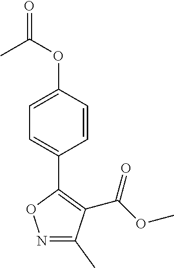 [SMILES: COC(=O)c1c(C)noc1-c1ccc(OC(C)=O)cc1] | 4- acetoxybenzoic acid | LC-MS (ESI): m/z (M + 1): 276.0 (Method 13) |
| | methyl 5-(4- | | |
| | hydroxyphenyl)-3- | | |
| | methylisoxazole-4- | | |
| | carboxylate | | |
| A7 | 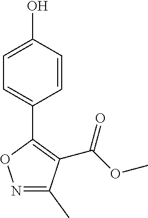 [SMILES: COC(=O)c1c(C)noc1-c1ccc(O)cc1] | 4- acetoxybenzoic acid | LC-MS (ESI): m/z (M + 1): 234.5 (Method 2) ¹H NMR (300 MHZ, DMSO-d₆) δ 10.27 (s, 1H), 7.83-7.67 (m, 2H), 7.01- 6.80 (m, 2H), 3.78 (s, 3H), 2.40 (s, 3H) |
| | methyl 5-(4- | | |
| | hydroxyphenyl)-3- | | |
| | methylisoxazole-4- | | |
| | carboxylate | | |

### Step 4: methyl 5-(5-tert-butoxycarbonyl)amino)-6-methylpyridin-2-yl)-3-methylisoxazole-4-carboxylate (Intermediate A8)

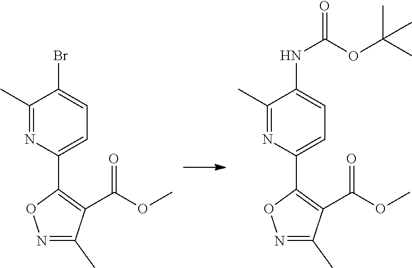

A two necked round bottomed flask equipped with a reflux condenser under argon atmosphere was loaded with XantPhos (2.13 g, 3.67 mmol), Cs₂CO₃ (26.6 g, 81.64 mmol), carbamic acid tert-butyl ester (7.17 g, 61.23 mmol), methyl 5-(5-bromo-6-methylpyridin-2-yl)-3-methyl-1,2-oxazole-4-carboxylate (Intermediate A3, 12.7 g, 40.82 mmol), 1,4-Dioxane (125 mL). The solution was degassed with argon for 10 min, then Pd₂(dba)₃ (1121.36 mg, 1.22 mmol) was added and the solution further degassed with argon for 5 min. The mixture was refluxed for 3 h at 110° C. The reaction was allowed to cool down to RT, then water (200 ml) was added and the reaction extracted with EtOAc (100 ml) for 3 times. The collected organic layers were dried over Na2SO4, filtered and concentrated. The crude was purified by flash chromatography using a gradient of CyHex/DCM/EtOAc from 80:10:10 to 60:20:20 to afford the target compound (12.8 g, 36.86 mmol, 90% yield).

LC-MS (ESI): m/z (M+1): 487.2 (Method 1)

¹H NMR (400 MHz, Chloroform-d) δ 8.46 (d, J=8.59 Hz, 1H), 7.84-7.77 (m, 1H), 6.50 (s, 1H), 3.85 (s, 3H), 2.59 (s, 3H), 2.51 (s, 3H), 1.48 (s, 9H)

### Step 5: 5-(5-((tert-butoxycarbonyl)amino)-6-methylpyridin-2-yl)-3-methylisoxazole-4-carboxylic acid (Intermediate A9)

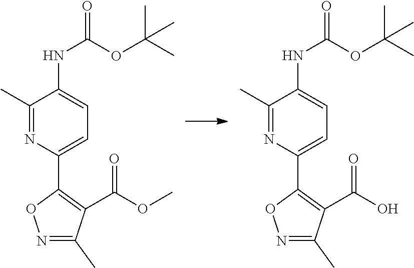 [SMILES: COC(=O)c1c(C)noc1-c1ccc(NC(=O)OC(C)(C)C)c(C)n1 ; Cc1nc(-c2onc(C)c2C(=O)O)ccc1NC(=O)OC(C)(C)C]

Methyl 5-(5-((tert-butoxycarbonyl)amino)-6-methylpyridin-2-yl)-3-methylisoxazole-4-carboxylate (Intermediate A8, 6000.0 mg, 17.27 mmol) was dissolved in THE (115.27 mL) and LiOH hydrate 1 M in water (24.18 mL, 24.18 mmol) was added dropwise at RT. The solution was stirred for 4 h at 50 C. The reaction was cooled to 0 C and neutralized by slow addition of 25 mL aqueous HCl 1 M. Then volatiles were removed and the solid dried to afford the title compound (6.4 g, 17.03 mmol, 98.62% yield) as a white solid.

LC-MS (ESI): m/z (M+1): 334.3 (Method 1)

The intermediates in the following table were prepared from reagents reported below following similar procedures as for intermediate A9.

| Intermediate | Structure & Name | Reagents | Analytical data |
|---|---|---|---|
| A10 | 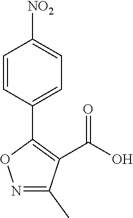 [SMILES: Cc1noc(-c2ccc([N+](=O)[O-])cc2)c1C(=O)O] | A4 | LC-MS (ESI): m/z (M + 1): 249.2 (Method 1) |
| | 3-methyl-5-(4- | | |
| | nitrophenyl)isoxazole-4- | | |
| | carboxylic acid | | |
| A11 | 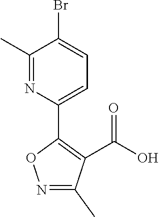 [SMILES: Cc1nc(-c2onc(C)c2C(=O)O)ccc1Br] | A3 | LC-MS (ESI): m/z (M + 1): 297.1 (Method 1) ¹H NMR (400 MHZ, DMSO-d6) δ 14.87 (s, 1H), 8.36 (d, J = 8.4 Hz, 1H), 7.90 (d, J = 8.4 Hz, 1H), 2.67 (s, 3H), 2.46 (s, 3H). |
| | 5-(5-bromo-6- | | |
| | methylpyridin-2-yl)-3- | | |
| | methylisoxazole-4- | | |
| | carboxylic acid | | |
| A12 | 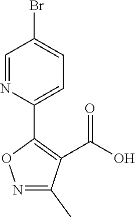 [SMILES: Cc1noc(-c2ccc(Br)cn2)c1C(=O)O] | A5 | LC-MS (ESI): m/z (M + 1): 285.1 (Method 12) |
| | 5-(5-bromopyridin-2-yl)- | | |
| | 3-methylisoxazole-4- | | |
| | carboxylic acid | | |
| A13 | 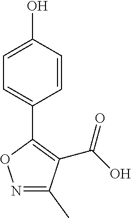 [SMILES: Cc1noc(-c2ccc(O)cc2)c1C(=O)O] | A7 | LC-MS (ESI): m/z (M + 1): 219.9 (Method 13) |
| | 5-(4-hydroxyphenyl)-3- | | |
| | methylisoxazole-4- | | |
| | carboxylic acid | | |

### Step 6: benzyl (5-(5-((tert-butoxycarbonyl)amino)-6-methylpyridin-2-yl)-3-methylisoxazol-4-yl)carbamate (Intermediate A14)

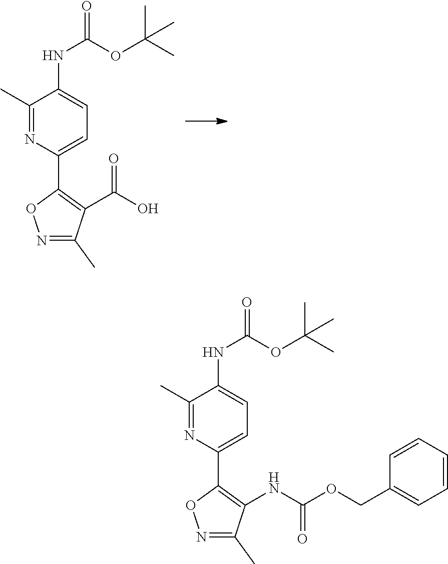 [SMILES: Cc1nc(-c2onc(C)c2C(=O)O)ccc1NC(=O)OC(C)(C)C ; Cc1nc(-c2onc(C)c2NC(=O)OCc2ccccc2)ccc1NC(=O)OC(C)(C)C]

In a 250 mL round bottom flask under N₂ atmosphere 5-(5-((tert-butoxycarbonyl)amino)-6-methylpyridin-2-yl)-3-methylisoxazole-4-carboxylic acid (Intermediate A9, 4.5 g, 13.5 mmol) was suspended in dry Toluene (90 mL). phenylmethanol (4.17 mL, 40.5 mmol), TEA (7.53 mL, 54 mmol) and DPPA (7.0 mL, 32.56 mmol) were added and the reaction was heated at reflux for 1 h. The mixture was cooled down at RT, EtOAc (50 mL) and sat. aq. NaHCO₃ (50 mL). The layers were separated and the aqueous phase was extracted with EtOAc (30 mL). The collected organic phases were washed with brine (50 mL), dried over Na2SO4, filtered and concentrated under reduced pressure. The product (5.9 g, crude) was used as a crude without any further purifications.

LC-MS (ESI): m/z (M+1): 439.3 (Method 1)

The intermediates in the following table were prepared from reagents reported below following similar procedures as for Intermediate A14.

| Intermediate | Structure & Name | Reagents | Analytical data |
|---|---|---|---|
| A15 | 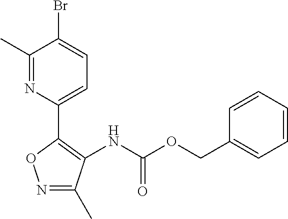 [SMILES: Cc1nc(-c2onc(C)c2NC(=O)OCc2ccccc2)ccc1Br] | A11 and phenylmethanol | LC-MS (ESI): m/z (M + 1): 404.1 (Method 1) 1H NMR (400 MHz, DMSO-d₆) δ 9.16 (s, 1H), 8.17 (d, J = 8.34 Hz, 1H), 7.58 (d, J = 8.29 Hz, 1H), 7.36 (s, 5H), 5.11 (s, 2H), 2.58 (s, 3H), 2.21 (s, 3H). |
| | benzyl (5-(5-bromo-6- | | |
| | methylpyridin-2-yl)-3- | | |
| | methylisoxazol-4- | | |
| | yl)carbamate | | |
| A16 | 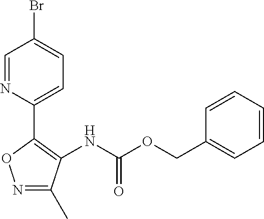 [SMILES: Cc1noc(-c2ccc(Br)cn2)c1NC(=O)OCc1ccccc1] | A12 and phenylmethanol | LC-MS (ESI): m/z (M + 1): 387.1 (Method 1) |
| | benzyl (5-(5- | | |
| | bromopyridin-2-yl)-3- | | |
| | methylisoxazol-4- | | |
| | yl)carbamate | | |
| A17 | 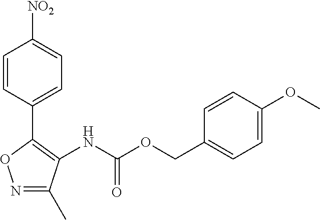 [SMILES: COc1ccc(COC(=O)Nc2c(C)noc2-c2ccc([N+](=O)[O-])cc2)cc1] | A10 and (4- methoxyphenyl) methanol | LC-MS (ESI): m/z (M + 1): 384.2 (Method 1) |
| | 4-methoxybenzyl (3- | | |
| | methyl-5-(4- | | |
| | nitrophenyl)isoxazol-4- | | |
| | yl)carbamate | | |
| A18 | 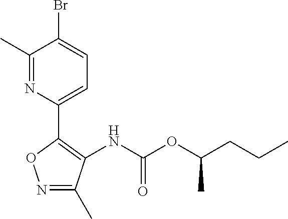 [SMILES: CCC[C@@H](C)OC(=O)Nc1c(C)noc1-c1ccc(Br)c(C)n1] | A11 and (R)-pentan- 2-ol | LC-MS (ESI): m/z (M + 1): 382.1 (Method 1) 1H NMR (400 MHz, DMSO-d6) δ ppm 9.03- 8.83 (m, 1 H), 8.20 (d, J = 8.36 Hz, 1 H), 7.58 (d, J = 8.58 Hz, 1 H), 4.78- 4.63 (m, 1 H), 2.63 (s, 3 H), 2.21 (s, 3 H), 1.66- 1.05 (m, 7 H), 0.94-0.78 (m, 3 H) |
| | (R)-pentan-2-yl (5-(5- | | |
| | bromo-6-methylpyridin- | | |
| | 2-yl)-3-methylisoxazol- | | |
| | 4-yl)carbamate | | |
| A19 | 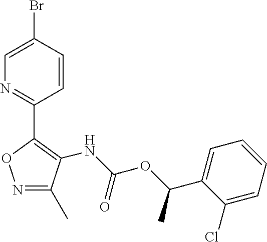 [SMILES: Cc1noc(-c2ccc(Br)cn2)c1NC(=O)O[C@H](C)c1ccccc1Cl] | A11 and (R)-1-(2- chlorophenyl) ethan-1-ol | LC-MS (ESI): m/z (M + 1): 438.1 (Method 14) |
| | (R)-1-(2- | | |
| | chlorophenyl)ethyl (5-(5- | | |
| | bromopyridin-2-yl)-3- | | |
| | methylisoxazol-4- | | |
| | yl)carbamate | | |
| A20 | 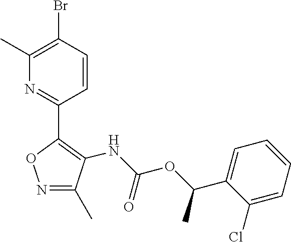 [SMILES: Cc1nc(-c2onc(C)c2NC(=O)O[C@H](C)c2ccccc2Cl)ccc1Br] | A11 and (R)-1-(2- chlorophenyl) ethan-1-ol | LC-MS (ESI): m/z (M + 1): 452.1 (Method 14) |
| | (R)-1-(2- | | |
| | chlorophenyl)ethyl (5-(5- | | |
| | bromo-6-methylpyridin- | | |
| | 2-yl)-3-methylisoxazol- | | |
| | 4-yl)carbamate | | |

### Step 7: tert-butyl (6-(4-amino-3-methylisoxazol-5-yl)-2-methylpyridin-3-yl)carbamate (Intermediate A21)

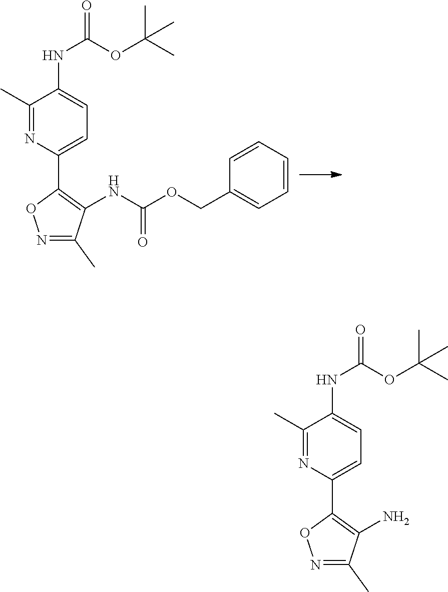 [SMILES: Cc1nc(-c2onc(C)c2NC(=O)OCc2ccccc2)ccc1NC(=O)OC(C)(C)C ; Cc1nc(-c2onc(C)c2N)ccc1NC(=O)OC(C)(C)C]

To a solution of benzyl (5-(5-((tert-butoxycarbonyl)amino)-6-methylpyridin-2-yl)-3-methylisoxazol-4-yl)carbamate (Intermediate A14, 2.6 g, 5.93 mmol) in MeOH (50 mL) was added palladium on charcoal (200.0 mg, 0.190 mmol). The mixture was stirred at RT for 1 h. The mixture was filtered over Celite, washing with MeOH (3×5 mL), and the filtrate was concentrated under reduced pressure. The crude was purified by flash chromatography using a gradient of CyHex/EtoAc from 80:20 to 0:100 to afford the target product (1.24 g, 41 mmol, 69% yield).

LC-MS (ESI): m/z (M+1): 305.3 (Method 1)

### Step 8: 2-chloro-6-cyclopropoxypyrazine (Intermediate A22)

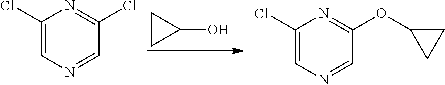

A suspension of sodium hydride (81.75 mg, 2.04 mmol) in anhydrous THE (1.053 mL) was cooled to 0° C. in an ice-water bath and cyclopropanol (0.08 mL, 1.68 mmol) was added dropwise. After 20 min 2,6-dichloropyrazine (200.0 mg, 1.34 mmol) was added and the reaction was stirred at RT overnight. The mixture was quenched with saturated aqueous NH₄Cl and EtOAc. The organic phase was washed with water, dried over Na2SO4, filtered and concentrated under reduced pressure to afford the title compound (218 mg, 1.278 mmol, 95.19% yield) as a pale yellow oil.

LC-MS (ESI): m/z (M+1): 171.0 (Method 1)

¹H NMR (400 MHz, Chloroform-d) δ ppm 8.21 (d, J=0.56 Hz, 1H), 8.13 (d, J=0.57 Hz, 1H), 4.32 (tt, J=6.16, 3.16 Hz, 1H), 1.02-0.78 (m, 4H)

The Intermediates in the following table were prepared from reagents reported below following similar procedures as for Intermediate A22.

| Intermediate | Structure & Name | Reagents | Analytical data |
|---|---|---|---|
| A23 | 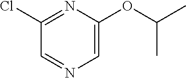 | 2,6- dichloropyrazine and propan-2-ol | LC-MS (ESI): m/z (M + 1): 173.0 (Method 1) ¹H NMR (400 MHz, Chloroform-d) δ 8.11 (d, J = 0.56 Hz, 1H), 8.07 (d, J = 0.56 Hz, 1H), 5.31 (p, J = 6.17 Hz, 1H), 1.39 (d, J = 6.17 Hz, 6H) |
| | 2-chloro-6- | | |
| | isopropoxypyrazine | | |
| A24 | 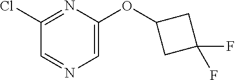 [SMILES: FC1(F)CC(Oc2cncc(Cl)n2)C1] | 2,6- dichloropyrazine and 3,3- difluorocyclobutan- 1-ol | LC-MS (ESI): m/z (M + 1): 220.9 (Method 1) ¹H NMR (400 MHz, Chloroform-d) δ 8.22 (s, 1H), 8.18 (s, 1H), 5.16 (qtd, J = 7.48, 5.59, 3.50 Hz, 1H), 3.19 (ddddd, J = 15.54, 11.78, 7.34, 3.99, 1.93 Hz, 2H), 2.90-2.69 (m, 2H) |
| | 2-chloro-6-(3,3- | | |
| | difluorocyclobutoxy) | | |
| | pyrazine | | |

### Step 9: tert-butyl (6-(4-((6-cyclopropoxypyrazin-2-yl)amino)-3-methylisoxazol-5-yl)-2-methylpyridin-3-yl)carbamate (Intermediate A25)

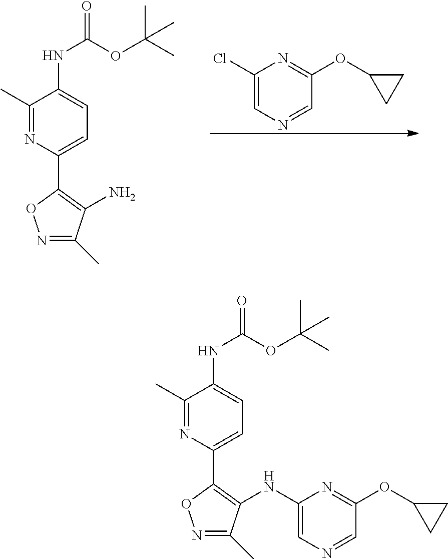 [SMILES: Cc1nc(-c2onc(C)c2N)ccc1NC(=O)OC(C)(C)C ; Clc1cncc(OC2CC2)n1 ; Cc1nc(-c2onc(C)c2Nc2cncc(OC3CC3)n2)ccc1NC(=O)OC(C)(C)C]

A 40 mL vial was charged with tert-butyl (6-(4-((5-cyclopropoxypyridin-3-yl)amino)-3-methylisoxazol-5-yl)-2-methylpyridin-3-yl)carbamate (Intermediate A21, 200.0 mg, 0.660 mmol), 2-chloro-6-cyclopropoxypyrazine (Intermediate A24, 84.08 mg, 0.490 mmol) and sodium tert-butoxide (101.04 mg, 1.05 mmol). The tube was sealed and three cycles of vacuum-N₂ back-filling were performed. Under nitrogen, Toluene (6.667 mL) was added and the mixture was degassed by performing three cycles of vacuum-N₂ back-filling. DavePhos (38.79 mg, 0.100 mmol) and Pd₂(dba)₃ (30.09 mg, 0.030 mmol) were added and the mixture was heated at 90° C. for 90 min. The crude mixture was filtered on Celite, washing with EtOAc and the filtrate was concentrated under reduced pressure. The crude was purified by flash chromatography using a gradient of EtOAc in CyHex from 0% to 70% affording title compound (99 mg, 0.226 mmol, 34.36% yield) as a yellow amorphous solid.

LC-MS (ESI): m/z (M+1): 439.2 (Method 1)

¹H NMR (400 MHz, Chloroform-d) δ ppm 8.39 (d, 1H), 8.10 (s, 1H), 7.76 (s, 1H), 7.71 (d, J=9.12 Hz, 1H), 6.39 (s, 1H), 4.16 (td, J=7.18, 6.53, 3.69 Hz, 1H), 2.56 (s, 3H), 2.47 (s, 3H), 1.56 (s, 9H), 0.86-0.68 (m, 4H)

The Intermediates in the following table were prepared from reagents reported below following similar procedures as for Intermediate A25.

| Intermediate | Structure & Name | Reagents | Analytical data |
|---|---|---|---|
| A26 | 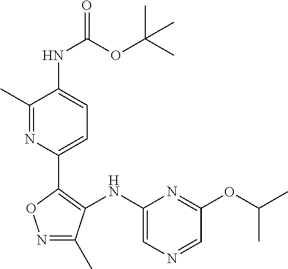 [SMILES: Cc1nc(-c2onc(C)c2Nc2cncc(OC(C)C)n2)ccc1NC(=O)OC(C)(C)C] | A21 and A23 | LC-MS (ESI): m/z (M + 1): 443.2 (Method 1) |
| | tert-butyl (6-(4-((6- | | |
| | isopropoxypyrazin-2- | | |
| | yl)amino)-3- | | |
| | methylisoxazol-5-yl)-2- | | |
| | methylpyridin-3- | | |
| | yl)carbamate | | |
| A27 | 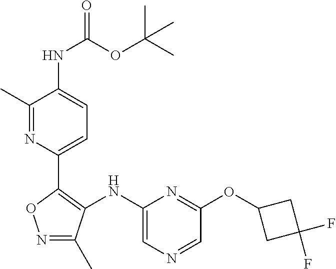 [SMILES: Cc1nc(-c2onc(C)c2Nc2cncc(OC3CC(F)(F)C3)n2)ccc1NC(=O)OC(C)(C)C] | A21 and A24 | LC-MS (ESI): m/z (M + 1): 488.2 (Method 1) |
| | tert-butyl (6-(4-((6-(3,3- | | |
| | difluorocyclobutoxy) | | |
| | pyrazin-2-yl)amino)-3- | | |
| | methylisoxazol-5-yl)-2- | | |
| | methylpyridin-3- | | |
| | yl)carbamate | | |
| A28 | 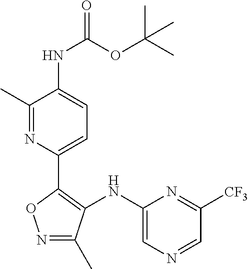 [SMILES: Cc1nc(-c2onc(C)c2Nc2cncc(C(F)(F)F)n2)ccc1NC(=O)OC(C)(C)C] | A21 and 2-chloro-6- (trifluoromethyl)pyrazine | LC-MS (ESI): m/z (M + 1): 450.2 (Method 1) |
| | tert-butyl (2-methyl-6-(3- | | |
| | methyl-4-((6- | | |
| | (trifluoromethyl)pyrazin- | | |
| | 2-yl)amino)isoxazol-5- | | |
| | yl)pyridin-3- | | |
| | yl)carbamate | | |
| A29 | 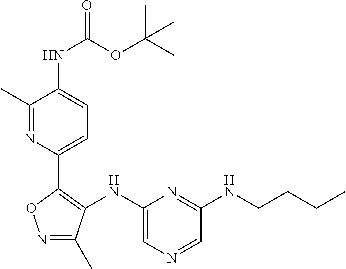 [SMILES: CCCCNc1cncc(Nc2c(C)noc2-c2ccc(NC(=O)OC(C)(C)C)c(C)n2)n1] | A21 and N-butyl-6- chloropyrazin- 2-amine | LC-MS (ESI): m/z (M + 1): 453.3 (Method 1) |
| | tert-butyl (6-(4-((6- | | |
| | (butylamino)pyrazin-2- | | |
| | yl)amino)-3- | | |
| | methylisoxazol-5-yl)-2- | | |
| | methylpyridin-3- | | |
| | yl)carbamate | | |
| A30 | 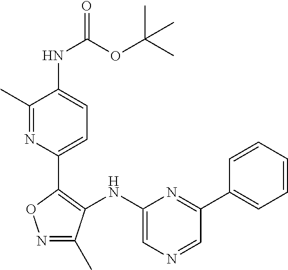 [SMILES: Cc1nc(-c2onc(C)c2Nc2cncc(-c3ccccc3)n2)ccc1NC(=O)OC(C)(C)C] | A21 and 2-chloro-6- phenylpyrazine | LC-MS (ESI): m/z (M + 1): 458.2 (Method 1) |
| | tert-butyl (2-methyl-6-(3- | | |
| | methyl-4-((6- | | |
| | phenylpyrazin-2- | | |
| | yl)amino)isoxazol-5- | | |
| | yl)pyridin-3- | | |
| | yl)carbamate | | |

### Step 10: 5-(5-amino-6-methylpyridin-2-yl)-N-(6-cyclopropoxypyrazin-2-yl)-3-methylisoxazol-4-amine (Intermediate A31)

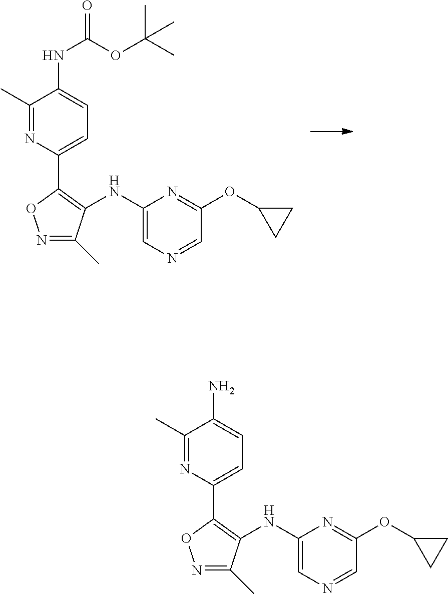 [SMILES: Cc1nc(-c2onc(C)c2Nc2cncc(OC3CC3)n2)ccc1NC(=O)OC(C)(C)C ; Cc1nc(-c2onc(C)c2Nc2cncc(OC3CC3)n2)ccc1N]

A solution of tert-butyl (6-(4-((6-cyclopropoxypyrazin-2-yl)amino)-3-methylisoxazol-5-yl)-2-methylpyridin-3-yl)carbamate (Intermediate A5, 99.0 mg, 0.230 mmol) in DCM (2.475 mL) was cooled at 0° C., then treated with TFA (0.825 mL). The mixture was let to warm up to RT and stirred for 3 h. The solvent was removed under vacuum. The crude was loaded on a SCX cartridge, washing with MeOH and then with 2N NH3. This last fraction was concentrated under reduced pressure recovering the title compound (68 mg, 0.201 mmol, 89.01% yield) as a yellow residue.

LC-MS (ESI): m/z (M+1): 339.2 (Method 1)

¹H NMR (400 MHz, Chloroform-d) δ ppm 8.12 (d, J=6.83 Hz, 1H), 7.75 (s, 1H), 7.68 (s, 1H), 7.57 (d, J=8.29 Hz, 1H), 7.02 (d, J=8.27 Hz, 1H), 4.17 (tt, J=6.27, 3.37 Hz, 1H), 3.85 (s, 2H), 2.46 (d, J=2.08 Hz, 3H), 1.28 (s, 3H), 0.82-0.64 (m, 4H)

The Intermediates in the following table were prepared from reagents reported below following similar procedures as for Intermediate A31.

| Intermediate | Structure & Name | Reagents | Analytical data |
|---|---|---|---|
| A32 | 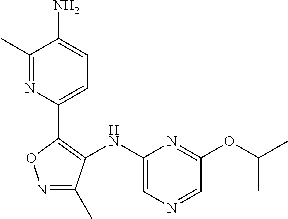 [SMILES: Cc1nc(-c2onc(C)c2Nc2cncc(OC(C)C)n2)ccc1N] | A26 | LC-MS (ESI): m/z (M + 1): 340.2 (Method 1) |
| | 5-(5-amino-6- | | |
| | methylpyridin-2-yl)-N- | | |
| | (6-isopropoxypyrazin-2- | | |
| | yl)-3-methylisoxazol-4- | | |
| | amine | | |
| A33 | 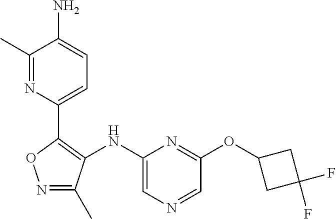 [SMILES: Cc1nc(-c2onc(C)c2Nc2cncc(OC3CC(F)(F)C3)n2)ccc1N] | A27 | LC-MS (ESI): m/z (M + 1): 388.2 (Method 1) |
| | 5-(5-amino-6- | | |
| | methylpyridin-2-yl)-N- | | |
| | (6-(3,3- | | |
| | difluorocyclobutoxy) | | |
| | pyrazin-2-yl)-3- | | |
| | methylisoxazol-4-amine | | |
| A34 | 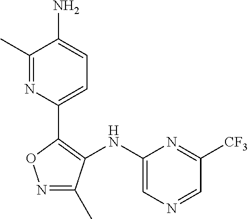 [SMILES: Cc1nc(-c2onc(C)c2Nc2cncc(C(F)(F)F)n2)ccc1N] | A28 | LC-MS (ESI): m/z (M + 1): 350.1 (Method 1) |
| | 5-(5-amino-6- | | |
| | methylpyridin-2-yl)-3- | | |
| | methyl-N-(6- | | |
| | (trifluoromethyl)pyrazin- | | |
| | 2-yl)isoxazol-4-amine | | |
| A36 | 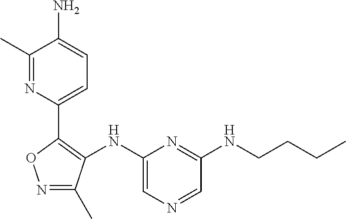 [SMILES: CCCCNc1cncc(Nc2c(C)noc2-c2ccc(N)c(C)n2)n1] | A29 | LC-MS (ESI): m/z (M + 1): 353.2 (Method 1) |
| | N2-(5-(5-amino-6- | | |
| | methylpyridin-2-yl)-3- | | |
| | methylisoxazol-4-yl)-N6- | | |
| | butylpyrazine-2,6- | | |
| | diamine | | |
| A38 | 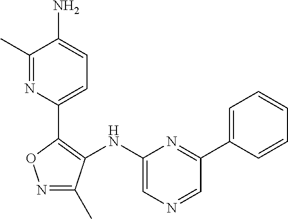 [SMILES: Cc1nc(-c2onc(C)c2Nc2cncc(-c3ccccc3)n2)ccc1N] | A30 | LC-MS (ESI): m/z (M + 1): 358.2 (Method 1) |
| | 5-(5-amino-6- | | |
| | methylpyridin-2-yl)-3- | | |
| | methyl-N-(6- | | |
| | phenylpyrazin-2- | | |
| | yl)isoxazol-4-amine | | |

### Step 11: (1S,2S)-2-((6-(4-((6-cyclopropoxypyrazin-2-yl)amino)-3-methylisoxazol-5-yl)-2-methylpyridin-3-yl)carbamoyl)cyclohexane-1-carboxylic acid (Compound 1)

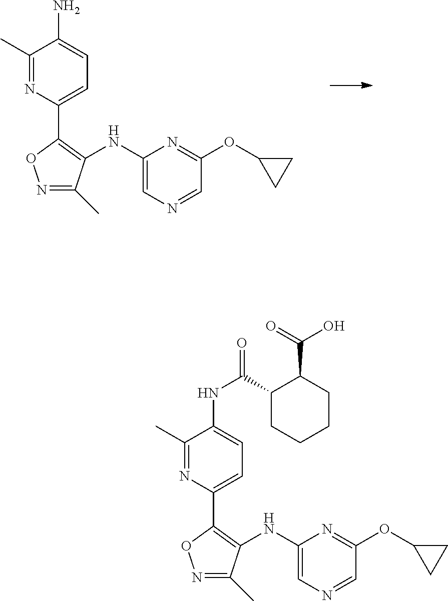 [SMILES: Cc1nc(-c2onc(C)c2Nc2cncc(OC3CC3)n2)ccc1N ; Cc1nc(-c2onc(C)c2Nc2cncc(OC3CC3)n2)ccc1NC(=O)[C@H]1CCCC[C@@H]1C(=O)O]

To a solution of 5-(5-amino-6-methylpyridin-2-yl)-N-(6-cyclopropoxypyrazin-2-yl)-3-methylisoxazol-4-amine (Intermediate A31, 68.0 mg, 0.200 mmol) in MeCN (1.758 mL), (−)-trans-1,2-Cyclohexanedicarboxylic anhydride (34.08 mg, 0.220 mmol) was added. The mixture was stirred at 40° C. for 24 h. An addition of (−)-trans-1,2-Cyclohexanedicarboxylic anhydride (34.08 mg, 0.220 mmol) was done and the reaction was stirred at 40° C. overnight. The solvent was removed under vacuum and the crude was purified by flash chromatography using a gradient of MeCN in water from 5% to 55% to give the final compound (30 mg, 0.061 mmol, 30.31% yield).

LC-MS (ESI): m/z (M+1): 493.3 (Method 1)

¹H NMR (400 MHz, DMSO-d₆) δ ppm 12.14 (br s, 1H), 9.69 (br s, 1H), 8.79 (s, 1H), 7.94 (br d, J=8.2 Hz, 1H), 7.78 (s, 1H), 7.64 (d, J=8.3 Hz, 1H), 7.55 (s, 1H), 3.90 (tt, J=5.9, 3.1 Hz, 1H), 2.76-2.60 (m, 1H), 2.47-2.39 (m, 1H), 2.33 (s, 3H), 2.23 (s, 3H), 2.12-1.92 (m, 2H), 1.75 (br s, 2H), 1.49-1.19 (m, 4H), 0.74-0.44 (m, 4H)

The Compounds in the following table were prepared from reagents reported below following similar procedures as for Compound 1.

| Compound | Structure & Name | Reagents | Analytical data |
|---|---|---|---|
| 2 | 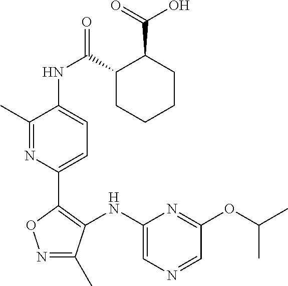 [SMILES: Cc1nc(-c2onc(C)c2Nc2cncc(OC(C)C)n2)ccc1NC(=O)[C@H]1CCCC[C@@H]1C(=O)O] | A32 and (−)- trans-1,2- Cyclohexane- dicarboxylic anhydride | LC-MS (ESI): m/z (M + 1): 495.3 (Method 1) ¹H NMR (400 MHz, DMSO-d₆) δ 9.80 (s, 1H), 8.68 (s, 1H), 7.99 (d, J = 8.4 Hz, 1H), 7.67 (s, 1H), 7.62 (d, J = 8.4 Hz, 1H), 7.41 (s, 1H), 4.73 (p, J = 6.2 Hz, 1H), 2.66 (td, J = 11.5, 3.7 Hz, 1H), 2.44- 2.35 (m, 1H), 2.33 (s, 3H), 2.23 (s, 3H), 2.00 (s, 1H), 1.93 (d, J = 12.4 Hz, 1H), 1.74 (s, 2H), 1.29 (dq, J = 19.2, 11.6, 11.0 Hz, 4H), 1.08 (d, J = 6.2 Hz, 6H) |
| | (1S,2S)-2-((6-(4-((6- | | |
| | isopropoxypyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylic acid | | |
| 3 | 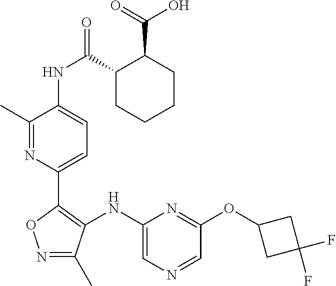 [SMILES: Cc1nc(-c2onc(C)c2Nc2cncc(OC3CC(F)(F)C3)n2)ccc1NC(=O)[C@H]1CCCC[C@@H]1C(=O)O] | A33 and (−)- trans-1,2- Cyclohexane- dicarboxylic anhydride | LC-MS (ESI): m/z (M + 1): 543.3 (Method 1) ¹H NMR (500 MHz, DMSO-d₆) δ ppm 12.12 (br s, 1 H), 9.61 (br s, 1 H), 8.82 (s, 1 H), 7.88 (d, J = 8.3 Hz, 1 H), 7.78 (s, 1 H), 7.63 (d, J = 8.3 Hz, 1 H), 7.51 (s, 1 H), 4.40- 4.61 (m, 1 H), 2.71-2.89 (m, 2 H), 2.61-2.71 (m, 1 H), 2.43-2.61 (m, 3 H), 2.30 (s, 3 H), 2.21 (s, 3 H), 1.93-2.08 (m, 2 H), 1.64- 1.84 (m, 2 H), 1.08-1.41 (m, 4 H) |
| | (1S,2S)-2-((6-(4-((6-(3,3- | | |
| | difluorocyclobutoxy) | | |
| | pyrazin-2-yl)amino)-3- | | |
| | methylisoxazol-5-yl)-2- | | |
| | methylpyridin-3- | | |
| | yl)carbamoyl)cyclohexane- | | |
| | 1-carboxylic acid | | |
| 4 | 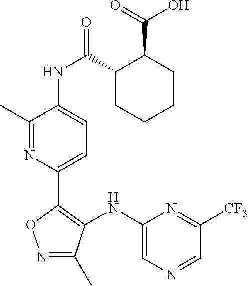 [SMILES: Cc1nc(-c2onc(C)c2Nc2cncc(C(F)(F)F)n2)ccc1NC(=O)[C@H]1CCCC[C@@H]1C(=O)O] | A34 and (−)- trans-1,2- Cyclohexane- dicarboxylic anhydride | LC-MS (ESI): m/z (M + 1): 505.3 (Method 1) ¹H NMR (500 MHz, DMSO-d₆) δ ppm 12.08 (br s, 1 H), 9.50-9.67 (m, 1 H), 9.47 (s, 1 H), 8.38 (s, 1 H), 8.31 (s, 1 H), 7.90- 7.98 (m, 1 H), 7.70 (d, J = 8.3 Hz, 1 H), 2.61-2.74 (m, 1 H), 2.48-2.56 (m, 1 H), 2.24 (s, 3 H), 2.18 (s, 3 H), 1.92-2.09 (m, 2 H), 1.64-1.84 (m, 2 H), 1.19- 1.41 (m, 4 H) |
| | (1S,2S)-2-((2-methyl-6-(3- | | |
| | methyl-4-((6- | | |
| | (trifluoromethyl)pyrazin- | | |
| | 2-yl)amino)isoxazol-5- | | |
| | yl)pyridin-3- | | |
| | yl)carbamoyl)cyclohexane- | | |
| | 1-carboxylic acid | | |
| 5 | 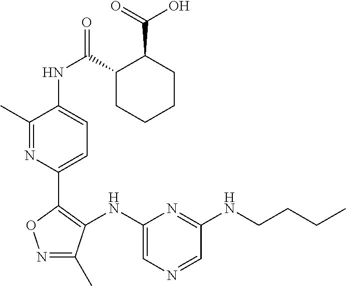 [SMILES: CCCCNc1cncc(Nc2c(C)noc2-c2ccc(NC(=O)[C@H]3CCCC[C@@H]3C(=O)O)c(C)n2)n1] | A35 and (−)- trans-1,2- Cyclohexane- dicarboxylic anhydride | LC-MS (ESI): m/z (M + 1): 508.4 (Method 1) ¹H NMR (500 MHz, DMSO-d₆) δ ppm 12.10 (br s, 1 H), 9.62 (br s, 1 H), 8.22 (s, 1 H), 7.91 (d, J = 8.3 Hz, 1 H), 7.61 (d, J = 8.3 Hz, 1 H), 7.23 (s, 1 H), 7.20 (s, 1 H), 6.60 (t, J = 5.5 Hz, 1 H), 2.99 (q, J = 6.8 Hz, 2 H), 2.61-2.75 (m, 1 H), 2.47-2.56 (m, 1 H), 2.42 (s, 3 H), 2.20 (s, 3 H), 1.92-2.09 (m, 2 H), 1.75 (br s, 2 H), 1.16- 1.48 (m, 8 H), 0.80 (t, J = 7.3 Hz, 3 H) |
| | (1S,2S)-2-((6-(4-((6- | | |
| | (butylamino)pyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylic acid | | |
| 6 | 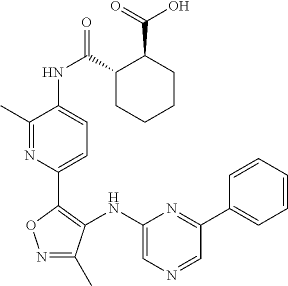 [SMILES: Cc1nc(-c2onc(C)c2Nc2cncc(-c3ccccc3)n2)ccc1NC(=O)[C@H]1CCCC[C@@H]1C(=O)O] | A36 and (−)- trans-1,2- Cyclohexane- dicarboxylic anhydride | LC-MS (ESI): m/z (M + 1): 513.3 (Method 1) ¹H NMR (500 MHz, DMSO-d₆) δ ppm 12.09 (br s, 1 H), 9.57 (br s, 1 H), 8.95 (s, 1 H), 8.49 (s, 1 H), 8.18 (s, 1 H), 7.92 (d, J = 8.4 Hz, 1 H), 7.76-7.83 (m, 2 H), 7.68 (d, J = 8.3 Hz, 1 H), 7.36-7.47 (m, 3 H), 2.58-2.76 (m, 1 H), 2.42-2.49 (m, 1 H), 2.33 (s, 3 H), 2.24 (s, 3 H), 1.84- 2.06 (m, 2 H), 1.74 (br s, 2 H), 1.18-1.41 (m, 4 H) |
| | (1S,2S)-2-((2-methyl-6-(3- | | |
| | methyl-4-((6- | | |
| | phenylpyrazin-2- | | |
| | yl)amino)isoxazol-5- | | |
| | yl)pyridin-3- | | |
| | yl)carbamoyl)cyclohexane- | | |
| | 1-carboxylic acid | | |

### Example 2

### Process Synthesis of Compound 7

### (1S,2S)-2-((2-methyl-6-(3-methyl-4-((6-(pyridin-3-yl)pyrazin-2-yl)amino)isoxazol-5-yl)pyridin-3-yl)carbamoyl)cyclohexane-1-carboxylic acid

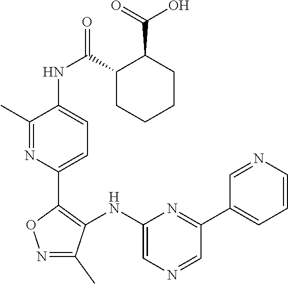 [SMILES: Cc1nc(-c2onc(C)c2Nc2cncc(-c3cccnc3)n2)ccc1NC(=O)[C@H]1CCCC[C@@H]1C(=O)O]

### Step 1: methyl 5-(5-amino-6-methylpyridin-2-yl)-3-methylisoxazole-4-carboxylate (Intermediate B1)

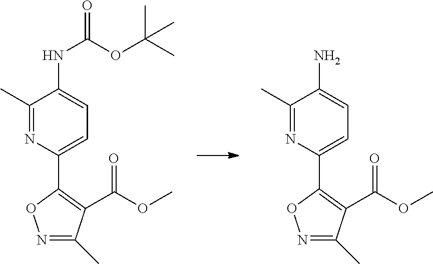

To a solution of methyl 5-(5-((tert-butoxycarbonyl)amino)-6-methylpyridin-2-yl)-3-methylisoxazole-4-carboxylate (Intermediate A8, 2.55 g, 7.34 mmol) in 1,4-Dioxane (10 mL), 4M HCl in 1,4-Dioxane (27.53 mL, 110.11 mmol) was added at RT and the resulting solution was stirred for 3 h at RT. Volatiles were evaporated and then DCM and water were added, then basified with ammonium hydroxide to pH 10. The layers were separated and water phase was washed with DCM. Combined organic layers were washed with brine and concentrated to give title compound (1.8 g, 7.28 mmol, 99% yield) as a crude that was used in the next step without any further purification.

LC-MS (ESI): m/z (M+1): 248.1 (Method 1)

### Step 2: (1S,2S)-2-((6-(4-(methoxycarbonyl)-3-methylisoxazol-5-yl)-2-methylpyridin-3-yl)carbamoyl)cyclohexane-1-carboxylic acid (Intermediate B2)

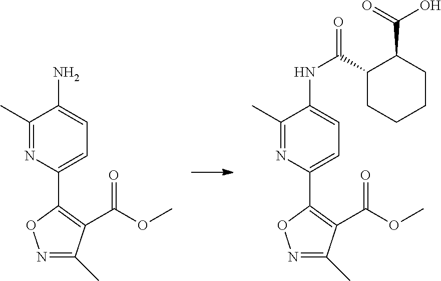

To a solution of methyl 5-(5-amino-6-methylpyridin-2-yl)-3-methylisoxazole-4-carboxylate (Intermediate B1, 1.8 g, 7.28 mmol) in DMF (30 mL), (−)-trans-1,2-Cyclohexanedicarboxylic anhydride (1.23 g, 8.01 mmol) was added. The mixture was stirred at 55° C. for 24 h. The crude was diluted with water and extracted with EtOAc (3 times). Combined organic layers were washed with 10% KHSO4 (2 times). Organic phases were dried over vacuum to give the product (3.15 g, crude) as a yellow foam.

LC-MS (ESI): m/z (M+1): 402.3 (Method 1)

### Step 3: methyl 5-(5-((1S,2S)-2-(tert-butoxycarbonyl)cyclohexane-1-carboxamido)-6-methylpyridin-2-yl)-3-methylisoxazole-4-carboxylate (Intermediate B3)

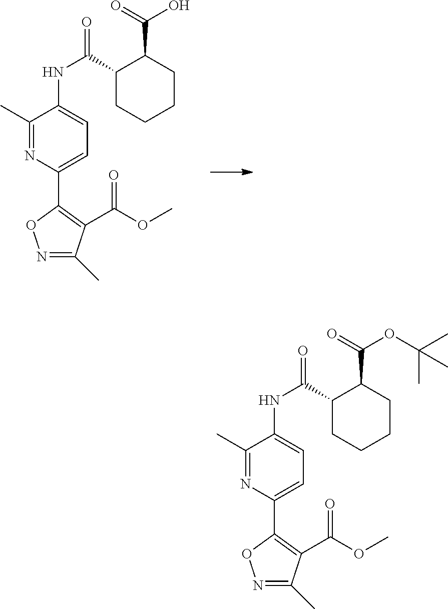 [SMILES: COC(=O)c1c(C)noc1-c1ccc(NC(=O)[C@H]2CCCC[C@@H]2C(=O)O)c(C)n1 ; COC(=O)c1c(C)noc1-c1ccc(NC(=O)[C@H]2CCCC[C@@H]2C(=O)OC(C)(C)C)c(C)n1]

A solution of (1S,2S)-2-((6-(4-(methoxycarbonyl)-3-methylisoxazol-5-yl)-2-methylpyridin-3-yl)carbamoyl)cyclohexane-1-carboxylic acid (Intermediate B2, 2.92 g, 7.28 mmol) in dry Toluene (21 mL) was heated to reflux. N,N-dimethyl-1,1-bis[(2-methylpropan-2-yl)oxy]methanamine (6.97 mL, 29.12 mmol) was added dropwise over 5 min to the refluxing mixture. The mixture was stirred at 110° C. for 6 h. The reaction was cooled down to RT was diluted with EtOAc and the organic layer was washed with sat. NaHCO₃ (3 times) and then brine. The organic layer was concentrated under reduced pressure. The crude was purified by flash chromatography using a gradient of EtOAc in cyclohexane from 0% to 40% affording the title compound (2.69 g, 5.88 mmol, 80.76% yield) a pale orange foam.

LC-MS (ESI): m/z (M+1): 458.3 (Method 1)

### Step 4: 5-(5-((1S,2S)-2-(tert-butoxycarbonyl)cyclohexane-1-carboxamido)-6-methylpyridin-2-yl)-3-methylisoxazole-4-carboxylic acid (Intermediate B4)

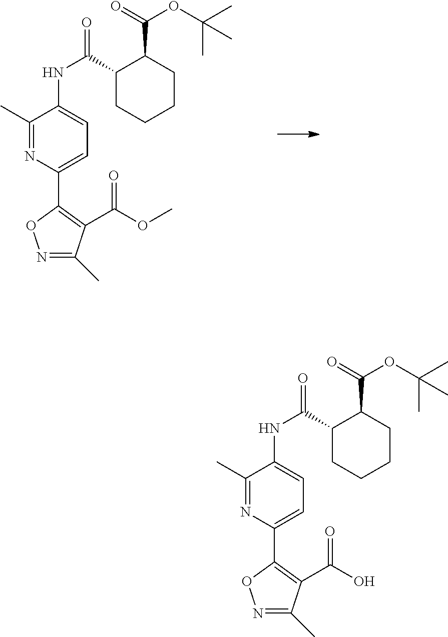 [SMILES: COC(=O)c1c(C)noc1-c1ccc(NC(=O)[C@H]2CCCC[C@@H]2C(=O)OC(C)(C)C)c(C)n1 ; Cc1nc(-c2onc(C)c2C(=O)O)ccc1NC(=O)[C@H]1CCCC[C@@H]1C(=O)OC(C)(C)C]

Methyl 5-(5-((1S,2S)-2-(tert-butoxycarbonyl)cyclohexane-1-carboxamido)-6-methylpyridin-2-yl)-3-methylisoxazole-4-carboxylate (Intermediate B3, 2.69 g, 5.88 mmol) was dissolved in Methanol (30 mL) and Water (5 mL). LiOH (281.63 mg, 11.76 mmol) was then added and the mixture was stirred at RT for 12h. Volatiles were evaporated and water was added. The mixture was acidified with 1 M HCl to pH ˜2-3.

The solid was filtered and washed with water. The isolated white solid was recovered by adding DCM and then concentrated under vacuum to give the corresponding acid (2.58 g, 5.817 mmol, 98.94% yield) as a yellow solid.

LC-MS (ESI): m/z (M+1): 444.3 (Method 1)

### Step 5: tert-butyl (1S,2S)-2-((2-methyl-6-(3-methyl-4-(((2-(trimethylsilyl)ethoxy)carbonyl)amino)isoxazol-5-yl)pyridin-3-yl)carbamoyl)cyclohexane-1-carboxylate (intermediate B5)

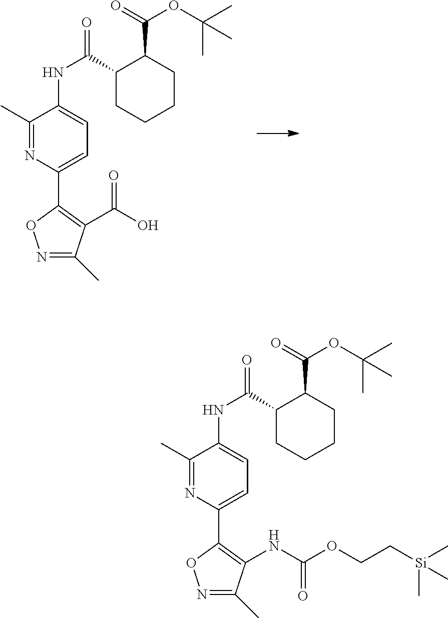

5-(5-((1S,2S)-2-(tert-butoxycarbonyl)cyclohexane-1-carboxamido)-6-methylpyridin-2-yl)-3-methylisoxazole-4-carboxylic acid (Intermediate B4, 2.58 g, 5.82 mmol) was suspended in dry Toluene (100 mL). 2-trimethylsilylethanol (1.0 mL, 6.98 mmol) was added. The system was closed and degassed in N₂ stream. TEA (1.62 mL, 11.63 mmol) and DPPA (1.88 mL, 8.73 mmol) were added and the solution was slowly heated at 110° C. for 12 h. The crude was concentrated under reduced pressure. The crude was purified by flash chromatography using a gradient of EtOAc in cyclohexane from 0% to 35% affording the desired compound (2.36 g, 4.224 mmol, 72.6% yield) as a white solid.

LC-MS (ESI): m/z (M+1): 559.4 (Method 1)

### Step 6: tert-butyl (1S,2S)-2-((6-(4-amino-3-methylisoxazol-5-yl)-2-methylpyridin-3-yl)carbamoyl)cyclohexane-1-carboxylate (Intermediate B6)

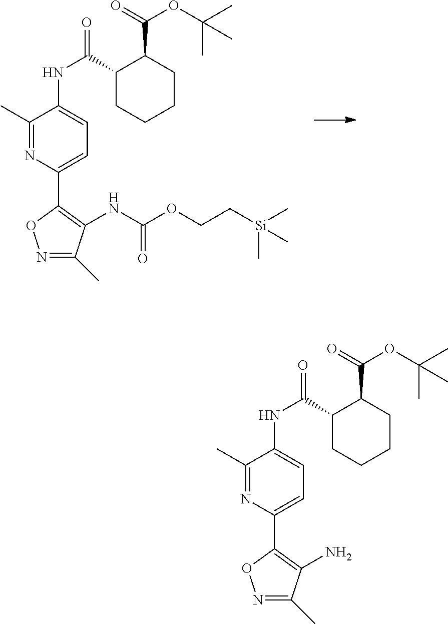

tert-butyl (1S,2S)-2-((2-methyl-6-(3-methyl-4-(((2-(trimethylsilyl)ethoxy)carbonyl)amino)isoxazol-5-yl)pyridin-3-yl)carbamoyl)cyclohexane-1-carboxylate (Intermediate B5, 2.36 g, 4.22 mmol) was dissolved in THE (50 mL) and TBAF trihydrate (1.47 g, 4.65 mmol) was added under stirring. The mixture was stirred at 50° C. for 2 h. Solvent was evaporated. The mixture was diluted with DCM/water. Layers were separated and water phase washed with DCM (2 times). Combined organic layers washed with water and dried under vacuum to give title compound (1.9 g, crude).

LC-MS (ESI): m/z (M+1): 415.3 (Method 1)

### Step 7: 2-chloro-6-(pyridin-3-yl)pyrazine (Intermediate B7)

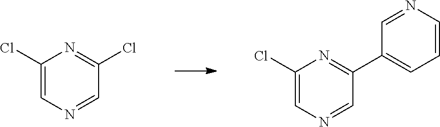 [SMILES: Clc1cncc(Cl)n1 ; Clc1cncc(-c2cccnc2)n1]

In a vial 2,6-dichloropyrazine (200.0 mg, 1.34 mmol) was dissolved in dry 1,4-Dioxane (0.600 mL)/Water (0.150 mL). 3-(4,4,5,5-tetramethyl-1,3,2-dioxaborolan-2-yl)pyridine (247.76 mg, 1.21 mmol), Na₂CO₃ (284.58 mg, 2.68 mmol) were added. The system was closed and three cycles of vacuum/nitrogen back-filling were applied. Pd(dppf)Cl₂ (167.45 mg, 0.230 mmol) was added and the solution was heated at 90 r C for 2h. Solvent was removed under vacuum. The crude was purified by flash chromatography using a gradient of EtOAc in CyHex from 000 to 70% affording the title compound (128.5 mg, 0.671 mmol, 49.95% yield) as a pink solid.

LC-MS (ESI): m/z (M+1): 192.0 (Method 1)

The Intermediates in the following table were prepared from reagents reported below by using methods analogous to Intermediate B7.

| Intermediate | Structure & Name | Reagents | Analytical data |
|---|---|---|---|
| B8 | 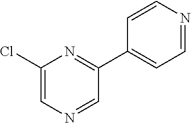 [SMILES: Clc1cncc(-c2ccncc2)n1] | 2,6-dichloropyrazine and 4-(4,4,5,5- tetramethyl-1,3,2- dioxaborolan- 2-yl)pyridine | LC-MS (ESI): m/z (M + 1): 192.0 (Method 1) ¹H NMR (400 MHz, Chloroform-d) δ 9.02 (d, J = 0.61 Hz, 1H), 8.88-8.77 (m, 2H), 8.67 (d, J = 0.59 Hz, 1H), 8.04-7.81 (m, 2H) |
| | 2-chloro-6-(pyridin-4- | | |
| | yl)pyrazine | | |
| B9 | 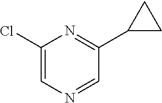 | 2,6-dichloropyrazine and 2-cyclopropyl- 4,4,5,5-tetramethyl- 1,3,2- dioxaborolane | LC-MS (ESI): m/z (M + 1): 154.8 (Method 1) ¹H NMR (400 MHz, Chloroform-d) δ 8.38 (s, 1H), 8.32 (s, 1H), 2.06 (tt, J = 7.1, 5.7 Hz, 1H), 1.16-1.06 (m, 4H) |
| | 2-chloro-6- | | |
| | cyclopropylpyrazine | | |
| B10 | 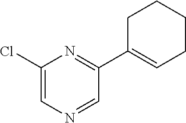 [SMILES: Clc1cncc(C2=CCCCC2)n1] | 2,6-dichloropyrazine and 2-(cyclohex-1- en-1-yl)-4,4,5,5- tetramethyl-1,3,2- dioxaborolane | LC-MS (ESI): m/z (M + 1): 195.0 (Method 1) |
| | 2-chloro-6-(cyclohex- | | |
| | 1-en-1-yl)pyrazine | | |
| B11 | 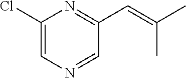 | 2,6-dichloropyrazine and 4,4,5,5-tetramethyl- 2-(2-methylprop-1-en- 1-yl)-1,3,2-dioxaborolane | LC-MS (ESI): m/z (M + 1): 169.0 (Method 1) ¹H NMR (400 MHz, Chloroform-d) δ 8.33 (s, 2H), 6.28 (p, J = 1.49 Hz, 1H), 2.20 (d, J = 1.36 Hz, 3H), 2.03 (d, J = 1.47 Hz, 3H) |
| | 2-chloro-6-(2- | | |
| | methylprop-1-en-1- | | |
| | yl)pyrazine | | |
| B12 | 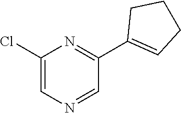 [SMILES: Clc1cncc(C2=CCCC2)n1] | 2,6-dichloropyrazine and 2-(cyclopent-1- en-1-yl)-4,4,5,5- tetramethyl-1,3,2- dioxaborolane | LC-MS (ESI): m/z (M + 1): 181.1 (Method 1) ¹H NMR (400 MHz, Chloroform-d) δ 8.55 (d, J = 1.15 Hz, 1H), 8.39 (s, 1H), 6.89-6.76 (m, 1H), 2.81 (ddt, J = 10.17, 4.54, 2.25 Hz, 2H), 2.63 (tq, J = 7.77, 2.65 Hz, 2H), 2.16-2.02 (m, 2H) |
| | 2-chloro-6- | | |
| | (cyclopent-1-en-1- | | |
| | yl)pyrazine | | |
| B13 | 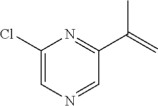 | 2,6-dichloropyrazine and 2-(cyclopent-1- en-1-yl)-4,4,5,5- tetramethyl-1,3,2- dioxaborolane | LC-MS (ESI): m/z (M + 1): 155.2 (Method 1) ¹H NMR (400 MHz, Chloroform-d) δ 8.67 (s, 1H), 8.46 (s, 1H), 6.03 (t, J = 1.0 Hz, 1H), 5.48 (t, J = 1.4 Hz 1H), 2.23 (dd, J = 1.5, 0.9 Hz, 3H) |
| | 2-chloro-6-(prop-1- | | |
| | en-2-yl)pyrazine | | |

### Step 8: tert-butyl (1S,2S)-2-((2-methyl-6-(3-methyl-4-((6-(pyridin-3-yl)pyrazin-2-yl)amino)isoxazol-5-yl)pyridin-3-yl)carbamoyl)cyclohexane-1-carboxylate (Intermediate B 14)

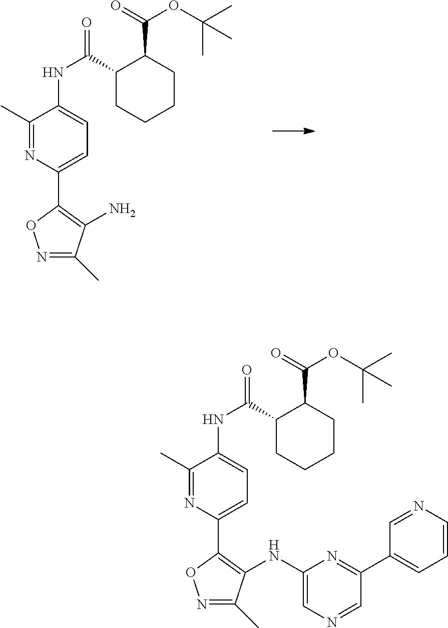 [SMILES: Cc1nc(-c2onc(C)c2N)ccc1NC(=O)[C@H]1CCCC[C@@H]1C(=O)OC(C)(C)C ; Cc1nc(-c2onc(C)c2Nc2cncc(-c3cccnc3)n2)ccc1NC(=O)[C@H]1CCCC[C@@H]1C(=O)OC(C)(C)C]

Intermediate B 14 (94 mg, 0.165 mmol, 34.2% yield) was obtained as a yellow solid from tert-butyl (1S,2S)-2-((6-(4-amino-3-methylisoxazol-5-yl)-2-methylpyridin-3-yl)carbamoyl)cyclohexane-1-carboxylate (Intermediate B6, 200.0 mg, 0.480 mmol), 2-chloro-6-(pyridin-3-yl)pyrazine (Intermediate B7, 78.59 mg, 0.410 mmol) using a similar method as for Intermediate A25.

LC-MS (ESI): m/z (M+1): 570.4 (Method 1)

The Intermediates in the following table were prepared from reagents reported below by using methods analogous to Intermediate B 14.

| Intermediate | Structure & Name | Reagents | Analytical data |
|---|---|---|---|
| B15 | 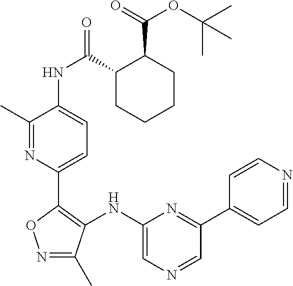 [SMILES: Cc1nc(-c2onc(C)c2Nc2cncc(-c3ccncc3)n2)ccc1NC(=O)[C@H]1CCCC[C@@H]1C(=O)OC(C)(C)C] | B6 and B8 | LC-MS (ESI): m/z (M + 1): 570.4 (Method 1) ¹H NMR (400 MHz, Chloroform-d) δ 8.77- 8.71 (m, 2H), 8.58 (s, 1H), 8.46 (d, J = 9.41 Hz, 2H), 8.25 (s, 1H), 7.87-7.81 (m, 2H), 7.75 (d, J = 8.53 Hz, 1H), 2.58 (d, J = 14.78 Hz, 5H), 2.42 (s, 3H), 2.15 (d, J = 8.62 Hz, 1H), 1.99 (d, J = 13.27 Hz, 1H), 1.88 (d, J = 13.54 Hz, 2H), 1.41 (s, 9H), 1.35 (m, 3H), 0.86 (s, 2H) |
| | tert-butyl (1S,2S)-2-((2- | | |
| | methyl-6-(3-methyl-4- | | |
| | ((6-(pyridin-4- | | |
| | yl)pyrazin-2- | | |
| | yl)amino)isoxazol-5- | | |
| | yl)pyridin-3- | | |
| | yl)carbamoyl)cyclohexane- | | |
| | 1-carboxylate | | |
| B16 | 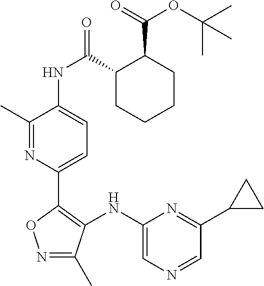 [SMILES: Cc1nc(-c2onc(C)c2Nc2cncc(C3CC3)n2)ccc1NC(=O)[C@H]1CCCC[C@@H]1C(=O)OC(C)(C)C] | B6 and B9 | LC-MS (ESI): m/z (M + 1): 533.2 (Method 1) ¹H NMR (400 MHz, Chloroform-d) δ 8.42 (d, J = 8.5 Hz, 1H), 8.12 (s, 1H), 7.91 (d, J = 17.5 Hz, 2H), 7.68 (d, J = 8.5 Hz, 1H), 7.44 (s, 1H), 2.61- 2.58 (m, 2H), 2.56 (s, 3H), 2.38 (s, 3H), 2.17-2.10 (m, 1H), 2.02-1.90 (m, 2H), 1.90-1.79 (m, 2H), 1.74-1.60 (m, 1H), 1.40 (s, 9H), 1.38-1.34 (m, 3H), 1.03-0.91 (m, 4H) |
| | tert-butyl (1S,2S)-2-((6- | | |
| | (4-((6- | | |
| | cyclopropylpyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylate | | |
| B17 | 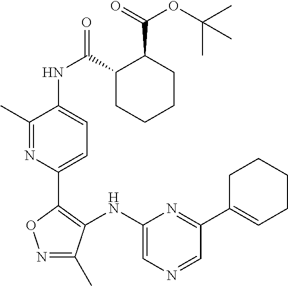 [SMILES: Cc1nc(-c2onc(C)c2Nc2cncc(C3=CCCCC3)n2)ccc1NC(=O)[C@H]1CCCC[C@@H]1C(=O)OC(C)(C)C] | B6 and B10 | LC-MS (ESI): m/z (M + 1): 573.4 (Method 1) |
| | tert-butyl (1S,2S)-2-((6- | | |
| | (4-((6-(cyclohex-1-en-1- | | |
| | yl)pyrazin-2-yl)amino)- | | |
| | 3-methylisoxazol-5-yl)- | | |
| | 2-methylpyridin-3- | | |
| | yl)carbamoyl)cyclohexane- | | |
| | 1-carboxylate | | |
| B18 | 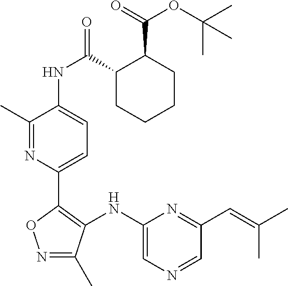 [SMILES: CC(C)=Cc1cncc(Nc2c(C)noc2-c2ccc(NC(=O)[C@H]3CCCC[C@@H]3C(=O)OC(C)(C)C)c(C)n2)n1] | B6 and B11 | LC-MS (ESI): m/z (M + 1): 546.3 (Method 1) |
| | tert-butyl (1S,2S)-2-((2- | | |
| | methyl-6-(3-methyl-4- | | |
| | ((6-(2-methylprop-1-en-1- | | |
| | 1-yl)pyrazin-2- | | |
| | yl)amino)isoxazol-5- | | |
| | yl)pyridin-3- | | |
| | yl)carbamoy)cyclohexane- | | |
| | 1-carboxylate | | |
| B19 | 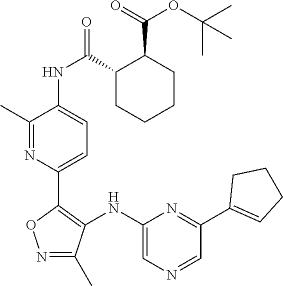 [SMILES: Cc1nc(-c2onc(C)c2Nc2cncc(C3=CCCC3)n2)ccc1NC(=O)[C@H]1CCCC[C@@H]1C(=O)OC(C)(C)C] | B6 and B12 | LC-MS (ESI): m/z (M + 1): 559.4 (Method 1) |
| | tert-butyl (1S,2S)-2-((6- | | |
| | (4-((6-(cyclopent-1-en-1- | | |
| | yl)pyrazin-2-yl)amino)- | | |
| | 3-methylisoxazol-5-yl)- | | |
| | 2-methylpyridin-3- | | |
| | yl)carbamoyl)cyclohexane- | | |
| | 1-carboxylate | | |
| B20 | 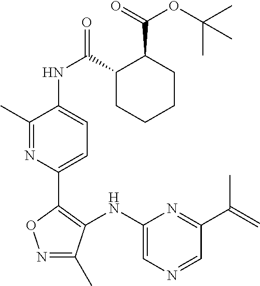 [SMILES: C=C(C)c1cncc(Nc2c(C)noc2-c2ccc(NC(=O)[C@H]3CCCC[C@@H]3C(=O)OC(C)(C)C)c(C)n2)n1] | B6 and B13 | LC-MS (ESI): m/z (M + 1): 532.3 (Method 1) |
| | tert-butyl (1S,2S)-2-((2- | | |
| | methyl-6-(3-methyl-4- | | |
| | ((6-(prop-1-en-2- | | |
| | yl)pyrazin-2- | | |
| | yl)amino)isoxazol-5- | | |
| | yl)pyridin-3- | | |
| | yl)carbamoyl)cyclohexane- | | |
| | 1-carboxylate | | |

### Step 9: (1S,2S)-2-((2-methyl-6-(3-methyl-4-((6-(pyridin-3-yl)pyrazin-2-yl)amino)isoxazol-5-yl)pyridin-3-yl)carbamoyl)cyclohexane-1-carboxylic acid (Compound 7)

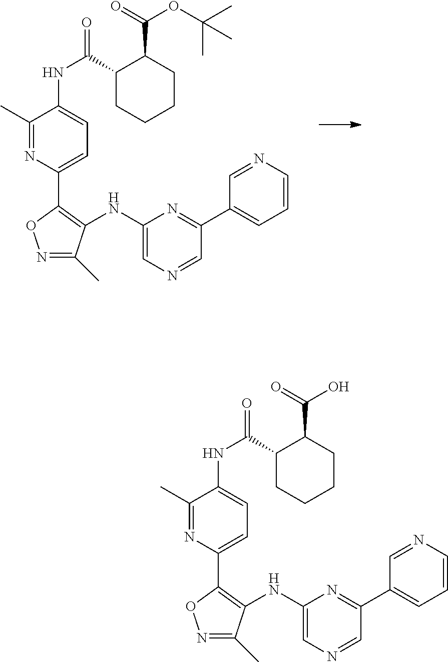 [SMILES: Cc1nc(-c2onc(C)c2Nc2cncc(-c3cccnc3)n2)ccc1NC(=O)[C@H]1CCCC[C@@H]1C(=O)OC(C)(C)C ; Cc1nc(-c2onc(C)c2Nc2cncc(-c3cccnc3)n2)ccc1NC(=O)[C@H]1CCCC[C@@H]1C(=O)O]

To a solution of tert-butyl (1S,2S)-2-((2-methyl-6-(3-methyl-4-((6-(pyridin-3-yl)pyrazin-2-yl)amino)isoxazol-5-yl)pyridin-3-yl)carbamoyl)cyclohexane-1-carboxylate (Intermediate B14, 94.0 mg, 0.170 mmol) in DCM (1.492 mL), 4 M HCl in 1,4-dioxane (0.41 mL, 1.65 mmol) was added at RT and the resulting solution was stirred for 12 h. Volatiles were evaporated. The crude was by flash chromatography using a gradient of MeCN in water from 5% to 40% to give the desired product (383 mg, 0.990 mmol, 80.35% yield) as a whitenish solid.

LC-MS (ESI): m/z (M+1): 514.3 (Method 1)

¹H NMR (600 MHz, DMSO-d₆) δ ppm 12.08 (br s, 1H), 9.52 (br s, 1H), 9.05 (s, 1H), 8.98 (d, J=1.6 Hz, 1H), 8.58 (dd, J=4.8, 1.5 Hz, 1H), 8.56 (s, 1H), 8.23 (s, 1H), 8.07 (dt, J=8.0, 1.9 Hz, 1H), 7.90 (d, J=8.4 Hz, 1H), 7.68 (d, J=8.4 Hz, 1H), 7.44 (ddd, J=8.0, 4.9, 0.7 Hz, 1H), 2.70-2.59 (m, 1H), 2.50 (dt, J=3.6, 1.8 Hz, 1H), 2.29 (s, 3H), 2.25 (s, 3H), 2.03-1.92 (m, 2H), 1.78-1.71 (m, 2H), 1.38-1.20 (m, 4H)

The Compounds in the following table were prepared from reagents reported below by using methods analogous to Compound 7.

| Compound | Structure & Name | Reagents | Analytical data |
|---|---|---|---|
| 8 | 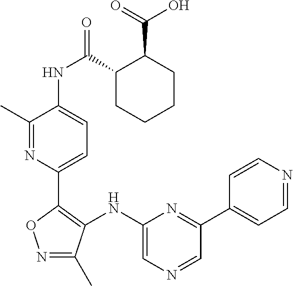 [SMILES: Cc1nc(-c2onc(C)c2Nc2cncc(-c3ccncc3)n2)ccc1NC(=O)[C@H]1CCCC[C@@H]1C(=O)O] | B15 | LC-MS (ESI): m/z (M + 1): 514.3 (Method 1) 1H NMR (400 MHz, DMSO-d₆) δ ppm 12.10 (br s, 1 H), 9.58 (br s, 1 H), 9.11 (s, 1 H), 8.59- 8.65 (m, 3 H), 8.28 (s, 1 H), 7.92 (d, J = 8.3 Hz, 1 H), 7.70-7.73 (m, 2 H), 7.68 (d, J = 8.6 Hz, 1 H), 2.58-2.69 (m, 1 H), 2.38- 2.48 (m, 1 H), 2.29 (s, 3 H), 2.25 (s, 3 H), 1.87- 2.08 (m, 2 H), 1.65-1.82 (m, 2 H), 1.18-1.39 (m, 4 H) |
| | (1S,2S)-2-((2-methyl-6- | | |
| | (3-methyl-4-((6-(pyridin- | | |
| | 4-yl)pyrazin-2- | | |
| | yl)amino)isoxazol-5- | | |
| | yl)pyridin-3- | | |
| | yl)carbamoyl)cyclohexane- | | |
| | 1-carboxylic acid | | |
| 9 | 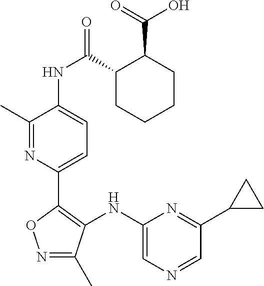 [SMILES: Cc1nc(-c2onc(C)c2Nc2cncc(C3CC3)n2)ccc1NC(=O)[C@H]1CCCC[C@@H]1C(=O)O] | B16 | LC-MS (ESI): m/z (M + 1): 477.2 (Method 1) 1H NMR (400 MHz, DMSO-d₆) δ ppm 11.62- 12.52 (m, 1 H), 9.47-9.77 (m, 1 H), 8.65 (s, 1 H), 7.88-7.94 (m, 2 H), 7.84 (s, 1 H), 7.61 (d, J = 8.4 Hz, 1 H), 2.61-2.70 (m, 1 H), 2.34 (m, 3 H), 2.18 (s, 3 H), 1.93-2.05 (m, 2 H), 1.85-1.92 (m, 1 H), 1.68- 1.82 (m, 2 H), 1.16-1.45 (m, 4 H), 0.73-0.84 (m, 2 H), 0.49-0.60 (m, 2 H) |
| | (1S,2S)-2-((6-(4-((6- | | |
| | cyclopropylpyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylic acid | | |
| 10 | 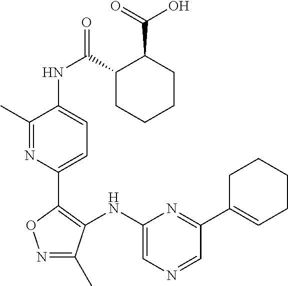 [SMILES: Cc1nc(-c2onc(C)c2Nc2cncc(C3=CCCCC3)n2)ccc1NC(=O)[C@H]1CCCC[C@@H]1C(=O)O] | B17 | LC-MS (ESI): m/z (M + 1): 517.2 (Method 1) 1H NMR (500 MHz, DMSO-d₆) δ ppm 11.78- 12.54 (m, 1 H), 9.42-9.92 (m, 1 H), 8.75 (s, 1 H), 8.03-8.10 (m, 1 H), 7.99 (s, 1 H), 7.89-7.97 (m, 1 H), 7.61 (d, J = 8.4 Hz, 1 H), 6.42-6.50 (m, 1 H), 2.65-2.73 (m, 1 H), 2.49- 2.55 (m, 1 H), 2.32-2.39 (m, 3 H), 2.16-2.24 (m, 2 H), 2.08-2.15 (m, 2 H), 1.68-2.05 (m, 4 H), 1.58- 1.68 (m, 2 H), 1.49-1.57 (m, 2 H), 1.20-1.41 (m, 4 H) |
| | (1S,2S)-2-((6-(4-((6- | | |
| | (cyclohex-1-en-1- | | |
| | yl)pyrazin-2-yl)amino)- | | |
| | 3-methylisoxazol-5-yl)- | | |
| | 2-methylpyridin-3- | | |
| | yl)carbamoyl)cyclohexane- | | |
| | 1-carboxylic acid | | |

### Example 3

### Process Synthesis of Compound 11

### (1S,2S)-2-((6-(4-((6-cyclopentylpyrazin-2-yl)amino)-3-methylisoxazol-5-yl)-2-methylpyridin-3-yl)carbamoyl)cyclohexane-1-carboxylic acid

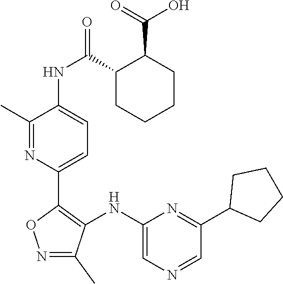 [SMILES: Cc1nc(-c2onc(C)c2Nc2cncc(C3CCCC3)n2)ccc1NC(=O)[C@H]1CCCC[C@@H]1C(=O)O]

### Step 1: tert-butyl (1S,2S)-2-((6-(4-((6-cyclopentylpyrazin-2-yl)amino)-3-methylisoxazol-5-yl)-2-methylpyridin-3-yl)carbamoyl)cyclohexane-1-carboxylate (Intermediate B21)

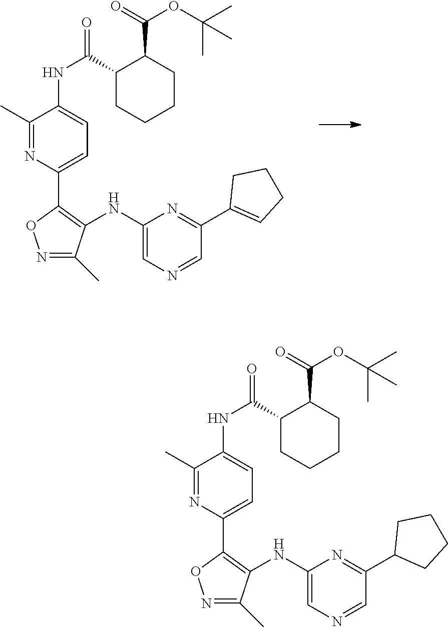 [SMILES: Cc1nc(-c2onc(C)c2Nc2cncc(C3=CCCC3)n2)ccc1NC(=O)[C@H]1CCCC[C@@H]1C(=O)OC(C)(C)C ; Cc1nc(-c2onc(C)c2Nc2cncc(C3CCCC3)n2)ccc1NC(=O)[C@H]1CCCC[C@@H]1C(=O)OC(C)(C)C]

Intermediate B21 (74 mg, 0.132 mmol, 61.44% yield) was obtained from tert-butyl (1S,2S)-2-((6-(4-((6-(cyclopent-1-en-1-yl)pyrazin-2-yl)amino)-3-methylisoxazol-5-yl)-2-methylpyridin-3-yl)carbamoyl)cyclohexane-1-carboxylate (Intermediate B19, 120.0 mg, 0.210 mmol) using a similar method as for Intermediate A21.

LC-MS (ESI): m/z (M+1): 561.4 (Method 1)

### Step 2: (1S,2S)-2-((6-(4-((6-cyclopentylpyrazin-2-yl)amino)-3-methylisoxazol-5-yl)-2-methylpyridin-3-yl)carbamoyl)cyclohexane-1-carboxylic acid (Compound 11)

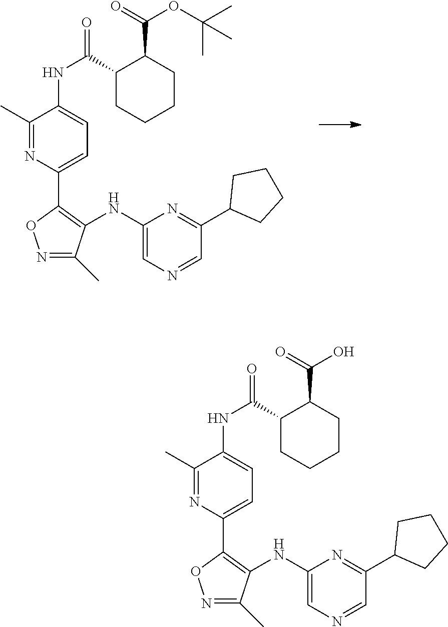 [SMILES: Cc1nc(-c2onc(C)c2Nc2cncc(C3CCCC3)n2)ccc1NC(=O)[C@H]1CCCC[C@@H]1C(=O)OC(C)(C)C ; Cc1nc(-c2onc(C)c2Nc2cncc(C3CCCC3)n2)ccc1NC(=O)[C@H]1CCCC[C@@H]1C(=O)O]

Compound 11 (40 mg, 0.079 mmol, 60.06% yield) was obtained from tert-butyl (1S,2S)-2-((6-(4-((6-cyclopentylpyrazin-2-yl)amino)-3-methylisoxazol-5-yl)-2-methylpyridin-3-yl)carbamoyl)cyclohexane-1-carboxylate (Intermediate B21, 74 mg, 0.132 mmol) using a similar method as for Compound 7.

LC-MS (ESI): m/z (M+1): 505.3 (Method 1)

1H NMR (400 MHz, DMSO-d₆) δ ppm 12.10 (br s, 1H), 9.56 (br s, 1H), 8.72 (s, 1H), 7.98 (s, 1H), 7.89 (d, J=8.3 Hz, 1H), 7.77 (s, 1H), 7.62 (d, J=8.3 Hz, 1H), 2.92 (quin, J=8.2 Hz, 1H), 2.73-2.61 (m, 1H), 2.56-2.49 (m, 1H), 2.30 (s, 3H), 2.22 (s, 3H), 2.09-1.16 (m, 16H)

The Compounds in the following table were prepared from reagents reported below by using methods analogous to Compound 7.

| Compound | Structure & Name | Reagents | Analytical data |
|---|---|---|---|
| 12 | 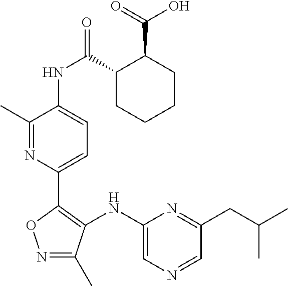 [SMILES: Cc1nc(-c2onc(C)c2Nc2cncc(CC(C)C)n2)ccc1NC(=O)[C@H]1CCCC[C@@H]1C(=O)O] | B18 | LC-MS (ESI): m/z (M + 1): 493.3 (Method 1) 1H NMR (600 MHz, DMSO-d₆) δ ppm 10.76 (br s, 1 H), 8.71 (s, 1 H), 8.33 (br d, J = 7.1 Hz, 1 H), 7.96 (s, 1 H), 7.71 (s, 1 H), 7.58 (d, J = 8.4 Hz, 1 H), 2.59-2.68 (m, 1 H), 2.48- 2.52 (m, 1 H), 2.36 (s, 3 H), 2.31 (d, J = 7.2 Hz, 2 H), 2.19 (s, 3 H), 1.93- 2.06 (m, 2 H), 1.61-1.81 (m, 3 H), 1.09-1.33 (m, 4 H), 0.76 (d, J = 6.8 Hz, 6 H) |
| | (1S,2S)-2-((6-(4-((6- | | |
| | isobutylpyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylic acid | | |
| 13 | 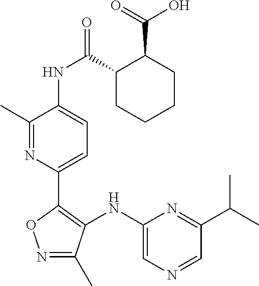 [SMILES: Cc1nc(-c2onc(C)c2Nc2cncc(C(C)C)n2)ccc1NC(=O)[C@H]1CCCC[C@@H]1C(=O)O] | B20 | LC-MS (ESI): m/z (M + 1): 477.2 (Method 1) 1H NMR (400 MHz, DMSO-d₆) δ ppm 9.56- 9.74 (m, 1 H), 8.72 (s, 1 H), 7.99 (s, 1 H), 7.92 (d, J = 8.3 Hz, 1 H), 7.79 (s, 1 H), 7.63 (d, J = 8.4 Hz, 1 H), 2.72-2.84 (m, 1 H), 2.60-2.70 (m, 1 H), 2.38- 2.47 (m, 1 H), 2.29 (s, 3 H), 2.23 (s, 3 H), 1.91- 2.07 (m, 2 H), 1.68-1.84 (m, 2 H), 1.19-1.43 (m, 4 H), 1.03 (d, J = 6.9 Hz, 6 H) |
| | (1S,2S)-2-((6-(4-((6- | | |
| | isopropylpyrazin-2- | | |
| | yl)amino)-3- | | |
| | methylisoxazol-5-yl)-2- | | |
| | methylpyridin-3- | | |
| | yl)carbamoyl)cyclohexane- | | |
| | 1-carboxylic acid | | |

### Example 4

### Process Synthesis of Compound 14

### (1S,2S)-2-((2-methyl-6-(3-methyl-4-((6-(pyridin-2-yl)pyrazin-2-yl)amino)isoxazol-5-yl)pyridin-3-yl)carbamoyl)cyclohexane-1-carboxylic acid

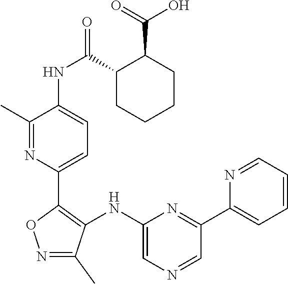 [SMILES: Cc1nc(-c2onc(C)c2Nc2cncc(-c3ccccn3)n2)ccc1NC(=O)[C@H]1CCCC[C@@H]1C(=O)O]

### Step 1: 2-chloro-6-(pyridin-2-yl)pyrazine (Intermediate B22)

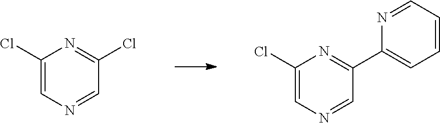 [SMILES: Clc1cncc(Cl)n1 ; Clc1cncc(-c2ccccn2)n1]

A vial equipped with a stir bar was charged with tributyl(2-pyridinyl)stannane (0.48 mL, 1.51 mmol) and 2,6-dichloropyrazine (250.0 mg, 1.68 mmol) in dry Toluene (4.5 mL) The vial was sealed and three cycles of vacuum/nitrogen back-filling were applied. Pd(PPh₃)₄ (193.91 mg, 0.170 mmol) was added, the tube was sealed and the mixture was heated at 80° C. for 24h. Aqueous KF solution was added, followed by EtOAc. The organic layer was washed with water and then concentrated under reduced pressure. The crude was purified by flash chromatography using a gradient of EtOAc in CyHex from 0% to 30% affording the title compound (205.8 mg, 1.074 mmol, 64% yield) as a whitenish solid.

LC-MS (ESI): m/z (M+1): 341.0 (Method 1)

### Step 2: tert-butyl (1S,2S)-2-((2-methyl-6-(3-methyl-4-((6-(pyridin-2-yl)pyrazin-2-yl)amino)isoxazol-5-yl)pyridin-3-yl)carbamoyl)cyclohexane-1-carboxylate (Intermediate B23)

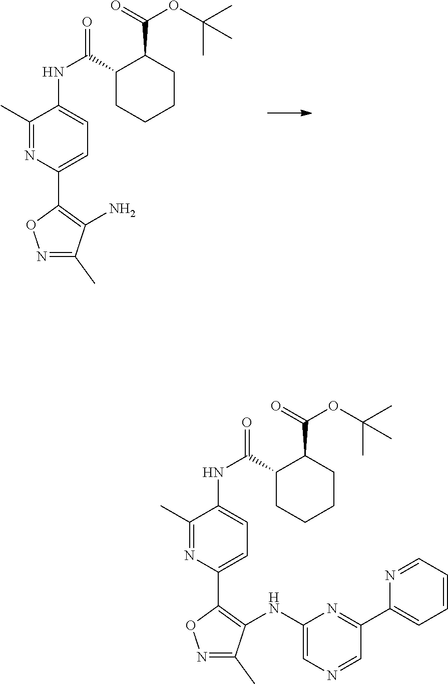 [SMILES: Cc1nc(-c2onc(C)c2N)ccc1NC(=O)[C@H]1CCCC[C@@H]1C(=O)OC(C)(C)C ; Cc1nc(-c2onc(C)c2Nc2cncc(-c3ccccn3)n2)ccc1NC(=O)[C@H]1CCCC[C@@H]1C(=O)OC(C)(C)C]

Intermediate B23 (137.5 mg, 0.241 mmol, 40.02% yield) was obtained from 2-chloro-6-(pyridin-2-yl)pyrazine (Intermediate B22, 8.24 mg, 0.510 mmol) and tert-butyl (1S,2S)-2-((6-(4-amino-3-methylisoxazol-5-yl)-2-methylpyridin-3-yl)carbamoyl)cyclohexane-1-carboxylate (Intermediate B6, 250.0 mg, 0.600 mmol) using a similar method as for Intermediate B14.

LC-MS (ESI): m/z (M+1): 570.3 (Method 1)

### Step 3: (1S,2S)-2-((2-methyl-6-(3-methyl-4-((6-(pyridin-2-yl)pyrazin-2-yl)amino)isoxazol-5-yl)pyridin-3-yl)carbamoyl)cyclohexane-1-carboxylic acid (Compound 14)

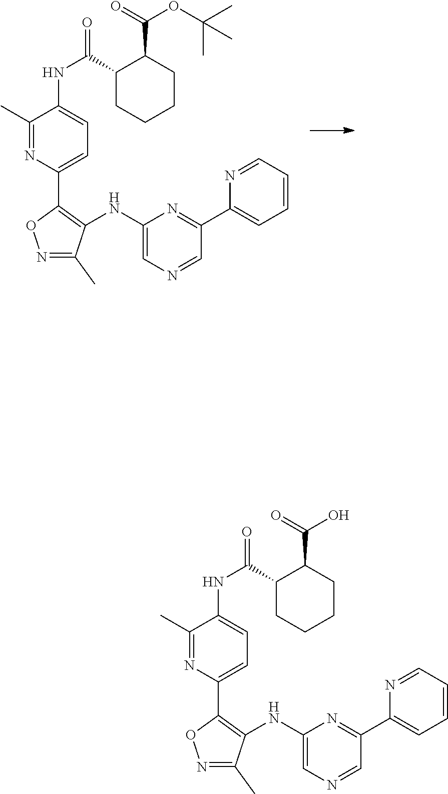 [SMILES: Cc1nc(-c2onc(C)c2Nc2cncc(-c3ccccn3)n2)ccc1NC(=O)[C@H]1CCCC[C@@H]1C(=O)OC(C)(C)C ; Cc1nc(-c2onc(C)c2Nc2cncc(-c3ccccn3)n2)ccc1NC(=O)[C@H]1CCCC[C@@H]1C(=O)O]

Compound 14 (383 mg, 0.990 mmol, 80.35% yield) was obtained from tert-butyl (1S,2S)-2-((2-methyl-6-(3-methyl-4-((6-(pyridin-2-yl)pyrazin-2-yl)amino)isoxazol-5-yl)pyridin-3-yl)carbamoyl)cyclohexane-1-carboxylate (Intermediate B23, 137.5 mg, 0.241 mmol) using a similar method as for Compound 7.

LC-MS (ESI): m/z (M+1): 514.2 (Method 1)

¹H NMR (400 MHz, DMSO-d₆) δ ppm 12.07 (br s, 1H), 9.51 (s, 1H), 9.04 (s, 1H), 8.80 (s, 1H), 8.64 (dd, J=4.7, 0.8 Hz, 1H), 8.27 (s, 1H), 7.90 (d, J=8.4 Hz, 1H), 7.82-7.88 (m, 1H), 7.75 (d, J=7.9 Hz, 1H), 7.69 (d, J=8.4 Hz, 1H), 7.41 (ddd, J=7.5, 4.8, 1.1 Hz, 1H), 2.59-2.67 (m, 1H), 2.42-2.48 (m, 1H), 2.31 (s, 3H), 2.26 (s, 3H), 1.92-2.05 (m, 2H), 1.65-1.85 (m, 2H), 1.18-1.45 (m, 4H)

### Example 5

### Process Synthesis of Compound 15

### (1S,2S)-2-((6-(4-((6-(cyclopentylamino)pyrazin-2-yl)amino)-3-methylisoxazol-5-yl)-2-methylpyridin-3-yl)carbamoyl)cyclohexane-1-carboxylic acid

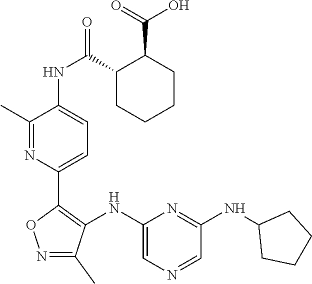 [SMILES: Cc1nc(-c2onc(C)c2Nc2cncc(NC3CCCC3)n2)ccc1NC(=O)[C@H]1CCCC[C@@H]1C(=O)O]

### Step 1: 6-chloro-N-cyclopentylpyrazin-2-amine (Intermediate B24)

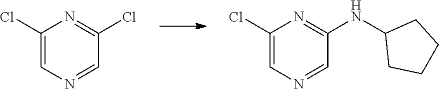 [SMILES: Clc1cncc(Cl)n1 ; Clc1cncc(NC2CCCC2)n1]

2,6-dichloropyrazine (400.0 mg, 2.68 mmol) was suspended in THE (7 mL), then cyclopentanamine (0.341 mL, 3.49 mmol) and TEA (0.486 mL 3.49 mmol) were added. The mixture was heated at reflux for 24 h, and then a 2^{nd} addition of cyclopentanamine (0.341 mL, 3.49 mmol) and TEA (0.486 mL, 3.49 mmol) was done. The mixture was heated at reflux for 2 days, then cooled at RT, concentrated under reduced pressure and recovered with EtOAc. The organic fraction was washed with sat. aq. NH4Cl, water and brine, dried over Na2SO4, filtered and concentrated. The residue was purified by column chromatography (gradient EtOAc 5-30% in CyHex) to afford the target compound (400 mg, 2.024 mmol, 75.37% yield) as a light brown solid.

LC-MS (ESI): m/z (M+1): 198.0 (Method 1)

### Step 2: tert-butyl (1S,2S)-2-((6-(4-((6-(cyclopentylamino)pyrazin-2-yl)amino)-3-methylisoxazol-5-yl)-2-methylpyridin-3-yl)carbamoyl)cyclohexane-1-carboxylate (Intermediate B25)

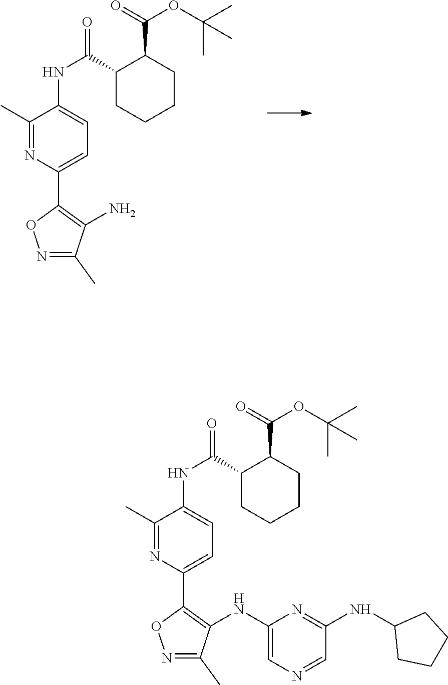 [SMILES: Cc1nc(-c2onc(C)c2N)ccc1NC(=O)[C@H]1CCCC[C@@H]1C(=O)OC(C)(C)C ; Cc1nc(-c2onc(C)c2Nc2cncc(NC3CCCC3)n2)ccc1NC(=O)[C@H]1CCCC[C@@H]1C(=O)OC(C)(C)C]

Intermediate B25 (99.6 mg, 0.173 mmol, 71.71% yield) was obtained from 6-chloro-N-cyclopentylpyrazin-2-amine (Intermediate B24, 47.69 mg, 0.240 mmol) and tert-butyl (1S,2S)-2-((6-(4-amino-3-methylisoxazol-5-yl)-2-methylpyridin-3-yl)carbamoyl)cyclohexane-1-carboxylate (Intermediate B6, 100.0 mg, 0.240 mmol) using a similar method as for Intermediate B14.

LC-MS (ESI): m/z (M+1): 576.3 (Method 1)

### Step 3: (1S,2S)-2-((6-(4-((6-(cyclopentylamino)pyrazin-2-yl)amino)-3-methylisoxazol-5-yl)-2-methylpyridin-3-yl)carbamoyl)cyclohexane-1-carboxylic acid (Compound 15)

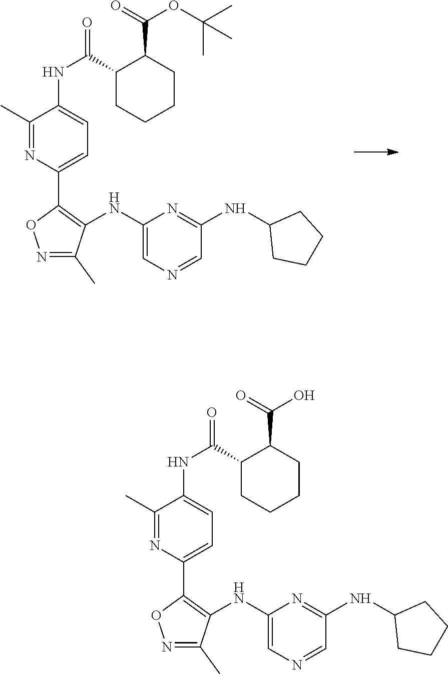 [SMILES: Cc1nc(-c2onc(C)c2Nc2cncc(NC3CCCC3)n2)ccc1NC(=O)[C@H]1CCCC[C@@H]1C(=O)OC(C)(C)C ; Cc1nc(-c2onc(C)c2Nc2cncc(NC3CCCC3)n2)ccc1NC(=O)[C@H]1CCCC[C@@H]1C(=O)O]

Compound 15 (62 mg, 0.119 mmol, 68.97% yield) was obtained from tert-butyl (1S,2S)-2-((6-(4-((6-(cyclopentylamino)pyrazin-2-yl)amino)-3-methylisoxazol-5-yl)-2-methylpyridin-3-yl)carbamoyl)cyclohexane-1-carboxylate 9Intermediate B25, 99.6 mg, 0.170 mmol) using a similar method as for Compound 7.

LC-MS (ESI): m/z (M+1): 520.3 (Method 1)

¹H NMR (400 MHz, DMSO-d₆) δ ppm 11.78 (br s, 1H), 9.57 (s, 1H), 8.21 (s, 1H), 7.89 (d, J=8.3 Hz, 1H), 7.61 (d, J=8.3 Hz, 1H), 7.23 (s, 1H), 7.20 (s, 1H), 6.58 (d, J=6.8 Hz, 1H), 3.76 (sxt, J=6.7 Hz, 1H), 2.72-2.61 (m, 1H), 2.58-2.50 (m, 1H), 2.42 (s, 3H), 2.21 (s, 3H), 2.07-1.92 (m, 2H), 1.85-1.65 (m, 4H), 1.65-1.51 (m, 2H), 1.50-1.39 (m, 2H), 1.38-1.20 (m, 6H)

### Example 6

### Process Synthesis of Compound 16

### 2-((4-(4-((6-isopropoxypyrazin-2-yl)amino)-3-methylisoxazol-5-yl)phenyl)carbamoyl)cyclohexane-1-carboxylic acid (trans racemate)

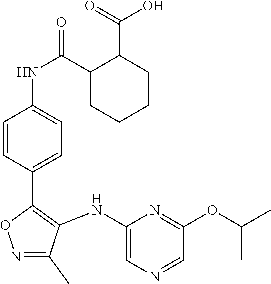 [SMILES: Cc1noc(-c2ccc(NC(=O)C3CCCCC3C(=O)O)cc2)c1Nc1cncc(OC(C)C)n1]

### Step 1: 3-methyl-5-(4-nitrophenyl)isoxazol-4-amine (Intermediate C1)

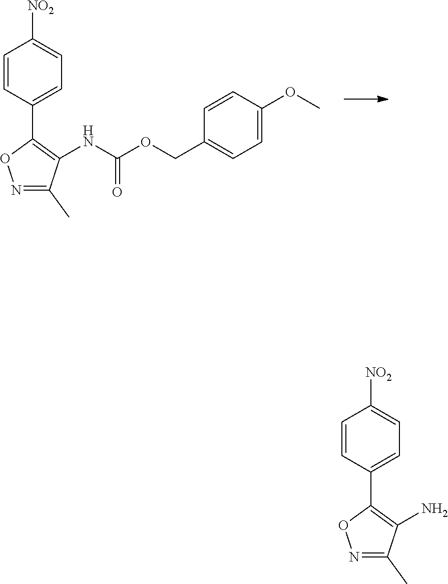 [SMILES: COc1ccc(COC(=O)Nc2c(C)noc2-c2ccc([N+](=O)[O-])cc2)cc1 ; Cc1noc(-c2ccc([N+](=O)[O-])cc2)c1N]

Intermediate C1 ((264 mg, 1.204 mmol, 49.81% yield) was obtained from 4-methoxybenzyl (3-methyl-5-(4-nitrophenyl)isoxazol-4-yl)carbamate (Intermediate A17, 926.94 mg, 2.42 mmol) using a similar method as for Intermediate A31.

LC-MS (ESI): m/z (M+1): 220.1 (Method 1)

### Step 2: N-(6-isopropoxypyrazin-2-yl)-3-methyl-5-(4-nitrophenyl)isoxazol-4-amine (Intermediate C2)

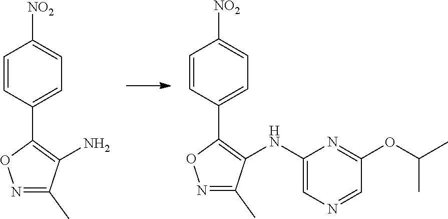 [SMILES: Cc1noc(-c2ccc([N+](=O)[O-])cc2)c1N ; Cc1noc(-c2ccc([N+](=O)[O-])cc2)c1Nc1cncc(OC(C)C)n1]

A suspension of 3-methyl-5-(4-nitrophenyl)isoxazol-4-amine (Intermediate C1, 140.0 mg, 0.640 mmol) and 2-chloro-6-isopropoxypyrazine (Intermediate A23, 121.27 mg, 0.700 mmol) in DCE (4.667 mL), K₃PO₄ (338.93 mg, 1.6 mmol) was added. After applying three cycles nitrogen/vacuum, XPhos Pd G2 (50.19 mg, 0.060 mmol) and Pd₂(dba)₃ (58.49 mg, 0.060 mmol) were added. The reaction was heated at 105° C. for 5h. After cooling down the temperature, the mixture was diluted with DCM and filtered to remove the solid. The crude was purified by flash chromatography using a gradient of EtOAc in cCyHex from 0% to 55% affording the desired product (135 mg, 0.380 mmol, 59.48% yield) as a yellow solid.

LC-MS (ESI): m/z (M+1): 356.0 (Method 1)

### Step 3: 5-(4-aminophenyl)-N-(6-isopropoxypyrazin-2-yl)-3-methylisoxazol-4-amine (Intermediate C3)

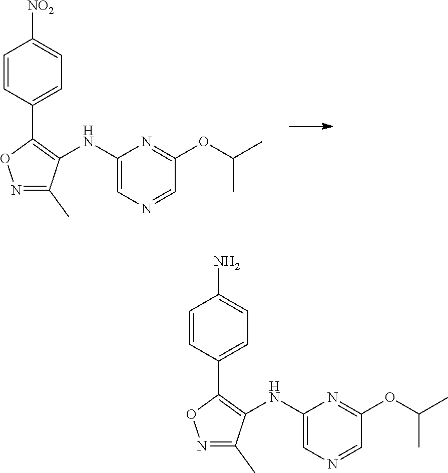 [SMILES: Cc1noc(-c2ccc([N+](=O)[O-])cc2)c1Nc1cncc(OC(C)C)n1 ; Cc1noc(-c2ccc(N)cc2)c1Nc1cncc(OC(C)C)n1]

N-(6-isopropoxypyrazin-2-yl)-3-methyl-5-(4-nitrophenyl)isoxazol-4-amine (Intermediate C2, 135.0 mg, 0.380 mmol) was dissolved in DCM (0.647 mL), MeOH (0.259 mL) and concentrated HCl (13.85 mg, 0.380 mmol), then Fe⁰ (148.52 mg, 2.66 mmol) was added and the mixture so obtained was stirred at RT for 1 h. The mixture was concentrated under reduced pressure. The residue was basified with a 2N aqueous NaOH solution and extracted with EtOAc and the organic phase were washed with brine and concentrated in vacuo to give the crude product (113 mg, 0.347 mmol, 91.42% yield) as yellow oil. It was used in the following step without any purifications.

LC-MS (ESI): m/z (M+1): 326.3 (Method 1)

### Step 4: methyl 2-((4-(4-((6-isopropoxypyrazin-2-yl)amino)-3-methylisoxazol-5-yl)phenyl)carbamoyl)cyclohexane-1-carboxylate (trans racemate) (Intermediate C4)

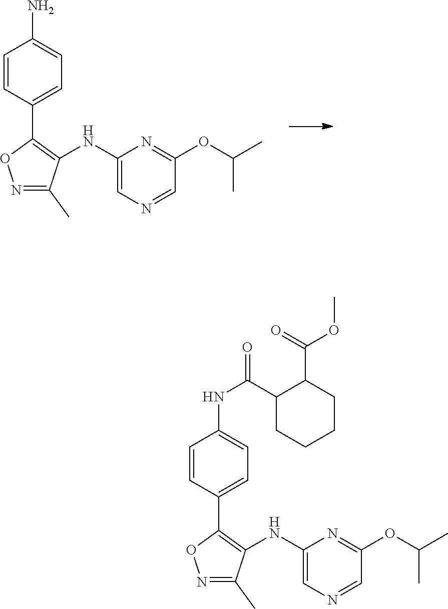 [SMILES: Cc1noc(-c2ccc(N)cc2)c1Nc1cncc(OC(C)C)n1 ; COC(=O)C1CCCCC1C(=O)Nc1ccc(-c2onc(C)c2Nc2cncc(OC(C)C)n2)cc1]

To a solution of trans-rac 2-methoxycarbonylcyclohexane-1-carboxylic acid (58.84 mg, 0.320 mmol) in DMF (0.250 mL), oxalyl dichloride (0.04 mL, 0.470 mmol) was added dropwise at 0° C., DCM (0.500 mL) was added, then the mixture was stirred at RT 1 h. The mixture was then concentrated under vacuum. A solution of this chloride in THE (1 mL) was added dropwise to a solution of 5-(4-aminophenyl)-N-(6-isopropoxypyrazin-2-yl)-3-methylisoxazol-4-amine (Intermediate C3, 113.09 mg, 0.350 mmol) in TEA (0.13 mL, 0.950 mmol) at 0° C., then the mixture was stirred at RT for 2 h. The mixture was concentrated under vacuum and the whole crude was purified by flash chromatography using a gradient of MeOH in DCM from 0% to 30% to afford the target compound (60.5 mg, 0.123 mmol, 38.79% yield) as an amorphous orange solid.

LC-MS (ESI): m/z (M+1): 494.4 (Method 1)

### Step 5: 2-((4-(4-((6-isopropoxypyrazin-2-yl)amino)-3-methylisoxazol-5-yl)phenyl)carbamoyl)cyclohexane-1-carboxylic acid (trans racemate) (Compound 16)

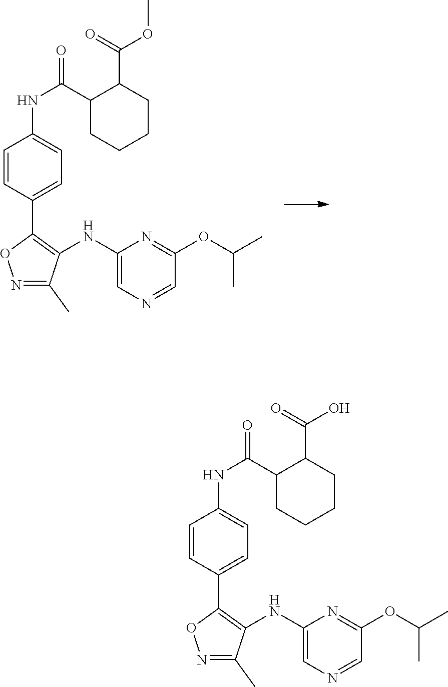 [SMILES: COC(=O)C1CCCCC1C(=O)Nc1ccc(-c2onc(C)c2Nc2cncc(OC(C)C)n2)cc1 ; Cc1noc(-c2ccc(NC(=O)C3CCCCC3C(=O)O)cc2)c1Nc1cncc(OC(C)C)n1]

Compound 16 (13 mg, 0.027 mmol, 22.12% yield) was obtained from methyl 2-((4-(4-((6-isopropoxypyrazin-2-yl)amino)-3-methylisoxazol-5-yl)phenyl)carbamoyl)cyclohexane-1-carboxylate (trans racemate) (Intermediate C4, 60.5 mg, 0.120 mmol) using a similar method as for Intermediate B4.

LC-MS (ESI): m/z (M+1): 480.5 (Method 1)

¹H NMR (400 MHz, DMSO-d₆) δ ppm 12.05 (br s, 1H), 10.20 (br s, 1H), 8.70 (s, 1H), 7.68 (s, 4H), 7.61 (s, 1H), 7.42 (s, 1H), 4.74 (dt, J=12.2, 6.1 Hz, 1H), 2.60-2.52 (m, 2H), 2.15-2.13 (m, 1H), 2.13 (s, 3H), 2.07-1.66 (m, 4H), 1.38-1.18 (m, 4H), 1.10 (d, J=6.1 Hz, 6H)

### Example 7

### Process of Synthesis of Compound 17

### (1S,2S)-2-((6-(5-(((4-isopropoxypyrimidin-2-yl)amino)methyl)-1-methyl-1H-pyrazol-4-yl)-2-methylpyridin-3-yl)carbamoyl)cyclohexane-1-carboxylic acid

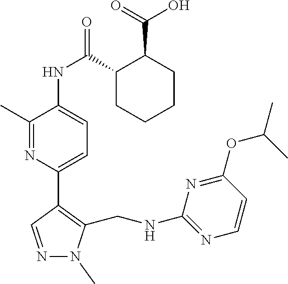 [SMILES: Cc1nc(-c2cnn(C)c2CNc2nccc(OC(C)C)n2)ccc1NC(=O)[C@H]1CCCC[C@@H]1C(=O)O]

### Step 1: methyl 1-methyl-4-(4,4,5,5-tetramethyl-1,3,2-dioxaborolan-2-yl)-1H-pyrazole-5-carboxylate (Intermediate D1)

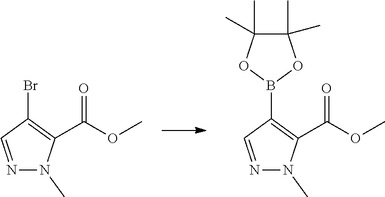

A mixture of methyl 4-bromo-1-methyl-1H-pyrazole-5-carboxylate (5.0 g, 22.83 mmo), KOAc (6.72 g, 68.48 mmol) and B₂pin₂ (8.69 g, 34.24 mmol) in 1,4-Dioxane (40 mL) was degassed under N2 for 5 minutes, then Pd(dppf)Cl2 (1.87 g, 2.28 mmol) was added. The mixture was submitted to 3 MW cycles of 30 min at 100° C. The crude was filtered, water was added and the mixture was extracted with EtOAc for 3 times, collected organic fractions were dried over Na2SO4, filtered and evaporated to give title compound (8 g, crude) as brown oil which was used in the next step without further purification.

LC-MS (ESI): m/z (M+1): 267.0 (Method 1)

### Step 2: tert-butyl (6-bromo-2-methylpyridin-3-yl)carbamate (Intermediate D2)

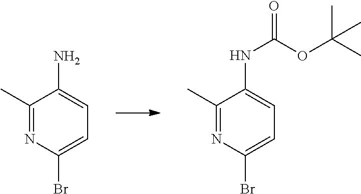

To a suspension of 6-bromo-2-methylpyridin-3-amine (7.0 g, 37.43 mmol) in propan-2-ol (14.0 mL, 232.95 mmol) and Water (5 mL), (Boc)₂O (9.39 g, 43.04 mmol) was added portionwise at 0° C., then the reaction was warmed to RT and stirred overnight. Further (Boc)₂O (9.39 g, 43.04 mmol) was added at RT. After 24 h the mixture was concentrated under vacuum, water was added and the reaction extracted with EtOAc (3 times). The collected organic layers were dried over Na₂SO₄, filtered and concentrated to afford the crude that was purified by flash chromatography using a gradient of CyHex/EtOAc from 10/0 to 8/2 to afford the target in compound (8.51 g, 29.64 mmol, 79.19% yield) as white solid.

LC-MS (ESI): m/z (M+1): 289.1 (Method 1)

### Step 3: methyl 4-(5-((tert-butoxycarbonyl)amino)-6-methylpyridin-2-yl)-1-methyl-1H-pyrazole-5-carboxylate (Intermediate D3)

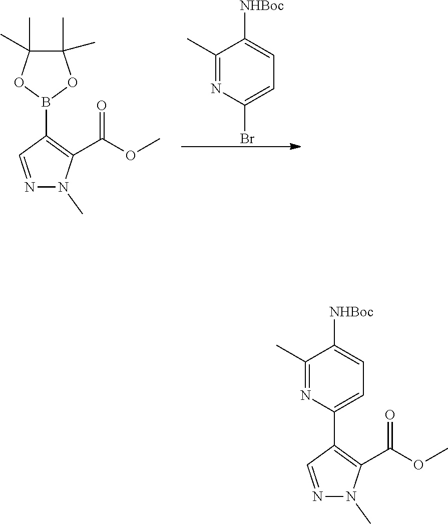

To a solution of methyl 1-methyl-4-(4,4,5,5-tetramethyl-1,3,2-dioxaborolan-2-yl)-1H-pyrazole-5-carboxylate (Intermediate D1, 6.08 g, 22.83 mmol) in 1,4-Dioxane (48 mL), 6 tert-butyl N-(6-bromo-2-methylpyridin-3-yl)carbamate (Intermediate D2, 7.21 g, 25.11 mmol), K₂HPO₄ (11.93 g, 68.49 mmol), Water (48 mL and X-Phos Pd G2 (1.79 g, 2.28 mmol) were added under N2. The mixture was stirred at 60° C. overnight. Brine was added and the mixture was extracted with EtOAc for 3 times, collected organic phases were dried over Na₂SO₄, filtered and evaporated under reduced pressure. The crude was purified by flash chromatography eluting with a gradient of CyHex/EtOAc from 100/0 to 0/100 to afford title compound (4.89 g, 14.12 mmol, 62% yield) as an orange oil.

LC-MS (ESI): m/z (M+1): 346 (Method 1)

The Intermediates in the following table were prepared from reagents reported below by using methods analogous to Intermediate D3.

| Intermediate | Structure & Name | Reagents | Analytical data |
|---|---|---|---|
| D4 | 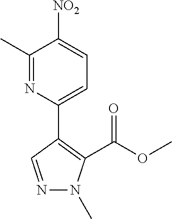 | D1 and 6- bromo-2- methyl-3- nitropyridine | LC-MS (ESI): m/z (M + 1): 277.1 (Method 1) ¹H NMR (400 MHz, DMSO-d₆) d ppm 8.45 (d, J = 8.58 Hz, 1 H) 8.07 (s, 1 H) 7.79 (d, J = 8.58 Hz, 1 H) 4.02 (s, 3 H) 3.86 (s, 3 H) 2.76 (s, 3 H) |
| | methyl 1-methyl-4-(6- | | |
| | methyl-5-nitropyridin-2- | | |
| | yl)-1H-pyrazole-5- | | |
| | carboxylate | | |

### Step 4: 4-(5-((tert-butoxycarbonyl)amino)-6-methylpyridin-2-yl)-1-methyl-1H-pyrazole-5-carboxylic acid (Intermediate D5)

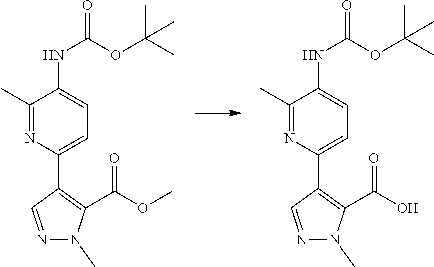 [SMILES: COC(=O)c1c(-c2ccc(NC(=O)OC(C)(C)C)c(C)n2)cnn1C ; Cc1nc(-c2cnn(C)c2C(=O)O)ccc1NC(=O)OC(C)(C)C]

Intermediate D5 (1.84 g, 5.536 mmol, 45.6% yield) was obtained from methyl 4-(5-((tert-butoxycarbonyl)amino)-6-methylpyridin-2-yl)-1-methyl-1H-pyrazole-5-carboxylate (Intermediate D3, 4.89 g, 12.14 mmol) using a similar method as for Intermediate B4.

LC-MS (ESI): m/z (M+1): 333.2 (Method 1)

### Step 5: tert-butyl (6-(5-(hydroxymethyl)-1-methyl-1H-pyrazol-4-yl)-2-methylpyridin-3-yl)carbamate (Intermediate D6)

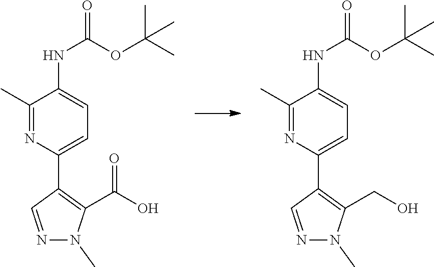 [SMILES: Cc1nc(-c2cnn(C)c2C(=O)O)ccc1NC(=O)OC(C)(C)C ; Cc1nc(-c2cnn(C)c2CO)ccc1NC(=O)OC(C)(C)C]

To a solution of 4-(5-((tert-butoxycarbonyl)amino)-6-methylpyridin-2-yl)-1-methyl-1H-pyrazole-5-carboxylic acid (Intermediate D5, 1.84 g, 5.54 mmol) in THF (32 mL), CDI (1.8 g, 11.07 mmol) was added and the mixture was stirred overnight at 65° C. The mixture was then cooled with and ice bath and NaBH₄ (523.6 mg, 13.84 mmol) and Water (20 mL) were added. The mixture was allowed to reach RT and stirred for 30 minutes. The mixture was diluted with EtOAc and washed with brine. The organic phase was separated, dried over Na2SO4, filtered and evaporated under reduced pressure. The crude was purified by flash chromatography eluting with a gradient of CyHex/EtOAc from 50/50 to 0/100 to provide title compound (706 mg, 2.218 mmol, 40.05% yield) as a pale yellow solid.

LC-MS (ESI): m/z (M+1): 319.2 (Method 1)

The Intermediates in the following table were prepared from reagents reported below by using methods analogous to Intermediate D6.

| Inter- | | | |
|---|---|---|---|
| mediate | Structure & Name | Reagents | Analytical data |
| D7 | 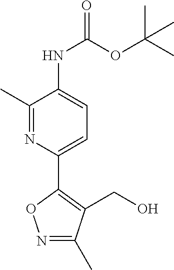 [SMILES: Cc1nc(-c2onc(C)c2CO)ccc1NC(=O)OC(C)(C)C] | A9 | LC-MS (ESI): m/z (M + 1): 302.2 (Method 1) ¹H NMR (400 MHz, DMSO-d₆) d ppm 8.97 (s, 1 H), 8.02 (d, J = 8.58 Hz, 1 H), 7.72 (d, J = 8.58 Hz, 1 H), 5.20 (t, J = 5.72 Hz, 1 H), 4.73 (d, J = 5.72 Hz, 2 H), 2.50 (s, 3H), 2.31 (s, 3 H), 1.49 (s, 9 H) |
| | tert-butyl (6-(4- | | |
| | (hydroxymethyl)-3- | | |
| | methylisoxazol-5-yl)-2- | | |
| | methylpyridin-3- | | |
| | yl)carbamate | | |
| D8 | 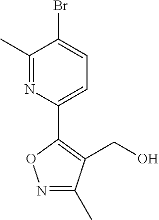 [SMILES: Cc1nc(-c2onc(C)c2CO)ccc1Br] | A11 | LC-MS (ESI): m/z (M + 1): 285.0 (Method 1) ¹H NMR (400 MHz, Chloroform- d) δ 8.03 (d, J = 8.33 Hz, 1H), 7.73 (d, J = 8.30 Hz, 1H), 5.95 (t, J = 6.80 Hz, 1H), 4.64 (d, J = 6.81 Hz, 2H), 2.77 (s, 3H), 2.38 (s, 3H). |
| | (5-(5-bromo-6- | | |
| | methylpyridin-2-yl)-3- | | |
| | methylisoxazol-4- | | |
| | yl)methanol | | |

### Step 6 tert-butyl (6-(5-(chloromethyl)-1-methyl-1H-pyrazol-4-yl)-2-methylpyridin-3-yl)carbamate (Intermediate D9)

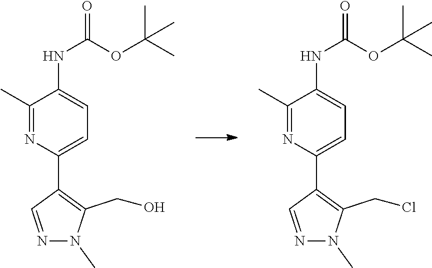 [SMILES: Cc1nc(-c2cnn(C)c2CO)ccc1NC(=O)OC(C)(C)C ; Cc1nc(-c2cnn(C)c2CCl)ccc1NC(=O)OC(C)(C)C]

To an ice-cooled solution of tert-butyl N-[6-[5-(hydroxymethyl)-1-methylpyrazol-4-yl]-2-methylpyridin-3-yl]carbamate (Intermediate D6, 706.0 mg, 2.22 mmol) in DCM (15 mL), TEA (0.93 mL, 6.65 mmol) was added followed by the addition of methanesulfonyl chloride (0.34 mL, 4.44 mmol). The mixture was stirred at RT for 2h then volatiles were removed at reduced pressure to provide the product (2.4 g, crude).

LC-MS (ESI): m/z (M+C): 337.1 (Method 1)

The Intermediates in the following table were prepared from reagents reported below by using methods analogous to Intermediate D9.

| Intermediate | Structure & Name | Reagents | Analytical data |
|---|---|---|---|
| D10 | 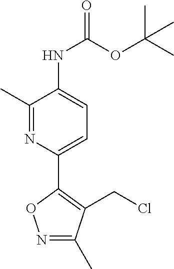 [SMILES: Cc1nc(-c2onc(C)c2CCl)ccc1NC(=O)OC(C)(C)C] | D7 | LC-MS (ESI): m/z (M + 1): 338.2 (Method 1) |
| | ter-butyl (6-(4- | | |
| | (chloromethyl)-3- | | |
| | methylisoxazol-5-yl)-2- | | |
| | methylpyridin-3- | | |
| | yl)carbamate | | |

### Step 7: tert-butyl (6-(5-(aminomethyl)-1-methyl-1H-pyrazol-4-yl)-2-methylpyridin-3-yl)carbamate (Intermediate D11)

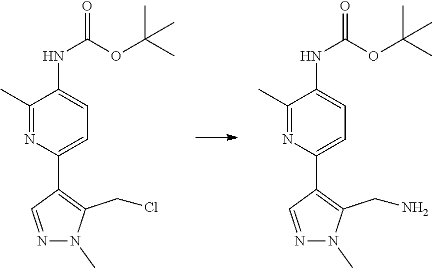 [SMILES: Cc1nc(-c2cnn(C)c2CCl)ccc1NC(=O)OC(C)(C)C ; Cc1nc(-c2cnn(C)c2CN)ccc1NC(=O)OC(C)(C)C]

To a solution of tert-butyl N-[6-[5-(chloromethyl)-1-methylpyrazol-4-yl]-2-methylpyridin-3-yl]carbamate (Intermediate D9, 747 mg, 2.22 mmol) in 1,4-Dioxane (20 mL), ammonium hydroxide (15.42 mL, 110.9 mmol) was added and the mixture was stirred at RT overnight. The mixture was extracted with EtOAc (3 times) and organics were collected, dried over Na₂SO₄, filtered and concentrated at reduced pressure. The crude was purified by flash chromatography eluting with a gradient of DCM/MeOH from 100/0 to 98/2. Fractions containing the product were concentrated and further purified by flash chromatography using a gradient of Water/MeCN with 0.1% of formic acid from 97/3 to 80/20 to give the title compound (421 mg, 1.326 mmol, 59.8% yield) as a white solid.

LC-MS (ESI): m/z (M+1): 318.1 (Method 1)

The Intermediates in the following table were prepared from reagents reported below by using methods analogous to Intermediate D11.

| Inter- | | | |
|---|---|---|---|
| mediate | Structure & Name | Reagents | Analytical data |
| D12 | 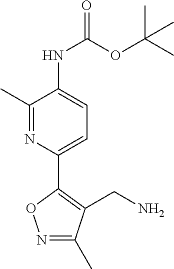 [SMILES: Cc1nc(-c2onc(C)c2CN)ccc1NC(=O)OC(C)(C)C] | D10 | LC-MS (ESI): m/z 319.2 (M + 1): (Method 1) ¹H NMR (400 MHz, DMSO-d6) δ ppm 9.12-8.80 (m, 1 H) 8.00 (d, J = 8.36 Hz, 1 H) 7.71 (d, J = 8.58 Hz, 1 H) 3.88-3.73 (m, 2 H) 3.31 (s, 3 H) 2.31 (s, 3 H) 1.97- 1.73 (m, 2 H) 1.50 (s, 9 H) |
| | tert-butyl (6-(4- | | |
| | (aminomethyl)-3- | | |
| | methylisoxazol-5-yl)-2- | | |
| | methylpyridin-3- | | |
| | yl)carbamate | | |

### Step 8: 2-chloro-4-isopropoxypyrimidine (Intermediate D13)

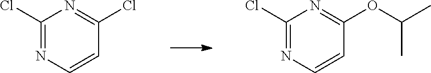

A mixture of 2,4-dichloropyrimidine (300.0 mg, 2.01 mmol) and Cs₂CO₃ (721.71 mg, 2.22 mmol) in propan-2-ol (4.0 mL, 52.32 mmol) was vigorously stirred at 80° C. for 5 h. Volatiles were removed at reduced pressure and the residue was partitioned between DCM and saturated NaHCO₃ aqueous solution. The organic phase was separated, filtered through a hydrophobic phase separator and concentrated at reduced pressure. The crude was purified by flash chromatography with a gradient of CyHex/EtOAc from 100:0 to 93:7 to provide the target compound (173 mg, 1.002 mmol, 49.77% yield) as a colorless oil.

LC-MS (ESI): m/z (M+1): 173 (Method 1)

### Step 9: tert-butyl (6-(5-(((4-isopropoxypyrimidin-2-yl)amino)methyl)-1-methyl-1H-pyrazol-4-yl)-2-methylpyridin-3-yl)carbamate (Intermediate D14)

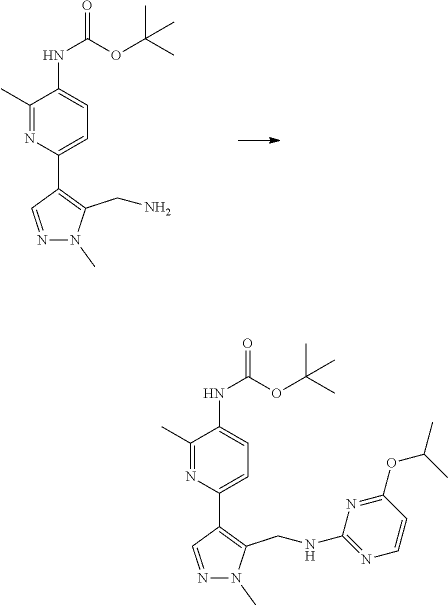 [SMILES: Cc1nc(-c2cnn(C)c2CN)ccc1NC(=O)OC(C)(C)C ; Cc1nc(-c2cnn(C)c2CNc2nccc(OC(C)C)n2)ccc1NC(=O)OC(C)(C)C]

A mixture of tert-butyl (6-(5-(aminomethyl)-1-methyl-1H-pyrazol-4-yl)-2-methylpyridin-3-yl)carbamate (Intermediate D11, 100.0 mg, 0.320 mmol), 2-chloro-4-isopropoxypyrimidine (Intermediate D13, 59.82 mg, 0.350 mmol) and K₂CO₃ (65.32 mg, 0.470 mmol) in DMF (1.5 mL) was stirred at 80° C. overnight. The mixture was allowed to reach RT, poured into saturated NaHCO₃ aqueous solution and extracted with EtOAc. The organic phase was separated, filtered through a hydrophobic phase separator and concentrated at reduced pressure. The crude was purified by flash chromatography with a gradient of DCM/MeOH from 100:0 to 96:4 to provide the desired compound (142 mg, 0.313 mmol, 99.37% yield) as a colorless oil.

LC-MS (ESI): m/z (M+1): 454.3 (Method 1)

The Intermediates in the following table were prepared from reagents reported below by using methods analogous to Intermediate D14.

| Intermediate | Structure & Name | Reagents | Analytical data |
|---|---|---|---|
| D15 | 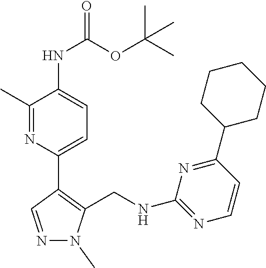 [SMILES: Cc1nc(-c2cnn(C)c2CNc2nccc(C3CCCCC3)n2)ccc1NC(=O)OC(C)(C)C] tert-butyl (6-(5-(((4- cyclohexylpyrimidin-2- yl)amino)methyl)-1- methyl-1H-pyrazol-4-yl)- 2-methylpyridin-3- yl)carbamate | D12 and 2- chloro-4- cyclohexyl- pyrimidine | LC-MS (ESI): m/z (M + 1): 478.3 (Method 1) 1H NMR (500 MHz, DMSO-d6) δ ppm 12.11 (br s, 1 H), 8.97-10.16 (m, 1 H), 8.17 (br s, 1 H), 7.87 (s, 1 H), 7.74 (br s, 1 H), 7.51 (d, J = 8.4 Hz, 1 H), 7.40 (t, J = 6.0 Hz, 1 H), 6.50 (d, J = 5.1 Hz, 1 H), 4.77-4.93 (m, 2 H), 3.98 (s, 3 H), 2.60-2.69 (m, 1 H), 2.44 (s, 3 H), 2.34- 2.43 (m, 2 H), 1.92-2.07 (m, 2 H), 1.60-1.83 (m, 7 H), 1.10-1.48 (m, 9 H) |
| D16 | 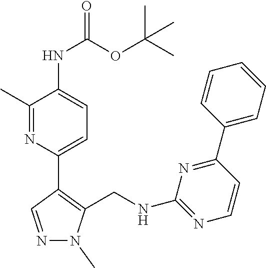 [SMILES: Cc1nc(-c2cnn(C)c2CNc2nccc(-c3ccccc3)n2)ccc1NC(=O)OC(C)(C)C] tert-butyl (2-methyl-6- (1-methyl-5-(((4- phenylpyrimidin-2- yl)amino)methyl)-1H- pyrazol-4-yl)pyridin-3- yl)carbamate | D12 and 2- chloro-4- phenyl- pyrimidine | LC-MS (ESI): m/z (M + 1): 472.3 (Method 1) ¹H NMR (400 MHz, Chloroform-d) δ 8.36 (d, J = 5.17 Hz, 1H), 8.16 (d, J = 8.60 Hz, 1H), 8.04 (dd, J = 6.65, 3.03 Hz, 2H), 7.77 (s, 1H), 7.49 (d, J = 6.53 Hz, 3H), 7.40 (d, J = 8.53 Hz, 1H), 7.16 (s, 1H), 6.99 (d, J = 5.21 Hz, 1H), 6.29 (s, 1H), 5.03 (d, J = 6.27 Hz, 2H), 4.11 (s, 3H), 2.64 (s, 3H), 1.57 (s, 9H) |

### Step 10: N-((4-(5-amino-6-methylpyridin-2-yl)-1-methyl-1H-pyrazol-5-yl)methyl)-4-isopropoxypyrimidin-2-amine (Intermediate D17)

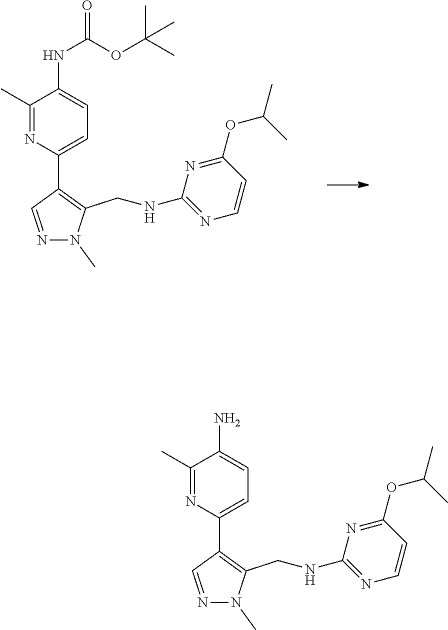

Intermediate D17 (72 mg, 0.204 mmol, 65.07% yield) was obtained as a yellow solid from tert-butyl (6-(5-(((4-isopropoxypyrimidin-2-yl)amino)methyl)-1-methyl-1H-pyrazol-4-yl)-2-methylpyridin-3-yl)carbamate (Intermediate D14, 142.0 mg, 0.310 mmol) using a similar method as for Intermediate A31.

LC-MS (ESI): m/z (M+1): 354.2 (Method 1)

### Step 11: (1S,2S)-2-((6-(5-(((4-isopropoxypyrimidin-2-yl)amino)methyl)-1-methyl-1H-pyrazol-4-yl)-2-methylpyridin-3-yl)carbamoyl)cyclohexane-1-carboxylic acid (Compound 17)

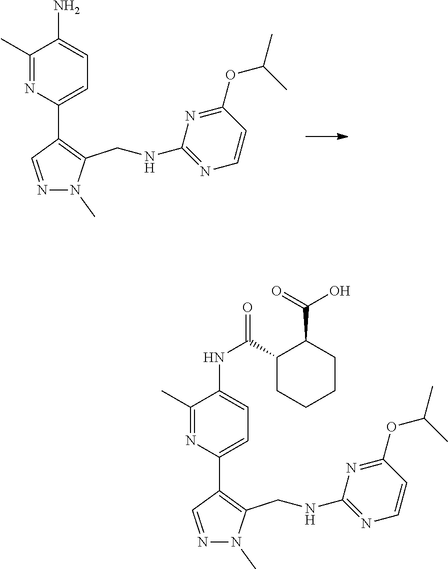

To a solution of N-((4-(5-amino-6-methylpyridin-2-yl)-1-methyl-1H-pyrazol-5-yl)methyl)-4-isopropoxypyrimidin-2-amine (Intermediate D17, 72.0 mg, 0.200 mmol) in MeCN (1 mL),), (−)-trans-1,2-Cyclohexanedicarboxylic anhydride (34.55 mg, 0.220 mmol) was added and the mixture was shaken at 40° C. overnight. Volatiles were removed at reduced pressure and the crude was purified by flash chromatography with a gradient of Water/MeCN with 0.1% of formic acid from 97:3 to 70:30 to provide a solid that was further purified by flash chromatography using a gradient of DCM in MeOH from 100:0 to 80:20. Evaporation of proper fractions provided the title compound (36 mg, 0.071 mmol, 34.81% yield) as a white solid.

LC-MS (ESI): m/z (M+1): 508.3 (Method 1)

¹H NMR (400 MHz, DMSO-d₆) δ ppm 12.11 (br s, 1H), 9.50 (br s, 1H), 8.00 (br s, 1H), 7.87 (s, 1H), 7.67 (d, J=8.4 Hz, 1H), 7.54 (br s, 1H), 7.50 (d, J=8.4 Hz, 1H), 5.96 (br d, J=4.8 Hz, 1H), 5.09 (br s, 1H), 4.87 (br s, 2H), 3.93 (s, 3H), 2.73-2.59 (m, 1H), 2.53-2.49 (m, 1H), 2.41 (s, 3H), 2.09-1.94 (m, 2H), 1.83-1.62 (m, 2H), 1.43-1.22 (m, 4H), 1.17 (br s, 6H)

The Compounds in the following table were prepared from reagents reported below by using methods analogous to Compound 17 (Step 9 and Step 10).

| Compound | Structure & Name | Reagents | Analytical data |
|---|---|---|---|
| 18 | 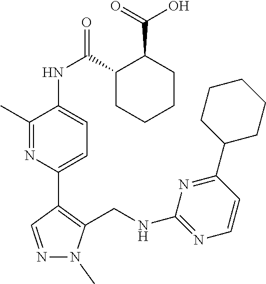 [SMILES: Cc1nc(-c2cnn(C)c2CNc2nccc(C3CCCCC3)n2)ccc1NC(=O)[C@H]1CCCC[C@@H]1C(=O)O] ((1S,2S)-2-((6-(5-(((4- cyclohexylpyrimidin-2- yl)amino)methyl)-1- methyl-1H-pyrazol-4-yl)- 2-methylpyridin-3- yl)carbamoyl)cyclohexane- 1-carboxylic acid | D15 | LC-MS (ESI): m/z (M + 1): 532.2 (Method 1) 1H NMR (500 MHz, DMSO-d6) δ ppm 12.11 (br s, 1 H), 8.97-10.16 (m, 1 H), 8.17 (br s, 1 H), 7.87 (s, 1 H), 7.74 (br s, 1 H), 7.51 (d, J = 8.4 Hz, 1 H), 7.40 (t, J = 6.0 Hz, 1 H), 6.50 (d, J = 5.1 Hz, 1 H), 4.77-4.93 (m, 2 H), 3.98 (s, 3 H), 2.60-2.69 (m, 1 H), 2.44 (s, 3 H), 2.34- 2.43 (m, 2 H), 1.92-2.07 (m, 2 H), 1.60-1.83 (m, 7 H), 1.10-1.48 (m, 9 H) |
| 19 | 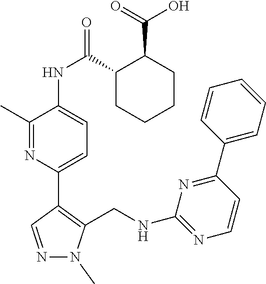 [SMILES: Cc1nc(-c2cnn(C)c2CNc2nccc(-c3ccccc3)n2)ccc1NC(=O)[C@H]1CCCC[C@@H]1C(=O)O] (1S,2S)-2-((2-methyl-6- (1-methyl-5-(((4- phenylpyrimidin-2- yl)amino)methyl)-1H- pyrazol-4-yl)pyridin-3- yl)carbamoyl)cyclohexane- 1-carboxylic acid | D16 | LC-MS (ESI): m/z (M + 1): 526.3 (Method 1) 1H NMR (400 MHz, DMSO-d6) δ ppm 12.11 (br s, 1 H), 9.52 (br s, 1 H), 8.38 (br d, J = 3.9 Hz, 1 H), 8.05 (dd, J = 7.9, 1.8 Hz, 2 H), 7.88 (s, 1 H), 7.63-7.73 (m, 2 H), 7.42- 7.56 (m, 4 H), 7.20 (d, J = 5.3 Hz, 1 H), 4.89-5.11 (m, 2 H), 3.96 (s, 3 H), 2.60-2.66 (m, 1 H), 2.51- 2.55 (m, 1 H), 2.43 (s, 3 H), 2.00 (br d, J = 11.0 Hz, 2 H), 1.69-1.84 (m, 2 H), 1.19-1.46 (m, 4 H) |

### Example 8

### Process Synthesis of Compound 20

### (1S,2S)-2-((4-(5-((6-isopropoxypyrazin-2-yl)amino)-1-methyl-1H-pyrazol-4-yl)phenyl)carbamoyl)cyclohexane-1-carboxylic acid

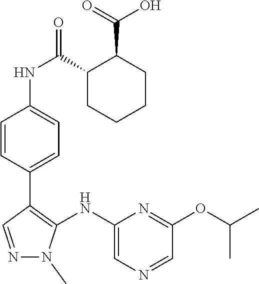 [SMILES: CC(C)Oc1cncc(Nc2c(-c3ccc(NC(=O)[C@H]4CCCC[C@@H]4C(=O)O)cc3)cnn2C)n1]

### Step 1: 1-methyl-4-(4-nitrophenyl)-1H-pyrazol-5-amine (Intermediate E1)

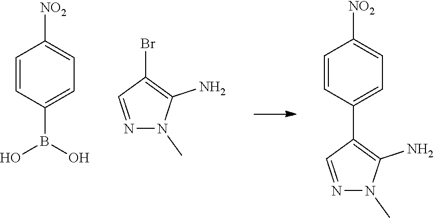

A mixture of 4-bromo-2-methylpyrazol-3-amine (1.0 g, 5.68 mmol), (4-nitrophenyl)boronic acid (1.42 g, 8.52 mmol), Pd(dppf)Cl₂ (557.15 mg, 0.850 mmol) and K₃PO₄ (2.41 g, 11.36 mmol) was degassed. Water (10 mL) and 1,4-Dioxane (30 mL) were added and the mixture was stirred at 120° C. for 2 h. The mixture was cooled to RT and filtered on a celite pad, washing with EtOAc. The filtrate was washed with water and dried over Na2SO4, then it was concentrated to afford the title product (1.89 g, crude) as brown solid which was used in the next step without further purification.

LC-MS (ESI): m/z (M+1): 219.0 (Method 1)

### Step 2: 6-isopropoxy-N-(1-methyl-4-(4-nitrophenyl)-1H-pyrazol-5-yl)pyrazin-2-amine (Intermediate E2)

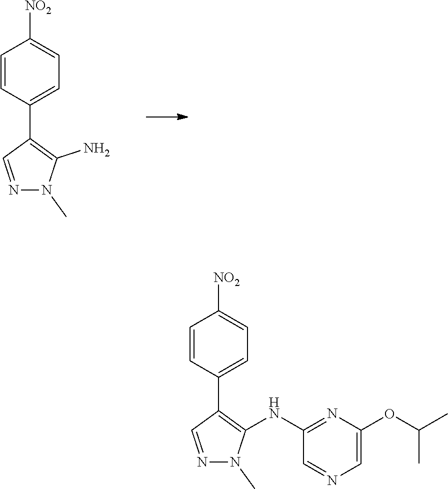 [SMILES: Cn1ncc(-c2ccc([N+](=O)[O-])cc2)c1N ; CC(C)Oc1cncc(Nc2c(-c3ccc([N+](=O)[O-])cc3)cnn2C)n1]

To a suspension of 1-methyl-4-(4-nitrophenyl)-1H-pyrazol-5-amine (Intermediate E1, 600.0 mg, 2.75 mmol) and 2-chloro-6-isopropoxypyrazine (Intermediate A23, 522.08 mg, 3.02 mmol) in 1,2-dimethoxyethane (10 mL), K₃PO₄ (1.46 g, 6.87 mmol) was added. After applying three cycles nitrogen/vacuum, XPhos Pd G2 (216.07 mg, 0.270 mmol) and Pd₂(dba)₃ (251.79 mg, 0.270 mmol) were added. The tube was sealed and the reaction was heated at 100° C. 6 h. The reaction was cooled down to RT and then filtered through a pad of celite to remove the solid, washing with EtOAc. The filtrate was concentrated and purified by flash chromatography (eluent CyHex/EtOAc from 3/7 to 0/10) to afford the target compound (307 mg, 0.866 mmol, 31.51% yield) as brown solid.

LC-MS (ESI): m/z (M+1): 355.1 (Method 1)

### Step 3: N-(4-(4-aminophenyl)-1-methyl-1H-pyrazol-5-yl)-6-isopropoxypyrazin-2-amine (Intermediate E3)

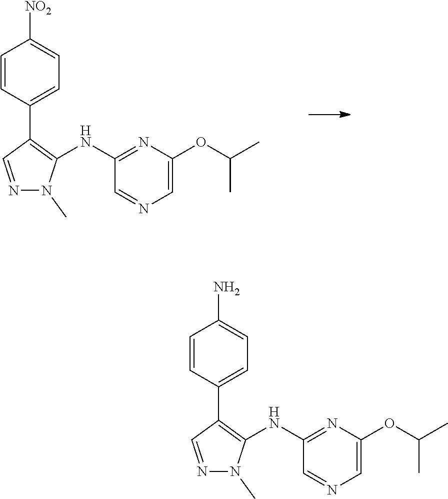 [SMILES: CC(C)Oc1cncc(Nc2c(-c3ccc([N+](=O)[O-])cc3)cnn2C)n1 ; CC(C)Oc1cncc(Nc2c(-c3ccc(N)cc3)cnn2C)n1]

6-isopropoxy-N-(1-methyl-4-(4-nitrophenyl)-1H-pyrazol-5-yl)pyrazin-2-amine (Intermediate E2, 307.0 mg, 0.870 mmol) was dissolved in DCM (5 mL), MeOH (2 mL) and concentrated HCl (1.0 mL, 0.870 mmol), then Fe⁰ (338.7 mg, 6.06 mmol) was added and the mixture was stirred at RT for 3 h then concentrated reduced pressure. The residue was basified with a 2N aqueous NaOH solution and extracted with EtOAc, the mixed organic phases were washed with brine, dried over Na2SO4, filtered and concentrated in vacuo to give the title compound (222 mg, 0.684 mmol, 79% yield) as brownish solid, which was used in the next step without further purification.

LC-MS (ESI): m/z (M+1): 325.2 (Method 1)

### Step 4: (1S,2S)-2-((4-(5-((6-isopropoxypyrazin-2-yl)amino)-1-methyl-1H-pyrazol-4-yl)phenyl)carbamoyl)cyclohexane-1-carboxylic acid (Compound 20)

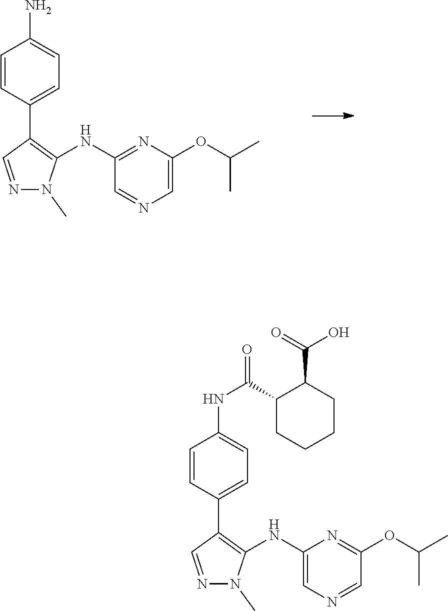 [SMILES: CC(C)Oc1cncc(Nc2c(-c3ccc(N)cc3)cnn2C)n1 ; CC(C)Oc1cncc(Nc2c(-c3ccc(NC(=O)[C@H]4CCCC[C@@H]4C(=O)O)cc3)cnn2C)n1]

Compound 20 (183.65 mg, 0.384 mmol, 56.08% yield) was obtained from N-(4-(4-aminophenyl)-1-methyl-1H-pyrazol-5-yl)-6-isopropoxypyrazin-2-amine (Intermediate E3, 222 mg, 0.684 mmol) and (−)-trans-1,2-Cyclohexanedicarboxylic anhydride (116.05 mg, 0.750 mmol) using a similar method as for Compound 1.

LC-MS (ESI): m/z (M+1): 479.2 (Method 1)

¹H NMR (400 MHz, DMSO-d6) δ ppm 12.00 (br s, 1H), 9.87 (s, 1H), 8.99 (s, 1H), 7.79 (s, 1H), 7.49 (d, J=8.6 Hz, 1H), 7.48 (br s, 1H), 7.46 (s, 1H), 7.37 (d, J=8.7 Hz, 2H), 4.81 (spt, J=6.1 Hz, 1H), 3.64 (s, 3H), 2.59-2.41 (m, 2H), 2.05-1.86 (m, 2H), 1.73 (br d, J=5.4 Hz, 2H), 1.37-1.20 (m, 4H), 1.14 (d, J=6.1 Hz, 6H)

### Example 9

### Process Synthesis of Compound 21

### (1S,2S)-2-((6-(5-(((isopentyl(methyl)carbamoyl)oxy)methyl)-1-methyl-1H-pyrazol-4-yl)-2-methylpyridin-3-yl)carbamoyl)cyclohexane-1-carboxylic acid

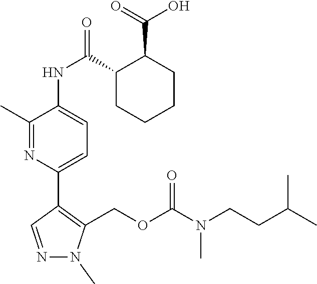 [SMILES: Cc1nc(-c2cnn(C)c2COC(=O)N(C)CCC(C)C)ccc1NC(=O)[C@H]1CCCC[C@@H]1C(=O)O]

### Step 1: (4-bromo-1-methyl-1H-pyrazol-5-yl)methanol (Intermediate F1)

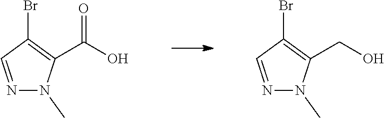

Under N2 was charged with 4-bromo-1-methyl-1H-pyrazole-5-carboxylic acid (5.0 g, 24.39 mmol) was dissolved in dry THE (50 mL). oxolan-1-ium-1-ylboranuide (36.58 mL, 36.58 mmol) was added carefully and the mixture was stirred at RT for 2 h, then at 50° C. for 42 h. A second aliquot of oxolan-1-ium-1-ylboranuide (36.58 mL, 36.58 mmol) was added and the suspension was stirred at 50° C. for additional 24 h. After cooling down at RT HCl (1.2 M in H2O) was added and the suspension was stirred for 1 h. Then the mixture was diluted with EtOAc and Brine. The aqueous phase was extracted twice with EtOAc. The combined organic phase was filtered through a hydrophobic funnel and the solvent was removed in vacuo. The crude was purified through flash column chromatography using a gradient of EtOAc in cyclohexane from 0% to 100% affording the desired product (2.59 g, 13.56 mmol, 55.59% yield) as a white solid.

LC-MS (ESI): m/z (M+1): 192.9 (Method 1)

### Step 2: (1-methyl-4-(4,4,5,5-tetramethyl-1,3,2-dioxaborolan-2-yl)-1H-pyrazol-5-yl)methanol (Intermediate F2)

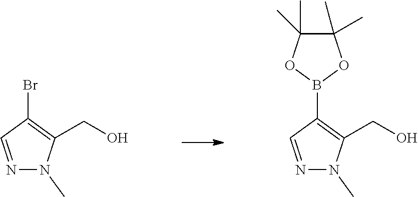

A mixture of 4-bromo-1-methyl-1H-pyrazol-5-yl)methanol (Intermediate F1, 200.0 mg, 1.01 mmol), KOAc (295.91 mg, 3.02 mmol) and B2pin2 (382.84 mg, 1.51 mmol) in 1,4-Dioxane (1.536 mL) was degassed under N2 for 5 minutes, then Pd(dppf)Cl2, complex with dichloromethane (82.28 mg, 0.100 mmol) was added. The tube was sealed and the mixture was heated at 100° C. for 12h. Catalyst and salts were removed by filtration, washing with EtOAc. The solvent was removed under vacuum. The crude product (360 mg, 1.512 mmol, 150.44% yield) was used as this in the following step without any additional purification.

LC-MS (ESI): m/z (M+1): 239.1 (Method 1)

### Step 3: tert-butyl (2-methyl-6-(1-methyl-5-((((4-nitrophenoxy)carbonyl)oxy)methyl)-1H-pyrazol-4-yl)pyridin-3-yl)carbamate (Intermediate F3)

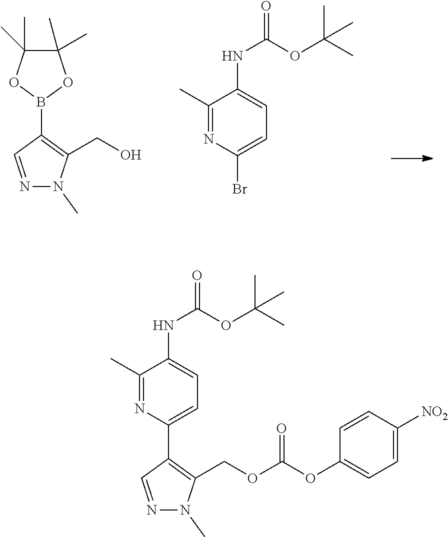 [SMILES: Cn1ncc(B2OC(C)(C)C(C)(C)O2)c1CO ; Cc1nc(Br)ccc1NC(=O)OC(C)(C)C ; Cc1nc(-c2cnn(C)c2COC(=O)Oc2ccc([N+](=O)[O-])cc2)ccc1NC(=O)OC(C)(C)C]

To a solution of (1-methyl-4-(4,4,5,5-tetramethyl-1,3,2-dioxaborolan-2-yl)-1H-pyrazol-5-yl)methanol (Intermediate F2, 239.28 mg, 1.01 mmol) in 1,4-Dioxane (2.175 mL) were added tert-butyl (6-bromo-2-methylpyridin-3-yl)carbamate (Intermediate D2, 346.3 mg, 1.21 mmol), K3PO4 (0.53 g, 3.01 mmol), Water (2.175 mL) and XPhos Pd G2 (0.08 g, 0.100 mmol)under nitrogen. The mixture was stirred at 90° C. overnight. Brine and EtOAc were added and the aqueous layer was extracted with EtOAc and then the collected organic phases were evaporated. The crude mixture was dissolved in DCM (0.619 mL) and Pyridine (0.04 mL, 0.470 mmol). The mixture was cooled at 0° C. carbonochloridic acid (4-nitrophenyl) ester (31.34 mg, 0.160 mmol) was slowly added to the mixture. The mixture was let to warm up to RT. After 12h aq. NaHCO₃sat. solution was added and the aq. phase was extracted with DCM (3 times). The collected organics fractions were collected and then concentrated under reduced pressure. The crude was purified by flash chromatography using a gradient of EtOAc in CyHex from 0% to 50% affording the desired product (25 mg, 0.052 mmol, 66.52% yield).

LC-MS (ESI): m/z (M+1): 484.2 (Method 1)

### Step 4: (4-(5-((tert-butoxycarbonyl)amino)-6-methylpyridin-2-yl)-1-methyl-1H-pyrazol-5-yl)methyl isopentyl(methyl)carbamate (Intermediate F4)

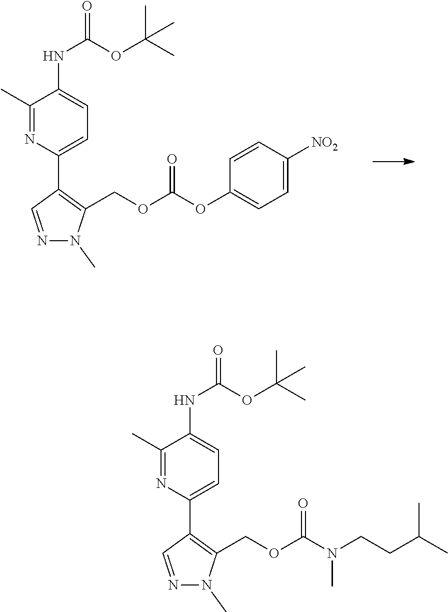 [SMILES: Cc1nc(-c2cnn(C)c2COC(=O)Oc2ccc([N+](=O)[O-])cc2)ccc1NC(=O)OC(C)(C)C ; Cc1nc(-c2cnn(C)c2COC(=O)N(C)CCC(C)C)ccc1NC(=O)OC(C)(C)C]

DIPEA (0.02 mL, 0.130 mmol) and N,3-dimethylbutan-1-amine hydrochloride (8.54 mg, 0.060 mmol) were added to a solution of tert-butyl (2-methyl-6-(1-methyl-5-((((4-nitrophenoxy)carbonyl)oxy)methyl)-1H-pyrazol-4-yl)pyridin-3-yl)carbamate (Intermediate F3, 25.0 mg, 0.050 mmol) in dry THE (0.473 mL). The mixture was stirred overnight at RT for 6 hours. The mixture was concentrated in vacuo and residue was dissolved in EtOAc and washed with sat. NaHCO3 (2 times). The organic phase was concentrated in vacuo. The crude was purified by flash chromatography using a gradient of EtOAc in cyclohexane from 0% to 75% to give the desired product (13 mg, 0.029 mmol, 56.42% yield).

LC-MS (ESI): m/z (M+1): 446.3 (Method 1)

### Step 5: (4-(5-amino-6-methylpyridin-2-yl)-1-methyl-1H-pyrazol-5-yl)methyl isopentyl(methyl)carbamate (Intermediate F5)

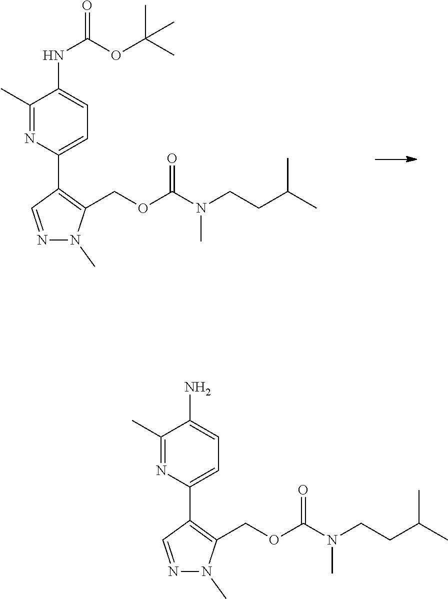

To a solution of (4-(5-((tert-butoxycarbonyl)amino)-6-methylpyridin-2-yl)-1-methyl-1H-pyrazol-5-yl)methyl isopentyl(methyl)carbamate (Intermediate F5, 13.0 mg, 0.030 mmol) in 1,4-Dioxane (0.056 mL), HCl 4N in 1,4-Dioxane (0.02 mL, 0.090 mmol) was added dropwise. The mixture was stirred at RT for 4 h. The crude was concentrated under reduced pressure. The crude was loaded on a SCX cartridge, washing with MeOH and then with 2N NH3 in MeOH. This last fraction was concentrated under reduced pressure recovering the title compound (8 mg, 0.023 mmol, 79.37% yield).

LC-MS (ESI): m/z (M+1): 346.3 (Method 1)

### Step 8: (1S,2S)-2-((6-(5-(((isopentyl(methyl)carbamoyl)oxy)methyl)-1-methyl-1H-pyrazol-4-yl)-2-methylpyridin-3-yl)carbamoyl)cyclohexane-1-carboxylic acid (Compound 21) O O

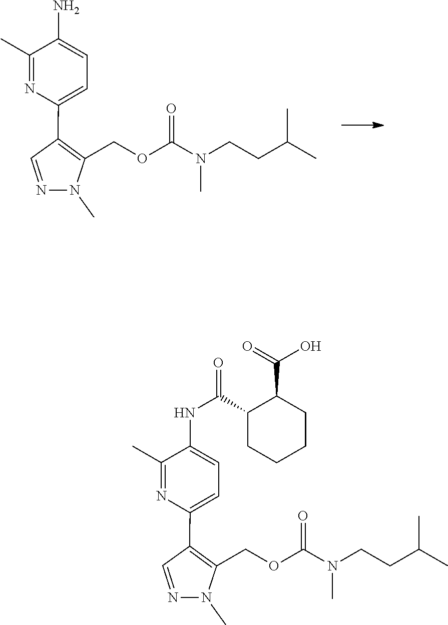

Compound 21 (10 mg, 0.020 mmol, 86.43% yield) was obtained from of (4-(5-amino-6-methylpyridin-2-yl)-1-methyl-1H-pyrazol-5-yl)methyl isopentyl(methyl)carbamate (Intermediate F5, 8.0 mg, 0.020 mmol) and (−)-trans-1,2-Cyclohexanedicarboxylic anhydride (3.93 mg, 0.030 mmol) using a similar method as for Compound 1.

LC-MS (ESI): m/z (M+1): 500.4 (Method 1)

¹H NMR (500 MHz, DMSO-d6) δ ppm 12.05 (br s, 1H), 10.29-8.58 (m, 1H), 7.90 (s, 1H), 7.67 (br d, J=8.4 Hz, 1H), 7.46 (d, J=8.4 Hz, 1H), 5.56 (s, 2H), 3.89 (s, 3H), 3.26-3.03 (m, 2H), 2.84-2.69 (m, 3H), 2.68-2.58 (m, 1H), 2.99-2.50 (m, 1H), 2.43-2.32 (m, 3H), 2.16-1.93 (m, 2H), 1.82-1.16 (m, 9H), 0.96-0.58 (m, 6H)

### Example 10

### Process of Synthesis of Compound 22

### (1S,2S)-2-((6-(4-((2-(2-chlorophenyl)ethyl)sulfonamido)-3-methylisoxazol-5-yl)-2-methylpyridin-3-yl)carbamoyl)cyclohexane-1-carboxylic acid

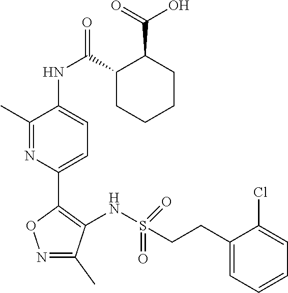 [SMILES: Cc1nc(-c2onc(C)c2NS(=O)(=O)CCc2ccccc2Cl)ccc1NC(=O)[C@H]1CCCC[C@@H]1C(=O)O]

### Step 1: tert-butyl (1S,2S)-2-((6-(4-((2-(2-chlorophenyl)ethyl)sulfonamido)-3-methylisoxazol-5-yl)-2-methylpyridin-3-yl)carbamoyl)cyclohexane-1-carboxylate (Intermediate G1)

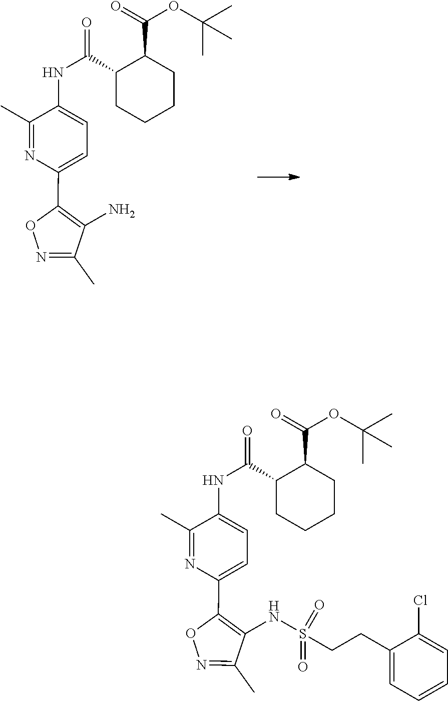 [SMILES: Cc1nc(-c2onc(C)c2N)ccc1NC(=O)[C@H]1CCCC[C@@H]1C(=O)OC(C)(C)C ; Cc1nc(-c2onc(C)c2NS(=O)(=O)CCc2ccccc2Cl)ccc1NC(=O)[C@H]1CCCC[C@@H]1C(=O)OC(C)(C)C]

Under N₂ atmosphere, tert-butyl (1S,2S)-2-((6-(4-amino-3-methylisoxazol-5-yl)-2-methylpyridin-3-yl)carbamoyl)cyclohexane-1-carboxylate (Intermediate B6, 65.0 mg, 0.160 mmol) was dissolved in MeCN (1.000 mL) and Pyridine (24.81 mg, 0.310 mmol) was added. Then, 2-(2-chlorophenyl)ethanesulfonyl chloride (43.12 mg, 0.180 mmol) was added and the reaction was stirred at RT for 16 h. The solvent was removed under vacuum and the crude was purified by flash chromatography using a gradient of EtOAc in CyHex from 0% to 40% to provide title compound (66.4 mg, 0.108 mmol, 68.61% yield) as white solid.

LC-MS (ESI): m/z (M+1): 617.3 (Method 1)

### Step 2: (1S,2S)-2-((6-(4-((2-(2-chlorophenyl)ethyl)sulfonamido)-3-methylisoxazol-5-yl)-2-methylpyridin-3-yl)carbamoyl)cyclohexane-1-carboxylic acid (Compound 22)

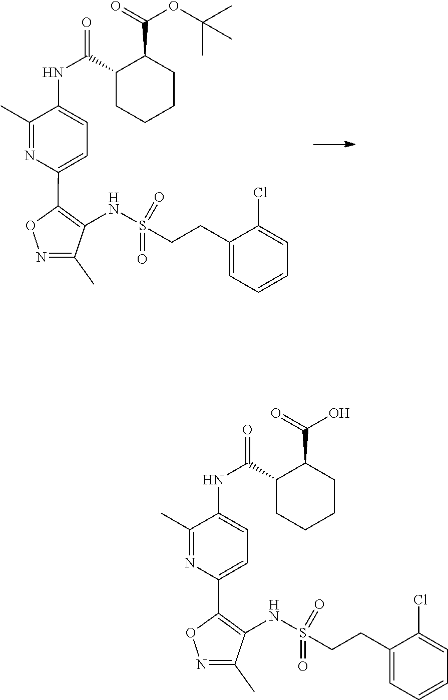 [SMILES: Cc1nc(-c2onc(C)c2NS(=O)(=O)CCc2ccccc2Cl)ccc1NC(=O)[C@H]1CCCC[C@@H]1C(=O)OC(C)(C)C ; Cc1nc(-c2onc(C)c2NS(=O)(=O)CCc2ccccc2Cl)ccc1NC(=O)[C@H]1CCCC[C@@H]1C(=O)O]

Under N₂ atmosphere, tert-butyl (1S,2S)-2-((6-(4-((2-(2-chlorophenyl)ethyl)sulfonamido)-3-methylisoxazol-5-yl)-2-methylpyridin-3-yl)carbamoyl)cyclohexane-1-carboxylate (Intermediate Gi, 66.4 mg, 0.110 mmol) was dissolved in 1,4-Dioxane (1 mL). Then, HCl 4M in 1,4-dioxane (0.27 mL, 1.08 mmol) was added and the reaction was stirred at 60° C. for 16 h. The solvent was removed under vacuum and the crude was purified by flash chromatography using a gradient of MeCN in water from 10% to 60% to provide title compound (34.8 mg, 0.062 mmol, 57.65% yield) as white solid.

LC-MS (ESI): m/z (M+1): 561.2 (Method 1)

¹H NMR (400 MHz, DMSO-d6) δ ppm 12.10 (br s, 1H), 9.55 (s, 1H), 9.45 (br s, 1H), 7.98 (d, J=8.3 Hz, 1H), 7.79 (br d, J=8.1 Hz, 1H), 7.39 (dd, J=7.5, 1.8 Hz, 1H), 7.18-7.28 (m, 2H), 7.17-7.12 (m, 1H), 3.48-3.37 (m, 2H), 3.13-3.04 (m, 2H), 2.74-2.65 (m, 1H), 2.58-2.51 (m, 1H), 2.31 (s, 3H), 2.30 (s, 3H), 2.103-1.94 (H, 2H), 185-1.69 (m, 2H), 1.41-1.19 (m, 4H).

The Compounds in the following table were prepared from reagents reported below following similar procedures as for Compound 22 (Step 1 and Step 2).

| Compound | Structure & Name | Reagents | Analytical data |
|---|---|---|---|
| 23 | 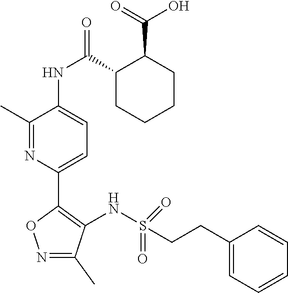 [SMILES: Cc1nc(-c2onc(C)c2NS(=O)(=O)CCc2ccccc2)ccc1NC(=O)[C@H]1CCCC[C@@H]1C(=O)O] (1S,2S)-2-((2-methyl-6- (3-methyl-4-((2- phenylethyl)sulfonamido) isoxazol-5-yl)pyridin-3- yl)carbamoyl)cyclohexane- 1-carboxylic acid | B6 and 2- phenylethane- 1-sulfonyl chloride | LC-MS (ESI): m/z (M + 1): 524.9 (Method 10) ¹H NMR (300 MHz, DMSO-d₆) δ 9.84 (s, 1H), 8.20 (d, J = 8.4 Hz, 1H), 7.95 (d, J = 8.5 Hz, 1H), 7.25-7.08 (m, 3H), 7.05- 6.96 (m, 2H), 2.95 (d, J = 11.3 Hz, 2H), 2.91- 2.81 (m, 2H), 2.70-2.58 (m, 1H), 2.39 (s, 3H), 2.25 (s, 3H), 2.01 (d, J = 10.5 Hz, 1H), 1.90 (d, J = 11.9 Hz, 1H), 1.73 (s, 2H), 1.45- 1.17 (m, 5H |
| 24 | 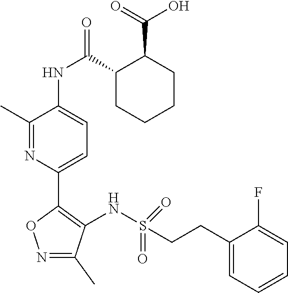 [SMILES: Cc1nc(-c2onc(C)c2NS(=O)(=O)CCc2ccccc2F)ccc1NC(=O)[C@H]1CCCC[C@@H]1C(=O)O] (1S,2S)-2-((6-(4-((2-(2- fluorophenyl)ethyl) sulfonamido)-3- methylisoxazol-5-yl)-2- methylpyridin-3- yl)carbamoyl)cyclohexane- 1-carboxylic acid | B6 and 2-(2- fluorophenyl) ethane- sulfonyl chloride | LC-MS (ESI): m/z (M + 1): 545.2 (Method 1) ¹H NMR (500 MHz, DMSO-d₆) δ ppm 12.09 (br s, 1 H), 9.55 (s, 2 H), 7.99 (d, J = 8.6 Hz, 1 H), 7.79 (d, J = 8.3 Hz, 1 H), 7.21-7.30 (m, 1 H), 7.01- 7.16 (m, 3 H), 3.38-3.49 (m, 2 H), 2.95-3.08 (m, 2 H), 2.52-2.77 (m, 2 H), 2.33 (s, 3 H), 2.30 (s, 3 H), 1.20-2.09 (m, 8 H) |
| 25 | 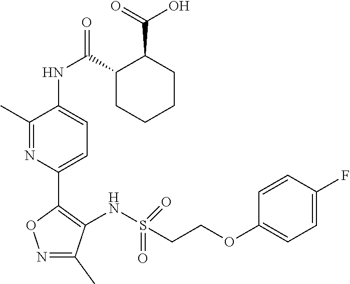 [SMILES: Cc1nc(-c2onc(C)c2NS(=O)(=O)CCOc2ccc(F)cc2)ccc1NC(=O)[C@H]1CCCC[C@@H]1C(=O)O] (1S,2S)-2-((6-(4-((2-(4- fluorophenoxy)ethyl) sulfonamido)-3- methylisoxazol-5-yl)-2- methylpyridin-3- yl)carbamoyl)cyclohexane- 1-carboxylic acid | B6 and 2-(4- fluorophenoxy) ethane- 1-sulfonyl chloride | LC-MS (ESI): m/z (M + 1): 561.3 (Method 1) ¹H NMR (400 MHz, DMSO-d₆) δ ppm 12.12 (br s, 1 H), 9.59 (s, 1 H), 9.48 (br s, 1 H), 8.00 (d, J = 8.3 Hz, 1 H), 7.79 (d, J = 8.3 Hz, 1 H), 7.05-7.13 (m, 2 H), 6.82-6.90 (m, 2 H), 4.26 (t, J = 6.1 Hz, 2 H), 3.65 (t, J = 6.1 Hz, 2 H), 2.65-2.78 (m, 1 H), 2.52- 2.59 (m, 1 H), 2.45 (s, 3 H), 2.29 (s, 3 H), 1.19- 2.11 (m, 8 H) |
| 26 | 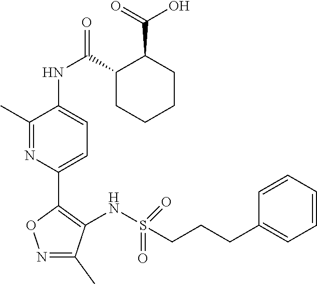 [SMILES: Cc1nc(-c2onc(C)c2NS(=O)(=O)CCCc2ccccc2)ccc1NC(=O)[C@H]1CCCC[C@@H]1C(=O)O] (1S,2S)-2-((2-methyl-6- (3-methyl-4-((3- phenylpropyl)sulfonamido) isoxazol-5-yl)pyridin- 3- yl)carbamoyl)cyclohexane- 1-carboxylic acid | B6 and 3- phenylpropane- 1- sulfonyl chloride | LC-MS (ESI): m/z (M + 1): 541.3 (Method 1) ¹H NMR (400 MHz, DMSO-d₆) δ ppm 12.12 (br s, 1 H), 9.63 (s, 1 H), 9.19 (br s, 1 H), 8.03 (d, J = 8.4 Hz, 1 H), 7.78 (d, J = 8.4 Hz, 1 H), 7.17-7.23 (m, 2 H), 7.09-7.17 (m, 1 H), 7.00 (d, J = 6.8 Hz, 2 H), 2.94-3.10 (m, 2 H), 2.69-2.85 (m, 1 H), 2.51- 2.59 (m, 3 H), 2.42 (s, 3 H), 2.26 (s, 3 H), 1.98- 2.13 (m, 2 H), 1.85-1.96 (m, 2 H), 1.69-1.83 (m, 2 H), 1.21-1.52 (m, 4 H) |
| 27 | 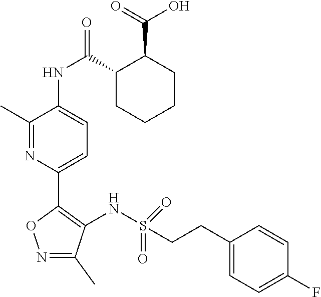 [SMILES: Cc1nc(-c2onc(C)c2NS(=O)(=O)CCc2ccc(F)cc2)ccc1NC(=O)[C@H]1CCCC[C@@H]1C(=O)O] (1S,2S)-2-((2-methyl-6- (3-methyl-4-((2- phenylethyl)sulfonamido) isoxazol-5-yl)pyridin-3- yl)carbamoyl)cyclohexane- 1-carboxylic acid | B6 and 2-(4- fluorophenyl) ethane-1- sulfonyl chloride | LC-MS (ESI): m/z (M + 1): 545.3 (Method 1) ¹H NMR (400 MHz, DMSO-d₆) δ ppm 12.09 (br s, 1 H), 9.59 (br s, 1 H), 9.52 (br s, 1 H), 8.00 (br d, J = 8.4 Hz, 1 H), 7.75- 7.91 (m, 1 H), 6.91-7.18 (m, 4 H), 3.33-3.45 (m, 2 H), 2.92 (br dd, J = 10.4, 5.5 Hz, 2 H), 2.64-2.80 (m, 1 H), 2.47-2.60 (m, 1 H), 2.34 (s, 3 H), 2.30 (s, 3 H), 1.92-2.12 (m, 2 H), 1.69-1.84 (m, 2 H), 1.20- 1.44 (m, 4 H) |
| 28 | 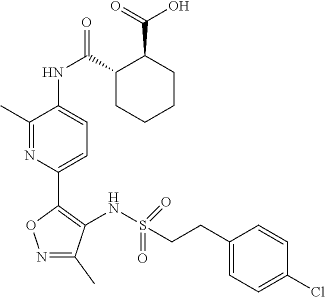 (1S,2S)-2-((6-(4-((2-(4- chlorophenyl)ethyl) sulfonamido)-3- methylisoxazol-5-yl)-2- methylpyridin-3- yl)carbamoyl)cyclohexane- 1-carboxylic acid | B6 and 2-(4- chlorophenyl) ethane-1- sulfonyl chloride | LC-MS (ESI): m/z (M + 1): 599.4 (Method 1) ¹H NMR (400 MHz, DMSO-d₆) δ ppm 12.12 (br s, 1 H), 9.58 (s, 1 H), 9.42 (br s, 1 H), 8.01 (d, J = 8.4 Hz, 1 H), 7.81 (br d, J = 8.2 Hz, 1 H), 7.27 (d, J = 8.3 Hz, 2 H), 7.04 (d, J = 8.3 Hz, 2 H), 3.41 (br t, J = 7.1 Hz, 2 H), 2.85-3.05 (m, 2 H), 2.64-2.78 (m, 1 H), 2.52-2.60 (m, 1 H), 2.35 (s, 3 H), 2.30 (s, 3 H), 1.20-2.10 (m, 8 H) |
| 29 | 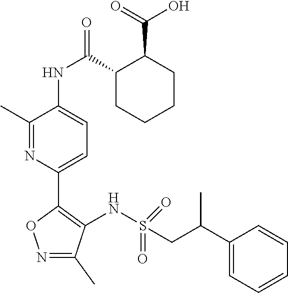 [SMILES: Cc1nc(-c2onc(C)c2NS(=O)(=O)CC(C)c2ccccc2)ccc1NC(=O)[C@H]1CCCC[C@@H]1C(=O)O] rac-(1S,2S)-2-((2- methyl-6-(3-methyl-4- ((2- phenylpropyl)sulfonamido) isoxazol-5-yl)pyridin- 3- yl)carbamoyl)cyclohexane- 1-carboxylic acid | B6 and Rac 2- phenylpropane- 1- sulfonyl chloride | LC-MS (ESI): m/z (M + 1): 541.2 (Method 1) 1H NMR (500 MHz, DMSO-d₆) δ ppm 11.70- 12.43 (m, 1 H), 9.57 (s, 1 H), 9.06-9.45 (m, 1 H), 7.99 (d, J = 8.4 Hz, 1 H), 7.76 (d, J = 8.4 Hz, 1 H), 7.20-7.27 (m, 2 H), 7.14- 7.20 (m, 1 H), 7.07-7.12 (m, 2 H), 3.38-3.57 (m, 2 H), 3.17-3.28 (m, 1 H), 2.66-2.81 (m, 1 H), 2.54 (s, 1 H), 2.29 (d, J = 13.2 Hz, 6 H), 1.93-2.08 (m, 2 H), 1.71-1.86 (m, 2 H), 1.20-1.49 (m, 7 H) |

Compound 29 was submitted to chiral semipreparative chromatography.

Conditions: Column Chiralcel OJ-H (25×2.0 cm), 5μ Mobile phase n-Hexane/(Ethanol+0.1% formic acid) 55/45% v/v Flow rate 17 ml/min DAD detection 310 nm Loop 500 μL.

### Peak 1: Compound 30

### Single Diastereomer 1 of (1S,2S)-2-((2-methyl-6-(3-methyl-4-((2-phenylpropyl)sulfonamido)isoxazol-5-yl)pyridin-3-yl)carbamoyl)cyclohexane-1l-carboxylic acid

LC-MS (ESI): m/z (M+1): 541.2 (Method 1)

¹H NMR (400 MHz, DMSO-d₆) δ ppm 12.11 (br s, 1H), 9.56 (s, 1H), 9.30 (br s, 1H), 7.99 (d, J=8.4 Hz, 1H), 7.76 (d, J=8.4 Hz, 1H), 7.28-7.20 (m, 2H), 7.20-7.14 (m, 1H), 7.13-7.05 (m, 2H), 3.57-3.39 (m, 2H), 3.29-3.15 (m, 1H), 2.81-2.64 (m, 1H), 2.59-2.51 (m, 1H), 2.30 (s, 3H), 2.28 (s, 3H), 2.09-1.93 (m, 2H), 1.89-1.68 (m, 2H), 1.43-1.27 (m, 4H), 1.25 (d, J=6.9 Hz, 3H)

### Peak 2: Compound 31

### Single Diastereomer 2 of (1S,2S)-2-((2-methyl-6-(3-methyl-4-((2-phenylpropyl)sulfonamido)isoxazol-5-yl)pyridin-3-yl)carbamoyl)cyclohexane-1-carboxylic acid

LC-MS (ESI): m/z (M+1): 541.2 (Method 1)

¹H NMR (400 MHz, DMSO-d₆) δ ppm 12.07 (br s, 1H), 9.58 (s, 1H), 9.36 (br s, 1H), 7.99 (d, J=8.4 Hz, 1H), 7.78 (d, J=8.4 Hz, 1H), 7.28-7.19 (m, 2H), 7.19-7.12 (m, 1H), 7.09 (d, J=7.1 Hz, 2H), 3.54-3.37 (m, 2H), 3.28-3.15 (m, 1H), 2.79-2.65 (m, 1H), 2.59-2.51 (m, 1H), 2.31 (s, 3H), 2.28 (s, 3H), 2.15-1.93 (m, 2H), 1.87-1.71 (m, 2H), 1.41-1.27 (m, 4H), 1.25 (d, J=6.9 Hz, 3H)

### Example 11

### Process of Synthesis of Compound 32

### (1S,2S)-2-((6-(4-((((benzyloxy)carbonyl)amino)methyl)-3-methylisoxazol-5-yl)-2-methylpyridin-3-yl)carbamoyl)cyclohexane-1-carboxylic acid

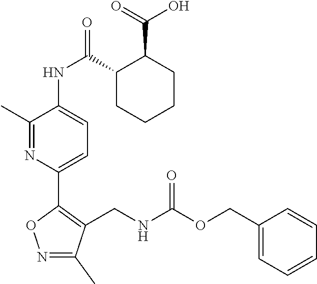 [SMILES: Cc1nc(-c2onc(C)c2CNC(=O)OCc2ccccc2)ccc1NC(=O)[C@H]1CCCC[C@@H]1C(=O)O]

### Step 1: tert-butyl (6-(4-((((benzyloxy)carbonyl)amino)methyl)-3-methylisoxazol-5-yl)-2-methylpyridin-3-yl)carbamate (Intermediate H1)

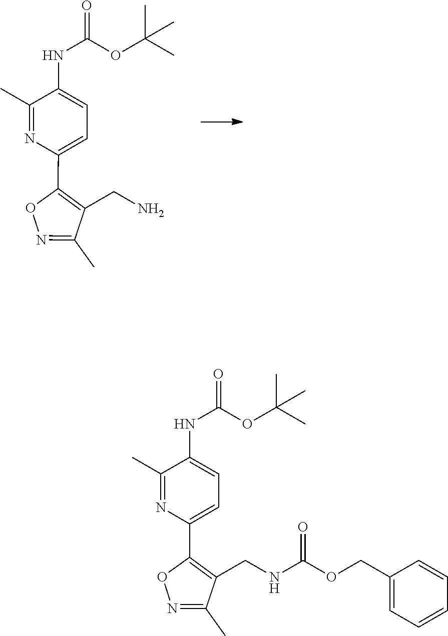 [SMILES: Cc1nc(-c2onc(C)c2CN)ccc1NC(=O)OC(C)(C)C ; Cc1nc(-c2onc(C)c2CNC(=O)OCc2ccccc2)ccc1NC(=O)OC(C)(C)C]

To a solution of tert-butyl (6-(4-(aminomethyl)-3-methylisoxazol-5-yl)-2-methylpyridin-3-yl)carbamate (Intermediate D12, 40.0 mg, 0.130 mmol), and K₂CO₃ (34.73 mg, 0.250 mmol) in THE (2 mL) and Water (1 mL), carbonochloridic acid (phenylmethyl) ester (0.02 mL, 0.130 mmol) was added at 0° C. The resulting solution was stirred and allowed to warm to RT for 1 hour. The mixture was quenched with NH4Cl (aq). The two phases were separated and the aqueous phase was extracted with EtOAc (2 times). The combined organic extracts were dried over Na2SO4, filtered and concentrated under reduced pressure. The crude was purified by flash chromatography using a gradient of CyHex/EtOAc from 10/0 to 6/4) to afford title compound (45.9 mg, 0.101 mmol, 80.74% yield) as colorless oil.

LC-MS (ESI): m/z (M+1): 453.3 (Method 1)

### Step 2: benzyl ((5-(5-amino-6-methylpyridin-2-yl)-3-methylisoxazol-4-yl)methyl)carbamate (Intermediate H2)

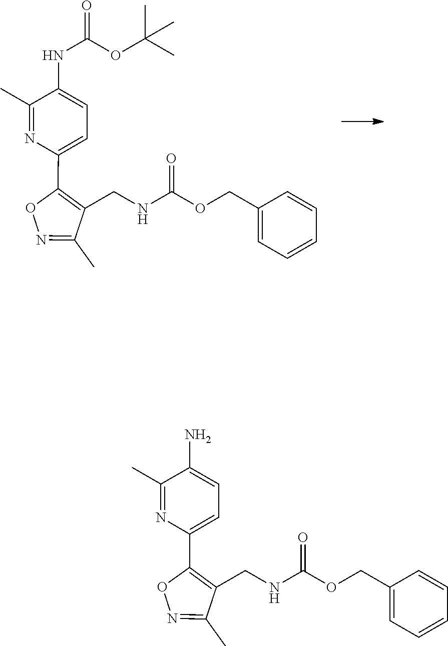 [SMILES: Cc1nc(-c2onc(C)c2CNC(=O)OCc2ccccc2)ccc1NC(=O)OC(C)(C)C ; Cc1nc(-c2onc(C)c2CNC(=O)OCc2ccccc2)ccc1N]

To a solution of tert-butyl (6-(4-((((benzyloxy)carbonyl)amino)methyl)-3-methylisoxazol-5-yl)-2-methylpyridin-3-yl)carbamate (Intermediate H1, 45.9 mg, 0.100 mmol) in DCM (3 mL), 4M HCl in 1,4-dioxane (0.25 mL, 1.01 mmol) was added and the resulting solution was stirred 4 h at RT. The solvent was evaporated, then the residue was purified by SCX eluting with MeOH, then recovering the product eluting with NH3 4 M in MeOH, obtaining the target product (30.4 mg, 0.086 mmol, 85.05% yield) as a colorless oil.

LC-MS (ESI): m/z (M+1): 353.2 (Method 1)

### Step 3: (1S,2S)-2-((6-(4-((((benzyloxy)carbonyl)amino)methyl)-3-methylisoxazol-5-yl)-2-methylpyridin-3-yl)carbamoyl)cyclohexane-1-carboxylic acid (Compound 32)

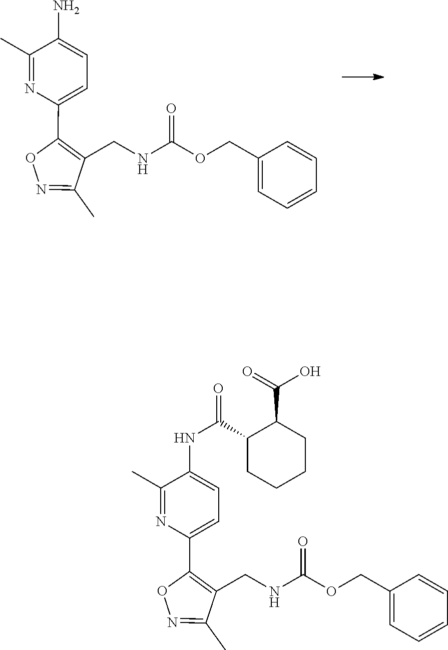 [SMILES: Cc1nc(-c2onc(C)c2CNC(=O)OCc2ccccc2)ccc1N ; Cc1nc(-c2onc(C)c2CNC(=O)OCc2ccccc2)ccc1NC(=O)[C@H]1CCCC[C@@H]1C(=O)O]

Compound 32 (30.2 mg, 0.060 mmol, 69.11% yield) was obtained as white solid from benzyl ((5-(5-amino-6-methylpyridin-2-yl)-3-methylisoxazol-4-yl)methyl)carbamate (Intermediate H2, 30.4 mg, 0.090 mmol and (−)-trans-1,2-Cyclohexanedicarboxylic anhydride (14.63 mg, 0.090 mmol) using a similar method as for Compound 1.

LC-MS (ESI): m/z (M+1): 507.3 (Method 1)

¹H NMR (600 MHz, DMSO-d₆) δ ppm 12.14 (br s, 1H), 9.66 (br s, 1H), 7.98 (d, J=8.4 Hz, 1H), 7.71 (br d, J=8.4 Hz, 1H), 7.52 (br t, J=4.9 Hz, 1H), 7.40-7.11 (m, 5H), 5.02 (s, 2H), 4.53 (br d, J=5.4 Hz, 2H), 2.70 (td, J=11.4, 3.3 Hz, 1H), 2.56-2.50 (m, 1H), 2.48 (s, 3H), 2.27 (s, 3H), 2.03 (br d, J=8.9 Hz, 1H), 2.00 (br d, J=13.7 Hz, 1H), 1.83-1.72 (m, 2H), 1.20-1.41 (m, 4H)

### Example 12

### Process Synthesis of Compound 33

### (1S,2S)-2-((6-(4-(((((R)-1-(2-chlorophenyl)ethoxy)carbonyl)amino)methyl)-3-methylisoxazol-5-yl)-2-methylpyridin-3-yl)carbamoyl)cyclohexane-1-carboxylic acid

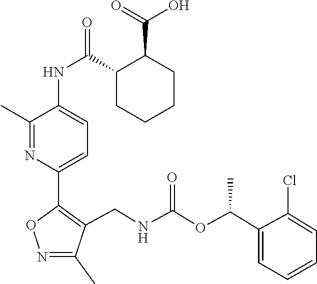 [SMILES: Cc1nc(-c2onc(C)c2CNC(=O)O[C@H](C)c2ccccc2Cl)ccc1NC(=O)[C@H]1CCCC[C@@H]1C(=O)O]

### Step 1: (R)-1-(2-chlorophenyl)ethyl (4-nitrophenyl) carbonate (Intermediate I1)

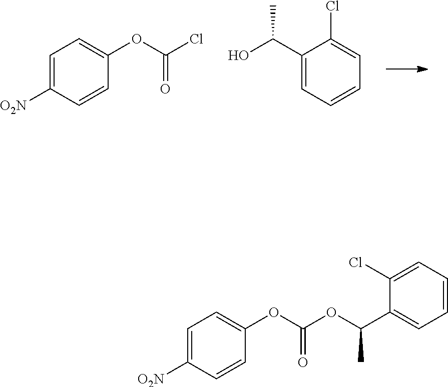

To a solution of (R)-1-(2-chlorophenyl)ethan-1-ol (0.42 mL, 3.19 mmol) in 10 ml of DCM, Pyridine (1.28 mL, 15.96 mmol) was added, followed by a solution of carbonochloridic acid (4-nitrophenyl) ester (1.29 g, 6.39 mmol) in 10 ml of DCM. The mixture was stirred at RT for 2 h. The resulting suspension was concentrated, then it was partioned between NaHCO3 satd. sol. and EtOAc. The resulting organic phase was separated, dried over Na2SO4, filtered and evaporated to give the crude which was purified by flash chromatography using CyHex/EtOAc from 10/0 to 8/2 to afford the tile compound (937 mg, 2.913 mmol, 91.23% yield) as colorless oil.

¹H NMR (400 MHz, DMSO-d₆) δ ppm 8.30 (s, 2H) 7.56 (d, J=9.02 Hz, 6H) 6.11 (d, J=6.60 Hz, 1H) 1.46-1.79 (in, 3H).

The Intermediates in the following table were prepared by using methods analogous to Intermediate I1.

| Intermediate | Structure & Name | Reageants | Analytical data |
|---|---|---|---|
| I2 | 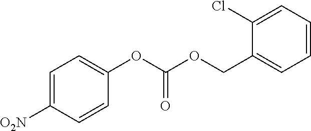 [SMILES: O=C(OCc1ccccc1Cl)Oc1ccc([N+](=O)[O-])cc1] (2-chlorophenyl)methyl (4- nitrophenyl) carbonate | (2- chlorophenyl) methanol | ¹H NMR (400 MHz, DMSO-d₆) d ppm 8.33 (d, J = 9.24 Hz, 2 H) 7.67-7.52 (m, 4 H) 7.51-7.39 (m, 2 H) 5.41 (s, 2 H) |
| I3 | 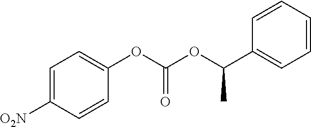 [SMILES: C[C@@H](OC(=O)Oc1ccc([N+](=O)[O-])cc1)c1ccccc1] (R)-4-nitrophenyl (1- phenylethyl) carbonate | (R)-1- phenylethan- 1-ol | ¹H NMR (400 MHz, DMSO-d₆) d ppm 8.38-8.23 (m, 2 H) 7.59-7.51 (m, 2 H) 7.50-7.32 (m, 5 H) 5.84 (q, J = 6.53 Hz, 1 H) 1.63 (d, J = 6.60 Hz, 3 H) |
| I4 | 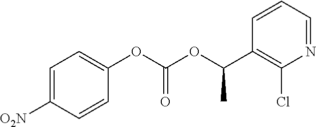 (R)-1-(2-chloropyridin-3- yl)ethyl (4-nitrophenyl) carbonate | (R)-1-(2- chloropyridin- 3-yl)ethan- 1-ol | LC-MS (ESI): m/z (M + 1): 319.1 (Method 4) |
| I5 | 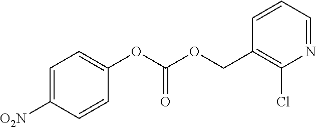 [SMILES: O=C(OCc1cccnc1Cl)Oc1ccc([N+](=O)[O-])cc1] (2-chloropyridin-3-yl)methyl (4-nitrophenyl) carbonate | (2- chloropyridin- 3- yl)methanol | LC-MS (ESI): m/z (M + 1): 309.1 (Method 4) |

### Step 2: tert-butyl (R)-(6-(4-((((1-(2-chlorophenyl)ethoxy)carbonyl)amino)methyl)-3-methylisoxazol-5-yl)-2-methylpyridin-3-yl)carbamate (Intermediate 16)

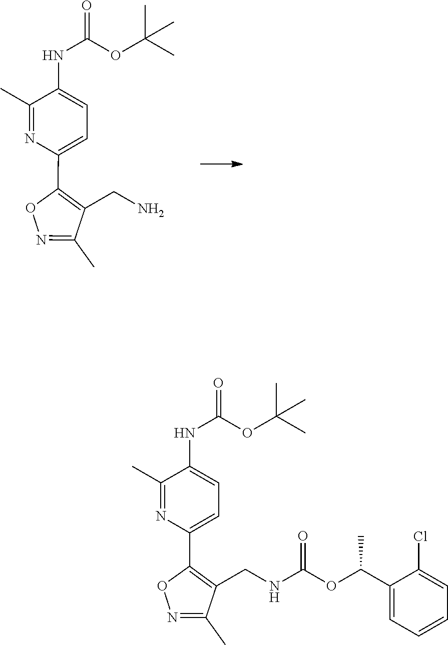 [SMILES: Cc1nc(-c2onc(C)c2CN)ccc1NC(=O)OC(C)(C)C ; Cc1nc(-c2onc(C)c2CNC(=O)O[C@H](C)c2ccccc2Cl)ccc1NC(=O)OC(C)(C)C]

To a solution of tert-butyl N-[6-[4-(aminomethyl)-3-methyl-1,2-oxazol-5-yl]-2-methylpyridin-3-yl]carbamate (Intermediate D12, 44.0 mg, 0.140 mmol) in THE (3 mL) was added DIPEA (0.1 mL, 0.550 mmol), followed by [(1R)-1-(2-chlorophenyl)ethyl](4-nitrophenyl) carbonate (Intermediate I1, 88.92 mg, 0.280 mmol). The mixture was stirred at RT overnight. NaHCO₃satd. sol. and EtOAc were added and the mixture was extracted with EtOAc, collected organic phases were dried over Na2SO4, filtered and evaporated. The crude was purified by flash chromatography (CyHex/EtOAc from 10/0 to 8/2) to afford the title compound (57 mg, 0.114 mmol, 82.33% yield) as white solid.

LC-MS (ESI): m/z (M+1): 501.3 (Method 1)

### Step 3: (R)-1-(2-chlorophenyl)ethyl ((5-(5-amino-6-methylpyridin-2-yl)-3-methylisoxazol-4-yl)meth 1 carbamate (Intermediate I7)

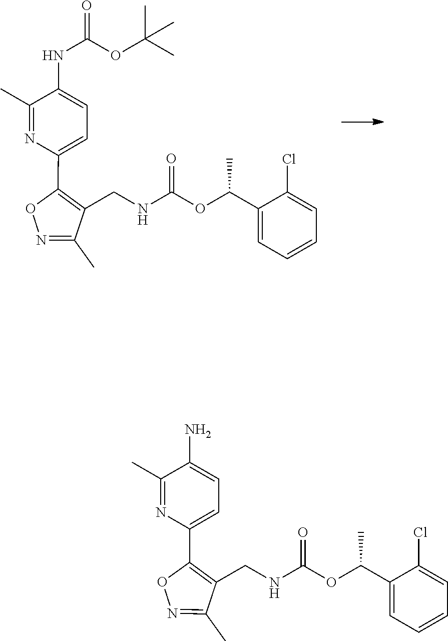 [SMILES: Cc1nc(-c2onc(C)c2CNC(=O)O[C@H](C)c2ccccc2Cl)ccc1NC(=O)OC(C)(C)C ; Cc1nc(-c2onc(C)c2CNC(=O)O[C@H](C)c2ccccc2Cl)ccc1N]

Intermediate 17 (41 mg, 0.102 mmol, 89.89% yield) was obtained from tert-butyl (R)-(6-(4-((((1-(2-chlorophenyl)ethoxy)carbonyl)amino)methyl)-3-methylisoxazol-5-yl)-2-methylpyridin-3-yl)carbamate (Intermediate 16, 57 mg, 0.114 mmol) using a similar method as for Intermediate F5.

LC-MS (ESI): m/z (M+1): 401.2 (Method 1)

### Step 4: tert-(R)-1-(2-chlorophenyl)ethyl ((5-(5-amino-6-methylpyridin-2-yl)-3-methylisoxazol-4-yl)methyl)carbamate (Compound 33)

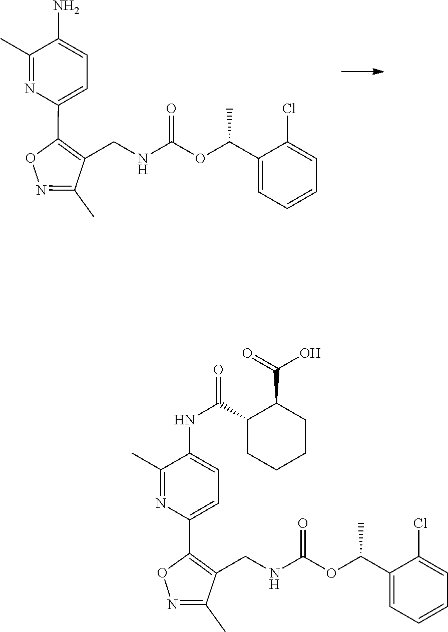 [SMILES: Cc1nc(-c2onc(C)c2CNC(=O)O[C@H](C)c2ccccc2Cl)ccc1N ; Cc1nc(-c2onc(C)c2CNC(=O)O[C@H](C)c2ccccc2Cl)ccc1NC(=O)[C@H]1CCCC[C@@H]1C(=O)O]

Compound 33 (40.5 mg, 0.073 mmol, 71.34% yield) was obtained from (R)-1-(2-chlorophenyl)ethyl ((5-(5-amino-6-methylpyridin-2-yl)-3-methylisoxazol-4-yl)methyl)carbamate (Intermediate 17, 41 mg, 0.102 mmol,) and (−)-trans-1,2-Cyclohexanedicarboxylic anhydride (17.34 mg, 0.110 mmol) using a similar method as for Compound 1.

LC-MS (ESI): m/z (M+1): 555.3 (Method 1)

¹H NMR (400 MHz, DMSO-d₆) δ ppm 12.19 (br s, 1H), 9.66 (br s, 1H), 7.98 (d, J=8.3 Hz, 1H), 7.70 (d, J=8.3 Hz, 1H), 7.63 (br t, J=5.1 Hz, 1H), 7.51-7.06 (m, 4H), 5.94 (q, J=6.4 Hz, 1H), 4.76-4.34 (m, 2H), 2.76-2.65 (m, 1H), 2.56-2.49 (m, 1H), 2.47 (s, 3H), 2.23 (s, 3H), 2.10-1.95 (m, 2H), 1.85-1.69 (m, 2H), 1.42 (br d, J=6.5 Hz, 3H), 1.46-1.11 (m, 4H)

The Compounds in the following table were prepared from reagents reported below by using methods analogous to Compound 33.

| Compound | Structure & Name | Reagents | Analytical data |
|---|---|---|---|
| 34 | 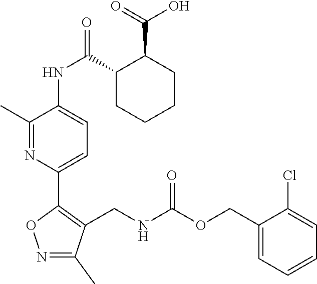 [SMILES: Cc1nc(-c2onc(C)c2CNC(=O)OCc2ccccc2Cl)ccc1NC(=O)[C@H]1CCCC[C@@H]1C(=O)O] (1S,2S)-2-((6-(4-(((((2- chlorobenzyl)oxy)carbonyl) amino)methyl)-3- methylisoxazol-5-yl)-2- methylpyridin-3- yl)carbamoyl)cyclohexane- 1-carboxylic acid | D12 and I2 | LC-MS (ESI): m/z (M + 1): 541.3 (Method 1) ¹H NMR (600 MHz, DMSO-d₆) δ ppm 12.14 (br s, 1 H), 9.63 (br s, 1 H), 7.98 (br d, J = 8.2 Hz, 1 H), 7.71 (d, J = 8.4 Hz, 1 H), 7.63 (br t, J = 5.1 Hz, 1 H), 7.39-7.51 (m, 2 H), 7.23-7.39 (m, 2 H), 5.10 (s, 2 H), 4.54 (br d, J = 5.4 Hz, 2 H), 2.70 (td, J = 11.3, 3.2 Hz, 1 H), 2.51-2.55 (m, 1 H), 2.48 (s, 3 H), 2.28 (s, 3 H), 1.95-2.10 (m, 2 H), 1.77 (br d, J = 10.9 Hz, 2 H), 1.23- 1.46 (m, 4 H) |
| 35 | 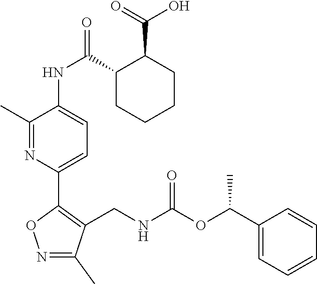 [SMILES: Cc1nc(-c2onc(C)c2CNC(=O)O[C@H](C)c2ccccc2)ccc1NC(=O)[C@H]1CCCC[C@@H]1C(=O)O] (1S,2S)-2-((2-methyl-6- (3-methyl-4-(((((R)-1- phenylethoxy)carbonyl) amino)methyl)isoxazol-5- yl)pyridin-3- yl)carbamoyl)cyclohexane- 1-carboxylic acid | D12 and I3 | LC-MS (ESI): m/z (M + 1): 521.3 (Method 1) 1H NMR (400 MHz, DMSO-d₆) δ ppm 11.05- 13.14 (m, 1 H), 9.32- 10.55 (m, 1 H), 8.05 (br d, J = 8.2 Hz, 1 H), 7.69 (d, J = 8.3 Hz, 1 H), 6.65-7.60 (m, 6 H), 5.68 (q, J = 6.2 Hz, 1 H), 4.39-4.75 (m, 2 H), 2.64-2.77 (m, 1 H), 2.50 (br s, 3 H), 2.35- 2.46 (m, 1 H), 2.23 (s, 3 H), 1.90-2.09 (m, 2 H), 1.75 (br s, 2 H), 1.16- 1.54 (m, 7 H) |

### Example 13

### Process Synthesis of Compound 36

### (1S,2S)-2-((6-(4-(((4-ethoxypyrimidin-2-yl)amino)methyl)-3-methylisoxazol-5-yl)-2-methylpyridin-3-yl)carbamoyl)cyclohexane-1-carboxylic acid

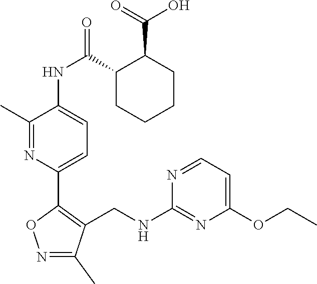 [SMILES: CCOc1ccnc(NCc2c(C)noc2-c2ccc(NC(=O)[C@H]3CCCC[C@@H]3C(=O)O)c(C)n2)n1]

### Step 1: tert-butyl (6-(4-(((4-ethoxypyrimidin-2-yl)amino)methyl)-3-methylisoxazol-5-yl)-2-methylpyridin-3-yl)carbamate (Intermediate J1)

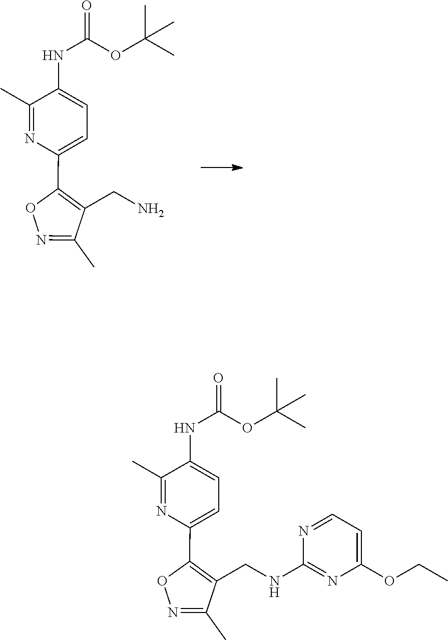 [SMILES: Cc1nc(-c2onc(C)c2CN)ccc1NC(=O)OC(C)(C)C ; CCOc1ccnc(NCc2c(C)noc2-c2ccc(NC(=O)OC(C)(C)C)c(C)n2)n1]

tert-butyl (6-(4-(aminomethyl)-3-methylisoxazol-5-yl)-2-methylpyridin-3-yl)carbamate (Intermediate D12, 89.0 mg, 0.280 mmol) was suspended in MeCN (1 mL), then DIPEA (0.15 mL, 0.840 mmol) was added, followed by 2-chloro-4-ethoxypyrimidine (53.03 mg, 0.330 mmol) and the reaction was heated at 50° C. for 3h and then 80° C. overnight. Solvent was removed under vacuum and sat NH₄Cl solution was added with EtOAc. The organic layer was washed with brine and then concentrated under vacuum. The crude was purified by flash chromatography using a gradient of EtOAc in CyHex from 000 to 45% affording the desired product (45 mg, 0.102 mmol, 36.6600 yield) as a whitenish solid.

LC-MS (ESI): m/z (M+1): 441.3 (Method 1)

The Intermediates in the following table were prepared from reagents reported below by using methods analogous to Intermediate J1.

| Intermediate | Structure & Name | Reagents | Analytical data |
|---|---|---|---|
| J2 | 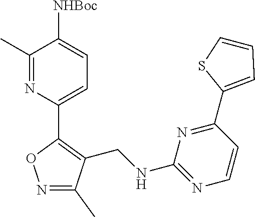 [SMILES: Cc1nc(-c2onc(C)c2CNc2nccc(-c3cccs3)n2)ccc1NC(=O)OC(C)(C)C] tert-butyl (2-methyl-6-(3- methyl-4-(((4-(thiophen- 2-yl)pyrimidin-2- yl)amino)methyl)isoxazol- 5-yl)pyridin-3- yl)carbamate | D12 and 2- chloro-4- (thiophen-2- yl)pyrimidine | LC-MS (ESI): m/z (M + 1): 479.1 (Method 7) ¹H NMR (400 MHz, DMSO-d₆) δ 9.02 (s, 1H), 8.29 (d, J = 5.2 Hz, 1H), 8.02 (d, J = 8.5 Hz, 1H), 7.85 (s, 1H), 7.74 (d, J = 8.5 Hz, 1H), 7.71-7.68 (m, 1H), 7.51 (s, 1H), 7.17 (dd, J = 5.0, 3.7 Hz, 1H), 7.09 (d, J = 5.2 Hz, 1H), 4.76 (s, 2H), 2.34 (s, 3H), 1.49 (s, 9H) |
| J3 | 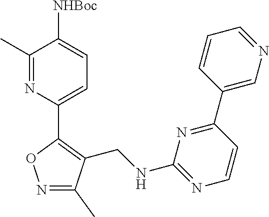 tert-butyl (2-methyl-6-(3- methyl-4-(((4-(pyridin-3- yl)pyrimidin-2- yl)amino)methyl)isoxazol- 5-yl)pyridin-3- yl)carbamate | D12 and 2- chloro-4- (pyridin-3- yl)pyrimidine | LC-MS (ESI): m/z (M + 1): 546.2 (Method 7) ¹H NMR (400 MHz, DMSO-d₆) δ 10.26 (s, 1H), 9.21 (d, J = 2.3 Hz, 1H), 8.65 (dd, J = 4.6, 1.6 Hz, 1H), 8.41 (d, J = 5.0 Hz, 1H), 8.29 (s, 1H), 8.18 (d, J = 8.5 Hz, 1H), 7.78 (d, J = 8.5 Hz, 1H), 7.62 (t, J = 5.7 Hz, 1H), 7.51-7.39 (m, 1H), 7.27 (d, J = 5.1 Hz, 1H), 4.87 (s, 2H), 3.75 (s, 3H), 3.74-3.67 (m, 1H), 3.24 (dd, J = 13.2, 7.9 Hz, 1H), 2.54 (s, 3H), 2.33 (s, 3H) |
| J4 | 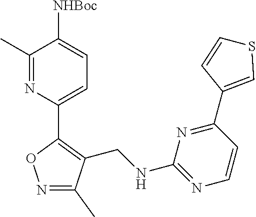 [SMILES: Cc1nc(-c2onc(C)c2CNc2nccc(-c3ccsc3)n2)ccc1NC(=O)OC(C)(C)C] tert-butyl (2-methyl-6-(3- methyl-4-(((4-(thiophen- 3-yl)pyrimidin-2- yl)amino)methyl)isoxazol- 5-yl)pyridin-3- yl)carbamate | D12 and 2- chloro-4- (thiophen-3- yl)pyrimidine | LC-MS (ESI): m/z (M + 1): 479.3 (Method 7) ¹H NMR (300 MHz, DMSO-d₆) δ 9.03 (s, 1H), 8.29 (d, J = 5.1 Hz, 1H), 8.03 (d, J = 8.5 Hz, 1H), 7.91-7.82 (m, 1H), 7.75 (d, J = 8.5 Hz, 1H), 7.71 (d, J = 4.9 Hz, 1H), 7.53 (t, J = 5.5 Hz, 1H), 7.17 (q, J = 5.0, 3.7 Hz, 1H), 7.10 (d, J = 5.2 Hz, 1H), 4.93- 4.54 (m, 2H), 2.34 (s, 3H), 1.50 (s, 9H) |
| J5 | 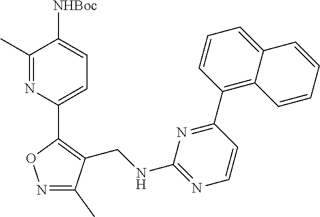 [SMILES: Cc1nc(-c2onc(C)c2CNc2nccc(-c3cccc4ccccc34)n2)ccc1NC(=O)OC(C)(C)C] tert-butyl (2-methyl-6-(3- methyl-4-(((4- (naphthalen-1- yl)pyrimidin-2- yl)amino)methyl)isoxazol- 5-yl)pyridin-3- yl)carbamate | D12 and 2- chloro-4- (naphthalen- 1- yl)pyrimidine | LC-MS (ESI): m/z (M + 1): 523.6 (Method 7) |
| J6 | 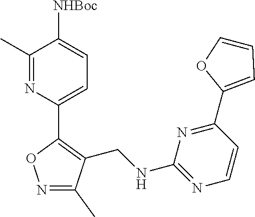 [SMILES: Cc1nc(-c2onc(C)c2CNc2nccc(-c3ccco3)n2)ccc1NC(=O)OC(C)(C)C] tert-butyl (6-(4-(((4- (furan-2-yl)pyrimidin-2- yl)amino)methyl)-3- methylisoxazol-5-yl)-2- methylpyridin-3- yl)carbamate | D12 and 2- chloro-4- (furan-2- yl)pyrimidine | LC-MS (ESI): m/z (M + 1): 463.3 (Method 7) ¹H NMR (300 MHz, DMSO-d₆) δ 9.02 (s, 1H), 8.32 (d, J = 5.1 Hz, 1H), 8.03 (d, J = 8.5 Hz, 1H), 7.88 (dd, J = 1.7, 0.8 Hz, 1H), 7.74 (d, J = 8.5 Hz, 1H), 7.46 (t, J = 5.8 Hz, 1H), 7.01 (s, 1H), 6.90 (d, J = 5.1 Hz, 1H), 6.64 (dd, J = 3.5, 1.8 Hz, 1H), 4.75 (d, J = 5.8 Hz, 2H), 2.34 (s, 3H), 1.49 (s, 9H) |

### Step 2: N-((5-(5-amino-6-methylpyridin-2-yl)-3-methylisoxazol-4-yl)methyl)-4-ethoxypyrimidin-2-amine (Intermediate J7)

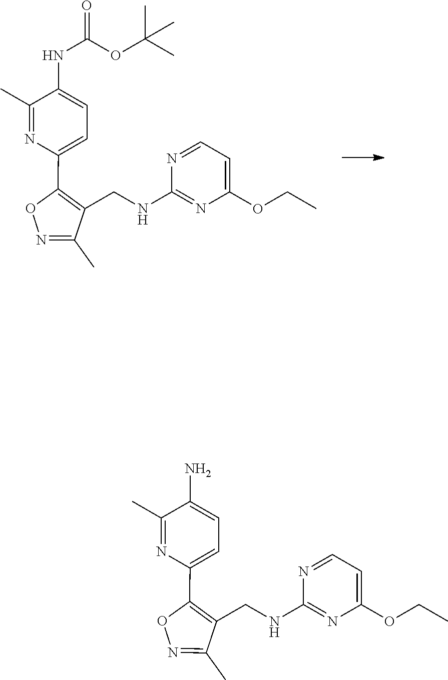

Intermediate J7 (31 mg, 0.091 mmol, 89.15% yield) was obtained from tert-butyl (6-(4-(((4-ethoxypyrimidin-2-yl)amino)methyl)-3-methylisoxazol-5-yl)-2-methylpyridin-3-yl)carbamate (Intermediate J1, 45 mg, 0.102 mmol) using a similar method as for Intermediate F5.

LC-MS (ESI): m/z (M+1): 401.2 (Method 1)

The Intermediates in the following table were prepared from reagents reported below by using methods analogous to Intermediate P7.

| Intermediate | Structure & Name | Reagents | Analytical data |
|---|---|---|---|
| J8 | 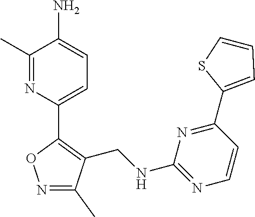 [SMILES: Cc1nc(-c2onc(C)c2CNc2nccc(-c3cccs3)n2)ccc1N] N-((5-(5-amino-6- methylpyridin-2-yl)-3- methylisoxazol-4- yl)methyl)-4-(thiophen- 2-yl)pyrimidin-2-amine | J2 | LC-MS (ESI): m/z (M + 1): 379.1 (Method 5) ¹H NMR (300 MHz, DMSO-d₆) δ 8.30 (s, 1H), 7.91-7.84 (m, 1H), 7.75- 7.69 (m, 1H), 7.59 (s, 1H), 7.50 (d, J = 8.4 Hz, 1H), 7.18 (dd, J = 5.0, 3.7 Hz, 1H), 7.10 (d, J = 5.2 Hz, 1H), 7.04 (d, J = 8.3 Hz, 1H), 5.70 (s, 2H), 4.67 (s, 2H), 2.38 (s, 3H), 2.31 (s, 3H) |
| J9 | 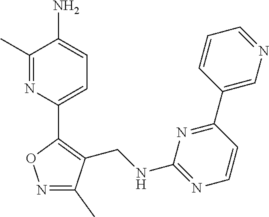 [SMILES: Cc1nc(-c2onc(C)c2CNc2nccc(-c3cccnc3)n2)ccc1N] N-((5-(5-amino-6- methylpyridin-2-yl)-3- methylisoxazol-4- yl)methyl)-4-(pyridin-3- yl)pyrimidin-2-amine | J3 | LC-MS (ESI): m/z (M + 1): 374.5 (Method 5) ¹H NMR (300 MHz, DMSO-d₆) δ 9.25 (s, 1H), 8.67 (dd, J = 4.8, 1.7 Hz, 1H), 8.41 (s, 1H), 8.34 (d, J = 8.1 Hz, 1H), 7.67 (s, 1H), 7.50 (d, J = 8.3 Hz, 2H), 7.27 (d, J = 5.1 Hz, 1H), 7.04 (d, J = 8.4 Hz, 1H), 5.70 (s, 2H), 4.76 (s, 2H), 2.36 (s, 3H), 2.29 (s, 3H) |
| J10 | 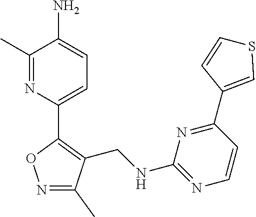 [SMILES: Cc1nc(-c2onc(C)c2CNc2nccc(-c3ccsc3)n2)ccc1N] N-((5-(5-amino-6- methylpyridin-2-yl)-3- methylisoxazol-4- yl)methyl)-4-(thiophen- 3-yl)pyrimidin-2-amine | J4 | LC-MS (ESI): m/z (M + 1): 379.2 (Method 5) ¹H NMR (300 MHz, Chloroform-d) δ 8.24 (d, J = 4.9 Hz, 1H), 7.72 (d, J = 3.3 Hz, 1H), 7.67 (d, J = 8.3 Hz, 1H), 7.52 (d, J = 5.0 Hz, 1H), 7.39 (bs, 1H), 7.16 (t, J = 4.4 Hz, 1H), 7.04 (d, J = 8.3 Hz, 1H), 6.88 (d, J = 5.3 Hz, 1H), 4.81 (s, 2H), 3.93 (s, 2H), 2.60 (s, 3H), 2.51 (s, 3H) |
| J11 | 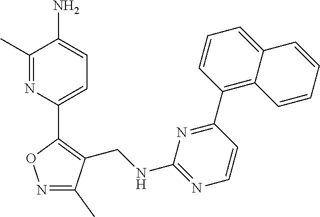 [SMILES: Cc1nc(-c2onc(C)c2CNc2nccc(-c3cccc4ccccc34)n2)ccc1N] N-((5-(5-amino-6- methylpyridin-2-yl)-3- methylisoxazol-4- yl)methyl)-4- (naphthalen-1- yl)pyrimidin-2-amine | J5 | LC-MS (ESI): m/z (M + 1): 423.3 (Method 5) ¹H NMR (300 MHz, DMSO-d₆) δ 8.45 (s, 1H), 8.20 (d, J = 8.5 Hz, 1H), 8.03 (t, J = 8.9 Hz, 3H), 7.67-7.53 (m, 5H), 7.50 (d, J = 8.6 Hz, 1H), 7.02 (d, J = 8.4 Hz, 1H), 6.89 (d, J = 5.0 Hz, 1H), 5.68 (s, 2H), 4.68 (d, J = 5.7 Hz, 2H), 2.35 (s, 3H), 2.22 (s, 3H) |
| J12 | 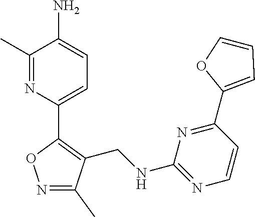 [SMILES: Cc1nc(-c2onc(C)c2CNc2nccc(-c3ccco3)n2)ccc1N] N-((5-(5-amino-6- methylpyridin-2-yl)-3- methylisoxazol-4- yl)methyl)-4-(furan-2- yl)pyrimidin-2-amine | J6 | LC-MS (ESI): m/z (M + 1): 363.3 (Method 5) ¹H NMR (300 MHz, DMSO-d₆) δ 8.32 (s, 1H), 7.89 (dd, J = 1.8, 0.8 Hz, 1H), 7.50 (d, J = 8.3 Hz, 2H), 7.13 (d, J = 3.4 Hz, 1H), 7.03 (d, J = 8.4 Hz, 1H), 6.90 (d, J = 5.1 Hz, 1H), 6.66 (dd, J = 3.4, 1.8 Hz, 1H), 5.70 (s, 2H), 4.66 (d, J = 5.9 Hz, 2H), 2.37 (s, 3H), 2.32 (s, 3H) |

### Step 3: (1S,2S)-2-((6-(4-(((4-ethoxypyrimidin-2-yl)amino)methyl)-3-methylisoxazol-5-yl)-2-methylpyridin-3-yl)carbamoyl)cyclohexane-1-carboxylic acid (Compound 36)

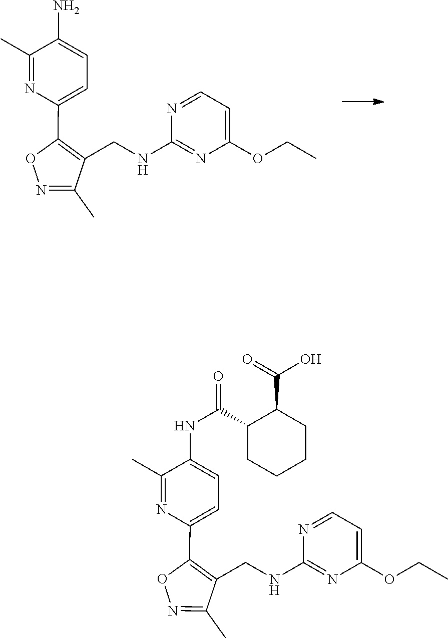

Compound 36 (13 mg, 0.026 mmol, 28.86% yield) was obtained from N-((5-(5-amino-6-methylpyridin-2-yl)-3-methylisoxazol-4-yl)methyl)-4-ethoxypyrimidin-2-amine (Intermediate J7, 31 mg, 0.091 mmol) and (−)-trans-1,2-Cyclohexanedicarboxylic anhydride (21.06 mg, 0.140 mmol) using a similar method as for Compound 1.

LC-MS (ESI): m/z (M+1): 555.3 (Method 1)

¹H NMR (400 MHz, DMSO-d₆) δ ppm 12.14 (br d, J=2.0 Hz, 1H), 9.69 (br s, 1H), 8.07-7.93 (m, 2H), 7.73 (d, J=8.3 Hz, 1H), 7.38 (br d, J=2.0 Hz, 1H), 5.99 (br d, J=4.8 Hz, 1H), 4.73 (br d, J=1.8 Hz, 2H), 4.42-3.81 (m, 2H), 3.41-3.34 (m, 1H), 2.81-2.61 (m, 1H), 2.53-2.51 (m, 3H), 2.30 (s, 3H), 2.09-1.93 (m, 2H), 1.76 (br s, 2H), 1.44-1.05 (m, 7H)

The Compounds in the following table were prepared from reagents reported below by using methods analogous to Compound 36.

| Compound | Structure & Name | Reagents | Analytical data |
|---|---|---|---|
| 37 | 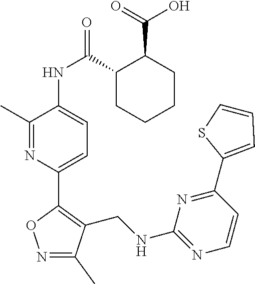 [SMILES: Cc1nc(-c2onc(C)c2CNc2nccc(-c3cccs3)n2)ccc1NC(=O)[C@H]1CCCC[C@@H]1C(=O)O] (1S,2S)-2-((2-methyl-6- (3-methyl-4-(((4- (thiophen-2- yl)pyrimidin-2- yl)amino)methyl)isoxazol- 5-yl)pyridin-3- yl)carbamoyl)cyclohexane- 1-carboxylic acid | J8 and (-)- trans-1,2- Cyclohexane- dicarboxylic | LC-MS (ESI): m/z (M + 1): 533.2 (Method 8) ¹H NMR (300 MHz, DMSO- d₆) δ 12.17 (s, 1H), 9.67 (s, 1H), 8.31 (d, J = 5.1 Hz, 1H), 8.28-8.14 (m, 1H), 8.01 (d, J = 8.4 Hz, 1H), 7.76 (d, J = 8.4 Hz, 1H), 7.65-7.53 (m, 2H), 7.46 (t, J = 5.9 Hz, 1H), 7.06 (d, J = 5.1 Hz, 1H), 4.82 (s, 2H), 2.73 (s, 1H), 2.63-2.56 (m, 1H), 2.50 (s, 3H) 2.34 (s, 3H), 2.11-1.95 (m, 2H), 1.78 (s, 2H), 1.49-1.20 (m, 4H) |
| 38 | 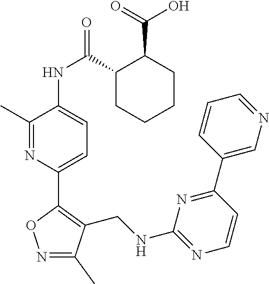 [SMILES: Cc1nc(-c2onc(C)c2CNc2nccc(-c3cccnc3)n2)ccc1NC(=O)[C@H]1CCCC[C@@H]1C(=O)O] (1S,2S)-2-((2-methyl-6- (3-methyl-4-(((4- (pyridin-3- yl)pyrimidin-2- yl)amino)methyl)isoxazol- 5-yl)pyridin-3- yl)carbamoyl)cyclohexane- 1-carboxylic acid | J9 and (-)- trans-1,2- Cyclohexane- dicarboxylic | LC-MS (ESI): m/z (M + 1): 528.2 (Method 9) ¹H NMR (300 MHz, DMSO- d₆) δ 12.17 (s, 1H), 9.66 (s, 1H), 9.23 (s, 1H), 8.66 (d, J = 4.8 Hz, 1H), 8.41 (d, J = 4.7 Hz, 1H), 8.27 (bs, 1H), 8.01 (d, J = 8.4 Hz, 1H), 7.75 (d, J = 8.4 Hz, 1H), 7.64 (t, J = 5.9 Hz, 1H), 7.44 (s, 1H), 7.28 (d, J = 5.2 Hz, 1H), 4.87 (s, 2H), 2.82-2.71 (m, 1H), 2.62- 2.55 (m, 1H), 2.33 (s, 3H), 2.14-1.91 (m, 2H), 1.78 (s, 2H), 1.32 (s, 4H) |
| 39 | 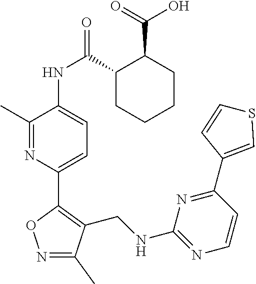 [SMILES: Cc1nc(-c2onc(C)c2CNc2nccc(-c3ccsc3)n2)ccc1NC(=O)[C@H]1CCCC[C@@H]1C(=O)O] (1S,2S)-2-((2-methyl-6- (3-methyl-4-(((4- (thiophen-3- yl)pyrimidin-2- yl)amino)methyl)isoxazol- 5-yl)pyridin-3- yl)carbamoyl)cyclohexane- 1-carboxylic acid | J10 and (-)- trans- 1,2- Cyclohexane- dicarboxylic | LC-MS (ESI): m/z (M + 1): 533.2 (Method 9) ¹H NMR (300 MHz, DMSO- d₆) δ 12.18 (s, 1H), 9.68 (s, 1H), 8.29 (d, J = 5.1 Hz, 1H), 7.99 (d, J = 8.4 Hz, 1H), 7.85 (s, 1H), 7.76 (d, J = 8.3 Hz, 1H), 7.59-7.49 (m, 1H), 7.16 (t, J = 4.4 Hz, 1H), 7.10 (d, J = 5.2 Hz, 1H), 4.79 (s, 2H), 2.78- 2.68 (m, 1H), 2.64-2.53 (m, 1H), 2.34 (s, 3H), 2.08-1.94 (m, 2H), 1.78 (s, 2H), 1.31 (s, 4H) |
| 40 | 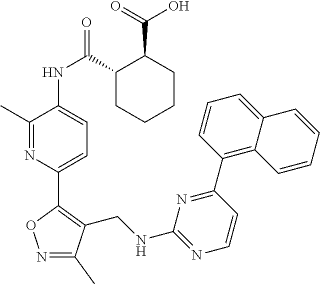 [SMILES: Cc1nc(-c2onc(C)c2CNc2nccc(-c3cccc4ccccc34)n2)ccc1NC(=O)[C@H]1CCCC[C@@H]1C(=O)O] (1S,2S)-2-((2-methyl-6- (3-methyl-4-(((4- (naphthalen-1- yl)pyrimidin-2- yl)amino)methyl)isoxazol- 5-yl)pyridin-3- yl)carbamoyl)cyclohexane- 1-carboxylic acid | J11 and (-)- trans- 1,2- Cyclohexane- dicarboxylic | LC-MS (ESI): m/z (M + 1): 577.2 (Method 9) ¹H NMR (300 MHz, DMSO- d₆) δ 9.97 (s, 1H), 8.44 (d, J = 5.0 Hz, 1H), 8.16 (d, J = 8.2 Hz, 2H), 8.06-7.94 (m, 2H), 7.70 (d, J = 8.4 Hz, 1H), 7.65- 7.45 (m, 5H), 6.88 (d, J = 5.0 Hz, 1H), 4.75 (d, J = 5.6 Hz, 2H), 2.70-2.64 (m, 1H), 2.40- 2.18 (m, 4H), 2.09-1.97 (m, 1H), 1.97-1.84 (m, 1H), 1.73 (s, 2H), 1.43-1.20 (m, 4H) |
| 41 | 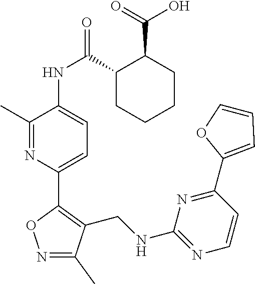 [SMILES: Cc1nc(-c2onc(C)c2CNc2nccc(-c3ccco3)n2)ccc1NC(=O)[C@H]1CCCC[C@@H]1C(=O)O] (1S,2S)-2-((6-(4-(((4- (furan-2-yl)pyrimidin- 2-yl)amino)methyl)-3- methylisoxazol-5-yl)-2- methylpyridin-3- yl)carbamoyl)cyclohexane- 1-carboxylic acid | J12 and (-)- trans- 1,2- Cyclohexane- dicarboxylic | LC-MS (ESI): m/z (M + 1): 517.3 (Method 6) ¹H NMR (300 MHz, DMSO- d₆) δ 12.17 (s, 1H), 9.67 (s, 1H), 8.38-8.22 (m, 1H), 8.00 (d, J = 8.4 Hz, 1H), 7.88 (s, 1H), 7.76 (d, J = 8.4 Hz, 1H), 7.49 (t, J = 5.8 Hz, 1H), 7.00 (bs, 1H), 6.91 (d, J = 5.2 Hz, 1H), 6.64 (s, 1H), 4.84-4.71 (m, 2H), 2.78-2.67 (m, 1H), 2.66-2.58 (m, 1H), 2.35 (s, 3H), 2.02 (s, 2H), 1.78 (s, 2H), 1.45-1.22 (m, 4H) |

### Example 14

### Process Synthesis of Compound 42

### (1S,2S)-2-((6-(5-(((((R)-1-(2-chlorophenyl)ethoxy)carbonyl)amino)methyl)-1-methyl-1H-1,2,3-triazol-4-yl)-2-methylpyridin-3-yl)carbamoyl)cyclohexane-1-carboxylic acid

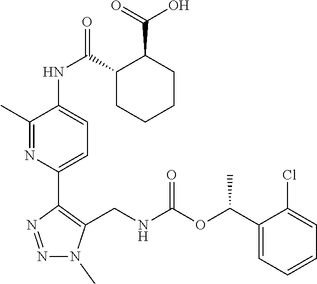 [SMILES: Cc1nc(-c2nnn(C)c2CNC(=O)O[C@H](C)c2ccccc2Cl)ccc1NC(=O)[C@H]1CCCC[C@@H]1C(=O)O]

### Step 1: benzyl prop-2-yn-1-ylcarbamate (intermediate K1)

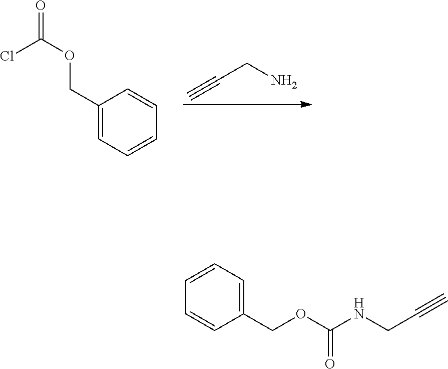

To a solution of 2-propyn-1-amine (250.0 mg, 4.54 mmol), and K₂CO₃ (1254.63 mg, 9.08 mmol) in THE (24 mL) and Water (12 mL), carbonochloridic acid (phenylmethyl) ester (0.64 mL, 4.54 mmol) was added at 5° C. The resulting solution was stirred and allowed to warm to RT and stirred overnight and then quenched with aq. NH₄Cl. The two phases were separated and the aqueous phase was extracted with EtOAc (2 times). The combined organic extracts were filtered through a phase separator and concentrated under reduced pressure. The crude was purified by flash chromatography with isocratic elution with DCM. Collected fractions afforded the desired product (555 mg, 2.933 mmol, 64.63% yield) as a colorless oil.

LC-MS (ESI): m/z (M+1): 190.1 (Method 1)

### Step 2: benzyl (3-(6-methyl-5-nitropyridin-2-yl)prop-2-yn-1-yl)carbamate (Intermediate K2)

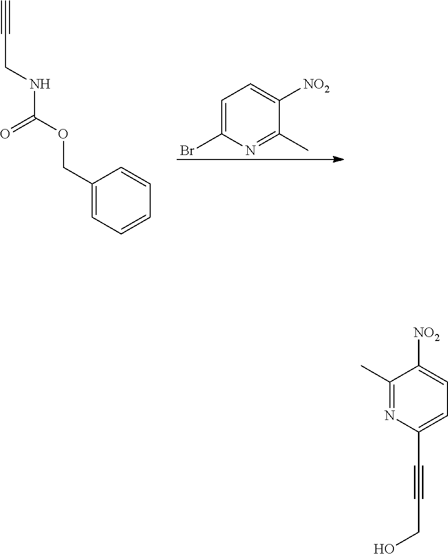

To a solution of 6-bromo-2-methyl-3-nitropyridine (325.0 mg, 1.5 mmol) and benzyl prop-2-yn-1-ylcarbamate (Intermediate K1, 425.03 mg, 2.25 mmol) in dry MeCN (8 mL), under inert atmosphere, TEA (0.5 mL, 3.59 mmol) was added at 0° C. After degassing the solution with N2 for 5 minutes PdCl₂(PPh₃)₂ (44.15 mg, 0.060 mmol) was added, followed by CuI (11.98 mg, 0.060 mmol). The reaction mixture was stirred at RT for 2 h, then salts were filtered off through a celite pad and washed with EtOAc. The filtrated was concentrated under reduced pressure and purified by flash chromatography with a gradient of EtOAc in CyHex from 0% to 30%. Collected fractions afforded the target product (513 mg, 1.577 mmol, 105.3% yield) as a brown oil.

LC-MS (ESI): m/z (M+1): 326.0 (Method 1)

The Intermediates in the following table were prepared from reagents reported below by using methods analogous to Intermediate K2.

| Intermediate | Structure & Name | Reagents | Analytical data |
|---|---|---|---|
| K3 | 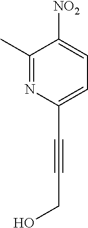 3-(6-methyl-5- nitropyridin-2-yl)prop-2- yn-1-ol | 6-bromo-2- methyl-3- nitropyridine and propargyl alcohol | LC-MS (ESI): m/z (M + 1): 193.0 (Method 1) ¹H NMR (400 MHz, Chloroform-d) δ 8.27 (d, J = 8.39 Hz, 1H), 7.44 (d, J = 8.39 Hz, 1H), 4.56 (d, J = 5.72 Hz, 2H), 2.87 (s, 3H), 1.88 (d, J = 12.55 Hz, 1H). 2.48 (s, 3 H), 2.28 (s, 3 H), 1.95-2.10 (m, 2 H), 1.77 (br d, J = 10.9 Hz, 2 H), 1.23-1.46 (m, 4 H) |
| K4 | 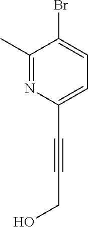 3-(5-bromo-6- methylpyridin-2-yl)prop- 2-yn-1-ol | 6-bromo-2- methyl-3- bromopyridine and propargyl alcohol | LC-MS (ESI): m/z (M + 1): 228.0 (Method 1) ¹H NMR (400 MHz, DMSO-d₆6) δ 8.02 (d, J = 8.17 Hz, 1H), 7.27 (dd, J = 8.24, 0.68 Hz, 1H), 5.42 (t, J = 5.98 Hz, 1H), 4.32 (d, J = 5.93 Hz, 2H), 2.56 (s, 3H) |
| K5 | 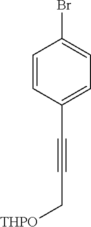 bromophenyl)prop-2-yn- 1-yl)oxy)tetrahydro-2H- pyran | 1-bromo-4- iodobenzene and 2- (prop-2-yn- 1- yloxy) tetrahydro-2H- pyran | LC-MS (ESI): m/z (M + 1): 297.1 (Method 2) ¹H NMR (300 MHz, DMSO-d₆) δ 7.64-7.54 (m, 1H), 7.47-7.35 (m, 1H), 4.84-4.78 (m, 1H), 4.44 (d, J = 13.0 Hz, 1H), 3.76 (ddd, J = 11.7, 8.2, 3.7 Hz, 1H), 3.53-3.43 (m, 1H), 1.78-1.61 (m, 2H), 1.61-1.42 (m, 4H) |

### Step 3: benzyl ((1-methyl-4-(6-methyl-5-nitropyridin-2-yl)-1H-1,2,3-triazol-5-yl)methyl)carbamate (intermediate K6)

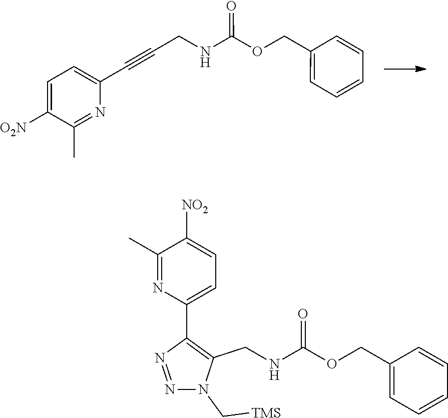 [SMILES: Cc1nc(C#CCNC(=O)OCc2ccccc2)ccc1[N+](=O)[O-] ; Cc1nc(-c2nnn(C[Si](C)(C)C)c2CNC(=O)OCc2ccccc2)ccc1[N+](=O)[O-]]

To a solution of benzyl (3-(6-methyl-S-nitropyridin-2-yl)prop-2-yn-1-yl)carbamate (Intermediate K2, 513.0 mg, 1.47 mmol) in dry 1,4-Dioxane (7.219 mL), Cp*RuCl(PPh3)2 (70.46 mg, 0.090 mmol) was added, followed by TMSN3 (1.47 mL, 2.2 mmol). The mixture was stirred at 50° C. for 3 hours. The mixture was cooled down at RT and the solvent was removed under reduced pressure. The residue was purified by flash chromatography with a gradient of EtOAc in CyHex from 0% to 15%. Collected fractions afforded the title compound (498 mg, 1.096 mmol, 74.71% yield) as a brown oil.

LC-MS (ESI): m/z (M+1): 455.3 (Method 1)

The Intermediates in the following table were prepared from reagents reported below by using methods analogous to Intermediate K6.

| Intermediate | Structure & Name | Reagents | Analytical data |
|---|---|---|---|
| K7 | 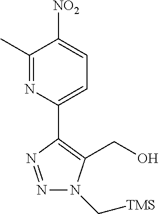 (4-(6-methyl-5- nitropyridin-2-yl)-1- ((trimethylsilyl)methyl)- 1H-1,2,3-triazol-5- yl)methanol | K3 | LC-MS (ESI): m/z (M + 1): 336.2 (Method 1) ¹H NMR (400 MHz, Chloroform-d) δ 8.48 (d, J = 8.66 Hz, 1H), 8.36 (dd, J = 8.68, 0.61 Hz, 1H), 6.20 (t, J = 6.95 Hz, 1H), 4.87 (d, J = 6.94 Hz, 2H), 4.51- 4.42 (m, 2H), 2.95 (s, 3H), 1.28-1.18 (m, 2H), 0.10 (s, 9H) |
| K8 | 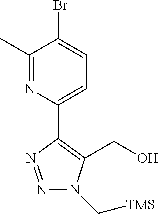 [SMILES: Cc1nc(-c2nnn(C[Si](C)(C)C)c2CO)ccc1Br] (4-(5-bromo-6- methylpyridin-2-yl)-1- ((trimethylsilyl)methyl)- 1H-1,2,3-triazol-5- yl)methanol | K4 | LC-MS (ESI): m/z (M + 1): 357.0 (Method 1) 1H NMR (400 MHz, DMSO-d₆) δ ppm 8.11 (d, J = 8.36 Hz, 1 H) 7.83 (d, J = 8.36 Hz, 1 H) 5.56 (s, 1 H) 5.03 (d, J = 5.72 Hz, 2 H) 3.99 (s, 2 H) 2.64 (s, 3 H) 0.15 (s, 9 H) |
| K9 | 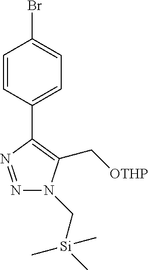 4-(4-bromophenyl)-5- (((tetrahydro-2H-pyran- 2-yl)oxy)methyl)-1- ((trimethylsilyl)methyl)- 1H-1,2,3-triazole | K5 | LC-MS (ESI): m/z (M + 1): 427.4 (Method 4) ¹H NMR (300 MHz, DMSO-d₆) δ 7.70 (s, 4H), 4.84 (d, J = 12.9 Hz, 1H), 4.77-4.72 (m, 1H), 4.69 (d, J = 12.9 Hz, 1H), 3.93 (s, 2H), 3.78-3.64 (m, 1H), 3.52-3.40 (m, 1H), 1.72-1.58 (m, 2H), 1.58- 1.41 (m, 4H), 0.16 (s, 9H) |

### Step 4: benzyl ((1-methyl-4-(6-methyl-5-nitropyridin-2-yl)-1H-1,2,3-triazol-5-yl)methyl)carbamate (Intermediate K10)

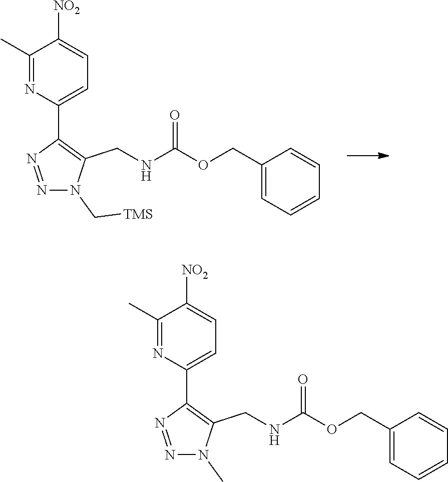 [SMILES: Cc1nc(-c2nnn(C[Si](C)(C)C)c2CNC(=O)OCc2ccccc2)ccc1[N+](=O)[O-] ; Cc1nc(-c2nnn(C)c2CNC(=O)OCc2ccccc2)ccc1[N+](=O)[O-]]

To a solution of benzyl ((1-methyl-4-(6-methyl-5-nitropyridin-2-yl)-1H-1,2,3-triazol-5-yl)methyl)carbamate (Intermediate K6, 398.0 mg, 0.880 mmol) in dry THE (8.379 mL), TBAF (0.92 mL, 0.920 mmol) 1M in THE was added at 0° C. The reaction mixture was stirred for 60 min and then a sat. sol of NaHCO₃ was added and the mixture was vigorously stirred at RT for 15 min. The mixture was diluted with EtOAc and then organic phase was washed with water and brine, filtered over a phase separator and evaporated to dryness. This was purified by flash chromatography using a gradient of MeCN in water from 0% to 60%. Collected fractions afforded the tile compound (218 mg, 0.570 mmol, 65.11% yield) as a brown solid.

LC-MS (ESI): m/z (M+1): 383.3 (Method 1)

The Intermediates in the following table were prepared from reagents reported below by using methods analogous to Intermediate K10.

| Intermediate | Structure & Name | Reagents | Analytical data |
|---|---|---|---|
| K11 | 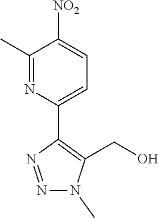 [SMILES: Cc1nc(-c2nnn(C)c2CO)ccc1[N+](=O)[O-]] (1-methyl-4-(6-methyl-5- nitropyridin-2-yl)-1H- 1,2,3-triazol-5- yl)methanol | K7 | LC-MS (ESI): m/z (M + 1): 250.1 (Method 1) ¹H NMR (400 MHz, Chloroform-d) δ ppm 8.53- 8.45 (m, 1 H), 8.36 (d, J = 8.58 Hz, 1 H), 6.15 (br. s., 1 H), 4.98-4.85 (m, 2 H), 4.14 (s, 3 H), 2.96 (s, 3 H) |
| K12 | 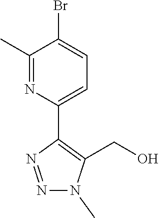 [SMILES: Cc1nc(-c2nnn(C)c2CO)ccc1Br] (4-(5-bromo-6- methylpyridin-2-yl)-1- methyl-1H-1,2,3-triazol- 5-yl)methanol | K8 | LC-MS (ESI): m/z (M + 1): 285.0 (Method 1) ¹H NMR (400 MHz, Chloroform-d) δ 8.06 (dd, J = 8.38, 0.68 Hz, 1H), 7.98 (d, J = 8.42 Hz, 1H), 6.86 (t, J = 6.73 Hz, 1H), 4.86 (d, J = 6.71 Hz, 2H), 4.11 (s, 3H), 2.74 (s, 3H) |
| K13 | 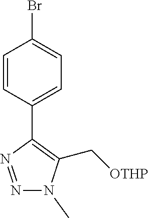 4-(4-bromophenyl)-1- methyl-5-(((tetrahydro- 2H-pyran-2- yl)oxy)methyl)-1H-1,2,3- triazole | K9 | LC-MS (ESI): m/z (M + 1): 353.4 (Method 4) ¹H NMR (300 MHz, DMSO-d₆) δ 7.70 (s, 4H), 4.86 (d, J = 13.1 Hz, 1H), 4.78-4.68 (m, 2H), 4.09 (s, 3H), 3.75-3.63 (m, 1H), 3.50-3.38 (m, 1H), 1.74-1.58 (m, 2H), 1.54- 1.39 (m, 4H) |

### Step 5: benzyl ((4-(5-amino-6-methylpyridin-2-yl)-1-methyl-1H-1,2,3-triazol-5-yl)methyl)carbamate (Intermediate K14)

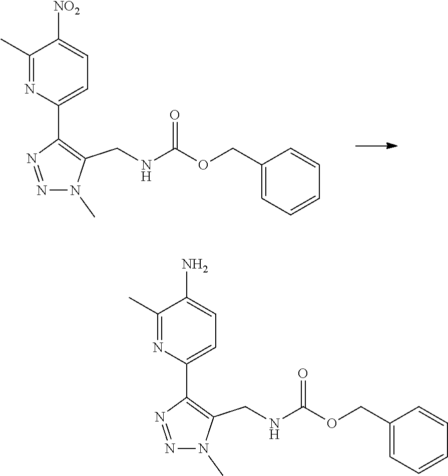 [SMILES: Cc1nc(-c2nnn(C)c2CNC(=O)OCc2ccccc2)ccc1[N+](=O)[O-] ; Cc1nc(-c2nnn(C)c2CNC(=O)OCc2ccccc2)ccc1N]

Intermediate K14 (168 mg, 0.477 mmol, 68.02% yield) was obtained from benzyl ((1-methyl-4-(6-methyl-5-nitropyridin-2-yl)-1H-1,2,3-triazol-5-yl)methyl)carbamate (Intermediate K10, 268.0 mg, 0.700 mmol) using a similar method as for Intermediate C3.

LC-MS (ESI): m/z (M+1): 353.3 (Method 1)

### Step 6: (1S,2S)-2-((6-(5-((((benzyloxy)carbonyl)amino)methyl)-1-methyl-1H-1,2,3-triazol-4-yl)-2-methylpyridin-3-yl)carbamoyl)cyclohexane-1-carboxylic acid (Intermediate K15)

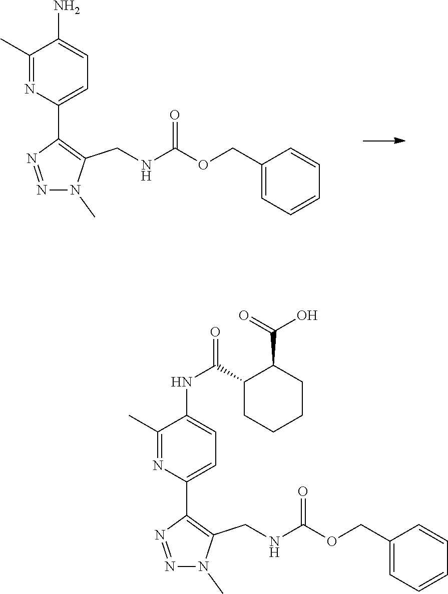 [SMILES: Cc1nc(-c2nnn(C)c2CNC(=O)OCc2ccccc2)ccc1N ; Cc1nc(-c2nnn(C)c2CNC(=O)OCc2ccccc2)ccc1NC(=O)[C@H]1CCCC[C@@H]1C(=O)O]

Intermediate K15 (240 mg, 0.474 mmol, 99.38% yield) was obtained as white off solid) from benzyl ((4-(5-amino-6-methylpyridin-2-yl)-1-methyl-1H-1,2,3-triazol-5-yl)methyl)carbamate (Intermediate K14, 168.0 mg, 0.480 mmol) and (−)-trans-1,2-Cyclohexanedicarboxylic anhydride (80.84 mg, 0.520 mmol) using a similar method as for Compound 1.

LC-MS (ESI): m/z (M+1): 507.3 (Method 1)

### Step 7: (1S,2S)-2-((6-(5-(aminomethyl)-1-methyl-1H-1,2,3-triazol-4-yl)-2-methylpyridin-3-yl)carbamoyl)cyclohexane-1-carboxylic acid (Intermediate K16)

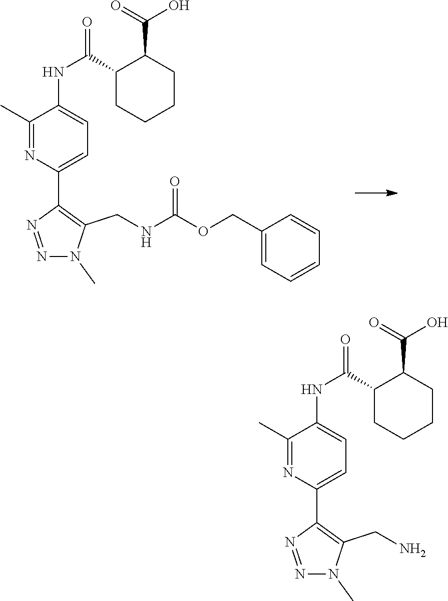 [SMILES: Cc1nc(-c2nnn(C)c2CNC(=O)OCc2ccccc2)ccc1NC(=O)[C@H]1CCCC[C@@H]1C(=O)O ; Cc1nc(-c2nnn(C)c2CN)ccc1NC(=O)[C@H]1CCCC[C@@H]1C(=O)O]

Intermediate K16 (150 mg, 0.367 mmol, 92.92% yield) was obtained from (1S,2S)-2-((6-(5-((((benzyloxy)carbonyl)amino)methyl)-1-methyl-1H-1,2,3-triazol-4-yl)-2-methylpyridin-3-yl)carbamoyl)cyclohexane-1-carboxylic acid (Intermediate K15, 200.0 mg, 0.390 mmol) using a similar method as for Intermediate A21.

LC-MS (ESI): m/z (M+1): 373.3 (Method 1)

### Step 8: (1S,2S)-2-((6-(5-(((((R)-1-(2-chlorophenyl)ethoxy)carbonyl)amino)methyl)-1-methyl-1H-1,2,3-triazol-4-yl)-2-methylpyridin-3-yl)carbamoyl)cyclohexane-1-carboxylic acid (Compound 42)

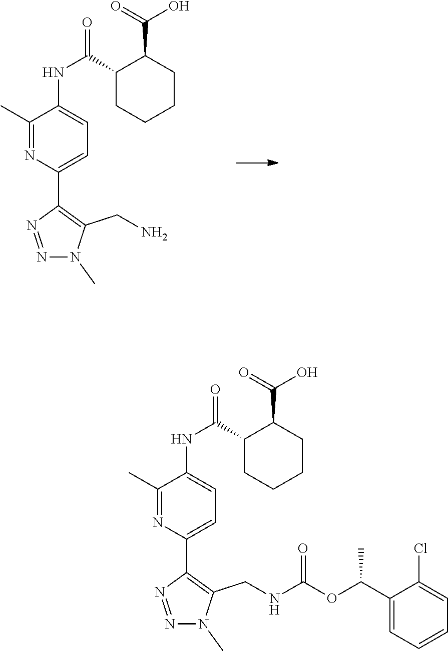 [SMILES: Cc1nc(-c2nnn(C)c2CN)ccc1NC(=O)[C@H]1CCCC[C@@H]1C(=O)O ; Cc1nc(-c2nnn(C)c2CNC(=O)O[C@H](C)c2ccccc2Cl)ccc1NC(=O)[C@H]1CCCC[C@@H]1C(=O)O]

To a suspension of ((1S,2S)-2-((6-(5-(aminomethyl)-1-methyl-1H-1,2,3-triazol-4-yl)-2-methylpyridin-3-yl)carbamoyl)cyclohexane-1-carboxylic acid (Intermediate K16, 50.0 mg, 0.130 mmol) and DIPEA (0.09 mL, 0.540 mmol) in THE (4 mL) was added at 0° C. [(1R)-1-(2-chlorophenyl)ethyl](4-nitrophenyl) carbonate (Intermediate I1, 51.83 mg, 0.160 mmol). After 2 hours of stirring DMF (3 mL) was added and the mixture was stirred at RT overnight. Satd. NaHCO₃ solution and EtOAc were added to the mixture. The two phases were separated and the organic phase was washed with water and brine (once). The organic phase was filtered over a phase separator and evaporated to give the crude material which by flash chromatography with a gradient of MeCN in water from 0% to 50%. Collected fractions afforded the target product (53.2 mg, 0.096 mmol, 71.39% yield) as a pale pink solid.

LC-MS (ESI): m/z (M+1): 555.3 (Method 1)

¹H NMR (400 MHz, DMSO-d6) δ ppm 12.11 (br s, 1H), 9.56 (br s, 1H), 7.95-7.71 (m, 3H), 7.49-7.23 (m, 4H), 5.93 (q, J=6.5 Hz, 1H), 5.14-4.70 (m, 2H), 4.02 (s, 3H), 2.74-2.61 (m, 1H), 2.58-2.48 (m, 1H), 2.43 (s, 3H), 2.11-1.94 (m, 2H), 1.88-1.67 (m, 2H), 1.41 (br d, J=6.4 Hz, 3H), 1.38-1.18 (m, 4H)

### Example 15

### Process Synthesis of Compound 43

### 2-((4-(5-((6-isopropoxypyrazin-2-yl)amino)-1-methyl-1H-1,2,3-triazol-4-yl)phenyl)carbamoyl)cyclohexane-1-carboxylic acid (cis racemate)

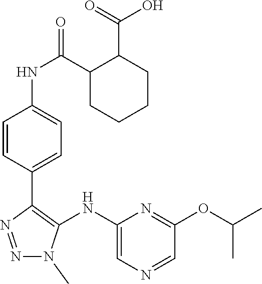 [SMILES: CC(C)Oc1cncc(Nc2c(-c3ccc(NC(=O)C4CCCCC4C(=O)O)cc3)nnn2C)n1]

### Step 1: N-benzyl-4-(1-methyl-5-(((tetrahydro-2H-pyran-2-yl)oxy)methyl)-1H-1,2,3-triazol-4-yl)aniline (Intermediate L1)

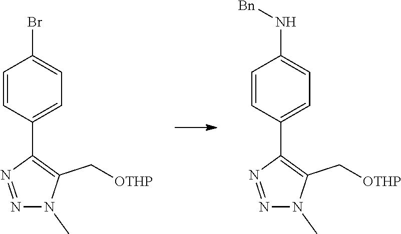

In the sealed tube, 4-(4-bromophenyl)-1-methyl-5-[(oxan-2-yloxy)methyl]-1H-1,2,3-triazole (Intermediate K13, 1.00 g, 2.84 mmol) was dissolved in degassed 1,4-dioxane (19 mL, in sealed tube with Cs₂CO₃ (2.775 g, 8.52 mmol), XantPhos (0.246 g, 0.43 mmol) and benzylamine (0.465 mL, 4.26 mmol) and the mixture were purged with argon. Lastly Pd₂(dba)₃ (0.130 g, 0.14 mmol) were added to mixture, that was heated at 90° C. with stirring overnight. The reaction mixture was filtered through a Celite® and washed with EtOAc (3×20 mL). Solvent was evaporated under vacuum. The crude product was purified by flash chromatography (0-55% of EtOAc in hexane). The product was collected as yellow foam (0.87 g, 2.30 mmol, 81%, yield).

LC-MS (ESI): m/z (M+1): 379.7 (Method 4)

### Step 2: 4-(1-methyl-5-(((tetrahydro-2H-pyran-2-yl)oxy)methyl)-1H-1,2,3-triazol-4-yl)aniline (Intermediate L2)

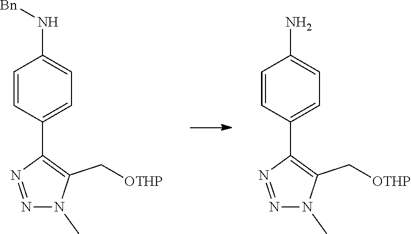

Intermediate L2 (0.60 g, 154 mmol, 67% yield) was obtained as a white solid from N-benzyl-4-(1-methyl-5-(((tetrahydro-2H-pyran-2-yl)oxy)methyl)-1H-1,2,3-triazol-4-yl)aniline (Intermediate L 1, 0.87 g, 2.30 mmol) using a similar method as for Intermediate A21.

LC-MS (ESI): m/z (M+1): 289.5 (Method 5)

### Step 3: (1S,2S)-2-((4-(1-methyl-5-(((tetrahydro-2H-pyran-2-yl)oxy)methyl)-1H-1,2,3-triazol-4-yl)phenyl)carbamoyl)cyclohexane-1-carboxylic acid (Intermediate L3)

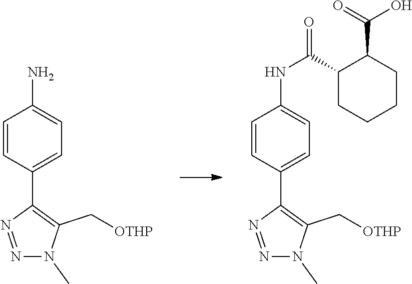

Intermediate L3 (0.60 g 1.35 mmol, 79% yield) was obtained from 4-(1-methyl-5-(((tetrahydro-2H-pyran-2-yl)oxy)methyl)-1H-1,2,3-triazol-4-yl)aniline (Intermediate L2, 0.494 g, 1.71 mmol) and (−)-trans-1,2-Cyclohexanedicarboxylic anhydride (0.291 g, 1.88 mmol) using a similar method as for Compound 1.

LC-MS (ESI): m/z (M+1): 441.5 (Method 4)

### Step 4: methyl (1S,2S)-2-((4-(5-(hydroxymethyl)-1-methyl-1H-1,2,3-triazol-4-yl)phenyl)carbamoyl)cyclohexane-1-carboxylate (Intermediate L4)

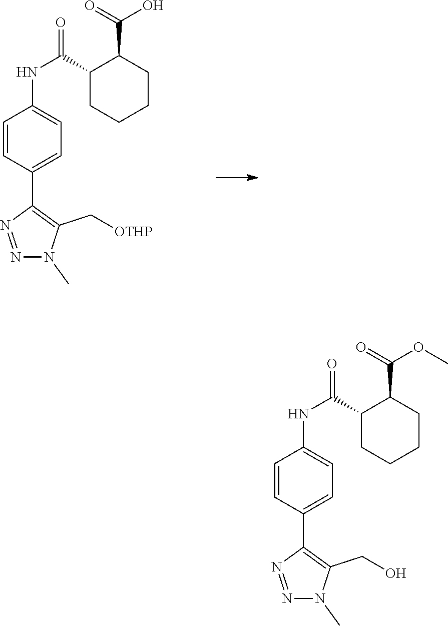

In sealed tube (1S,2S)-2-((4-(1-methyl-5-(((tetrahydro-2H-pyran-2-yl)oxy)methyl)-1H-1,2,3-triazol-4-yl)phenyl)carbamoyl)cyclohexane-1-carboxylic acid (Intermediate L3, 0.60 g, 1.35 mmoL) was dissolved in MeOH (34 mL) and PTSA (0.515 g, 2.71 mmol) was added to the mixture. The reaction mixture was stirred at 60° C. for 1 h. Solvent was removed under vacuum. The crude was diluted in water (100 mL) and then extracted with DCM (3×100 mL). The organic phase was collected and washed with brine, then dried over Na2SO4 to obtain the title compound (0.483 g, 1.30 mmol, 96% yield).

LC-MS (ESI): m/z (M+1): 371.4 (Method 4)

### Step 5: methyl (1S,2S)-2-((4-(5-formyl-1-methyl-1H-1,2,3-triazol-4-yl)phenyl)carbamoyl)cyclohexane-1-carboxylate (Intermediate L5)

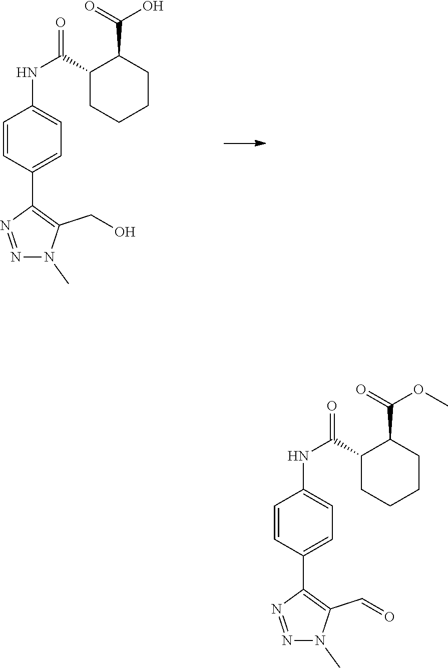 [SMILES: Cn1nnc(-c2ccc(NC(=O)[C@H]3CCCC[C@@H]3C(=O)O)cc2)c1CO ; COC(=O)[C@H]1CCCC[C@@H]1C(=O)Nc1ccc(-c2nnn(C)c2C=O)cc1]

Methyl (1S,2S)-2-((4-(5-(hydroxymethyl)-1-methyl-1H-1,2,3-triazol-4-yl)phenyl)carbamoyl)cyclohexane-1-carboxylate (Intermediate L4, 0.433 g, 1.16 mmol,) was dissolved in DMF (12 mL). To the reaction mixture pyridinium dichromate (0.525 g, 1.40 mmol) was added. The reaction mixture was left stirring overnight at 60° C. Solvent was evaporated. The reaction mixture was diluted with water (75 mL) and the pH was neutralized with saturated NaHCO₃. The water phase was washed with DCM (3×75 mL). The organic phase was collected and washed with brine, then dried over Na2SO4. The crude product was purified via flash column chromatography (0-50% of EtOAc in DCM) to give the title compound (0.372 g, 1 mmol, 86% yield).

LC-MS (ESI): m/z (M+1): 369. (Method 4)

### Step 6: 4-(4-((1S,2S)-2-(methoxycarbonyl)cyclohexane-1-carboxamido)phenyl)-1-methyl-1H-1,2,3-triazole-5-carboxylic acid (Intermediate L6)

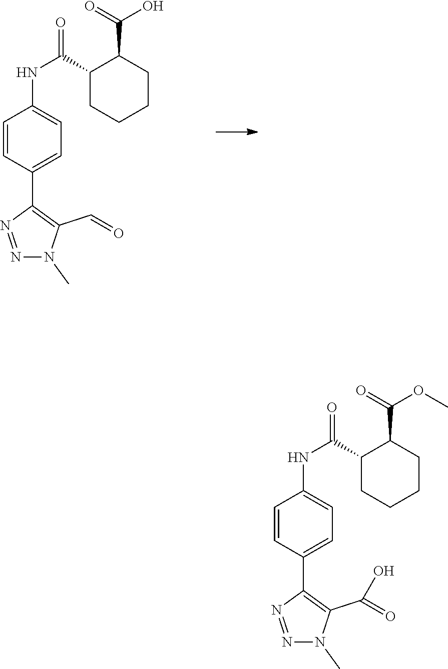 [SMILES: Cn1nnc(-c2ccc(NC(=O)[C@H]3CCCC[C@@H]3C(=O)O)cc2)c1C=O ; COC(=O)[C@H]1CCCC[C@@H]1C(=O)Nc1ccc(-c2nnn(C)c2C(=O)O)cc1]

Methyl (1S,2S)-2-((4-(5-formyl-1-methyl-1H-1,2,3-triazol-4-yl)phenyl)carbamoyl)cyclohexane-1-carboxylate (Intermediate L5, 0.372 g, 1.00 mmol) was dissolved in mixture of DMSO (0.86 mL), water (13 ml) and MeCN (40 mL) in round bottom flask, the temperature of the mixture was cooled to 0° C. To the mixture H₂SO₄ (conc.) (0.054 m) was slowly added, followed by addition of solution of sodium chlorite 25% (0.447 mL, 1.51 mmol). The temperature was kept at 0° C. for 30 min. The mixture was allowed to warm to RT. After water addition (100 mL) into the reaction, the product was extracted with EtOAc (3×100 mL). The combined organic layers were washed with water and brine, dried over Na2SO₄ and concentrated to dryness to acquire the title compound (0.356 g, 0.92 mmol, 92% yield).

LC-MS (ESI): m/z (M+1): 387.5 (Method 4)

### Step 7: methyl (1S,2S)-2-((4-(1-methyl-5-(((2-(trimethylsilyl)ethoxy)carbonyl)amino)-1H-1,2,3-triazol-4-yl)phenyl)carbamoyl)cyclohexane-1-carboxylate (Intermediate L7)

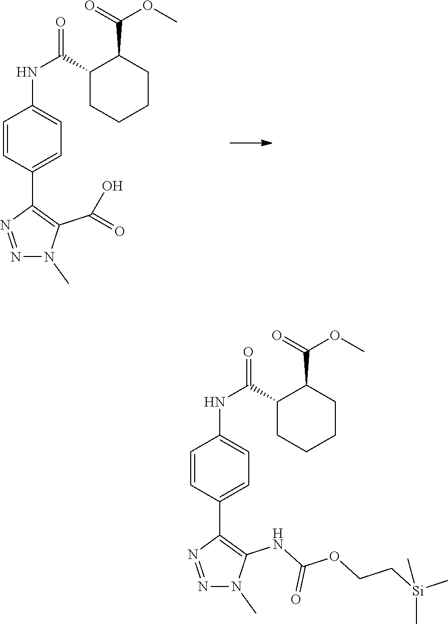 [SMILES: COC(=O)[C@H]1CCCC[C@@H]1C(=O)Nc1ccc(-c2nnn(C)c2C(=O)O)cc1 ; COC(=O)[C@H]1CCCC[C@@H]1C(=O)Nc1ccc(-c2nnn(C)c2NC(=O)OCC[Si](C)(C)C)cc1]

Intermediate L7 (0.300 g, 0.598 mmol, 74% yield) was obtained as a yellow foam from 4-(4-((1S,2S)-2-(methoxycarbonyl)cyclohexane-1-carboxamido)phenyl)-1-methyl-1H-1,2,3-triazole-5-carboxylic acid (Intermediate L6, 0.304 g, 0.787 mmol) and 2-(Trimethylsilyl)ethanol (0.135 mL, 0.944 mmol) using a similar method as for Intermediate B5.

LC-MS (ESI): m/z (M+1): 502. (Method 5)

### Step 8: methyl (1S,2S)-2-((4-(5-amino-1-methyl-1H-1,2,3-triazol-4-yl)phenyl)carbamoyl)cyclohexane-1-carboxylate (Intermediate L8)

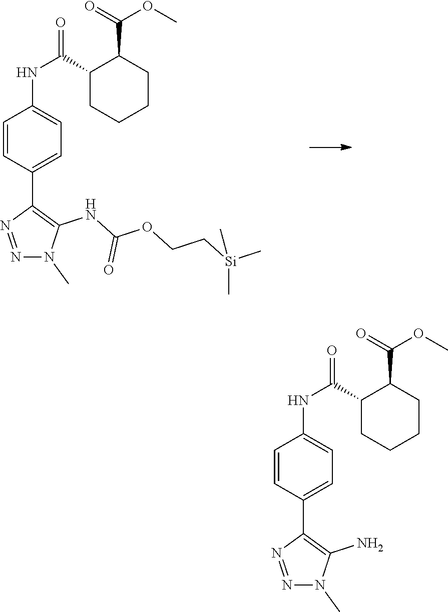 [SMILES: COC(=O)[C@H]1CCCC[C@@H]1C(=O)Nc1ccc(-c2nnn(C)c2NC(=O)OCC[Si](C)(C)C)cc1 ; COC(=O)[C@H]1CCCC[C@@H]1C(=O)Nc1ccc(-c2nnn(C)c2N)cc1]

Intermediate L8 (0.19 g, 0.532 mmol, 89% yield) was obtained from ethyl (1S,2S)-2-((4-(1-methyl-5-(((2-(trimethylsilyl)ethoxy)carbonyl)amino)-1H-1,2,3-triazol-4-yl)phenyl)carbamoyl)cyclohexane-1-carboxylate (Intermediate L7, 0.300 g, 0.598 mmol) using a similar method as for Intermediate B6.

LC-MS (ESI): m/z (M+1): 358.4 (Method 2)

### Step 9: 2-(4-(5-((6-isopropoxypyrazin-2-yl)amino)-1-methyl-1H-1,2,3-triazol-4-yl)phenyl)hexahydro-1H-isoindole-1,3(2H)-dione (Intermediate L9)

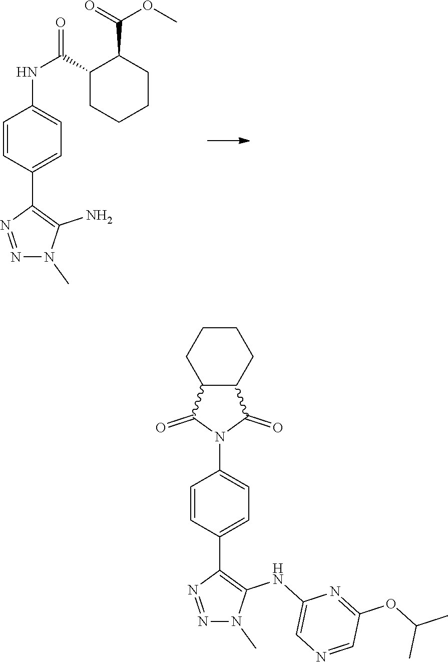 [SMILES: COC(=O)[C@H]1CCCC[C@@H]1C(=O)Nc1ccc(-c2nnn(C)c2N)cc1 ; CC(C)Oc1cncc(Nc2c(-c3ccc(N4C(=O)C5CCCCC5C4=O)cc3)nnn2C)n1]

Methyl (1S,2S)-2-((4-(5-amino-1-methyl-1H-1,2,3-triazol-4-yl)phenyl)carbamoyl)cyclohexane-1-carboxylate (Intermediate L8, 0.050 g, 0.140 mmol), Cs₂CO₃ (0.099 g, 0.280 mmol) and 2-chloro-6-isopropoxypyrazine (Intermediate A23, 0.026 g, 0.154 mmol) were suspended in 1,4-dioxane (2.5 mL) in pre-sealed tube. The reaction mixture was degassed with argon and Pd₂(dba)₃ (0.013 g, 0.014 mmol) was added. The reaction mixture was stirred for 1h in a sealed tube at 85° C. The reaction mixture was quenched with water (10 mL) and the product was extracted with EtOAc (3×10 mL). The combined organic layers were dried over Na₂SO₄, and then concentrated. The crude product was purified via preparative TLC (90% of EtOAc in hexane) to obtain the title compound (5 mg, 0.011 mmol, 8% yield).

LC-MS (ESI): m/z (M+1): 462.3 (Method 2)

### Step 10: 2-((4-(5-((6-isopropoxypyrazin-2-yl)amino)-1-methyl-1H-1,2,3-triazol-4-yl)phenyl)carbamoyl)cyclohexane-1-carboxylic acid (cis racemate) (Compound 43)

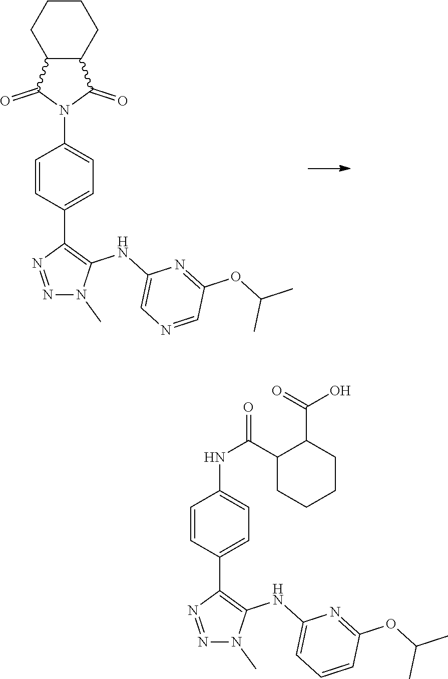 [SMILES: CC(C)Oc1cncc(Nc2c(-c3ccc(N4C(=O)C5CCCCC5C4=O)cc3)nnn2C)n1 ; CC(C)Oc1cccc(Nc2c(-c3ccc(NC(=O)C4CCCCC4C(=O)O)cc3)nnn2C)n1]

2M LiOH (0.013 mL, 0.026 mmol, 1.0 eq) was added dropwise to a solution of 2-(4-(5-((6-isopropoxypyrazin-2-yl)amino)-1-methyl-1H-1,2,3-triazol-4-yl)phenyl)hexahydro-1H-isoindole-1,3(2H)-dione (Intermediate L9, 0.012 g, 0.026 mmol) in 1,4-dioxane (0.13 mL) at 0° C. The reaction mixture was stirred for 2 h at RT. Solvent was evaporated, 2 mL of water was added and the mixture was acidified with 1M HCl to pH ˜3-4. The solid was filtered and washed with water (3×2 mL) to give the title compound (0.0057 g, 0.012 mmol, 46% yield) as a beige solid.

LC-MS (ESI): m/z (M+1): 480.3 (Method 2)

¹H NMR (300 MHz, DMSO-d6) δ 11.93 (s, 1H), 9.79 (s, 1H), 9.24 (s, 1H), 7.77-7.41 (m, 5H), 7.51-7.16 (m, 1H), 4.74-4.57 (m, 1H), 3.86 (s, 3H), 3.01-2.82 (m, 1H), 2.16-1.87 (m, 2H), 1.87-1.52 (m, 3H), 1.50-1.29 (m, 4H), 1.08 (d, J=6.2 Hz, 6H)

### Example 16

### Process Synthesis of Compound 44

### (1S,2S)-2-((6-(5-(((4-cyclohexylpyrimidin-2-yl)amino)methyl)-1-methyl-1H-1,2,3-triazol-4-yl)-2-methylpyridin-3-yl)carbamoyl)cyclohexane-1-carboxylic acid

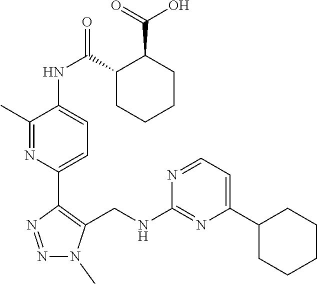 [SMILES: Cc1nc(-c2nnn(C)c2CNc2nccc(C3CCCCC3)n2)ccc1NC(=O)[C@H]1CCCC[C@@H]1C(=O)O]

### Step 1: 6-(5-(chloromethyl)-1-methyl-1H-1,2,3-triazol-4-yl)-2-methyl-3-nitropyridine (Intermediate M1)

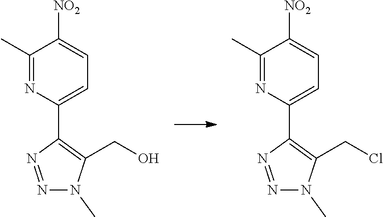 [SMILES: Cc1nc(-c2nnn(C)c2CO)ccc1[N+](=O)[O-] ; Cc1nc(-c2nnn(C)c2CCl)ccc1[N+](=O)[O-]]

Intermediate M1 (311 mg, crude) was obtained from (1-methyl-4-(6-methyl-5-nitropyridin-2-yl)-1H-1,2,3-triazol-5-yl)methanol (Intermediate K11, 160.0 mg, 0.640 mmol) using a similar method as for Intermediate D9.

LC-MS (ESI): m/z (M+1): 268.3 (Method 1)

### Step 2: (1-methyl-4-(6-methyl-5-nitropyridin-2-yl)-1H-1,2,3-triazol-5-yl)methanamine (Intermediate M2)

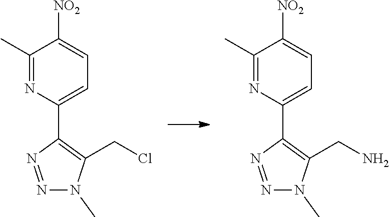 [SMILES: Cc1nc(-c2nnn(C)c2CCl)ccc1[N+](=O)[O-] ; Cc1nc(-c2nnn(C)c2CN)ccc1[N+](=O)[O-]]

Intermediate M2 (129 mg, 0.520 mmol, 80.94% yield) was obtained from 6-(5-(chloromethyl)-1-methyl-1H-1,2,3-triazol-4-yl)-2-methyl-3-nitropyridine (Intermediate M1, 311 mg, crude) using a similar method as for Intermediate D12.

LC-MS (ESI): m/z (M+1): 249.1 (Method 1)

### Step 3: 2-chloro-4-cyclohexylpyrimidine (intermediate M3)

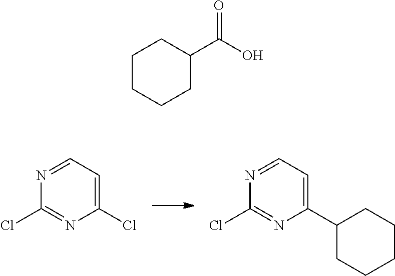 [SMILES: O=C(O)C1CCCCC1 ; Clc1ccnc(Cl)n1 ; Clc1nccc(C2CCCCC2)n1]

To a mixture of 2-chloropyrimidine (1.12 g, 9.75 mmol), cyclohexanecarboxylic acid (0.97 mL, 7.8 mmol) and silver nitrate (265.07 mg, 1.56 mmol) in DCM (58 mL) and Water (58 mL), dipotassium sulfonatooxy sulfate (2.12 g, 7.8 mmol) were added. The mixture was stirred at RT overnight. The solvent was concentrated under reduced pressure. The crude was diluted with EtOAc and washed with H₂O, the organic phase dried over Na2SO4, filtered and concentrated under reduced pressure then purified by flash chromatography eluting with CyHex:EtOAc from 0 to 50% to afford the title compound (502 mg, 2.552 mmol, 32.71% yield) as a colorless oil.

LC-MS (ESI): m/z (M+1): 197.0 (Method 1)

### Step 4: 4-cyclohexyl-N-((1-methyl-4-(6-methyl-5-nitropyridin-2-yl)-1H-1,2,3-triazol-5-yl)methyl)pyrimidin-2-amine (Intermediate M4)

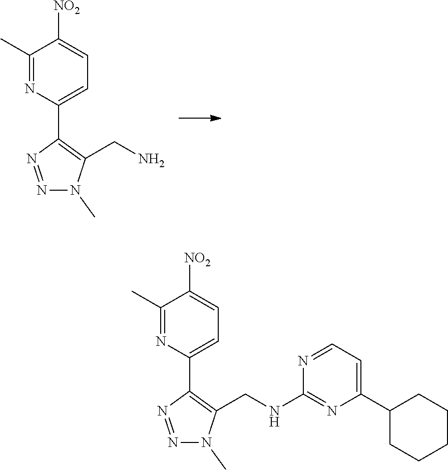 [SMILES: Cc1nc(-c2nnn(C)c2CN)ccc1[N+](=O)[O-] ; Cc1nc(-c2nnn(C)c2CNc2nccc(C3CCCCC3)n2)ccc1[N+](=O)[O-]]

To a mixture of (1-methyl-4-(6-methyl-5-nitropyridin-2-yl)-1H-1,2,3-triazol-5-yl)methanamine (Intermediate M2, 60.0 mg, 0.240 mmol) and 2-chloro-4-cyclohexylpyrimidine (Intermediate M3, 47.54 mg, 0.240 mmol) in DMF (1 mL), K₂CO₃ (50.11 mg, 0.360 mmol) was added and the mixture was stirred at 80° C. overnight. The mixture was allowed to cool to RT, poured into saturated NaHCO₃ aqueous solution and extracted with EtOAc. The organic phase was separated, filtered through a hydrophobic phase separator and concentrated at reduced pressure. The crude was purified by flash chromatography with Water/MeCN with 0.1% HCOOH from 95:5 to 0:100 as eluent. Evaporation of proper fractions provided the desired product (37 mg, 0.091 mmol, 37.48% yield) as a yellow solid.

LC-MS (ESI): m/z (M+1): 409.3 (Method 1)

### Step 5: N-((4-(5-amino-6-methylpyridin-2-yl)-1-methyl-1H-1,2,3-triazol-5-yl)methyl)-4-cyclohexylpyrimidin-2-amine (Intermediate M5)

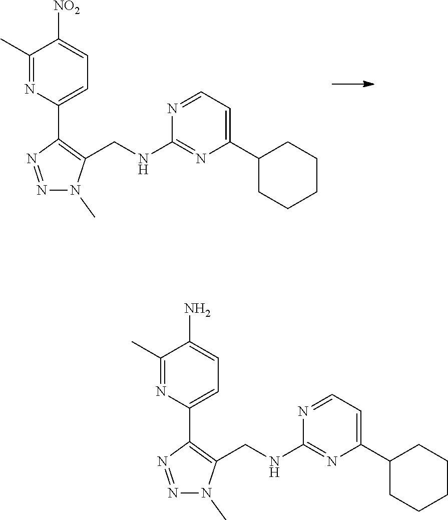 [SMILES: Cc1nc(-c2nnn(C)c2CNc2nccc(C3CCCCC3)n2)ccc1[N+](=O)[O-] ; Cc1nc(-c2nnn(C)c2CNc2nccc(C3CCCCC3)n2)ccc1N]

s37% v/v HCl (0.03 mL, 1.27 mmol) and SnCl₂·2H₂O (82.49 mg, 0.360 mmol) were added to a suspension of 4-cyclohexyl-N-((1-methyl-4-(6-methyl-5-nitropyridin-2-yl)-1H-1,2,3-triazol-5-yl)methyl)pyrimidin-2-amine (Intermediate M4, 37.0 mg, 0.090 mmol) in Diethyl ether (1 mL) and MeOH (0.250 mL). The mixture was stirred at RT for 1.5h. SnCl₂·2H₂O (82.49 mg, 0.360 mmol) was added and the mixture was stirred at RT for additional 30 min. The reaction mixture was diluted with Water and adjusted to pH=7 with NaOH 10%. The mixture was extracted with EtOAc and the organic phase was separated, filtered through a hydrophobic phase separator and concentrated at reduced pressure to provide the title compound (48 mg, crude) as an orange oil. The crude material was used in the next step without further purification.

LC-MS (ESI): m/z (M+1): 379.2 (Method 1)

### Step 6: (1S,2S)-2-((6-(5-(((4-cyclohexylpyrimidin-2-yl)amino)methyl)-1-methyl-1H-1,2,3-triazol-4-yl)-2-methylpyridin-3-yl)carbamoyl)cyclohexane-1-carboxylic acid (Compound 44)

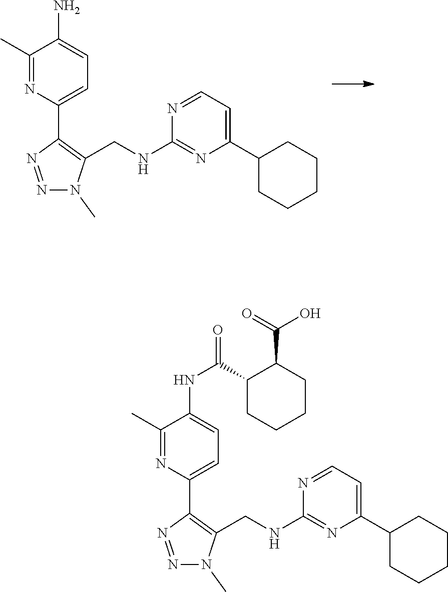 [SMILES: Cc1nc(-c2nnn(C)c2CNc2nccc(C3CCCCC3)n2)ccc1N ; Cc1nc(-c2nnn(C)c2CNc2nccc(C3CCCCC3)n2)ccc1NC(=O)[C@H]1CCCC[C@@H]1C(=O)O]

Compound 44 (20 mg, 0.038 mmol, 41.26% yield) was obtained ad an off-white solid from N-((4-(5-amino-6-methylpyridin-2-yl)-1-methyl-1H-1,2,3-triazol-5-yl)methyl)-4-cyclohexylpyrimidin-2-amine (Intermediate M5, 34.44 mg, 0.090 mmol) and trans-1,2-Cyclohexanedicarboxylic anhydride (15.43 mg, 0.100 mmol) using a similar method as for Compound 1.

LC-MS (ESI): m/z (M+1): 533.3 (Method 1)

1H NMR (400 MHz, DMSO-d₆) δ ppm 12.12 (br s, 1H), 9.57 (br s, 1H), 8.16 (br d, J=4.8 Hz, 1H), 7.95-7.78 (m, 2H), 7.42 (t, J=5.9 Hz, 1H), 6.50 (d, J=5.0 Hz, 1H), 5.11-4.84 (m, 2H), 4.15 (s, 3H), 2.74-2.63 (m, 1H), 2.55-2.47 (m, 1H), 2.46 (s, 3H), 2.40-2.26 (m, 1H), 2.00 (br d, J=10.4 Hz, 2H), 1.88-1.55 (m, 7H), 1.49-1.02 (m, 9H)

### Example 17

### Process Synthesis of Compound 45

### (1S,2S)-2-((6-(4-(((cyclopentyl(methyl)carbamoyl)oxy)methyl)-3-methylisoxazol-5-yl)-2-methylpyridin-3-yl)carbamoyl)cyclohexane-1-carboxylic acid

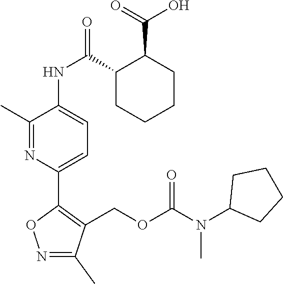 [SMILES: Cc1nc(-c2onc(C)c2COC(=O)N(C)C2CCCC2)ccc1NC(=O)[C@H]1CCCC[C@@H]1C(=O)O]

### Step 1: tert-butyl (2-methyl-6-(3-methyl-4-(((((4-nitrobenzyl)oxy)carbonyl)oxy)methyl)isoxazol-5-yl)pyridin-3-yl)carbamate (Intermediate N1)

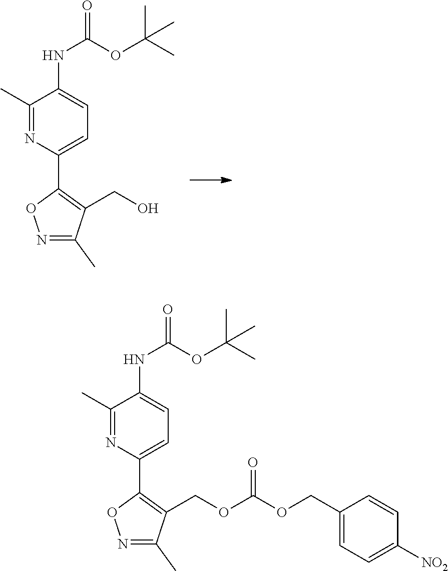 [SMILES: Cc1nc(-c2onc(C)c2CO)ccc1NC(=O)OC(C)(C)C ; Cc1nc(-c2onc(C)c2COC(=O)OCc2ccc([N+](=O)[O-])cc2)ccc1NC(=O)OC(C)(C)C]

Intermediate N1 (464 mg, 0.958 mmol, 93.83% yield) was obtained from tert-butyl (6-(4-(hydroxymethyl)-3-methylisoxazol-5-yl)-2-methylpyridin-3-yl)carbamate (Intermediate D7, 326.0 mg, 1.02 mmol) and carbonochloridic acid (4-nitrophenyl) ester (411.5 mg, 2.04 mmol) using a similar method as fro Intermediate I1.

LC-MS (ESI): m/z (M+1): 485.2 (Method 1)

### Step 2: (5-(5-((tert-butoxycarbonyl)amino)-6-methylpyridin-2-yl)-3-methylisoxazol-4-yl)methyl cyclopentyl(methyl)carbamate (Intermediate N2)

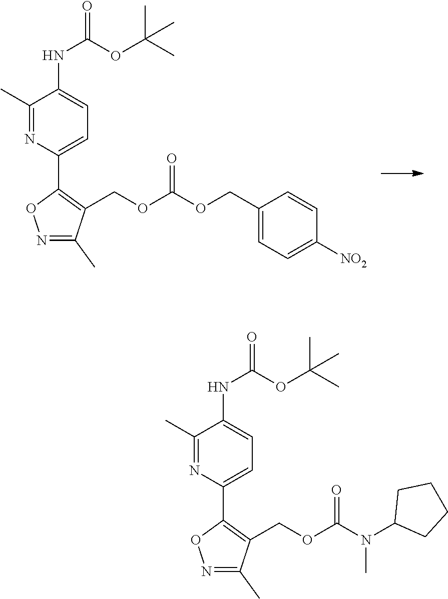 [SMILES: Cc1nc(-c2onc(C)c2COC(=O)OCc2ccc([N+](=O)[O-])cc2)ccc1NC(=O)OC(C)(C)C ; Cc1nc(-c2onc(C)c2COC(=O)N(C)C2CCCC2)ccc1NC(=O)OC(C)(C)C]

Intermediate N2 (114 mg, 0.26 mmol, 95.57% yield) was obtained from tert-butyl (2-methyl-6-(3-methyl-4-(((((4-nitrobenzyl)oxy)carbonyl)oxy)methyl)isoxazol-5-yl)pyridin-3-yl)carbamate (Intermediate N1, 130.0 mg, 0.270 mmol) and cyclopentyl-methyl-amine (53.22 mg, 0.540 mmol) using a similar method as for Intermediate F4.

LC-MS (ESI): m/z (M+1): 445.3 (Method 1)

The Intermediates in the following table were prepared from reagents reported below by using methods analogous to Intermediate N2.

| Intermediate | Structure & Name | Reagents | Analytical data |
|---|---|---|---|
| N3 | 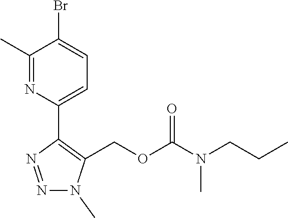 [SMILES: CCCN(C)C(=O)OCc1c(-c2ccc(Br)c(C)n2)nnn1C] (4-(5-bromo-6- methylpyridin-2-yl)-1- methyl-1H-1,2,3-triazol- 5-yl)methyl methyl(propyl)carbamate | K12 and N- methylpropan- 1-amine | LC-MS (ESI): m/z (M + 1): 382.0 (Method 1) 1H NMR (400 MHz, Chloroform-d) δ 7.95-7.82 (m, 2H), 5.77 (d, J = 8.14 Hz, 2H), 4.18 (s, 3H), 3.19 (dt, J = 56.06, 7.62 Hz, 2H), 2.88 (d, J = 37.74 Hz, 3H), 2.70 (s, 3H), 1.44 (q, J = 7.48 Hz, 1H), 0.85 (dt, J = 56.97, 7.41 Hz, 3H) |

### Step 3: (5-(5-amino-6-methylpyridin-2-yl)-3-methylisoxazol-4-yl)methyl cyclopentyl(methyl)carbamate (Intermediate N4)

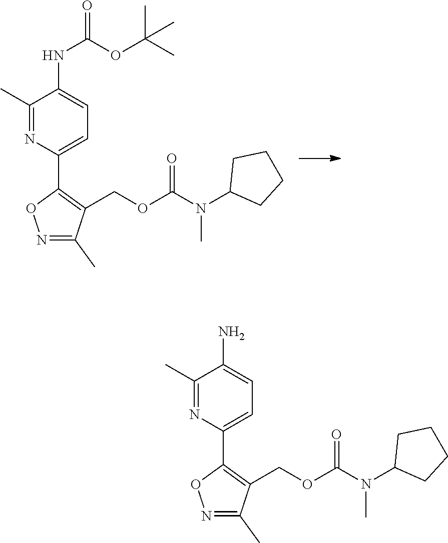 [SMILES: Cc1nc(-c2onc(C)c2COC(=O)N(C)C2CCCC2)ccc1NC(=O)OC(C)(C)C ; Cc1nc(-c2onc(C)c2COC(=O)N(C)C2CCCC2)ccc1N]

Intermediate N4 (72.3 mg, 0.210 mmol, 81.86% yield) was obtained from (5-(5-((tert-butoxycarbonyl)amino)-6-methylpyridin-2-yl)-3-methylisoxazol-4-yl)methyl cyclopentyl(methyl)carbamate (Intermediate N2, 114.0 mg, 0.260 mmol) using a similar method as for Intermediate F5.

LC-MS (ESI): m/z (M+1): 345.2 (Method 1)

### Step 4: (1S,2S)-2-((6-(4-(((cyclopentyl(methyl)carbamoyl)oxy)methyl)-3-methylisoxazol-5-yl)-2-methylpyridin-3-yl)carbamoyl)cyclohexane-1-carboxylic acid (Compound 45)

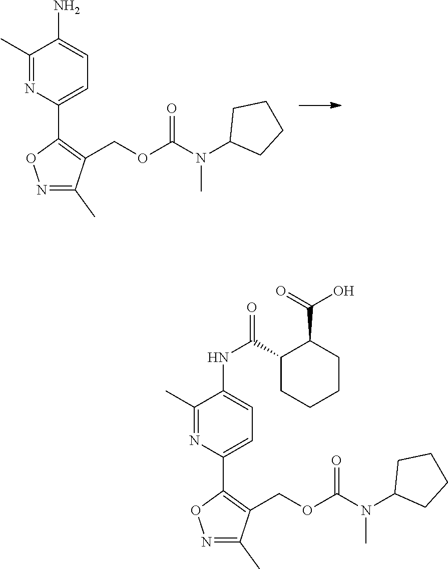 [SMILES: Cc1nc(-c2onc(C)c2COC(=O)N(C)C2CCCC2)ccc1N ; Cc1nc(-c2onc(C)c2COC(=O)N(C)C2CCCC2)ccc1NC(=O)[C@H]1CCCC[C@@H]1C(=O)O]

Compound 45 (76.3 mg, 0.153 mmol, 72.9% yield) was obtained as white solid from (5-(5-amino-6-methylpyridin-2-yl)-3-methylisoxazol-4-yl)methyl cyclopentyl(methyl)carbamate (Intermediate N4, 72.3 mg, 0.210 mmol) and (−)-trans-1,2-Cyclohexanedicarboxylic anhydride (35.6 mg, 0.230 mmol) using a similar method as for Compound 1.

LC-MS (ESI): m/z (M+1): 499.3 (Method 1)

¹H NMR (500 MHz, DMSO-d6) δ ppm 12.15 (br s, 1H), 9.63 (s, 1H), 8.00 (d, J=8.4 Hz, 1H), 7.74 (d, J=8.4 Hz, 1H), 5.38 (s, 2H), 4.65-3.97 (m, 1H), 2.71 (td, J=11.3, 3.2 Hz, 1H), 2.65 (br s, 3H), 2.56-2.49 (m, 1H), 2.47 (s, 3H), 2.32 (s, 3H), 2.08-1.96 (m, 2H), 1.77 (br d, J=8.9 Hz, 2H), 1.59 (br s, 4H), 1.51-1.38 (m, 4H), 1.37-1.20 (m, 4H)

### Example 18

### Process Synthesis of Compound 46

### (1S,2S)-2-(((6-(4-((6-cyclopentylpyrazin-2-yl)amino)-3-methylisoxazol-5-yl)-2-methylpyridin-3-yl)oxy)methyl)cyclohexane-1-carboxylic acid

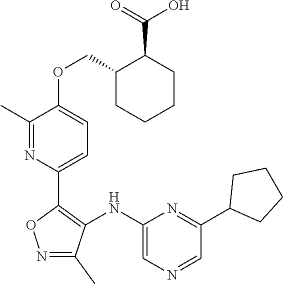 [SMILES: Cc1nc(-c2onc(C)c2Nc2cncc(C3CCCC3)n2)ccc1OC[C@H]1CCCC[C@@H]1C(=O)O]

### Step 1: benzyl (3-methyl-5-(6-methyl-5-(4,4,5,5-tetramethyl-1,3,2-dioxaborolan-2-yl)pyridin-2-yl)isoxazol-4-yl)carbamate (Intermediate O1)

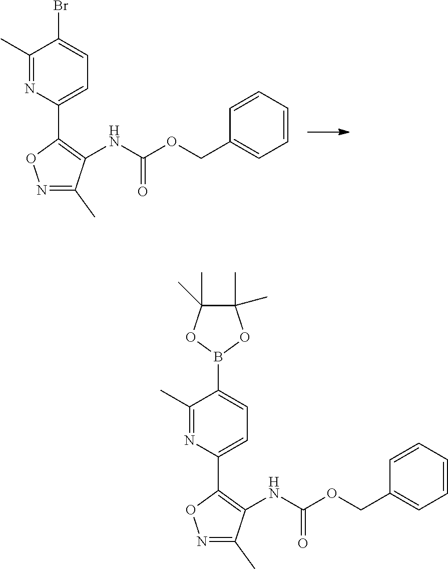 [SMILES: Cc1nc(-c2onc(C)c2NC(=O)OCc2ccccc2)ccc1Br ; Cc1nc(-c2onc(C)c2NC(=O)OCc2ccccc2)ccc1B1OC(C)(C)C(C)(C)O1]

B₂pin₂ (473.49 mg, 1.86 mmol) and KOAc (365.98 mg, 3.73 mmol) were added to a solution of benzyl (5-(5-bromo-6-methylpyridin-2-yl)-3-methylisoxazol-4-yl)carbamate (Intermediate A15, 500.0 mg, 1.24 mmol) in dry 1,4-dioxane (7.7 mL). The mixture was degassed by applying alternatively vacuum and nitrogen (3 times). Pd(dppf)Cl₂, complex with dichloromethane (50.88 mg, 0.060 mmol) was then added and the mixture was stirred at 90° C. for 9 h, then allowed to stand overnight at room temperature. EtOAc was added and the mixture was washed with water (2 times).

The organic phase was dried over Na2SO₄, filtered and concentrated in vacuo to give the crude product as a brown solid (782 mg, crude) that was used in the following experiment without further purification.

LC-MS (ESI): m/z (M+1): 450.2 (Method 1)

### Step 2: benzyl (5-(5-hydroxy-6-methylpyridin-2-yl)-3-methylisoxazol-4-yl)carbamate (Intermediate O2)

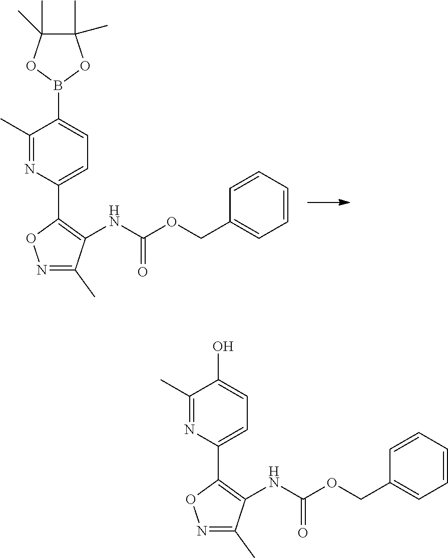 [SMILES: Cc1nc(-c2onc(C)c2NC(=O)OCc2ccccc2)ccc1B1OC(C)(C)C(C)(C)O1 ; Cc1nc(-c2onc(C)c2NC(=O)OCc2ccccc2)ccc1O]

hydrogen peroxide (0.53 mL, 5.22 mmol) (30% w/w) was added to a solution of benzyl (3-methyl-5-(6-methyl-5-(4,4,5,5-tetramethyl-1,3,2-dioxaborolan-2-yl)pyridin-2-yl)isoxazol-4-yl)carbamate (Intermediate O1, 782.0 mg, 1.74 mmol) in EtOAc (6.7 mL). The mixture was stirred at RT for 30 min. A saturated aqueous Na₂S₂O₃ solution was then added at 0° C. The mixture was stirred at this temperature for 5 min, then it was extracted with EtOAc (3 times). The mixed organic phases were washed with brine, dried over Na₂SO₄, filtered and concentrated in vacuo. The residue was purified by flash chromatography (gradient CyHex/EtOAc from 90/10 to 20/80) and then with a second column (eluent A: water+0.1% formic acid, eluent B: MeCN+0.1% formic acid, gradient A/B from 100/0 to 40/60) to give the target product as a white solid (346 mg, 1.02 mmol, 58.6% yield).

LC-MS (ESI): m/z (M+1): 340.1 (Method 1)

The Intermediates in the following table were prepared from reagents reported below by using methods analogous to Intermediate O2 (Step 1 and Step 2).

| Intermediate | Structure & Name | Reagents | Analytical data |
|---|---|---|---|
| O3 | 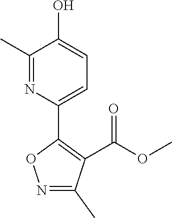 methyl 5-(5-hydroxy-6- methylpyridin-2-yl)-3- methylisoxazole-4- carboxylate | A11 | LC-MS (ESI): m/z (M + 1): 187.1 (Method 2) ¹H NMR (300 MHz, DMSO- d₆) δ 10.63 (s, 1H), 7.70 (d, J = 8.4 Hz, 1H), 7.24 (d, J = 8.4 Hz, 1H), 3.75 (s, 3H), 2.37 (d, J = 3.0 Hz, 6H) |
| O4 | 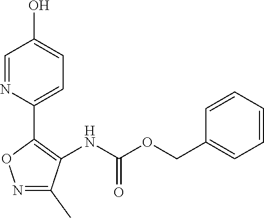 [SMILES: Cc1noc(-c2ccc(O)cn2)c1NC(=O)OCc1ccccc1] benzyl (5-(5- hydroxypyridin-2-yl)-3- methylisoxazol-4- yl)carbamate | A6 and A16 | LC-MS (ESI): m/z (M + 1): 325.1 (Method 14) |

### Step 3: (1S,2S)-2-(methoxycarbonyl)cyclohexane-1-carboxylic acid (Intermediate O5)

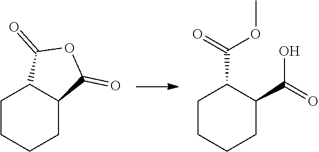

A suspension of (3aS,7aS)-hexahydroisobenzofuran-1,3-di one (5.00 g, 32.4 mmol) in MeOH (35 mL) was heated under vigorous reflux for 2 h (solution). The solvent was removed under reduced pressure to afford the desired compound (6.0 g, 32.2 mmol, 9900 yield) as a clear oil.

LC-MS (ESI): m/z (M+1): 187.1 (Method 14)

The Intermediates in the following table were prepared from reagents reported below by using methods analogous to Intermediate O5.

| Intermediate | Structure & Name | Reagents | Analytical data |
|---|---|---|---|
| O6 | 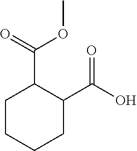 cis-2- (methoxycarbonyl) cyclohexane-1- carboxylic acid (racemate) | cis- hexahydro- isobenzofuran- 1,3-dione | LC-MS (ESI): m/z (M + 1): 187.1 (Method 14) |

### Step 4: methyl (1S,2S)-2-(hydroxymethyl)cyclohexane-1-carboxylate (Intermediate O7)

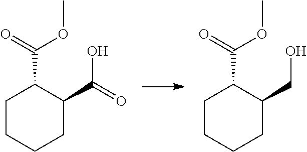

To a solution of (1S,2S)-2-(methoxycarbonyl)cyclohexane-1-carboxylic acid 05 (6.0 g, 32.2 mmol) and TEA (18 mL, 129 mmol) in THE (90 mL) cooled to −20° C., isobutyl chloroformate (6.35 mL, 48.3 mmol) was added dropwise maintaining the temperature below −15° C. and the resulting mixture was stirred at the same temperature for 2 h. A solution of NaBH₄ (7.31 g, 193 mmol) in water (58 mL) was added dropwise maintaining the temperature below −5° C. The mixture was allowed to reach RT. and stirred for 2 h. The reaction was cooled with an ice bath, quenched by addition of 1.2M HCl (pH 6) and extracted with EtOAc (2 times). The combined organic layers were washed with sat. NaHCO₃ and brine, dried over sodium sulfate, filtered and concentrated. The crude was purified by flash chromatography (hexane:EtOAc from 90:10 to 60:40) to afford the title compound (4.6 g, 26.7 mmol, 83% yield) as a colorless oil.

LC-MS (ESI): m/z (M+1): 173.2 (Method 14)

¹H NMR (300 MHz, DMSO-d₆) δ ppm 4.42 (t, J=5.2 Hz, 1H), 3.57 (s, 3H), 3.31-3.12 (m, 2H), 2.10 (td, J=11.4, 3.6 Hz, 1H), 1.85-1.72 (m, 2H), 1.72-1.52 (m, 3H), 1.44-1.26 (m, 1H), 1.12-1.26 (m, 2H), 1.09-0.93 (m, 1H)

[α]_{D}=+47°, c=1, ACN

The Intermediates in the following table were prepared from reagents reported below by using methods analogous to Intermediate O7.

| Intermediate | Structure & Name | Reagents | Analytical data |
|---|---|---|---|
| O8 | 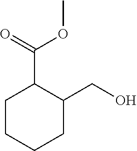 cis-methyl 2- (hydroxymethyl) cyclohexane-1- carboxylate (racemate) | O6 | LC-MS (ESI): m/z (M + 1): 173.1 (Method 14) ¹H NMR (300 MHz, DMSO-d₆) δ ppm 4.40 (t, J = 5.1 Hz, 1H), 3.56 (s, 3H), 3.43-3.26 (m, 2H), 2.70-2.57 (m, 1H), 1.79- 1.93 (m, 1H), 1.75-1.59 (m, 2H), 1.59-1.44 (m, 3H), 1.44-1.23 (m, 3H) |

### Step 5: methyl (1S,2S)-2-(((6-(4-(((benzyloxy)carbonyl)amino)-3-methylisoxazol-5-yl)-2-methylpyridin-3-yl)oxy)methyl)cyclohexane-1-carboxylate (Intermediate O9)

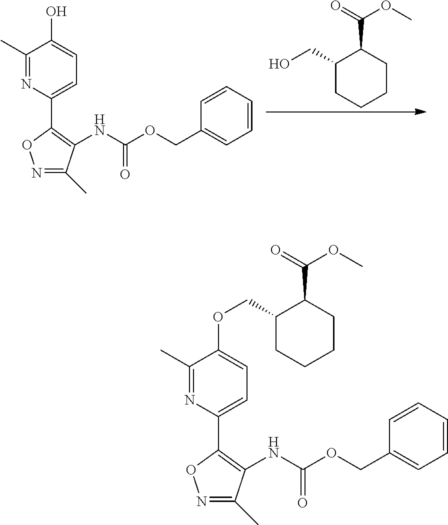 [SMILES: Cc1nc(-c2onc(C)c2NC(=O)OCc2ccccc2)ccc1O ; COC(=O)[C@H]1CCCC[C@@H]1CO ; COC(=O)[C@H]1CCCC[C@@H]1COc1ccc(-c2onc(C)c2NC(=O)OCc2ccccc2)nc1C]

A solution of methyl (1S,2S)-2-(hydroxymethyl)cyclohexane-1-carboxylate (Intermediate O7, 155.29 mg, 0.900 mmol) in dry THE (2.0 mL) was added to benzyl (5-(5-hydroxy-6-methylpyridin-2-yl)-3-methylisoxazol-4-yl)carbamate (Intermediate O2, 153.0 mg, 0.450 mmol). PPh3 (236.51 mg, 0.900 mmol) was then added at RT. After 5 min a solution of DBAD (207.63 mg, 0.900 mmol) in dry THE (2.0 mL) was added dropwise over 15 min. The mixture was then stirred at RT for 1 h. The mixture was then diluted with EtOAc and washed with water (2 times). The organic phase was dried over Na2SO4, filtered and concentrated in vacuo. The crude product was obtained as a white solid (368 mg, crude) and used as this without any further purification.

LC-MS (ESI): m/z (M+1): 492.4 (Method 1)

The Intermediates in the following table were prepared from reagents reported below by using methods analogous to Intermediate O9.

| Intermediate | Structure & Name | Reagents | Analytical data |
|---|---|---|---|
| O10 | 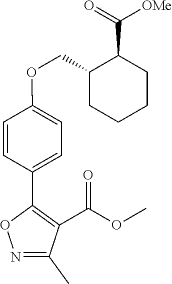 [SMILES: COC(=O)c1c(C)noc1-c1ccc(OC[C@H]2CCCC[C@@H]2C(=O)OC)cc1] | O7 and A7 | LC-MS (ESI): m/z (M + 1): 387.1 (Method 2) |
| | methyl 5-(4-(((1S,2S)-2- | | |
| | (methoxycarbonyl)cyclo | | |
| | hexyl)methoxy)phenyl)- | | |
| | 3-methylisoxazole-4- | | |
| | carboxylate | | |
| O11 | 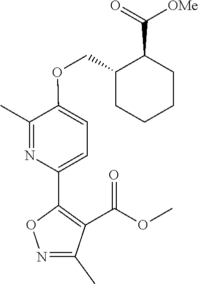 [SMILES: COC(=O)c1c(C)noc1-c1ccc(OC[C@H]2CCCC[C@@H]2C(=O)OC)c(C)n1] | O7 and O4 | LC-MS (ESI): m/z (M + 1): 402.2 (Method 2) |
| | methyl 5-(5-(((1S,2S)-2- | | |
| | (methoxycarbonyl)cyclo | | |
| | hexyl)methoxy)-6- | | |
| | methylpyridin-2-yl)-3- | | |
| | methylisoxazole-4- | | |
| | carboxylate | | |
| O12 | 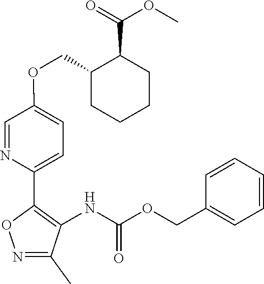 [SMILES: COC(=O)[C@H]1CCCC[C@@H]1COc1ccc(-c2onc(C)c2NC(=O)OCc2ccccc2)nc1] | O7 and O4 | LC-MS (ESI): m/z (M + 1): 479.2 (Method 14) |
| | methyl (1S,2S)-2-(((6-(4- | | |
| | (((benzyloxy)carbonyl) | | |
| | amino)-3-methylisoxazol- | | |
| | 5-yl)pyridin-3- | | |
| | yl)oxy)methyl)cyclo- | | |
| | hexane-1-carboxylate | | |

### Step 6: methyl (1S,2S)-2-(((6-(4-amino-3-methylisoxazol-5-yl)-2-methylpyridin-3-yl)oxy)methyl)cyclohexane-1-carboxylate (Intermediate O13)

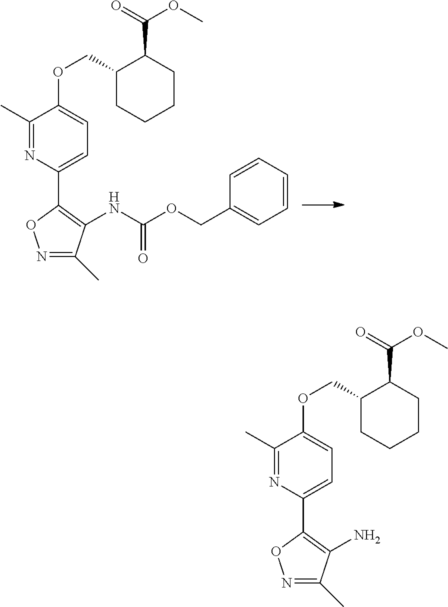

Intermediate O13 (264 mg, 0.735 mmol, 100% yield) was obtained as a yellow solid from methyl (1S,2S)-2-(((6-(4-(((benzyloxy)carbonyl)amino)-3-methylisoxazol-5-yl)-2-methylpyridin-3-yl)oxy)methyl)cyclohexane-1-carboxylate (Intermediate O9, 368.0 mg, 0.740 mmol, crude) using a similar method as for Intermediate A21.

LC-MS (ESI): m/z (M+1): 360.2 (Method 1)

The Intermediates in the following table were prepared from reagents reported below by using methods analogous to Intermediate O13.

| Intermediate | Structure & Name | Reagents | Analytical data |
|---|---|---|---|
| O14 | 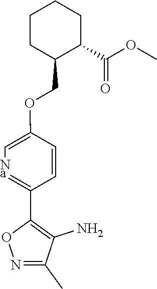 | O12 | LC-MS (ESI): m/z (M + 1): 346.2 (Method 14) |
| | methyl (1S,2S)-2-(((6-(4- | | |
| | amino-3-methylisoxazol- | | |
| | 5-yl)pyridin-3- | | |
| | yl)oxy)methyl)cyclo- | | |
| | hexane-1-carboxylate | | |

### Step 7: methyl (1S,2S)-2-(((6-(4-((6-(cyclopent-1-en-1-yl)pyrazin-2-yl)amino)-3-methylisoxazol-5-yl)-2-methylpyridin-3-yl)oxy)methyl)cyclohexane-1-carboxylate (Intermediate O15)

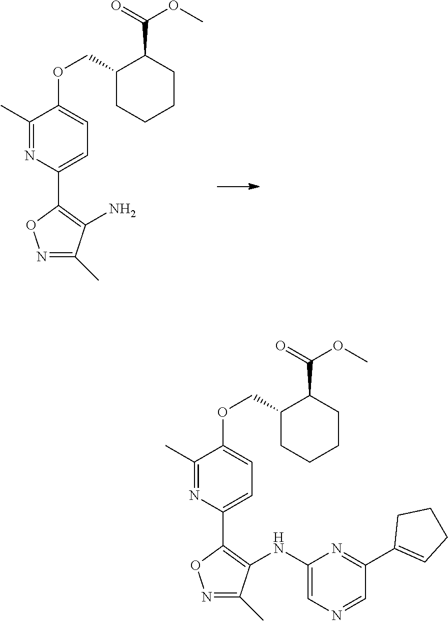

Intermediate O15 (34 mg, 0.068 mmol, 61% yield) was obtained from 2-chloro-6-(cyclopenten-1-yl)pyrazine (Intermediate B12, 20.1 mg, 0.110 mmol) and methyl (1 S,2S)-2-(((6-(4-amino-3-methylisoxazol-5-yl)-2-methylpyridin-3-yl)oxy)methyl)cyclohexane-1-carboxylate (Intermediate O13, 40.0 mg, 0.110 mmol) using a similar method as for Intermediate A25.

LC-MS (ESI): m/z (M+1): 504.3 (Method 1)

The Intermediates in the following table were prepared from reagents reported below by using methods analogous to Intermediate O15.

| Intermediate | Structure & Name | Reagents | Analytical data |
|---|---|---|---|
| O16 | 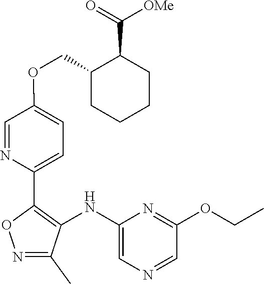 [SMILES: CCOc1cncc(Nc2c(C)noc2-c2ccc(OC[C@H]3CCCC[C@@H]3C(=O)OC)cn2)n1] | O14 and 2-chloro-6- ethoxy- pyrazine | LC-MS (ESI): m/z (M + 1): 468.3 (Method 14) |
| | (1S,2S)-2-(((6-(4-((6- | | |
| | ethoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylate | | |
| O17 | 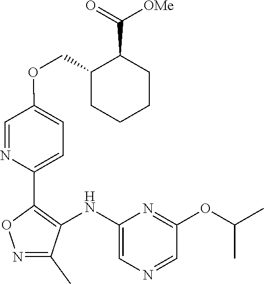 [SMILES: COC(=O)[C@H]1CCCC[C@@H]1COc1ccc(-c2onc(C)c2Nc2cncc(OC(C)C)n2)nc1] | O14 and A23 | LC-MS (ESI): m/z (M + 1): 482.3 (Method 14) |
| | (1S,2S)-2-(((6-(4-((6- | | |
| | isopropoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylate | | |
| O18 | 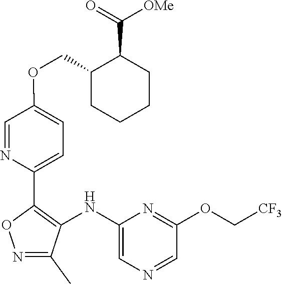 [SMILES: COC(=O)[C@H]1CCCC[C@@H]1COc1ccc(-c2onc(C)c2Nc2cncc(OCC(F)(F)F)n2)nc1] | O14 and 2-chloro-6- (2,2,2- trifluoro- ethoxy) pyrazine | LC-MS (ESI): m/z (M + 1): 522.3 (Method 14) |
| | (1S,2S)-2-(((6-(3-methyl- | | |
| | 4-((6-(2,2,2- | | |
| | trifluoroethoxy)pyrazin- | | |
| | 2-yl)amino)isoxazol-5- | | |
| | yl)pyridin-3- | | |
| | yl)oxy)methyl)cyclo- | | |
| | hexane-1-carboxylate | | |
| O19 | 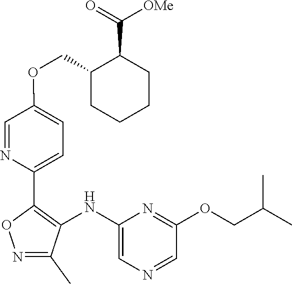 [SMILES: COC(=O)[C@H]1CCCC[C@@H]1COc1ccc(-c2onc(C)c2Nc2cncc(OCC(C)C)n2)nc1] | O14 and 2-chloro-6- isobutoxy- pyrazine | LC-MS (ESI): m/z (M + 1): 496.3 (Method 14) |
| | (1S,2S)-2-(((6-(4-((6- | | |
| | isobutoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylate | | |
| O20 | 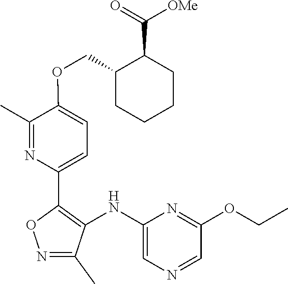 [SMILES: CCOc1cncc(Nc2c(C)noc2-c2ccc(OC[C@H]3CCCC[C@@H]3C(=O)OC)c(C)n2)n1] | O13 and 2- chloro-6- ethoxy- pyrazine | LC-MS (ESI): m/z (M + 1): 482.3 (Method 14) |
| | (1S,2S)-2-(((6-(4-((6- | | |
| | ethoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylate | | |
| O21 | 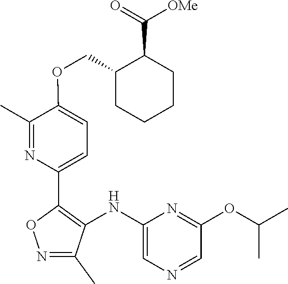 [SMILES: COC(=O)[C@H]1CCCC[C@@H]1COc1ccc(-c2onc(C)c2Nc2cncc(OC(C)C)n2)nc1C] | O13 and A23 | LC-MS (ESI): m/z (M + 1): 496.3 (Method 14) |
| | (1S,2S)-2-(((6-(4-((6- | | |
| | isopropoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylate | | |
| O22 | 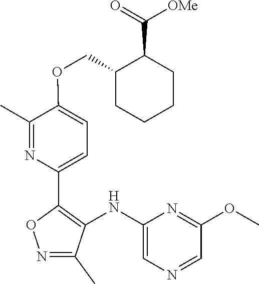 | O13 and 2- chloro-6- methoxy- pyrazine | LC-MS (ESI): m/z (M + 1): 468.3 (Method 14) |
| | (1S,2S)-2-(((6-(4-((6- | | |
| | methoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylate | | |
| O23 | 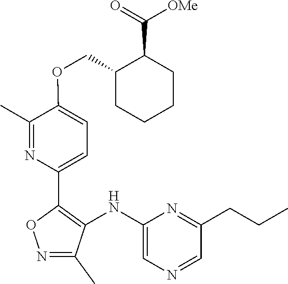 [SMILES: CCCc1cncc(Nc2c(C)noc2-c2ccc(OC[C@H]3CCCC[C@@H]3C(=O)OC)c(C)n2)n1] | O13 and 2- chloro-6- propyl- pyrazine | LC-MS (ESI): m/z (M + 1): 480.4 (Method 14) |
| | (1S,2S)-2-(((2-methyl- | | |
| | 6-(3-methyl-4-((6- | | |
| | propylpyrazin-2- | | |
| | yl)amino)isoxazol-5- | | |
| | yl)pyridin-3- | | |
| | yl)oxy)methyl)cyclo- | | |
| | hexane-1-carboxylate | | |

### Step 8: methyl (1S,2S)-2-(((6-(4-((6-cyclopentylpyrazin-2-yl)amino)-3-methylisoxazol-5-yl)-2-methylpyridin-3-yl)oxy)methyl)cyclohexane-1-carboxylate (Intermediate O24)

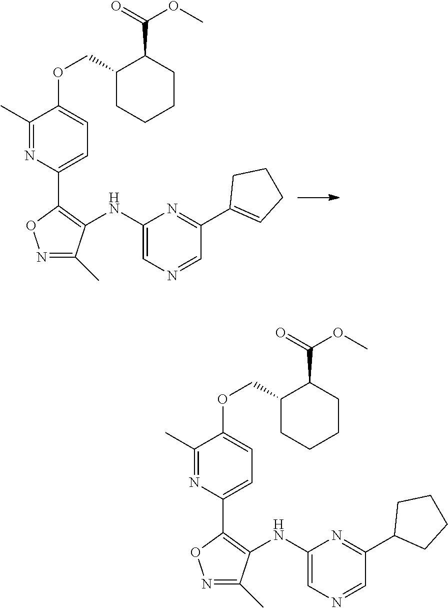 [SMILES: COC(=O)[C@H]1CCCC[C@@H]1COc1ccc(-c2onc(C)c2Nc2cncc(C3=CCCC3)n2)nc1C ; COC(=O)[C@H]1CCCC[C@@H]1COc1ccc(-c2onc(C)c2Nc2cncc(C3CCCC3)n2)nc1C]

Intermediate O24 (15 mg, 0.03 mmol, 43.9% yield) was obtained as a pale yellow oil from methyl (1S,2S)-2-(((6-(4-((6-(cyclopent-1-en-1-yl)pyrazin-2-yl)amino)-3-methylisoxazol-5-yl)-2-methylpyridin-3-yl)oxy)methyl)cyclohexane-1-carboxylate (Intermediate O15, 34.0 mg, 0.070 mmol) using a similar method as for Intermediate A21.

LC-MS (ESI): m/z (M+1): 506.3 (Method 1)

### Step 9: (1S,2S)-2-(((6-(4-((6-cyclopentylpyrazin-2-yl)amino)-3-methylisoxazol-5-yl)-2-methylpyridin-3-yl)oxy)methyl)cyclohexane-1-carboxylic acid (Compound 46)

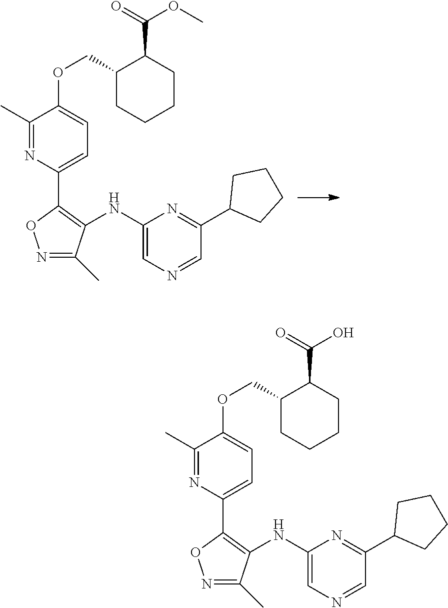 [SMILES: COC(=O)[C@H]1CCCC[C@@H]1COc1ccc(-c2onc(C)c2Nc2cncc(C3CCCC3)n2)nc1C ; Cc1nc(-c2onc(C)c2Nc2cncc(C3CCCC3)n2)ccc1OC[C@H]1CCCC[C@@H]1C(=O)O]

Compound 46 (10 mg, 0.02 mmol, 68.6% yield) was obtained as a pale yellow oil from methyl (1S,2S)-2-(((6-(4-((6-cyclopentylpyrazin-2-yl)amino)-3-methylisoxazol-5-yl)-2-methylpyridin-3-yl)oxy)methyl)cyclohexane-1-carboxylate (Intermediate O24, 15.0 mg, 0.030 mmol) using a similar method as for Intermediate A9.

LC-MS (ESI): m/z (M+1): 492.3 (Method 1)

¹H NMR (500 MHz, Chloroform-d) δ ppm 8.25 (s, 1H), 7.87 (s, 2H), 7.61 (d, J=8.5 Hz, 1H), 7.13 (d, J=8.6 Hz, 1H), 3.86-4.01 (m, 2H), 3.04 (quin, J=8.3 Hz, 1H), 2.44 (s, 3H), 2.40 (s, 3H), 2.37 (ddd, J=12.1, 11.0, 3.7 Hz, 1H), 2.13-2.23 (m, 1H), 2.08 (br dd, J=13.0, 2.2 Hz, 1H), 1.90-2.05 (m, 3H), 1.62-1.89 (m, 8H), 1.56 (qd, J=12.6, 3.4 Hz, 1H), 1.21-1.45 (m, 3H)

The Compounds in the following table were prepared from reagents reported below by using methods analogous to Compound 46.

| Compound | Structure & Name | Reagents | Analytical data |
|---|---|---|---|
| 47 | 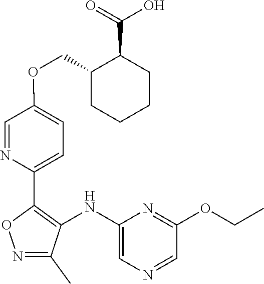 [SMILES: CCOc1cncc(Nc2c(C)noc2-c2ccc(OC[C@H]3CCCC[C@@H]3C(=O)O)cn2)n1] | O16 | LC-MS (ESI): m/z (M + 1): 454.3 (Method 14) ¹H NMR (300 MHz, DMSO-d₆) δ ppm 12.09 (br s, 1H), 8.77 (s, 1H), 8.32 (d, J = 2.8 Hz, 1H), 7.74 (d, J = 8.8 Hz, 1H), 7.68 (s, 1H), 7.49 (dd, J = 8.8, 2.8 Hz, 1H), 7.46 (s, 1H), 3.85-4.04 (m, 4H), 2.18 (s, 3H), 2.10- 2.26 (m, 1H), 1.77-2.04 (m, 3H), 1.57-1.77 (m, 2H), 1.29-1.46 (m, 1H), 1.17-1.29 (m, 3H), 1.14 (t, J = 7.0 Hz, 3H |
| | (1S,2S)-2-(((6-(4-((6- | | |
| | ethoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylic acid | | |
| 48 | 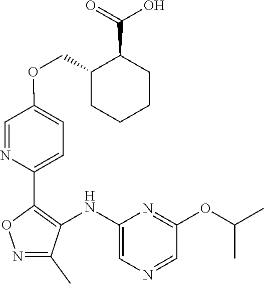 [SMILES: Cc1noc(-c2ccc(OC[C@H]3CCCC[C@@H]3C(=O)O)cn2)c1Nc1cncc(OC(C)C)n1] | O17 | LC-MS (ESI): m/z (M + 1): 468.2 (Method 14) ¹H NMR (300 MHz, DMSO-d₆) δ ppm 12.10 (br s, 1H), 8.74 (s, 1H), 8.32 (d, J = 2.8 Hz, 1H), 7.74 (d, J = 8.8 Hz, 1H), 7.67 (s, 1H), 7.49 (dd, J = 8.8, 2.8 Hz, 1H), 7.41 (s, 1H), 4.70 (quin, J = 6.2 Hz, 1H), 3.88-4.04 (m, 2H), 2.19 (s, 3H), 2.10- 2.25 (m, 1H), 1.77-2.04 (m, 3H), 1.60-1.77 (m, 2H), 1.27-1.44 (m, 1H), 1.12-1.27 (m, 3H), 1.08 (d, J = 6.2 Hz, 6H) |
| | (1S,2S)-2-(((6-(4-((6- | | |
| | isopropoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylic acid | | |
| 49 | 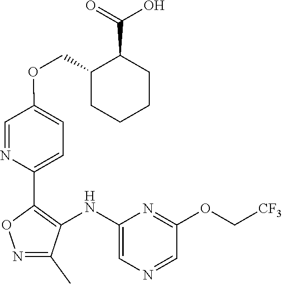 [SMILES: Cc1noc(-c2ccc(OC[C@H]3CCCC[C@@H]3C(=O)O)cn2)c1Nc1cncc(OCC(F)(F)F)n1] | O18 | LC-MS (ESI): m/z (M + 1): 508.2 (Method 14) ¹H NMR (300 MHz, DMSO-d₆) δ ppm 12.13 (br s, 1H), 9.04 (s, 1H), 8.29 (d, J = 2.6 Hz, 1H), 7.83 (s, 1H), 7.74 (d, J = 8.8 Hz, 1H), 7.62 (s, 1H), 7.49 (dd, J = 8.8, 2.8 Hz, 1H), 4.52 (q, J = 9.0 Hz, 2H), 3.83-4.04 (m, 2H), 2.19 (s, 3H), 2.10- 2.25 (m, 1H), 1.78-2.06 (m, 3H), 1.58-1.78 (m, 2H), 1.30-1.48 (m, 1H), 1.08-1.30 (m, 3H) |
| | (1S,2S)-2-(((6-(3-methyl- | | |
| | 4-((6-(2,2,2- | | |
| | trifluoroethoxy)pyrazin- | | |
| | 2-yl)amino)isoxazol-5- | | |
| | yl)pyridin-3- | | |
| | yl)oxy)methyl)cyclo- | | |
| | hexane-1-carboxylic acid | | |
| 50 | 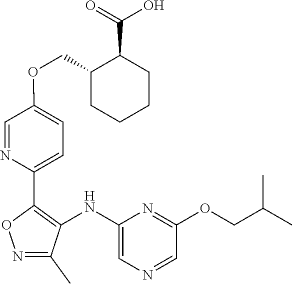 [SMILES: Cc1noc(-c2ccc(OC[C@H]3CCCC[C@@H]3C(=O)O)cn2)c1Nc1cncc(OCC(C)C)n1] | O19 | LC-MS (ESI): m/z (M + 1): 482.3 (Method 14) ¹H NMR (300 MHz, DMSO-d₆) δ ppm 12.13 (br s, 1H), 8.79 (s, 1H), 8.30 (d, J = 2.7 Hz, 1H), 7.72 (d, J = 8.8 Hz, 1H), 7.69 (s, 1H), 740-7.51 (m, 2H), 3.86-4.03 (m, 2H), 3.59 (d, J = 6.6 Hz, 2H), 2.22 (s, 3H), 2.10- 2.25 (m, 1H), 1.72-2.05 (m, 5H), 1.58-1.72 (m, 2H), 1.30-1.46 (m, 1H), 1.09-1.30 (m, 3H), 0.81 (d, J = 6.7 Hz, 6H) |
| | (1S,2S)-2-(((6-(4-((6- | | |
| | isobutoxypyrazin-2- | | |
| | yl)amino)-3- | | |
| | methylisoxazol-5- | | |
| | yl)pyridin-3- | | |
| | yl)oxy)methyl)cyclo- | | |
| | hexane-1-carboxylic acid | | |
| 51 | 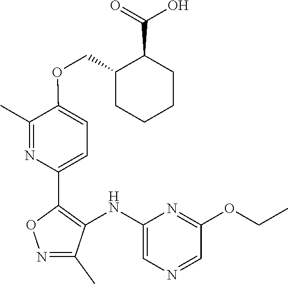 [SMILES: CCOc1cncc(Nc2c(C)noc2-c2ccc(OC[C@H]3CCCC[C@@H]3C(=O)O)c(C)n2)n1] | O20 | LC-MS (ESI): m/z (M + 1): 468.3 (Method 3) ¹H NMR (300 MHz, DMSO-d₆) δ ppm 12.08 (br s, 1H), 8.67 (s, 1H), 7.67 (s, 1H), 7.60 (d, J = 8.6 Hz, 1H), 7.46 (s, 1H), 7.40 (d, J = 8.6 Hz, 1H), 3.80-4.02 (m, 4H), 2.30 (s, 3H), 2.20 (s, 3H), 2.12-2.26 (m, 1H), 1.77- 2.04 (m, 3H), 1.61- 1.77 (m, 2H), 1.18-1.50 (m, 4H), 1.14 (t, J = 7.0 Hz, 3H) |
| | (1S,2S)-2-(((6-(4-((6- | | |
| | ethoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylic acid | | |
| 52 | 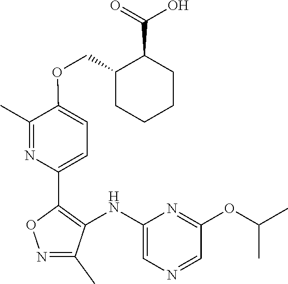 [SMILES: Cc1nc(-c2onc(C)c2Nc2cncc(OC(C)C)n2)ccc1OC[C@H]1CCCC[C@@H]1C(=O)O] | O21 | LC-MS (ESI): m/z (M + 1): 482.3 (Method 14) ¹H NMR (600 MHz, DMSO-d₆) δ ppm 12.11 (br s, 1H), 8.63 (s, 1H), 7.64 (s, 1H), 7.59 (d, J = 8.59 Hz, 1H), 7.37- 7.41 (m, 2H), 4.72 (quin, J = 6.19 Hz, 1H), 3.90- 3.96 (m, 1H), 3.87 (dd, J = 9.81, 5.83 Hz, 1H), 2.28 (s, 3H), 2.17- 2.22 (m, 4H), 1.94-2.01 (m, 1H), 1.87-1.93 (m, 1H), 1.81-1.86 (m, 1H), 1.71 (br d, J = 10.77 Hz, 2H), 1.32-1.44 (m, 1H), 1.13-1.29 (m, 3H), 1.07 (d, J = 6.03 Hz, 6H) |
| | (1S,2S)-2-(((6-(4-((6- | | |
| | isopropoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylic acid | | |
| 53 | 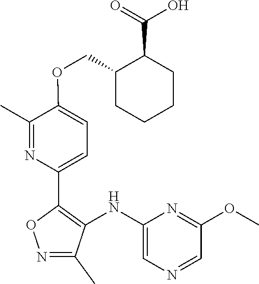 | O22 | LC-MS (ESI): m/z (M + 1): 454.3 (Method 14) ¹H NMR (300 MHz, DMSO-d₆) δ ppm 12.09 (br s, 1H), 8.71 (s, 1H), 7.67 (s, 1H), 7.61 (d, J = 8.6 Hz, 1H), 7.50 (s, 1H), 7.40 (d, J = 8.6 Hz, 1H), 3.82-4.02 (m, 2H), 3.59 (s, 3H), 2.30 (s, 3H), 2.21 (s, 3H), 2.12-2.26 (m, 1H), 1.77-2.05 (m, 3H), 1.61-1.78 (m, 2H), 1.34-1.49 (m, 1H), 1.12- 1.34 (m, 3H) |
| | (1S,2S)-2-(((6-(4-((6- | | |
| | methoxypyrazin-2- | | |
| | yl)amino)-3- | | |
| | methylisoxazol-5-yl)-2- | | |
| | methylpyridin-3- | | |
| | yl)oxy)methyl)cyclo- | | |
| | hexane-1-carboxylic acid | | |
| 54 | 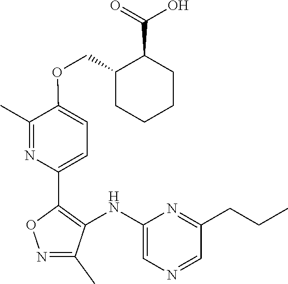 [SMILES: CCCc1cncc(Nc2c(C)noc2-c2ccc(OC[C@H]3CCCC[C@@H]3C(=O)O)c(C)n2)n1] | O23 | LC-MS (ESI): m/z (M + 1): 466.4 (Method 14) ¹H NMR (300 MHz, DMSO-d₆) δ ppm 12.08 (br s, 1H), 8.68 (s, 1H), 7.97 (s, 1H), 7.74 (s, 1H), 7.60 (d, J = 8.6 Hz, 1H), 7.39 (d, J = 8.6 Hz, 1H), 3.82-4.00 (m, 2H), 2.41 (t, J = 7.5 Hz, 2H), 2.27 (s, 3H), 2.14-2.25 (m, 1H), 2.19 (s, 3H), 1.79-2.05 (m, 3H), 1.62-1.79 (m, 2H), 1.15-1.51 (m, 6 H), 0.77 (t, J = 7.4 Hz, 3H) |
| | (1S,2S)-2-(((2-methyl-6- | | |
| | (3-methyl-4-((6- | | |
| | propylpyrazin-2- | | |
| | yl)amino)isoxazol-5- | | |
| | yl)pyridin-3- | | |
| | yl)oxy)methyl)cyclo- | | |
| | hexane-1-carboxylic acid | | |

### Example 19

### Process Synthesis of Compound 55

### (1S,2S)-2-(((6-(4-((2-(4-fluorophenoxy)ethyl)sulfonamido)-3-methylisoxazol-5-yl)-2-methylpyridin-3-yl)oxy)methyl)cyclohexane-1-carboxylic acid

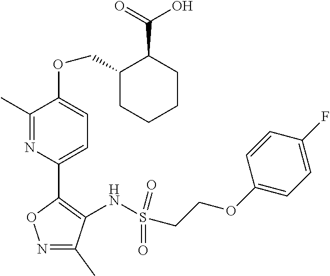 [SMILES: Cc1nc(-c2onc(C)c2NS(=O)(=O)CCOc2ccc(F)cc2)ccc1OC[C@H]1CCCC[C@@H]1C(=O)O]

### Step 1: methyl (1S,2S)-2-(((6-(4-((2-(4-fluorophenoxy)ethyl)sulfonamido)-3-methylisoxazol-5-yl)-2-methylpyridin-3-yl)oxy)methyl)cyclohexane-1-carboxylate (Intermediate P1)

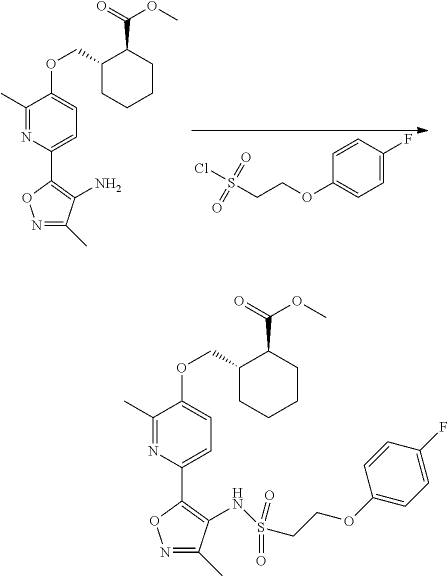

Methyl (1S,2S)-2-(((6-(4-amino-3-methylisoxazol-5-yl)-2-methylpyridin-3-yl)oxy)methyl)cyclohexane-1-carboxylate (Intermediate O13, 280.0 mg, 0.780 mmol) was dissolved in dry (3 mL). Pyridine (0.09 mL, 1.09 mmol) was added, followed by a solution of 2-(4-fluorophenoxy)ethane-1-sulfonyl chloride (157.0 mg, 0.660 mmol) in dry MeCN (2 mL). The mixture was then stirred overnight at RT. A 2^{nd} addition of pyridine (0.044, 0.05 mmol) and 2-(4-fluorophenoxy)ethane-1-sulfonyl chloride (79 mg, 0.33 mmol) was made and the mixture was stirred at rt for 2 days. The mixture was then evaporated in vacuo. The residue was partitioned between water and EtOAc. The organic phase was washed with water, dried over Na2SO4, filtered and concentrated in vacuo. The crude was purified by flash chromatography (gradient cyclohexane/EtOAc from 93/7 to 40/60). A 2^{nd} column was done (eluent A: water+0.1% HCOOH eluent B: acetonitrile+0.1% HCOOH, gradient A/B from 90/10 to 15/85) to give a white solid (149 mg, 0.265 mmol, 64.1% yield).

LC-MS (ESI): m/z (M+1): 562.2 (Method 1)

### Step 2: (1S,2S)-2-(((6-(4-((2-(4-fluorophenoxy)ethyl)sulfonamido)-3-methylisoxazol-5-yl)-2-methylpyridin-3-yl)oxy)methyl)cyclohexane-1-carboxylic acid (Compound 55)

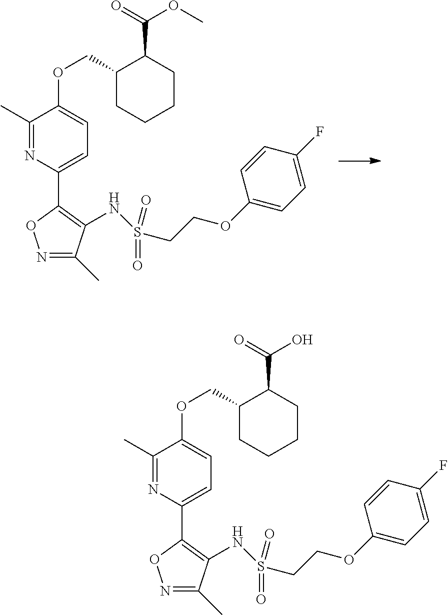 [SMILES: COC(=O)[C@H]1CCCC[C@@H]1COc1ccc(-c2onc(C)c2NS(=O)(=O)CCOc2ccc(F)cc2)nc1C ; Cc1nc(-c2onc(C)c2NS(=O)(=O)CCOc2ccc(F)cc2)ccc1OC[C@H]1CCCC[C@@H]1C(=O)O]

SnMe₃OH (34.77 mg, 0.190 mmol) was added to a solution of methyl (1S,2S)-2-(((6-(4-((2-(4-fluorophenoxy)ethyl)sulfonamido)-3-methylisoxazol-5-yl)-2-methylpyridin-3-yl)oxy)methyl)cyclohexane-1-carboxylate (Intermediate P1, 36.0 mg, 0.060 mmol) in dry DCE (0.800 mL) and the mixture was stirred at 60° C. After 3h, an addition of SnMe₃OH (34.77 mg, 0.190 mmol) was made and the mixture was stirred overnight at 60° C. A 3^{rd} addition of SnMe₃OH (34.77 mg, 0.190 mmol) was made and the temperature was increased to 80° C. After 4 days the mixture was then acidified with a 1N HCl aqueous solution and extracted with ethyl acetate (2 times). The mixed organic phases were washed with brine, dried over Na2SO4, filtered and concentrated in vacuo. The crude was purified by flash chromatography (eluent A: water+0.1% formic acid, eluent B: acetonitrile+0.1% formic acid, gradient A/B from 85/15 to 25/) to give the title compound as a white solid (16 mg, 0.029 mmol, 45.6% yield).

LC-MS (ESI): m/z (M+1): 548.2 (Method 1)

1H NMR (500 MHz, DMSO-d6) δ ppm 8.30-14.20 (m, 1H), 7.80 (d, J=8.5 Hz, 1H), 7.39 (d, J=8.6 Hz, 1H), 7.08 (t, J=8.9 Hz, 2H), 6.79-6.86 (m, 2H), 4.22 (t, J=6.4 Hz, 2H), 3.81-3.99 (m, 2H), 3.55 (br t, J=6.2 Hz, 2H), 2.37 (s, 3H), 2.27 (s, 3H), 2.21 (td, J=11.3, 3.6 Hz, 1H), 1.95-2.07 (m, 1H), 1.80-1.95 (m, 2H), 1.73 (br d, J=10.0 Hz, 2H), 1.35-1.47 (m, 1H), 1.10-1.33 (m, 4H).

The Compounds in the following table were prepared from reagents reported below by using methods analogous to Compound 55.

| Compound | Structure & Name | Reagents | Analytical data |
|---|---|---|---|
| 56 | 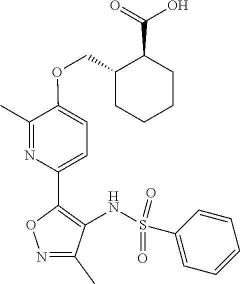 [SMILES: Cc1nc(-c2onc(C)c2NS(=O)(=O)c2ccccc2)ccc1OC[C@H]1CCCC[C@@H]1C(=O)O] | O13 and benzene- sulfonyl chloride | LC-MS (ESI): m/z (M + 1): 486.0 (Method 1) 1H NMR (400 MHz, DMSO-d6) δ ppm 12.16 (br s, 1 H), 9.63 (br s, 1 H), 7.46-7.58 (m, 3 H), 7.36- 7.44 (m, 1 H), 7.27-7.35 (m, 2 H), 7.20 (d, J = 8.7 Hz, 1 H), 3.81-3.99 (m, 2 H), 2.30 (s, 3 H), 2.24 (td, J = 11.2, 3.5 Hz, 1 H), 2.07 (s, 3 H), 1.97-2.03 (m, 1 H), 1.94 (br d, J = 13.7 Hz, 1 H), 1.87 (br d, J = 11.3 Hz, 1 H), 1.68-1.81 (m, 2 H), 1.18-1.50 (m, 4 H) |
| | (1S,2S)-2-(((2-methyl-6- | | |
| | (3-methyl-4- | | |
| | (phenylsulfonamido) | | |
| | isoxazol-5-yl)pyridin-3- | | |
| | yl)oxy)methyl)cyclo- | | |
| | hexane-1-carboxylic acid | | |
| 57 | 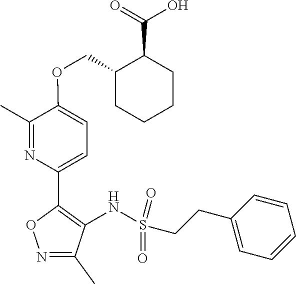 [SMILES: Cc1nc(-c2onc(C)c2NS(=O)(=O)CCc2ccccc2)ccc1OC[C@H]1CCCC[C@@H]1C(=O)O] | O13 and 2- phenylethane- 1-sulfonyl chloride | LC-MS (ESI): m/z (M + 1): 514.2 (Method 1) 1H NMR (400 MHz, DMSO-d6) δ ppm 12.11 (br s, 1 H), 9.48 (br s, 1 H), 7.79 (d, J = 8.6 Hz, 1 H), 7.44 (d, J = 8.7 Hz, 1 H), 7.11-7.27 (m, 3 H), 6.90- 7.05 (m, 2 H), 3.78-4.06 (m, 2 H), 3.33-3.38 (m, 2 H), 2.83-3.04 (m, 2 H), 2.29 (s, 3 H), 2.26 (s, 3 H), 2.21 (td, J = 11.2, 3.6 Hz, 1 H), 1.96-2.07 (m, 1 H), 1.89-1.96 (m, 1 H), 1.80- 1.88 (m, 1 H), 1.65-1.78 (m, 2 H), 1.32-1.48 (m, 1 H), 1.13-1.32 (m, 3 H) |
| | (1S,2S)-2-(((2-methyl-6- | | |
| | (3-methyl-4-((2- | | |
| | phenylethyl)sulfonamido) | | |
| | isoxazol-5-yl)pyridin-3- | | |
| | yl)oxy)methyl)cyclo- | | |
| | hexane-1-carboxylic acid | | |

### Example 20

### Process Synthesis of Compound 58

### (1S,2S)-2-(((6-(5-((((R)-1-(2-chlorophenyl)ethoxy)carbonyl)amino)-1-methyl-1H-pyrazol-4-yl)-2-methylpyridin-3-yl)oxy)methyl)cyclohexane-1-carboxylic acid

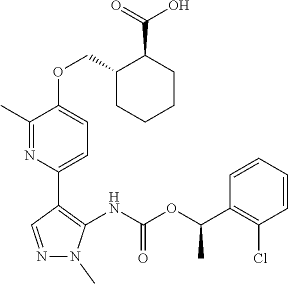 [SMILES: Cc1nc(-c2cnn(C)c2NC(=O)O[C@H](C)c2ccccc2Cl)ccc1OC[C@H]1CCCC[C@@H]1C(=O)O]

### Step 1: methyl 4-(5-amino-6-methylpyridin-2-yl)-1-methyl-1H-pyrazole-5-carboxylate (Intermediate Q1)

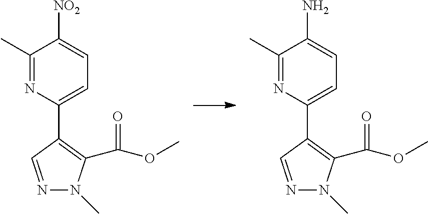

Intermediate Q1 (335 mg, 1.36 mmol, 71.31% yield) was obtained as a yellow solid from methyl 2-methyl-4-(6-methyl-5-nitropyridin-2-yl)pyrazole-3-carboxylate (Intermediate D4, 527.0 mg, 1.91 mmol) using a similar method as for Intermediate C3.

LC-MS (ESI): m/z (M+1): 247.1 (Method 1)

### Step 2: methyl 4-(5-hydroxy-6-methylpyridin-2-yl)-1-methyl-1H-pyrazole-5-carboxylate (Intermediate Q2)

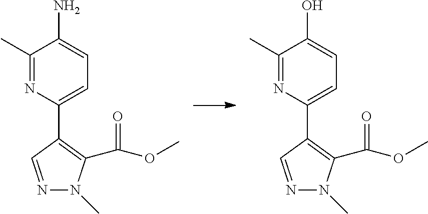

To a solution of methyl 4-(5-amino-6-methylpyridin-2-yl)-1-methyl-1H-pyrazole-5-carboxylate (Intermediate Q1, 525.0 mg, 2.12 mmol) in 2 M H₂SO₄ (31.7 mL, 63.41 mmol) at 0° C. was added a solution of sodium nitrite (0.164 g, 2.35 mmol) in Water (14 mL). The reaction mixture was stirred at RT for 1 h, then at 900 C 30 minutes. NaOH 2 M was added until pH 5, then the mixture was extracted with EtOAc (3 times), collected organic phase was dried over Na2SO4, filtered and evaporated to give the title product (323 mg, 1.31 mmol, 62% yield) as yellow solid, which was used in the next step without further purification.

LC-MS (ESI): m/z (M+1): 248.1 (Method 1)

### Step 3: methyl 4-(5-(methoxymethoxy)-6-methylpyridin-2-yl)-1-methyl-1H-pyrazole-5-carboxylate (Intermediate Q3)

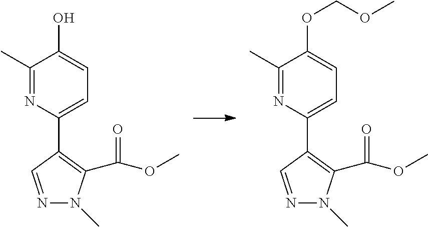

To a solution of methyl 4-(5-hydroxy-6-methylpyridin-2-yl)-1-methyl-1H-pyrazole-5-carboxylate (Intermediate Q2, 323.0 mg, 1.31 mmol) in DCM (6 mL) were added: DIPEA (0.46 mL, 2.61 mmol), DMAP (31.92 mg, 0.260 mmol) and chloro(methoxy)methane (0.2 mL, 2.61 mmol) at 0° C. The mixture was stirred at RT for 5 h. Water was added and the mixture was extracted with DCM, collected organic phases were dried over Na2SO4, filtered and evaporated to give the crude material that was purified by flash chromatography (CyHex/EtOAc from 7/3 to 2/8) to afford the title compound (263.3 mg, 0.904 mmol, 69.19% yield) as yellow oil.

LC-MS (ESI): m/z (M+1): 292.1 (Method 1)

### Step 4: 4-(5-(methoxymethoxy)-6-methylpyridin-2-yl)-1-methyl-1H-pyrazole-5-carboxylic acid (Intermediate Q4)

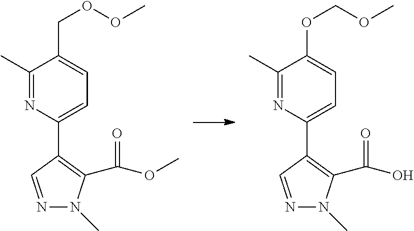

Intermediate Q4 (231 mg, 0.833 mmol, 92.17% yield) was obtained from methyl 4-(5-(methoxymethoxy)-6-methylpyridin-2-yl)-1-methyl-1H-pyrazole-5-carboxylate (Intermediate Q3, 263.3 mg, 0.900 mmol) using a similar method as for Intermediate B4.

LC-MS (ESI): m/z (M+1): 278.1 (Method 1)

The Intermediate in the following table was prepared from reagents reported below by using methods analogous as for Intermediate O4.

| Intermediate | Structure & Name | Reagents | Analytical data |
|---|---|---|---|
| Q5 | 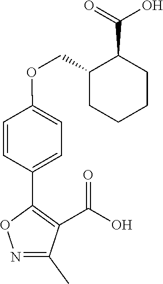 [SMILES: Cc1noc(-c2ccc(OC[C@H]3CCCC[C@@H]3C(=O)O)cc2)c1C(=O)O] | O10 | LC-MS (ESI): m/z (M + 1): 360.9 (Method 5) ¹H NMR (300 MHz, DMSO-d₆) δ 13.17 (s, 1H), 12.17 (s, 1H), 7.87 (d, J = 8.8 Hz, 2H), 7.06 (d, J = 8.8 Hz, 2H), 3.92 (d, J = 7.2 Hz, 2H), 2.40 (s, 3H), 2.03-1.86 (m, 4H), 1.81- 1.67 (m, 3H), 1.59- 1.21 (m, 3H) |
| | 5-(4-(((1S,2S)-2- | | |
| | carboxycyclohexyl) | | |
| | methoxy)phenyl)-3- | | |
| | methylisoxazole-4- | | |
| | carboxylic acid | | |
| Q6 | 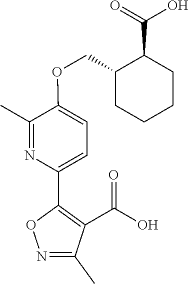 [SMILES: Cc1nc(-c2onc(C)c2C(=O)O)ccc1OC[C@H]1CCCC[C@@H]1C(=O)O] | O11 | LC-MS (ESI): m/z (M + 1): |
| | 5-(5-(((1S,2S)-2- | | 374.2 (Method 5) |
| | carboxycyclohexyl) | | |
| | methoxy)-6-methylpyridin- | | |
| | 2-yl)-3-methylisoxazole- | | |
| | 4-carboxylic acid | | |

### Step 5: (R)-1-(2-chlorophenyl)ethyl (4-(5-(methoxymethoxy)-6-methylpyridin-2-yl)-1-methyl-1H-pyrazol-5-yl)carbamate (Intermediate Q7)

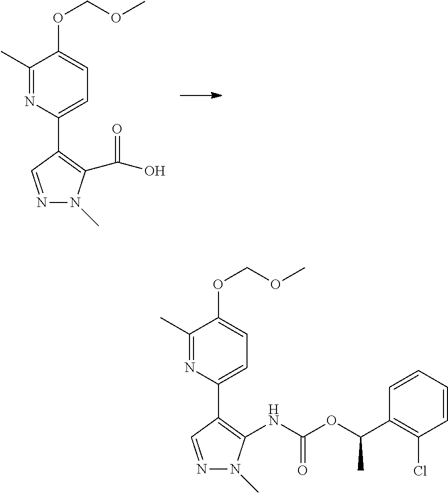 [SMILES: COCOc1ccc(-c2cnn(C)c2C(=O)O)nc1C ; COCOc1ccc(-c2cnn(C)c2NC(=O)O[C@H](C)c2ccccc2Cl)nc1C]

Intermediate Q7 (274.6 mg, 0.637 mmol, 76.5% yield) was obtained as yellow solid from 4-(5-(methoxymethoxy)-6-methylpyridin-2-yl)-1-methyl-1H-pyrazole-5-carboxylic acid (Intermediate Q4, 231.0 mg, 0.830 mmol) and (R)-1-(2-chlorophenyl)ethan-1-ol (0.17 mL, 1.25 mmol) using a similar method as for Intermediate A14.

LC-MS (ESI): m/z (M+1): 431.1 (Method 1)

### Step 6: (R)-1-(2-chlorophenyl)ethyl (4-(5-hydroxy-6-methylpyridin-2-yl)-1-methyl-1H-pyrazol-5-yl)carbamate (Intermediate Q8)

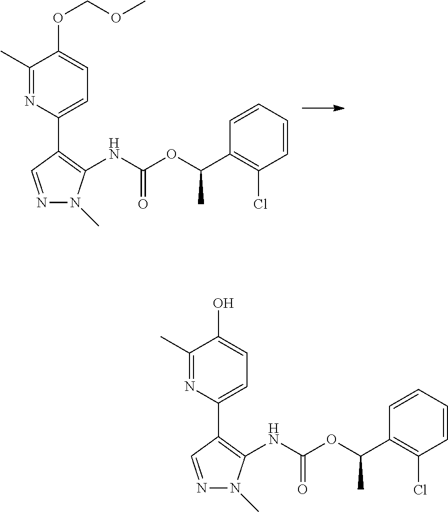 [SMILES: COCOc1ccc(-c2cnn(C)c2NC(=O)O[C@H](C)c2ccccc2Cl)nc1C ; Cc1nc(-c2cnn(C)c2NC(=O)O[C@H](C)c2ccccc2Cl)ccc1O]

(R)-1-(2-chlorophenyl)ethyl (4-(5-(methoxymethoxy)-6-methylpyridin-2-yl)-1-methyl-1H-pyrazol-5-yl)carbamate (Intermediate Q7, 274.6 mg, 0.640 mmol) was dissolved in THE (2.476 mL). A solution of HCl (4.9 mL, 3.19 mmol) (2% w/w, about 0.65M) in isopropyl alcohol was then added and the mixture was stirred at RT for 1 week. The mixture was then neutralized with a 2N aqueous NaOH solution and concentrated in vacuo. Water was added and the mixture was extracted with EtOAc (3 times). The mixed organic phases were dried over Na2SO4, filtered and concentrated in vacuo to give the crude material which was purified by flash chromatography (DCM/MeOH from 10/0 to 9/1) to afford the target compound (146 mg, 0.377 mmol, 59.22% yield) as white solid.

LC-MS (ESI): m/z (M+1): 387.2 (Method 1)

### Step 7: methyl (1S,2S)-2-(((6-(5-((((R)-1-(2-chlorophenyl)ethoxy)carbonyl)amino)-1-methyl-1H-pyrazol-4-yl)-2-methylpyridin-3-yl)oxy)methyl)cyclohexane-1-carboxylate (Intermediate Q9)

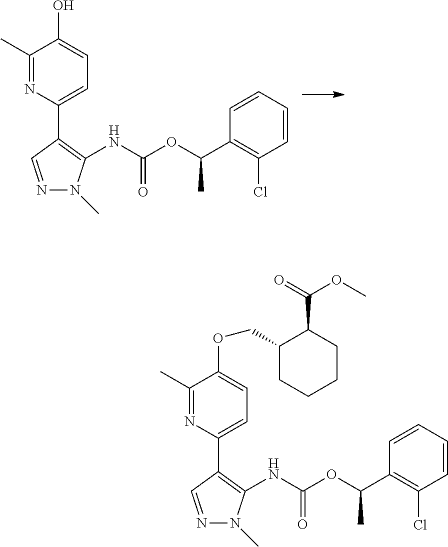 [SMILES: Cc1nc(-c2cnn(C)c2NC(=O)O[C@H](C)c2ccccc2Cl)ccc1O ; COC(=O)[C@H]1CCCC[C@@H]1COc1ccc(-c2cnn(C)c2NC(=O)O[C@H](C)c2ccccc2Cl)nc1C]

Intermediate Q9 (70 mg, 0.129 mmol, 83.41% yield) was obtained from methyl (1S,2S)-2-(hydroxymethyl)cyclohexane-1-carboxylate (Intermediate O7, 53.43 mg, 0.310 mmol) and (R)-1-(2-chlorophenyl)ethyl (4-(5-hydroxy-6-methylpyridin-2-yl)-1-methyl-1H-pyrazol-5-yl)carbamate (Intermediate Q8, 60.0 mg, 0.160 mmol) using a similar method as for Intermediate O9.

LC-MS (ESI): m/z (M+1): 541.3 (Method 1)

### Step 8: (1S,2S)-2-(((6-(5-((((R)-1-(2-chlorophenyl)ethoxy)carbonyl)amino)-1-methyl-1H-pyrazol-4-yl)-2-methylpyridin-3-yl)oxy)methyl)cyclohexane-1-carboxylic acid (Compound 58)

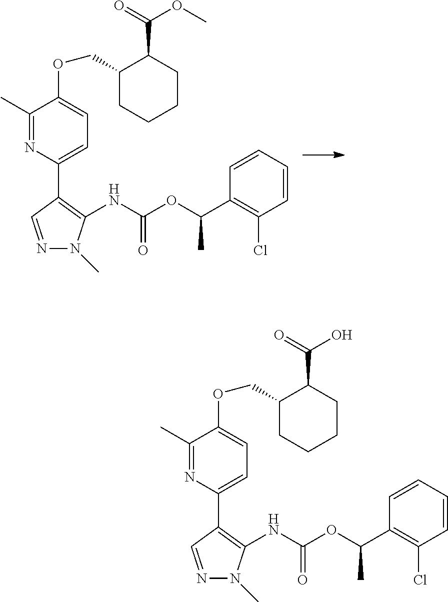 [SMILES: COC(=O)[C@H]1CCCC[C@@H]1COc1ccc(-c2cnn(C)c2NC(=O)O[C@H](C)c2ccccc2Cl)nc1C ; Cc1nc(-c2cnn(C)c2NC(=O)O[C@H](C)c2ccccc2Cl)ccc1OC[C@H]1CCCC[C@@H]1C(=O)O]

Compound 58 (29.4 mg, 0.056 mmol, 43.12% yield) was obtained as a white solid from methyl (1S,2S)-2-(((6-(5-((((R)-1-(2-chlorophenyl)ethoxy)carbonyl)amino)-1-methyl-1H-pyrazol-4-yl)-2-methylpyridin-3-yl)oxy)methyl)cyclohexane-1-carboxylate (Intermediate Q9, 70.0 mg, 0.130 mmol) using a similar method as for Intermediate B4.

LC-MS (ESI): m/z (M+1): 527.2 (Method 1)

¹H NMR (500 MHz, DMSO-d6) δ ppm 11.85-12.32 (m, 1H), 8.82-9.75 (m, 1H), 7.81 (s, 1H), 7.25-7.30 (m, 1H), 7.18-7.22 (m, 1H), 6.88-7.69 (m, 4H), 5.86-6.06 (m, 1H), 3.77-3.95 (m, 2H), 3.63 (br s, 3H), 2.33 (s, 3H), 2.22 (td, J=11.3, 3.6 Hz, 1H), 1.82-2.08 (m, 3H), 1.73 (br d, J=9.3 Hz, 2H), 1.35-1.66 (m, 3H), 1.19-1.45 (m, 4H)

### Example 21

### Process Synthesis of Compound 59

### (1S,2S)-2-((4-(3-methyl-4-((((R)-1-(pyridin-3-yl)ethoxy)carbonyl)amino)isoxazol-5-yl)phenoxy)methyl)cyclohexane-1-carboxylic acid

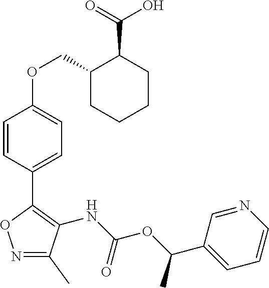 [SMILES: Cc1noc(-c2ccc(OC[C@H]3CCCC[C@@H]3C(=O)O)cc2)c1NC(=O)O[C@H](C)c1cccnc1]

### Step 1: 5-(4-(((1S,2S)-2-(methoxycarbonyl)cyclohexyl)methoxy)phenyl)-3-methylisoxazole-4-carboxylic acid (Intermediate R1)

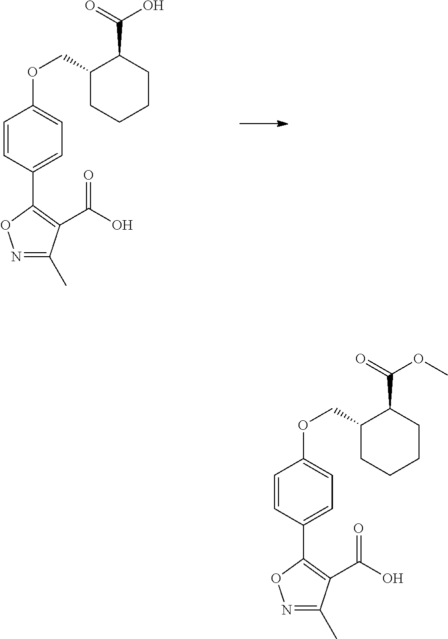 [SMILES: Cc1noc(-c2ccc(OC[C@H]3CCCC[C@@H]3C(=O)O)cc2)c1C(=O)O ; COC(=O)[C@H]1CCCC[C@@H]1COc1ccc(-c2onc(C)c2C(=O)O)cc1]

Intermediate R1 (0.08 g, 0.203 mmol, 70% yield was obtained from 5-(4-(((1S,2S)-2-carboxycyclohexyl)methoxy)phenyl)-3-methylisoxazole-4-carboxylic acid (Intermediate Q5, 0.105 g, 0.29 mmol) using a similar method as for Intermediate L4.

LC-MS (ESI): m/z (M+1): 374.4 (Method 5)

¹H NMR (400 MHz, DMSO-d6) δ 7.99 (d, J=8.4 Hz, 2H), 6.99 (d, J=8.5 Hz, 2H), 3.89 (d, J=5.6 Hz, 2H), 3.59 (s, 3H), 2.36 (s, 3H), 2.28 (td, J=11.3, 3.6 Hz, 1H), 2.09-1.97 (m, 1H), 1.86 (dd, J=12.8, 3.5 Hz, 2H), 1.72 (d, J=9.4 Hz, 2H), 1.49-1.34 (m, 1H), 1.34-1.09 (m, 3H)

The Intermediates in the following table were prepared from reagents reported below by using methods analogous to Intermediate R1.

| Intermediate | Structure & Name | Reagents | Analytical data |
|---|---|---|---|
| R2 | 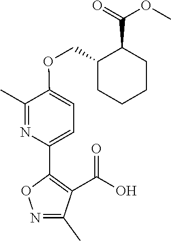 | Q6 | LC-MS (ESI): m/z (M + 1): 388.2 (Method 5) |
| | 5-(5-(((1S,2S)-2- | | |
| | (methoxycarbonyl)cyclo | | |
| | hexyl)methoxy)-6- | | |
| | methylpyridin-2-yl)-3- | | |
| | methylisoxazole-4- | | |
| | carboxylic acid | | |

### Step 2: methyl (1S,2S)-2-((4-(3-methyl-4-((((R)-1-(pyridin-3-yl)ethoxy)carbonyl)amino)isoxazol-5-yl)phenoxy)methyl)cyclohexane-1-carboxylate (Intermediate R3)

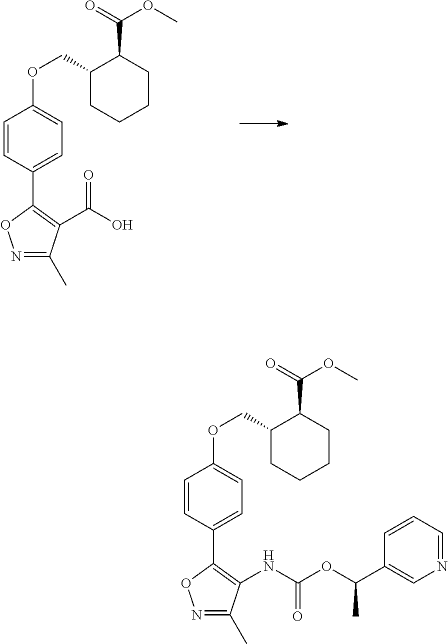 [SMILES: COC(=O)[C@H]1CCCC[C@@H]1COc1ccc(-c2onc(C)c2C(=O)O)cc1 ; COC(=O)[C@H]1CCCC[C@@H]1COc1ccc(-c2onc(C)c2NC(=O)O[C@H](C)c2cccnc2)cc1]

Intermediate R3 (0.023 g, 0.046 mmol, 54% yield) was obtained from 5-(4-(((1S,2S)-2-(methoxycarbonyl)cyclohexyl)methoxy)phenyl)-3-methylisoxazole-4-carboxylic acid (Intermediate R1, 0.032 g, 0.086 mmol) and (R)-1-(3-Pyridyl)ethanol (0.013 g, 0.103 mmol) using a similar method as for Intermediate A14.

LC-MS (ESI): m/z (M+1): 494.7 (Method 5)

¹H NMR (300 MHz, DMSO-d₆) δ 9.25 (s, 1H), 8.66 (s, 1H), 8.59-8.49 (m, 1H), 7.84 (d, J=8.2 Hz, 1H), 7.64 (d, J=8.5 Hz, 2H), 7.51-7.39 (m, 1H), 6.99 (d, J=8.4 Hz, 2H), 5.80 (q, J=6.8 Hz, 1H), 3.88 (d, J=5.6 Hz, 2H), 3.59 (s, 3H), 2.32-2.24 (m, 1H), 2.08 (s, 3H), 2.05-1.94 (m, 1H), 1.93-1.78 (m, 2H), 1.72 (d, J=8.7 Hz, 3H), 1.58 (d, J=6.6 Hz, 3H), 1.47-1.27 (m, 3H)

The Intermediate in the following table was prepared from reagents reported below by using methods analogous to Intermediate R3.

| Intermediate | Structure & Name | Reagents | Analytical data |
|---|---|---|---|
| R4 | 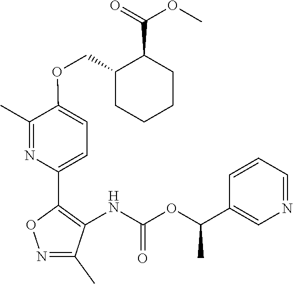 | R2 and (R)- 1-(3- Pyridyl) ethanol | LC-MS (ESI): m/z (M + 1): 509.9 (Method 5) ¹H NMR (300 MHz, DMSO-d₆) δ 9.12 (s, 1H), 8.65 (s, 1H), 8.52 (s, 1H), 7.83 (s, 1H), 7.59 (d, J = 8.5 Hz, 1H), 7.49-7.41 (m, 1H), 7.39 (d, J = 8.6 Hz, 1H), 5.79 (s, 1H), 3.99-3.77 (m, 2H), 3.56 (s, 3H), 2.33 (s, 3H), 2.15 (s, 3H), 2.14-2.04 (m, 1H), 1.94-1.84 (m, 2H), 1.78- 1.66 (m, 2H), 1.66- 1.49 (m, 3H), 1.45-1.24 (m, 5H) |
| | Methyl (1S,2S)-2-(((2- | | |
| | methyl-6-(3-methyl-4- | | |
| | ((((R)-1-(pyridin-3- | | |
| | yl)ethoxy)carbonyl)amino) | | |
| | isoxazol-5-yl)pyridin- | | |
| | 3- | | |
| | yl)oxy)methyl)cyclo- | | |
| | hexane-1-carboxylate | | |

### Step 3: (1S,2S)-2-((4-(3-methyl-4-((((R)-1-(pyridin-3-yl)ethoxy)carbonyl)amino)isoxazol-5-yl)phenoxy)methyl)cyclohexane-1-carboxylic acid (Compound 59)

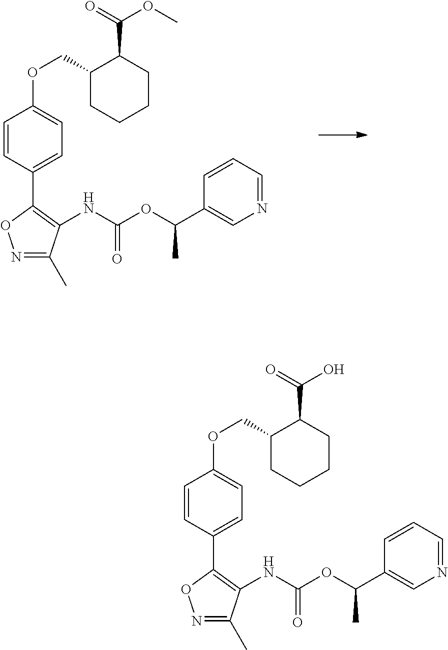 [SMILES: COC(=O)[C@H]1CCCC[C@@H]1COc1ccc(-c2onc(C)c2NC(=O)O[C@H](C)c2cccnc2)cc1 ; Cc1noc(-c2ccc(OC[C@H]3CCCC[C@@H]3C(=O)O)cc2)c1NC(=O)O[C@H](C)c1cccnc1]

Compound 59 (12 mg, 0.046 mmol, 31% yield) was obtained from methyl (1S,2S)-2-((4-(3-methyl-4-((((R)-1-(pyridin-3-yl)ethoxy)carbonyl)amino)isoxazol-5-yl)phenoxy)methyl)cyclohexane-1-carboxylate (Intermediate R3, 0.023 g, 0.147 mmol) using a similar method as for Compound 43.

LC-MS (ESI): m/z (M+1): 480.5 (Method 5)

¹H NMR (300 MHz, DMSO-d6) δ 12.28 (bs, 1H), 9.25 (s, 1H), 8.60 (d, J=32.1 Hz, 1H), 7.84 (d, J=7.9 Hz, 1H), 7.63 (d, J=8.4 Hz, 2H), 7.52-7.39 (m, 1H), 7.03 (d, J=8.6 Hz, 2H), 5.80 (q, J=6.7 Hz, 1H), 4.07-3.73 (m, 2H), 2.10 (d, J=7.7 Hz, 3H), 2.04-1.79 (m, 4H), 1.70 (s, 2H), 1.58 (d, J=6.6 Hz, 2H), 1.46-1.28 (m, 2H), 1.28-1.04 (m, 4H)

The Compound in the following table was prepared from reagents reported below by using methods analogous to Compound 59.

| Compound | Structure & Name | Reagents | Analytical data |
|---|---|---|---|
| 60 | 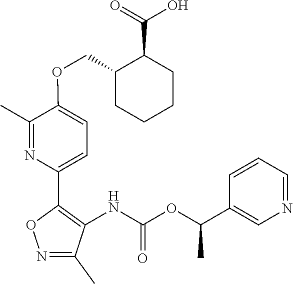 | R4 | LC-MS (ESI): m/z (M + 1): 509.9 (Method 11) ¹H NMR (300 MHz, DMSO-d6) δ 9.12 (s, 1H), 8.64 (s, 1H), 8.52 (s, 1H), 7.96-7.66 (m, 1H), 7.56 (t, J = 8.9 Hz, 2H), 7.46 (d, J = 23.6 Hz, 1H), 5.78 (s, 1H), 4.20-4.05 (m, 1H), 3.79 (t, J = 8.6 Hz, 1H), 2.34 (s, 3H), 2.14 (s, 3H), 1.89 (s, 4H), 1.73- 1.61 (m, 2H), 1.62-1.40 (m, 2H), 1.40-1.08 (m, 5H) |
| | methyl (1S,2S)-2-(((2- | | |
| | methyl-6-(3-methyl-4- | | |
| | ((((R)-1-(pyridin-3- | | |
| | yl)ethoxy)carbonyl)amino) | | |
| | isoxazol-5-yl)pyridin- | | |
| | 3- | | |
| | yl)oxy)methyl)cyclo- | | |
| | hexane-1-carboxylic acid | | |

### Example 22

### Process Synthesis of Compound 61

### (1S,2S)-2-(((2-methyl-6-(1-methyl-5-(((4-phenylpyrimidin-2-yl)amino)methyl)-1H-pyrazol-4-yl)pyridin-3-yl)oxy)methyl)cyclohexane-1-carboxylic acid

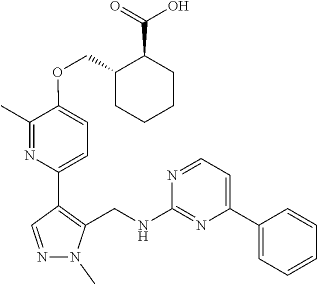 [SMILES: Cc1nc(-c2cnn(C)c2CNc2nccc(-c3ccccc3)n2)ccc1OC[C@H]1CCCC[C@@H]1C(=O)O]

### Step 1: (4-(5-(methoxymethoxy)-6-methylpyridin-2-yl)-1-methyl-1H-pyrazol-5-yl)methanol (Intermediate S1)

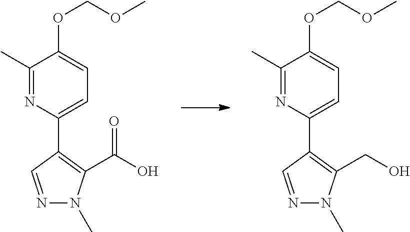

Intermediate S1 (97 mg, 0.368 mmol, 69.49% yield) was obtained from 4-(5-(methoxymethoxy)-6-methylpyridin-2-yl)-1-methyl-1H-pyrazole-5-carboxylic acid (Intermediate Q4, 147.0 mg, 0.530 mmol) using a similar method as for Intermediate D6.

LC-MS (ESI): m/z (M+1): 264.1 (Method 1)

The Intermediates in the following table were prepared from reagents reported below by using methods analogous Intermediate Si.

| Intermediate | Structure & Name | Reagents | Analytical data |
|---|---|---|---|
| S2 | 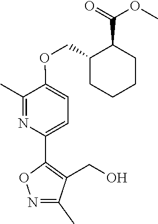 | R2 | LC-MS (ESI): m/z (M + 1): 375.2 (Method 2) |
| | methyl (1S,2S)-2-(((6-(4- | | |
| | (hydroxymethyl)-3- | | |
| | methylisoxazol-5-yl)-2- | | |
| | methylpyridin-3- | | |
| | yl)oxy)methyl)cyclo- | | |
| | hexane-1-carboxylate | | |

### Step 2: 6-(5-(chloromethyl)-1-methyl-1H-pyrazol-4-yl)-3-(methoxymethoxy)-2-methylpyridine (Intermediate S3)

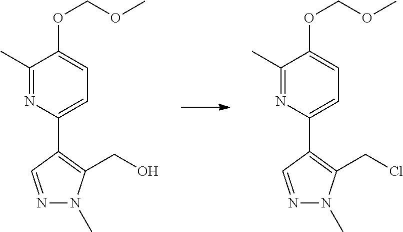

Intermediate S3 (500 mg, crude) was obtained from (4-(5-(methoxymethoxy)-6-methylpyridin-2-yl)-1-methyl-1H-pyrazol-5-yl)methanol (Intermediate Si, 97.0 mg, 0.370 mmol) using a similar method as for Intermediate D9.

LC-MS (ESI): m/z (M+1): 282.1 (Method 1)

### Step 3: (4-(5-(methoxymethoxy)-6-methylpyridin-2-yl)-1-methyl-1H-pyrazol-5-yl)methanamine (Intermediate S4)

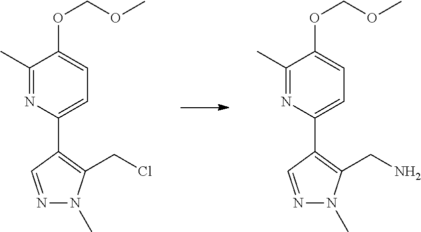

Intermediate S4 (500 mg, crude) was obtained from 6-(5-(chloromethyl)-1-methyl-1H-pyrazol-4-yl)-3-(methoxymethoxy)-2-methylpyridine (Intermediate S3, 500.0 mg, crude) using a similar method as for Intermediate D11.

LC-MS (ESI): m/z (M+1): 264.1 (Method 1)

The Intermediates in the following table were prepared from reagents reported below by using methods analogous Intermediate S4 (Step 2 and Step 3).

| Intermediate | Structure & Name | Reagents | Analytical data |
|---|---|---|---|
| S5 | 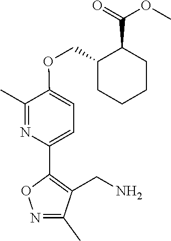 | S2 | LC-MS (ESI): m/z (M + 1): 374.2 (Method 2) |
| | methyl (1S,2S)-2-(((6-(4- | | |
| | (aminomethyl)-3- | | |
| | methylisoxazol-5-yl)-2- | | |
| | methylpyridin-3- | | |
| | yl)oxy)methyl)cyclo- | | |
| | hexane-1-carboxylate | | |

### Step 4: N-((4-(5-(methoxymethoxy)-6-methylpyridin-2-yl)-1-methyl-1H-pyrazol-5-yl)methyl)-4-phenylpyrimidin-2-amine (Intermediate S6)

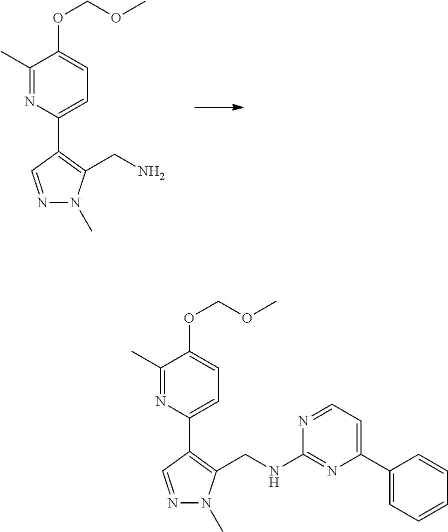

To a solution of (4-(5-(methoxymethoxy)-6-methylpyridin-2-yl)-1-methyl-1H-pyrazol-5-yl)methanamine (Intermediate S4, 50.0 mg, 0.190 mmol) in dry DMF (4 mL), K₂CO₃ (39.52 mg, 0.290 mmol) was added followed by 2-chloro-4-phenylpirimidine (39.97 mg, 0.210 mmol). The mixture was stirred at 80° C. overnight. Water and EtOAc were added and the mixture was extracted with EtOAc, collected organic phases were dried over Na2SO4, filtered and evaporated to give the crude material which was purified by flash chromatography using a gradient of EtOAc in CyHex from 40% to 80% affording the title compound (32 mg, 0.077 mmol, 40.31% yield) as colorless oil. LC-MS (ESI): m/z (M+1): 417.3 (Method 1)

### Step 5: 2-methyl-6-(1-methyl-5-(((4-phenylpyrimidin-2-yl)amino)methyl)-1H-pyrazol-4-yl)pyridin-3-ol (Intermediate S7)

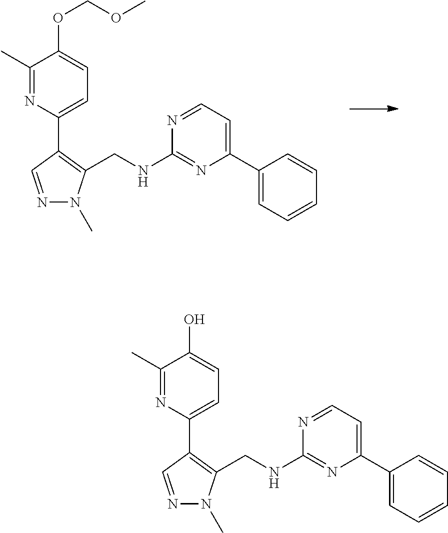 [SMILES: COCOc1ccc(-c2cnn(C)c2CNc2nccc(-c3ccccc3)n2)nc1C ; Cc1nc(-c2cnn(C)c2CNc2nccc(-c3ccccc3)n2)ccc1O]

Intermediate S7 (37 mg, crude)) was obtained from N-((4-(5-(methoxymethoxy)-6-methylpyridin-2-yl)-1-methyl-1H-pyrazol-5-yl)methyl)-4-phenylpyrimidin-2-amine (Intermediate S6, 32.0 mg, 0.080 mmol) using a similar method as for Intermediate Q8.

LC-MS (ESI): m/z (M+1): 373.1 (Method 1)

### Step 6: methyl (1S,2S)-2-(((2-methyl-6-(1-methyl-5-(((4-phenylpyrimidin-2-yl)amino)methyl)-1H-pyrazol-4-yl)pyridin-3-yl)oxy)methyl)cyclohexane-1-carboxylate (Intermediate S8)

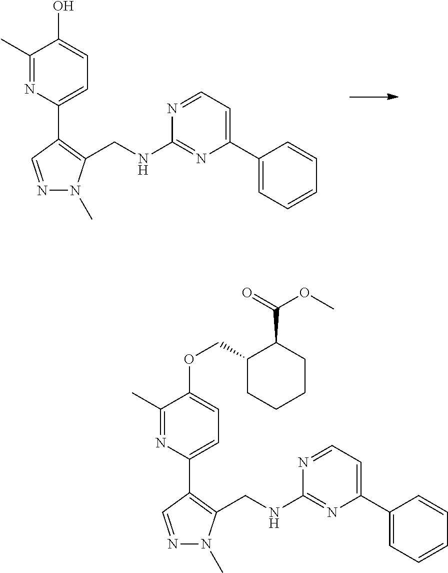 [SMILES: Cc1nc(-c2cnn(C)c2CNc2nccc(-c3ccccc3)n2)ccc1O ; COC(=O)[C@H]1CCCC[C@@H]1COc1ccc(-c2cnn(C)c2CNc2nccc(-c3ccccc3)n2)nc1C]

Intermediate S8 (90 mg, crude) was obtained from methyl (1S,2S)-2-(hydroxymethyl)cyclohexane-1-carboxylate (Intermediate O7, 34.22 mg, 0.200 mmol) and 2-methyl-6-(1-methyl-5-(((4-phenylpyrimidin-2-yl)amino)methyl)-1H-pyrazol-4-yl)pyridin-3-ol (Intermediate S7, 37.0 mg, 0.100 mmol) using a similar method as for Intermediate O9.

LC-MS (ESI): m/z (M+1): 527.3 (Method 1)

### Step 7: (1S,2S)-2-(((2-methyl-6-(1-methyl-5-(((4-phenylpyrimidin-2-yl)amino)methyl)-1H-pyrazol-4-yl)pyridin-3-yl)oxy)methyl)cyclohexane-1-carboxylic acid (Compound 61)

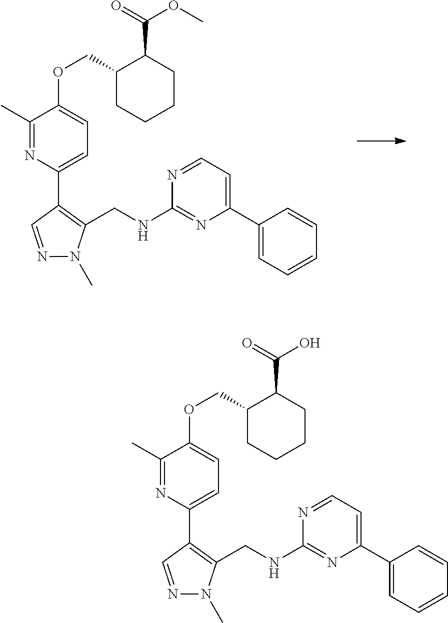 [SMILES: COC(=O)[C@H]1CCCC[C@@H]1COc1ccc(-c2cnn(C)c2CNc2nccc(-c3ccccc3)n2)nc1C ; Cc1nc(-c2cnn(C)c2CNc2nccc(-c3ccccc3)n2)ccc1OC[C@H]1CCCC[C@@H]1C(=O)O]

Compound 61 (1.3 mg, 0.003 mmol, 1.484% yield) was obtained as a white solid from methyl (1S,2S)-2-(((2-methyl-6-(1-methyl-5-(((4-phenylpyrimidin-2-yl)amino)methyl)-1H-pyrazol-4-yl)pyridin-3-yl)oxy)methyl)cyclohexane-1-carboxylate (Intermediate S8, 90.0 mg, 0.170 mmol) using a similar method as for Intermediate B4.

LC-MS (ESI): m/z (M+1): 513.2 (Method 1)

¹H NMR (500 MHz, Methanol-d4) δ ppm 8.30 (br d, J=5.1 Hz, 1H), 7.99-8.06 (m, 2H), 7.74 (s, 1H), 7.44-7.53 (m, 3H), 7.41 (d, J=8.5 Hz, 1H), 7.28 (d, J=8.5 Hz, 1H), 7.12 (d, J=5.4 Hz, 1H), 5.00 (s, 2H), 4.03 (s, 3H), 3.99 (dd, J=9.5, 4.5 Hz, 1H), 3.89 (dd, J=9.4, 6.1 Hz, 1H), 2.53 (s, 3H), 2.30 (td, J=11.4, 3.1 Hz, 1H), 2.11 (dt, J=10.1, 5.0 Hz, 1H), 2.01 (br s, 2H), 1.83 (br d, J=11.1 Hz, 2H), 1.47-1.61 (m, 1H), 1.22-1.46 (m, 3H)

### Example 23

### Process Synthesis of Compound 62

### (1S,2S)-2-(((6-(5-((((benzyloxy)carbonyl)amino)methyl)-1-methyl-1H-1,2,3-triazol-4-yl)-2-methylpyridin-3-yl)oxy)methyl)cyclohexane-1-carboxylic acid

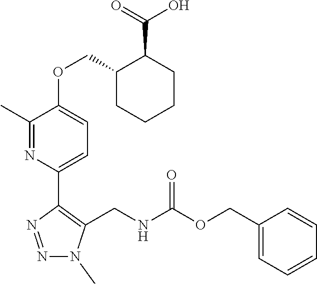 [SMILES: Cc1nc(-c2nnn(C)c2CNC(=O)OCc2ccccc2)ccc1OC[C@H]1CCCC[C@@H]1C(=O)O]

### Step 1: 3-bromo-2-methyl-6-(1-methyl-5-(((tetrahydro-2H-pyran-2-yl)oxy)methyl)-1H-1,2,3-triazol-4-yl)pyridine (Intermediate T1)

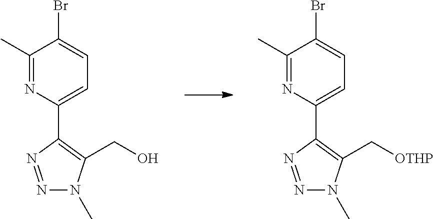

To a solution of (4-(5-bromo-6-methylpyridin-2-yl)-1-methyl-1H-1,2,3-triazol-5-yl)methanol (Intermediate K12, 356.0 mg, 1.26 mmol) in DCM (6 mL), at 0° C. were added 4-methylbenzenesulfonic acid hydrate (11.96 mg, 0.060 mmol) and 3,4-dihydro-2H-pyran (0.34 mL, 3.77 mmol). The mixture was then stirred at RT for 4h. NaHCO₃satd. sol. was added and the mixture was extracted with DCM, collected organic phases were dried over Na2SO4, filtered and evaporated to dryness. The crude was purified by flash chromatography (gradient CyHex/EtOAc from 9/1 to 1/1) to afford the title compound (414 mg, 1.127 mmol, 89.65% yield) as off-white solid.

LC-MS (ESI): m/z (M+1): 369.0 (Method 1)

### Step 2: (2-methyl-6-(1-methyl-5-(((tetrahydro-2H-pyran-2-yl)oxy)methyl)-1H-1,2,3-triazol-4-yl)pyridin-3-yl)boronic acid (Intermediate T2)

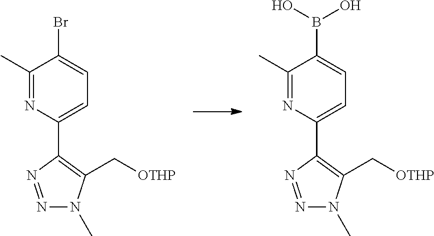

Intermediate T2 (970 mg, crude) was obtained from 3-bromo-2-methyl-6-(1-methyl-5-(((tetrahydro-2H-pyran-2-yl)oxy)methyl)-1H-1,2,3-triazol-4-yl)pyridine (Intermediate T1, 414.0 mg, 1.13 mmol) using a similar method as for Intermediate O1.

LC-MS (ESI): m/z (M+1): 333.1 (Method 1)

### Step 3: 2-methyl-6-(1-methyl-5-(((tetrahydro-2H-pyran-2-yl)oxy)methyl)-1H-1,2,3-triazol-4-yl)pyridin-3-ol (Intermediate T3)

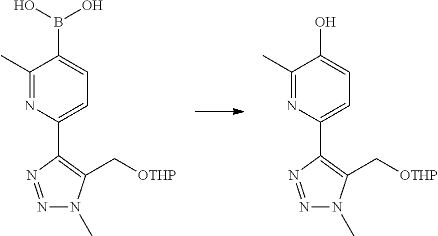

Intermediate T3 (266 mg, 0.874 mmol, 77.62% yield) was obtained from (2-methyl-6-(1-methyl-5-(((tetrahydro-2H-pyran-2-yl)oxy)methyl)-1H-1,2,3-triazol-4-yl)pyridin-3-yl)boronic acid (Intermediate T2, 374.0 mg, 1.13 mmol) using a similar method as for Intermediate O1.

LC-MS (ESI): m/z (M+1): 305.2 (Method 1)

### Step 4: methyl (1S,2S)-2-(((2-methyl-6-(1-methyl-5-(((tetrahydro-2H-pyran-2-yl)oxy)methyl)-1H-1,2,3-triazol-4-yl)pyridin-3-yl)oxy)methyl)cyclohexane-1-carboxylate (Intermediate T4)

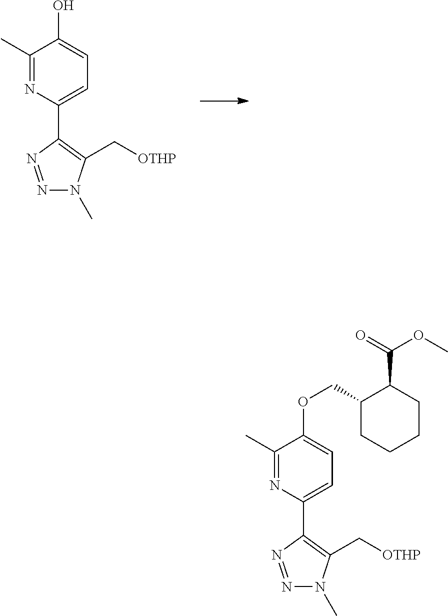

Intermediate T4 (389 mg, 0.848 mmol, 97.06% yield) was obtained as a colorless oil from methyl (1S,2S)-2-(hydroxymethyl)cyclohexane-1-carboxylate (Intermediate O7, 301.05 mg, 1.75 mmol) and 2-methyl-6-(1-methyl-5-(((tetrahydro-2H-pyran-2-yl)oxy)methyl)-1H-1,2,3-triazol-4-yl)pyridin-3-ol (Intermediate T3, 266.0 mg, 0.870 mmol) using a similar method as for Intermediate O9.

LC-MS (ESI): m/z (M+1): 459.3 (Method 1)

### Step 5: methyl (1S,2S)-2-(((6-(5-(hydroxymethyl)-1-methyl-1H-1,2,3-triazol-4-yl)-2-methylpyridin-3-yl)oxy)methyl)cyclohexane-1-carboxylate (Intermediate T5)

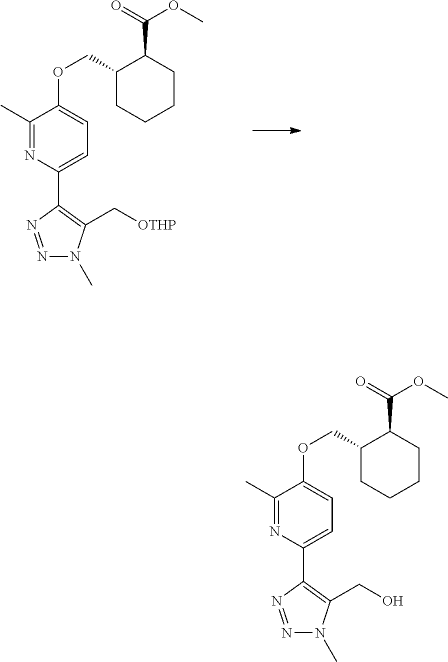

To a solution of methyl (1S,2S)-2-(((2-methyl-6-(1-methyl-5-(((tetrahydro-2H-pyran-2-yl)oxy)methyl)-1H-1,2,3-triazol-4-yl)pyridin-3-yl)oxy)methyl)cyclohexane-1-carboxylate (Intermediate T4, 389.0 mg, 0.850 mmol) in Meoh (8 mL) was added 4-methylbenzenesulfonate pyridin-1-ium (213.18 mg, 0.850 mmol). The mixture was stirred at 60° C. 3h. NaHCO₃satd solution (23 ml) was added for quenching, then MeOH was evaporated, water and et were added and the mixture was extracted with EtOAc, collected organic phases were dried over Na2SO4, filtered and evaporated to give the crude which was purified by flash chromatography (gradient CyHex/EtOAc from 6/4 to 0/10) to afford the target compound (230 mg, 0.614 mmol, 72.41% yield) as colorless oil.

LC-MS (ESI): m/z (M+1): 375.2 (Method 1)

### Step 6: methyl (1S,2S)-2-(((6-(5-(chloromethyl)-1-methyl-1H-1,2,3-triazol-4-yl)-2-methylpyridin-3-yl)oxy)methyl)cyclohexane-1-carboxylate (Intermediate T6)

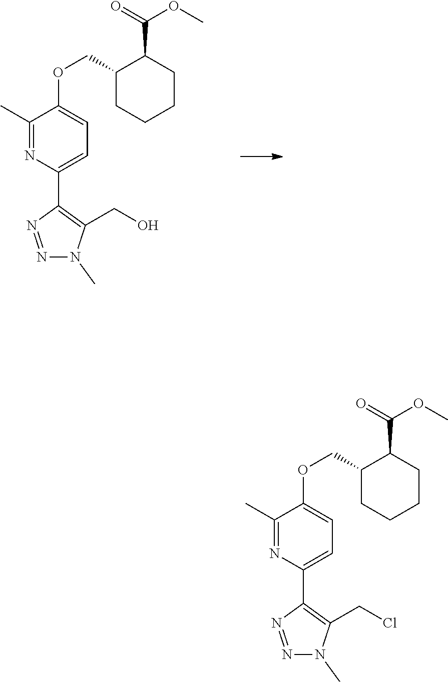

Intermediate T6 (700 mg, crude) was from methyl (1S,2S)-2-(((6-(5-(hydroxymethyl)-1-methyl-1H-1,2,3-triazol-4-yl)-2-methylpyridin-3-yl)oxy)methyl)cyclohexane-1-carboxylate (Intermediate T5, 170.0 mg, 0.450 mmol using a similar method as for Intermediate D9.

LC-MS (ESI): m/z (M+1): 393.2 (Method 1)

### Step 7: methyl (1S,2S)-2-(((6-(5-(azidomethyl)-1-methyl-1H-1,2,3-triazol-4-yl)-2-methylpyridin-3-yl)oxy)methyl)cyclohexane-1-carboxylate (Intermediate T7)

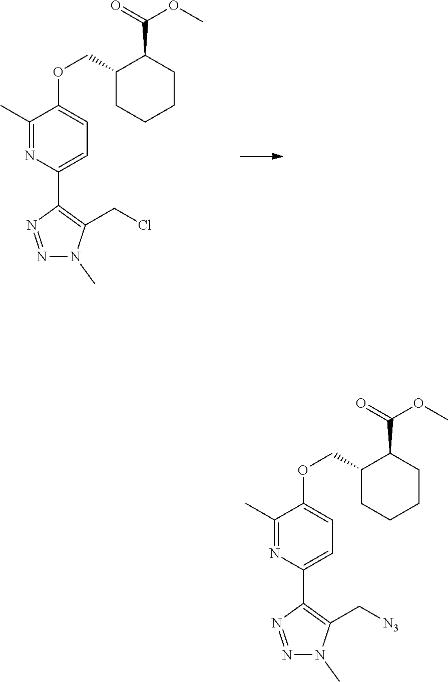

To a solution of methyl (1S,2S)-2-(((6-(5-(chloromethyl)-1-methyl-1H-1,2,3-triazol-4-yl)-2-methylpyridin-3-yl)oxy)methyl)cyclohexane-1-carboxylate (Intermediate T6, 178.0 mg, 0.450 mmol) in DMF (5 mL) was added NaN₃ (32.4 mg, 0.500 mmol) and the mixture was stirred at 80° C. for 5h. Water and EtOAc were added and the mixture was extracted with EtOAc, collected organic phases were dried over Na2SO₄, filtered and evaporated to give the title compound (156 mg, 0.391 mmol, 86.2% yield) as yellow oil which was used in the next step without further purification.

LC-MS (ESI): m/z (M+1): 400.2 (Method 1)

### Step 8: methyl (1S,2S)-2-(((6-(5-(aminomethyl)-1-methyl-1H-1,2,3-triazol-4-yl)-2-methylpyridin-3-yl)oxy)methyl)cyclohexane-1-carboxylate (Intermediate T8)

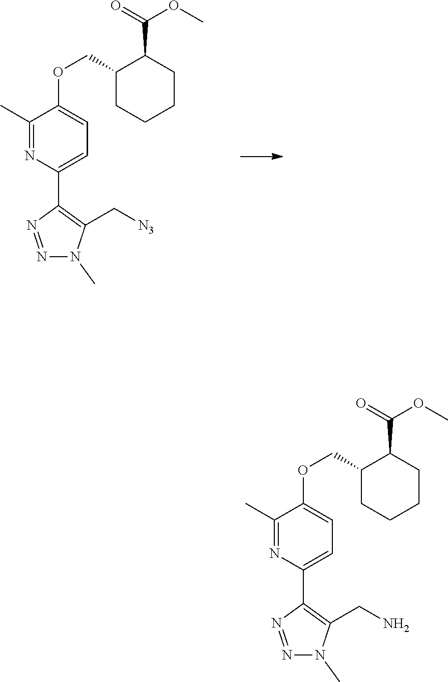

To a solution of methyl (1S,2S)-2-(((6-(5-(azidomethyl)-1-methyl-1H-1,2,3-triazol-4-yl)-2-methylpyridin-3-yl)oxy)methyl)cyclohexane-1-carboxylate (Intermediate T7, 156.0 mg, 0.390 mmol) in THE (3 mL) and Water (1 mL), PPh3 (153.65 mg, 0.590 mmol) was added and the mixture was stirred at RT overnight. Solvent was evaporated to give the crude which was purified by SCX and then by flash chromatography (gradient water/MeCN from 10/0 to 6/4) to afford the title compound (48 mg, 0.129 mmol, 32.91% yield) as colorless oil.

LC-MS (ESI): m/z (M+1): 360.9 (Method 1)

### Step 9: methyl (1S,2S)-2-(((6-(5-((((benzyloxy)carbonyl)amino)methyl)-1-methyl-1H-1,2,3-triazol-4-yl)-2-methylpyridin-3-yl)oxy)methyl)cyclohexane-1-carboxylate (Intermediate T9)

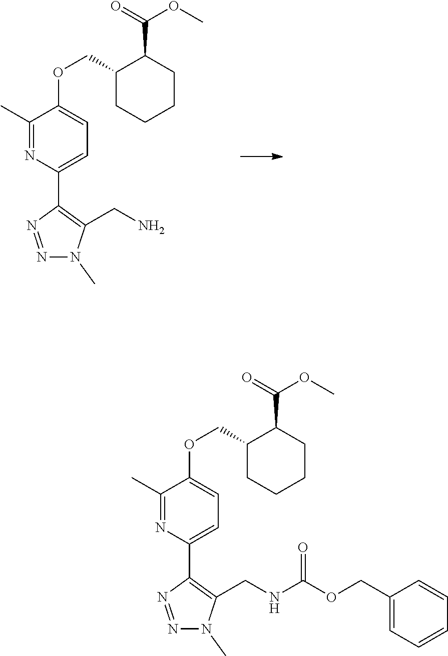

Intermediate T9 (40 mg, 0.079 mmol, 61.31% yield) was obtained from methyl (1S,2S)-2-(((6-(5-(aminomethyl)-1-methyl-1H-1,2,3-triazol-4-yl)-2-methylpyridin-3-yl)oxy)methyl)cyclohexane-1-carboxylate (Intermediate T8, 48.0 mg, 0.130 mmol), and carbonochloridic acid (phenylmethyl) ester (0.02 mL, 0.130 mmol) using a similar method as for Intermediate H1.

LC-MS (ESI): m/z (M+1): 508.3 (Method 1)

### Step 10: (1S,2S)-2-(((6-(5-((((benzyloxy)carbonyl)amino)methyl)-1-methyl-1H-1,2,3-triazol-4-yl)-2-methylpyridin-3-yl)oxy)methyl)cyclohexane-1-carboxylic acid (Compound 62)

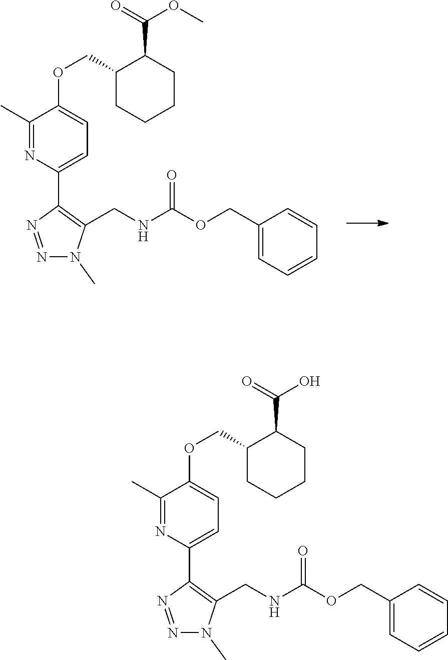 [SMILES: COC(=O)[C@H]1CCCC[C@@H]1COc1ccc(-c2nnn(C)c2CNC(=O)OCc2ccccc2)nc1C ; Cc1nc(-c2nnn(C)c2CNC(=O)OCc2ccccc2)ccc1OC[C@H]1CCCC[C@@H]1C(=O)O]

Compound 62 (17.1 mg, 0.035 mmol, 43.97% yield) was obtained as a white solid from methyl (1S,2S)-2-(((6-(5-((((benzyloxy)carbonyl)amino)methyl)-1-methyl-1H-1,2,3-triazol-4-yl)-2-methylpyridin-3-yl)oxy)methyl)cyclohexane-1-carboxylate (Intermediate T9, 40.0 mg, 0.080 mmol) using a similar method as for Intermediate B4.

LC-MS (ESI): m/z (M+1): 494.2 (Method 1)

¹H NMR (500 MHz, DMSO-d6) δ ppm 10.86-13.47 (m, 1H), 7.81 (d, J=8.5 Hz, 1H), 7.68 (br t, J=4.7 Hz, 1H), 7.41 (br d, J=8.5 Hz, 1H), 7.11-7.37 (m, 5H), 4.88-5.10 (m, 2H), 4.79 (br d, J=5.4 Hz, 2H), 4.04 (s, 4H), 3.85 (dd, J=9.6, 6.4 Hz, 1H), 2.40 (s, 3H), 2.09-2.22 (m, 1H), 1.94-2.07 (m, 1H), 1.82-1.94 (m, 2H), 1.72 (br d, J=9.1 Hz, 2H), 1.33-1.46 (m, 1H), 1.16-1.32 (m, 3H)

The Compounds in the following table were prepared from reagents reported below by using methods analogous Intermediate 16.

| Compound | Structure & Name | Reagents | Analytical data |
|---|---|---|---|
| 63 | 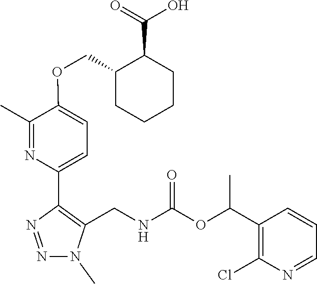 | T8 and I4 | LC-MS (ESI): m/z (M + 1): 556.7 (Method 2) ¹H NMR (300 MHz, DMSO-d₆) δ 8.59 (t, J = 5.4 Hz, 1H), 8.30 (dd, J = 4.7, 1.7 Hz, 1H), 7.89- 7.76 (m, 3H), 7.49 (dd, J = 8.0, 4.7 Hz, 1H), 7.41 (d, J = 8.6 Hz, 1H), 4.95 (d, J = 5.2 Hz, 2H), 4.69 (d, J = 5.9 Hz, 1H), 4.08 (s, 3H), 3.95 (dd, J = 9.8, 4.9 Hz, 2H), 3.84 (dd, J = 10.0, 5.6 Hz, 2H), 3.56 (d, J = 2.3 Hz, 4H), 2.41 (s, 4H), 2.33 (s, 3H), 2.09-1.96 (m, 2H), 1.93-1.83 (m, 3H), |
| | (1S,2S)-2-(((6-(5-(((((R)- | | 1.76-1.69 (m, 2H), 1.48- |
| | 1-(2-chloropyridin-3- | | 1.32 (m, 3H), 1.33- |
| | yl)ethoxy)carbonyl)amino) | | 1.18 (m, 7H) |
| | methyl)-1-methyl-1H- | | |
| | 1,2,3-triazol-4-yl)-2- | | |
| | methylpyridin-3- | | |
| | yl)oxy)methyl)cyclo- | | |
| | hexane-1-carboxylic acid | | |
| 64 | 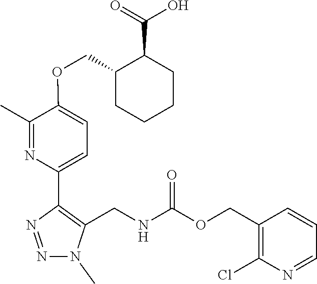 [SMILES: Cc1nc(-c2nnn(C)c2CNC(=O)OCc2cccnc2Cl)ccc1OC[C@H]1CCCC[C@@H]1C(=O)O] | T8 and I5 | LC-MS (ESI): m/z (M + 1): 530.0 (Method 2) ¹H NMR (300 MHz, DMSO-d₆) δ 12.20 (s, 1H), 8.37 (dd, J = 4.7, 1.9 Hz, 1H), 7.91-7.77 (m, 3H), 7.48-7.36 (m, 2H), 5.09 (s, 2H), 4.82 (d, J = 5.4 Hz, 2H), 4.04 (s, 3H), 3.96 (dd, J = 9.4, 4.2 Hz, 1H), 3.86 (q, J = 6.3 Hz, 1H), 2.40 (s, 3H), 2.20 (td, J = 10.9, 3.2 Hz, 1H), 2.03- 1.85 (m, 3H), 1.78-1.65 (m, 2H), 1.47-1.33 (m, 1H), 1.30-1.19 (m, 3H) |
| | (1S,2S)-2-(((6-(5-(((((2- | | |
| | chloropyridin-3- | | |
| | yl)methoxy)carbonyl) | | |
| | amino)methyl)-1-methyl- | | |
| | 1H-1,2,3-triazol-4-yl)-2- | | |
| | methylpyridin-3- | | |
| | yl)oxy)methyl)cyclo- | | |
| | hexane-1-carboxylic acid | | |

### Example 24

### Process Synthesis of Compound 65

### (1S,2S)-2-(((2-methyl-6-(1-methyl-5-(((4-(pyrazin-2-yl)pyrimidin-2-yl)amino)methyl)-1H-1,2,3-triazol-4-yl)pyridin-3-yl)oxy)methyl)cyclohexane-1-carboxylic acid

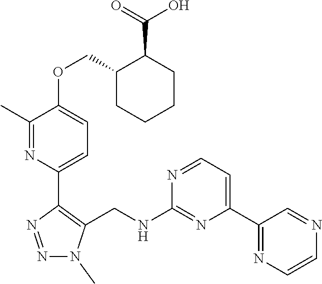 [SMILES: Cc1nc(-c2nnn(C)c2CNc2nccc(-c3cnccn3)n2)ccc1OC[C@H]1CCCC[C@@H]1C(=O)O]

### Step 1: methyl (1S,2S)-2-(((2-methyl-6-(1-methyl-5-(((4-(pyrazin-2-yl)pyrimidin-2-yl)amino)methyl)-1H-1,2,3-triazol-4-yl)pyridin-3-yl)oxy)methyl)cyclohexane-1-carboxylate (Intermediate U1)

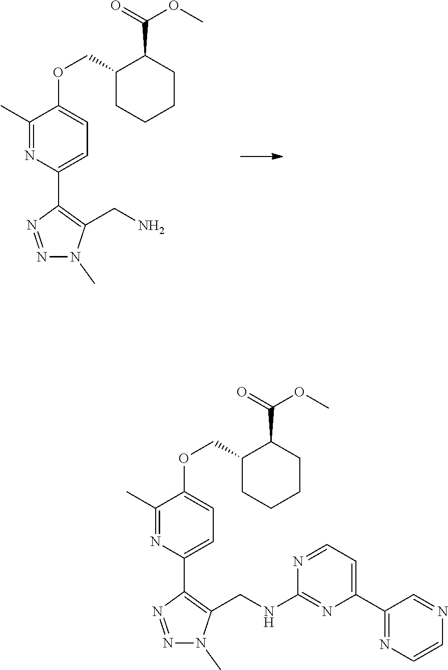

Intermediate U1 (0.012 g, 0.021 mmol) was obtained from methyl (1S,2S)-2-(((6-(5-(aminomethyl)-1-methyl-1H-1,2,3-triazol-4-yl)-2-methylpyridin-3-yl)oxy)methyl)cyclohexane-1-carboxylate (Intermediate T8, 0.065 g, 0.174 mmol) and 2-chloro-4-(pyrazin-2-yl)pyrimidine (0.033 g, 0.192 mmol) using a similar method as for Intermediate D14.

LC-MS (ESI): m/z (M+1): 529.7 (Method 3)

¹H NMR (300 MHz, DMSO-d₆) δ 7.87-7.75 (m, 2H), 7.47 (d, J=8.6 Hz, 1H), 7.32-7.17 (m, 3H), 5.06 (s, 2H), 4.81 (d, J=5.6 Hz, 2H), 4.04 (s, 3H), 3.60 (s, 3H), 2.78-2.67 (m, 1H), 2.41 (s, 3H), 2.09-1.99 (m, 1H), 1.92-1.74 (m, 3H), 1.69-1.44 (m, 4H)

The Intermediate in the following table was prepared from reagents reported below by using methods analogous as for Intermediate U1.

| Intermediate | Structure & Name | Reagents | Analytical data |
|---|---|---|---|
| U2 | 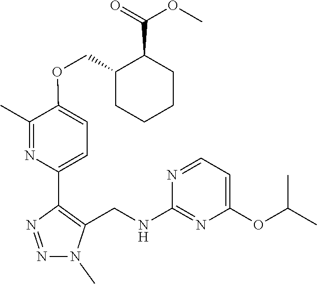 [SMILES: COC(=O)[C@H]1CCCC[C@@H]1COc1ccc(-c2nnn(C)c2CNc2nccc(OC(C)C)n2)nc1C] | T8 and D14 | LC-MS (ESI): m/z (M + 1): 510.7 (Method 4) ¹H NMR (300 MHz, DMSO-d₆) δ 7.85 (d, J = 8.6 Hz, 1H), 7.52 (d, J = 5.3 Hz, 1H), 7.50 (s, 1H), 7.42 (d, J = 8.7 Hz, 1H), 7.21 (s, 1H), 5.18-5.03 (m, 2H), 4.80 (p, J = 6.1 Hz, 1H), 4.06 (s, 3H), 3.95 (dd, J = 9.8, 4.7 Hz, 1H), 3.85 (dd, J = 9.8, 5.6 Hz, 1H), 3.57 (s, 3H), 2.42-2.30 (m, 4H), 2.11- 2.01 (m, 1H), 1.89 (d, J = 10.7 Hz, 2H), 1.73 (s, 2H), 1.48-1.35 (m, 1H), 1.32- |
| | methyl (1S,2S)-2-(((6-(5- | | 1.22 (m, 3H), 1.06 (dd, |
| | (((4- | | J = 6.1, 2.1 Hz, 6H) |
| | isopropoxypyrimidin-2- | | |
| | yl)amino)methyl)-1- | | |
| | methyl-1H-1,2,3-triazol- | | |
| | 4-yl)-2-methylpyridin-3- | | |
| | yl)oxy)methyl)cyclo- | | |
| | hexane-1-carboxylate | | |
| U3 | 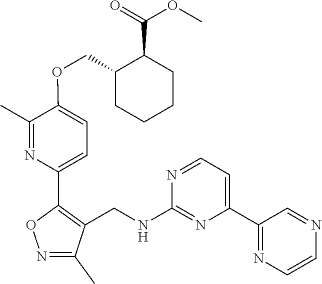 | S5 and 2- chloro-4- (pyrazin-2- yl)pyrimidine | LC-MS (ESI): m/z (M + 1): 516.2 (Method 5) ¹H NMR (300 MHz, DMSO-d₆) δ 12.17 (s, 1H), 9.30 (s, 1H), 8.76 (d, J = 1.3 Hz, 2H), 8.54-8.47 (m, 1H), 7.76-7.68 (m, 2H), 7.51-7.43 (m, 2H), 4.86 (s, 2H), 4.01 (dd, J = 9.9, 4.5 Hz, 1H), 3.94 (dd, J = 9.8, 5.7 Hz, 1H), 2.44 (s, 3H), 2.33 (s, 3H), 2.21 (dd, J = 11.2, 3.6 Hz, 1H), 2.02 (d, J = 5.4 Hz, 1H), 1.91 (s, 2H), 1.72 (s, 2H), 1.41 (d, J = 11.9 Hz, 1H), 1.26 (t, J = 7.9 Hz, 4H) |
| | methyl (1S,2S)-2-(((2- | | |
| | methyl-6-(3-methyl-4- | | |
| | (((4-(pyrazin-2- | | |
| | yl)pyrimidin-2- | | |
| | yl)amino)methyl)isoxazol- | | |
| | 5-yl)pyridin-3- | | |
| | yl)oxy)methyl)cyclo- | | |
| | hexane-1-carboxylate | | |

### Step 2: (1S,2S)-2-(((2-methyl-6-(1-methyl-5-(((4-(pyrazin-2-yl)pyrimidin-2-yl)amino)methyl)-1H-1,2,3-triazol-4-yl)pyridin-3-yl)oxy)methyl)cyclohexane-1l-carboxylic acid (Compound 65)

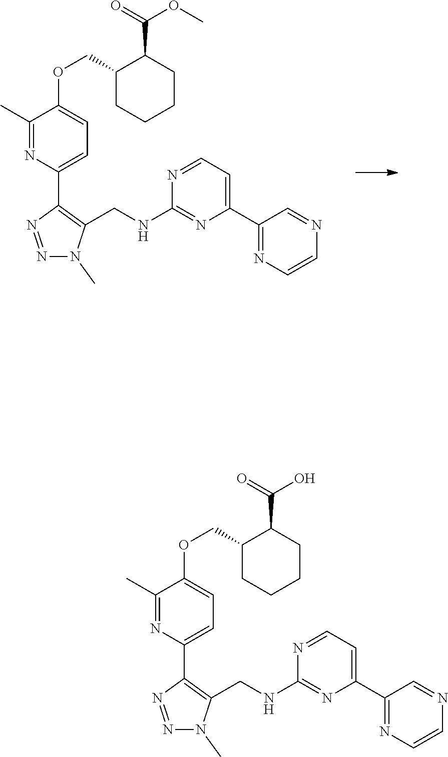 [SMILES: COC(=O)[C@H]1CCCC[C@@H]1COc1ccc(-c2nnn(C)c2CNc2nccc(-c3cnccn3)n2)nc1C ; Cc1nc(-c2nnn(C)c2CNc2nccc(-c3cnccn3)n2)ccc1OC[C@H]1CCCC[C@@H]1C(=O)O]

Compound 65 (0.0033 g, 0.0061 mmol, 29% yield) was obtained from methyl (1S,2S)-2-(((2-methyl-6-(1-methyl-5-(((4-(pyrazin-2-yl)pyrimidin-2-yl)amino)methyl)-1H-1,2,3-triazol-4-yl)pyridin-3-yl)oxy)methyl)cyclohexane-1-carboxylate (Intermediate U1, 0.012 g, 0.021 mmol) using a similar method as for Intermediate B4.

LC-MS (ESI): m/z (M+1): 543.2 (Method 6)

¹H NMR (400 MHz, DMSO-d₆) δ 8.34 (d, J=4.1 Hz, 1H), 7.88-7.78 (m, 3H), 7.47-7.36 (m, 2H), 5.85 (q, J=6.2 Hz, 1H), 4.77 (d, J=5.4 Hz, 2H), 4.00 (s, 3H), 3.98-3.94 (m, 1H), 3.89-3.81 (m, 1H), 2.38 (s, 3H), 2.23-2.12 (m, 1H), 2.03-1.95 (m, 1H), 1.94-1.86 (m, 2H), 1.76-1.68 (m, 2H), 1.44 (d, J=6.6 Hz, 3H), 1.37 (s, 1H), 1.29-1.19 (m, 4H)

The Compound in the following table was prepared from reagents reported below by using methods analogous as for Compound 65.

| Compound | Structure & Name | Reagents | Analytical data |
|---|---|---|---|
| 66 | 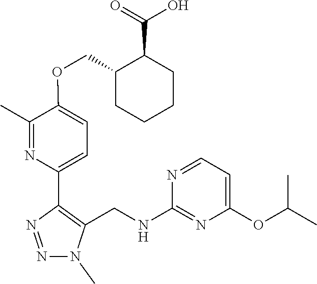 [SMILES: Cc1nc(-c2nnn(C)c2CNc2nccc(OC(C)C)n2)ccc1OC[C@H]1CCCC[C@@H]1C(=O)O] | U2 | LC-MS (ESI): m/z (M + 1): 496.2 (Method 6) ¹H NMR (300 MHz, DMSO-d₆) δ 12.15 (s, 1H), 8.05-7.92 (m, 1H), 7.85 (d, J = 8.5 Hz, 1H), 7.61 (s, 1H), 7.43 (d, J = 8.6 Hz, 1H), 5.96 (d, J = 5.5 Hz, 1H), 5.00 (s, 2H), 4.09 (s, 3H), 3.93 (dd, J = 10.9, 5.1 Hz, 2H), 2.42 (s, 3H), 2.26-2.16 (m, 1H), 2.09- 1.83 (m, 3H), 1.81- 1.69 (m, 2H), 1.43 (s, 1H), 1.34-1.20 (m, 4H), 1.16- 0.96 (m, 6H) |
| | (1S,2S)-2-(((6-(5-(((4- | | |
| | isopropoxypyrimidin-2- | | |
| | yl)amino)methyl)-1- | | |
| | methyl-1H-1,2,3-triazol- | | |
| | 4-yl)-2-methylpyridin-3- | | |
| | yl)oxy)methyl)cyclo- | | |
| | hexane-1-carboxylic acid | | |
| 67 | 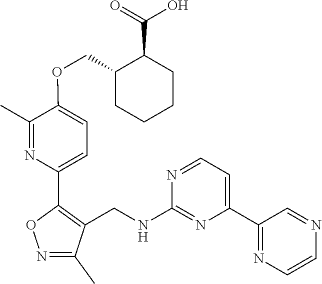 | U3 | LC-MS (ESI): m/z (M + 1): 480.0 (Method 11) ¹H NMR (300 MHz, DMSO-d₆) δ 9.29 (s, 1H), 8.75 (s, 2H), 8.50 (d, J = 5.0 Hz, 1H), 7.72 (d, J = 8.2 Hz, 2H), 7.51-7.38 (m, 2H), 4.85 (s, 2H), 4.00 (dd, J = 9.8, 4.9 Hz, 1H), 3.89 (dd, J = 9.9, 5.6 Hz, 1H), 3.55 (s, 3H), 2.42 (s, 3H), 2.33 (s, 4H), 2.05 (s, 1H), 1.89 (d, J = 12.2 Hz, 2H), 1.72 (s, 2H), 1.41 (d, J = 12.3 Hz, 1H), 1.32- 1.17 (m, 4H) |
| | (1S,2S)-2-(((2-methyl-6- | | |
| | (3-methyl-4-(((4- | | |
| | (pyrazin-2-yl)pyrimidin- | | |
| | 2- | | |
| | yl)amino)methyl)isoxazol- | | |
| | 5-yl)pyridin-3- | | |
| | yl)oxy)methyl)cyclo- | | |
| | hexane-1-carboxylic acid | | |

### Example 25

### Process Synthesis of Compound 68

### (1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylic acid

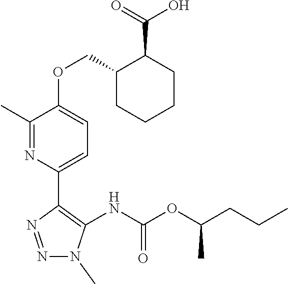 [SMILES: CCC[C@@H](C)OC(=O)Nc1c(-c2ccc(OC[C@H]3CCCC[C@@H]3C(=O)O)c(C)n2)nnn1C]

### Step 1: 4-(5-bromo-6-methylpyridin-2-yl)-1-methyl-1H-1,2,3-triazole-5-carboxylic acid (Intermediate V1)

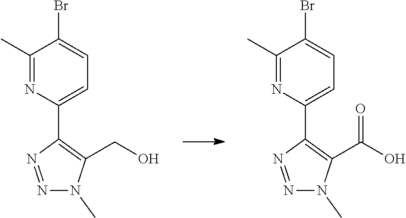 [SMILES: Cc1nc(-c2nnn(C)c2CO)ccc1Br ; Cc1nc(-c2nnn(C)c2C(=O)O)ccc1Br]

To a suspension of (4-(5-bromo-6-methylpyridin-2-yl)-1-methyl-1H-1,2,3-triazol-5-yl)methanol (Intermediate K12, 2.09 g, 7.39 mmol) in Water (25 mL), NaOH (591.41 mg, 14.79 mmol) was added followed by the addition of KMnO₄ (2.34 g, 14.79 mmol). The mixture was stirred at 100° C. for 1.5 h. The reaction mixture was allowed to cool to RT than adjusted to pH=2-3 with HCl 3N. The mixture was extracted with DCM (3 times). Combined organics were dried over Na2SO4, filtered through a hydrophobic phase separator and concentrated at reduced pressure to provide the title compound (1156 mg, 3.891 mmol, 52.63% yield) as a white solid. The material was used in the next step without further purification.

LC-MS (ESI): m/z (M+1): 299.0 (Method 1)

The Intermediate in the following table was prepared from reagents reported below by using methods analogous to intermediate V1.

| Intermediate | Structure & Name | Reagents | Analytical data |
|---|---|---|---|
| V2 | 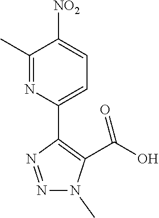 | K11 | LC-MS (ESI): m/z (M + 1): 260.0 (Method 1) 1H NMR (400 MHz, DMSO-d6) d ppm 8.75 (d, J = 8.80 Hz, 1 H), 8.35 (d, J = 8.58 Hz, 1 H), 4.32 (s, 3 H), 2.85 (s, 3 H) |
| | 1-methyl-4-(6-methyl-5- | | |
| | nitropyridin-2-yl)-1H- | | |
| | 1,2,3-triazole-5- | | |
| | carboxylic acid | | |
| V3 | 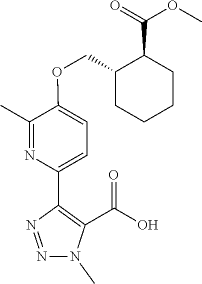 | T5 | LC-MS (ESI): m/z (M + 1): 361.6 (Method 4) ¹H NMR (300 MHz, DMSO-d₆) δ 7.86 (d, J = 8.6 Hz, 1H), 7.51 (d, J = 8.7 Hz, 1H), 5.77 (t, J = 5.8 Hz, 1H), 5.02 (d, J = 5.8 Hz, 2H), 4.84-4.77 (m, 1H), 4.06 (s, 3H), 3.61 (s, 3H), 2.78-2.64 (m, 1H), 2.44 (s, 3H), 2.11- 2.01 (m, 1H), 1.87-1.76 (m, 3H), 1.69-1.48 (m, 4H) |
| | 4-(5-(((1S,2S)-2- | | |
| | (methoxycarbonyl)cyclo | | |
| | hexyl)methoxy)-6- | | |
| | methylpyridin-2-yl)-1- | | |
| | methyl-1H-1,2,3- | | |
| | triazole-5-carboxylic acid | | |

### Step 2: (R)-pentan-2-yl (4-(5-bromo-6-methylpyridin-2-yl)-1-methyl-1H-1,2,3-triazol-5-yl)carbamate (Intermediate V4)

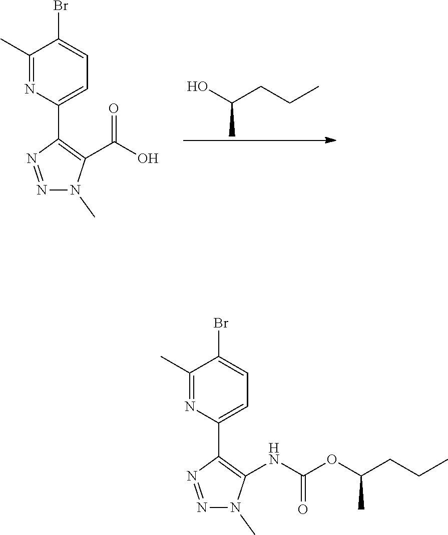

Intermediate V4 (93 mg, 0.243 mmol, 72.28% yield) was obtained as a white solid from 4-(5-bromo-6-methylpyridin-2-yl)-1-methyl-1H-1,2,3-triazole-5-carboxylic acid (Intermediate V1, 100.0 mg, 0.340 mmol) and (R)-pentan-2-ol (0.04 mL, 0.400 mmol) using a similar method as for Intermediate A14.

LC-MS (ESI): m/z (M+1): 384.1 (Method 1)

The Intermediate in the following table was prepared from reagents reported below by using methods analogous to Intermediate V4.

| Intermediate | Structure & Name | Reagents | Analytical data |
|---|---|---|---|
| V5 | 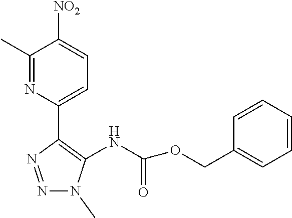 [SMILES: Cc1nc(-c2nnn(C)c2NC(=O)OCc2ccccc2)ccc1[N+](=O)[O-]] | V2 and phenyl- methanol | LC-MS (ESI): m/z (M + 1): 369.1 (Method 1) 1H NMR (400 MHz, DMSO-d6) δ ppm 9.72- 9.95 (m, 1 H) 8.39-8.57 (m, 1 H) 7.92-8.12 (m, 1 H) 6.97-7.55 (m, 5 H) 4.95-5.25 (m, 2 H) 3.94 (s, 3 H) 2.69 (s, 3 H) |
| | benzyl (1-methyl-4-(6- | | |
| | methyl-5-nitropyridin-2- | | |
| | yl)-1H-1,2,3-triazol-5- | | |
| | yl)carbamate | | |
| V6 | 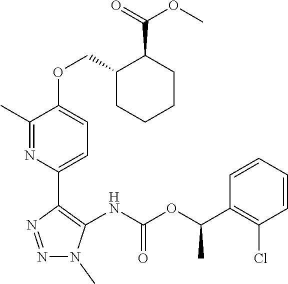 [SMILES: COC(=O)[C@H]1CCCC[C@@H]1COc1ccc(-c2nnn(C)c2NC(=O)O[C@H](C)c2ccccc2Cl)nc1C] | V3 and (R)- 1-(2- chlorophenyl) ethan-1-ol | LC-MS (ESI): m/z (M+1): 542.7 (Method 10) ¹H NMR (300 MHz, DMSO-d₆) δ 9.66 (s, 1H), 7.71 (d, J = 8.5 Hz, 1H), 7.67-7.10 (m, 5H), 6.06- 5.88 (m, 1H), 3.99- 3.90 (m, 1H), 3.86 (s, 3H), 3.56 (s, 3H), 2.40-2.34 (m, 1H), 2.28 (s, 3H), 2.11- 2.00 (m, 2H), 1.87 (s, 2H), 1.79-1.65 (m, 2H), 1.65-1.39 (m, 3H), 1.34- 1.24 (m, 4H) |
| | methyl (1S,2S)-2-(((6-(5- | | |
| | ((((R)-1-(2- | | |
| | chlorophenyl)ethoxy) | | |
| | carbonyl)amino)-1-methyl- | | |
| | 1H-1,2,3-triazol-4-y1)-2- | | |
| | methylpyridin-3- | | |
| | yl)oxy)methyl)cyclohexane- | | |
| | 1-carboxylate | | |

### Step 3: (R)-pentan-2-yl (4-(5-hydroxy-6-methylpyridin-2-yl)-1-methyl-1H-1,2,3-triazol-5-yl)carbamate (Intermediate V7)

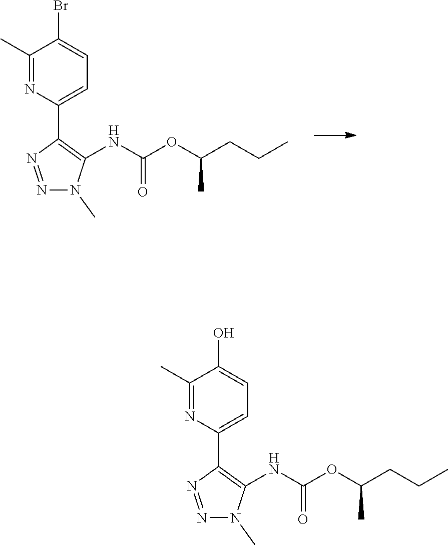

Intermediate V7 compound (132 mg, 0.413 mmol, 169.89% yield) was obtained from (4-(5-bromo-6-methylpyridin-2-yl)-1-methyl-1H-1,2,3-triazol-5-yl)carbamate (Intermediate V4, 93.0 mg, 0.240 mmol) using a similar method as for Intermediate O1.

LC-MS (ESI): m/z (M+1): 320.1 (Method 1)

The intermediate in the following table was prepared from reagents reported below by using methods analogous to Intermediate V7.

| Intermediate | Structure & Name | Reagents | Analytical data |
|---|---|---|---|
| V8 | 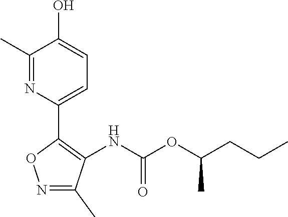 [SMILES: CCC[C@@H](C)OC(=O)Nc1c(C)noc1-c1ccc(O)c(C)n1] | A18 | LC-MS (ESI): m/z (M + 1): 320.1 (Method 1) 1H NMR (400 MHz, DMSO-d6) δ ppm 10.19- 10.69 (m, 1 H), 8.52-8.85 (m, 1 H), 7.51 (d, J = 8.36 Hz, 1 H), 7.22 (d, J = 8.36 Hz, 1 H), 4.61-4.81 (m, 1 H), 2.37 (s, 3 H), 2.17 (s, 3 H), 0.99-1.69 (m, 7 H), 0.66-0.99 (m, 3 H) |
| | (R)-pentan-2-yl (5-(5- | | |
| | hydroxy-6- | | |
| | methylpyridin-2-y1)-3- | | |
| | methylisoxazol-4- | | |
| | yl)carbamate | | |

### Step 4: methyl (1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylate (Intermediate V9)

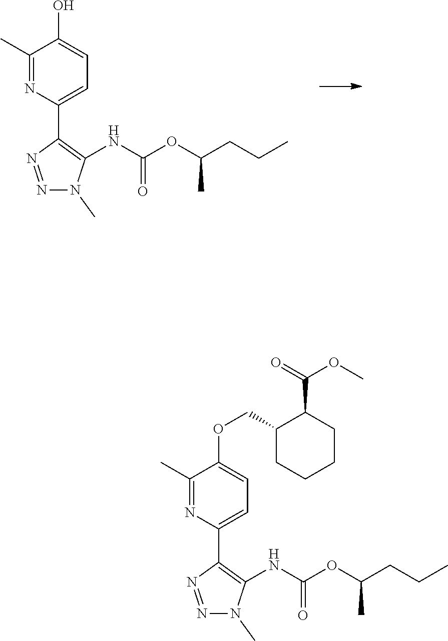

Intermediate V9 (124 mg, 0.262 mmol, 107.75% yield) was obtained from (4-(5-hydroxy-6-methylpyridin-2-yl)-1-methyl-1H-1,2,3-triazol-5-yl)carbamate (Intermediate V7, 77.6 mg, 0.240 mmol) and methyl (1S,2S)-2-(hydroxymethyl)cyclohexane-1-carboxylate (Intermediate O7, 54.4 mg, 0.320 mmol) using a similar method as for Intermediate O2.

LC-MS (ESI): m/z (M+1): 479.4 (Method 1)

The intermediate in the following table was prepared from reagents reported below by using methods analogous to Intermediate V9.

| Intermediate | Structure & Name | Reagents | Analytical data |
|---|---|---|---|
| V10 | 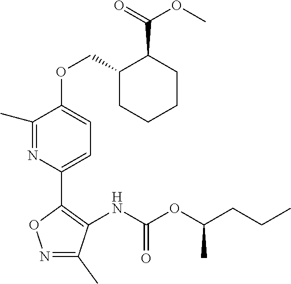 [SMILES: CCC[C@@H](C)OC(=O)Nc1c(C)noc1-c1ccc(OC[C@H]2CCCC[C@@H]2C(=O)OC)c(C)n1] | V8 | LC-MS (ESI): m/z (M + 1): 474.3 (Method 1) |
| | methyl (1S,2S)-2-(((2- | | |
| | methyl-6-(3-methyl-4- | | |
| | (((((R)-pentan-2- | | |
| | yl)oxy)carbonyl)amino) | | |
| | isoxazol-5-yl)pyridin-3- | | |
| | yl)oxy)methyl)cyclohexane- | | |
| | 1-carboxylate | | |

### Step 5: (1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylic acid (Compound 68)

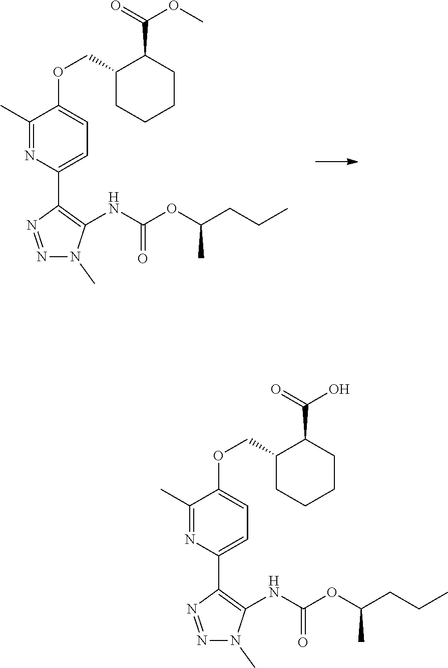

Compound 68 (16 mg, 0.035 mmol, 14.33% yield) was obtained as a white solid from methyl (1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylate (Intermediate V9, 115.08 mg, 0.240 mmol) using a similar method as for Intermediate A9.

LC-MS (ESI): m/z (M+1): 460.4 (Method 1)

¹H NMR (500 MHPz, DMSO-d6) δ ppm 11.63-12.51 (m, 1H), 8.85-9.58 (m, 1H), 7.71 (d, J=8.5 Hz, 1H), 7.37 (d, J=8.6 Hz, 1H), 4.51-4.87 (m, 1H), 3.91-3.97 (m, 1H), 3.85-3.89 (m, 1H), 3.85 (s, 3H), 2.37 (s, 3H), 2.21 (td, J=11.3, 3.6 Hz, 1H), 1.95-2.04 (m, 1H), 1.82-1.94 (m, 2H), 1.67-1.79 (m, 2H), 1.34-1.45 (m, 1H), 0.53-1.65 (m, 13H)

The Compounds in the following table were prepared from reagents reported below by using methods analogous to Compound 68.

| Compound | Structure & Name | Reagents | Analytical data |
|---|---|---|---|
| 69 | 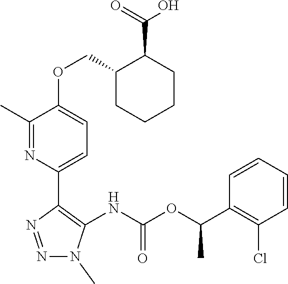 [SMILES: Cc1nc(-c2nnn(C)c2NC(=O)O[C@H](C)c2ccccc2Cl)ccc1OC[C@H]1CCCC[C@@H]1C(=O)O] | V6 | LC-MS (ESI): m/z (M + 1): 527.3 (Method 6) ¹H NMR (300 MHz, DMSO-d₆) δ 12.13 (s, 1H), 9.68 (s, 1H), 7.69 (d, J = 8.5 Hz, 2H), 7.65-7.21 (m, 6H), 5.99 (s, 1H), 3.86 (s, 5H), 2.31 (s, 3H), 2.27- 2.17 (m, 1H), 2.04- 1.84 (m, 4H), 1.78-1.69 (m, 2H), 1.58-1.49 (m, 1H), 1.45-1.37 (m, 1H), 1.30-1.22 (m, 4H) |
| | (1S,2S)-2-(((2-methyl-6- | | |
| | (1-methyl-5-(((((R)- | | |
| | pentan-2- | | |
| | yl)oxy)carbonyl)amino)- | | |
| | 1H-1,2,3-triazol-4- | | |
| | yl)pyridin-3- | | |
| | yl)oxy)methyl)cyclohexane- | | |
| | 1-carboxylic acid | | |
| 70 | 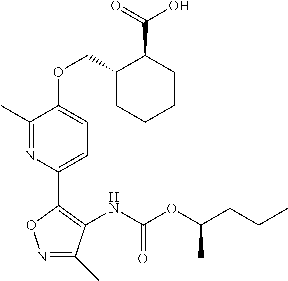 [SMILES: CCC[C@@H](C)OC(=O)Nc1c(C)noc1-c1ccc(OC[C@H]2CCCC[C@@H]2C(=O)O)c(C)n1] | V10 | MS (ESI): m/z (M + 1): 460.2 (Method 1) 1H NMR (500 MHz, DMSO-d6) δ ppm 12.20 (br s, 1 H), 8.35-9.21 (m, 1 H), 7.61 (d, J = 8.5 Hz, 1 H), 7.45 (d, J = 8.6 Hz, 1 H), 4.71 (br s, 1 H), 3.87- 4.03 (m, 2 H), 2.40 (s, 3 H), 2.18-2.25 (m, 1 H), 2.17 (s, 3 H), 1.96-2.07 (m, 1 H), 1.84-1.95 (m, 2 H), 1.72 (br d, J = 9.5 Hz, 2 H), 0.64-1.66 (m, 14 H) |
| | (1S,2S)-2-(((2-methyl-6- | | |
| | (1-methyl-5-(((((R)- | | |
| | pentan-2- | | |
| | yl)oxy)carbonyl)amino)- | | |
| | 1H-1,2,3-triazol-4- | | |
| | yl)pyridin-3- | | |
| | yl)oxy)methyl)cyclohexane- | | |
| | 1-carboxylic acid | | |

### Example 26

### Process Synthesis of Compound 71

### (1S,2S)-2-(((6-(5-((6-isopropoxypyrazin-2-yl)amino)-1-methyl-1H-1,2,3-triazol-4-yl)-2-methylpyridin-3-yl)oxy)methyl)cyclohexane-1-carboxylic acid

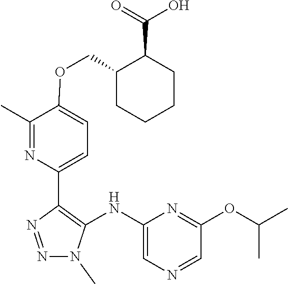 [SMILES: Cc1nc(-c2nnn(C)c2Nc2cncc(OC(C)C)n2)ccc1OC[C@H]1CCCC[C@@H]1C(=O)O]

### Step 1: benzyl (4-(5-amino-6-methylpyridin-2-yl)-1-methyl-1H-1,2,3-triazol-5-yl)carbamate (Intermediate W1)

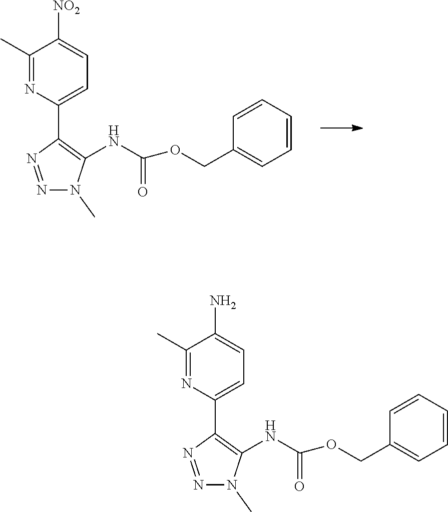 [SMILES: Cc1nc(-c2nnn(C)c2NC(=O)OCc2ccccc2)ccc1[N+](=O)[O-] ; Cc1nc(-c2nnn(C)c2NC(=O)OCc2ccccc2)ccc1N]

Intermediate W1 (150 mg, 0.443 mmol, 98.97% yield) was obtained from benzyl (1-methyl-4-(6-methyl-5-nitropyridin-2-yl)-1H-1,2,3-triazol-5-yl)carbamate (Intermediate V5, 165.0 mg, 0.450 mmol) using a similar method as for Intermediate C3.

LC-MS (ESI): m/z (M+1): 339.2 (Method 1)

### Step 2: benzyl (4-(5-hydroxy-6-methylpyridin-2-yl)-1-methyl-1H-1,2,3-triazol-5-yl)carbamate (Intermediate W2)

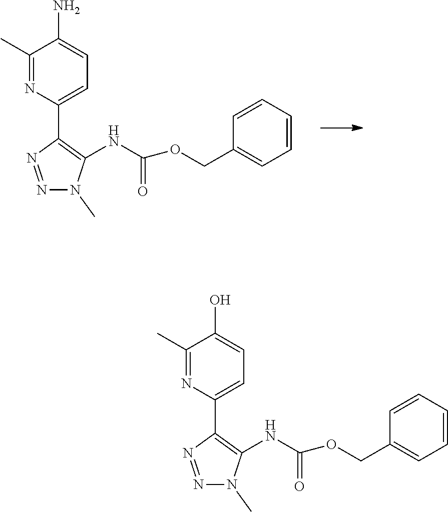 [SMILES: Cc1nc(-c2nnn(C)c2NC(=O)OCc2ccccc2)ccc1N ; Cc1nc(-c2nnn(C)c2NC(=O)OCc2ccccc2)ccc1O]

To a solution of benzyl (4-(5-amino-6-methylpyridin-2-yl)-1-methyl-1H-1,2,3-triazol-5-yl)carbamate (Intermediate W1, 150.0 mg, 0.440 mmol) in 2 M H2SO4 (6.59 mL, 13.19 mmol) at 0° C. was added a solution of sodium nitrite (0.03 g, 0.490 mmol) in Water (4 mL). The reaction mixture was stirred at RT for 1 h, then at 90° C. for 30 minutes. NaOH 2 M was added until pH 5, then the mixture was extracted with EtOAc (3 times), collected organic phase was dried over Na2SO4, filtered and evaporated to give the crude product (111 mg, 0.327 mmol, 73.78% yield) as yellow solid which was used in the next step without further purification.

LC-MS (ESI): m/z (M+1): 340.1 (Method 1)

### Step 3: methyl (1S,2S)-2-(((6-(5-(((benzyloxy)carbonyl)amino)-1-methyl-1H-1,2,3-triazol-4-yl)-2-methylpyridin-3-yl)oxy)methyl)cyclohexane-1-carboxylate (Intermediate W3)

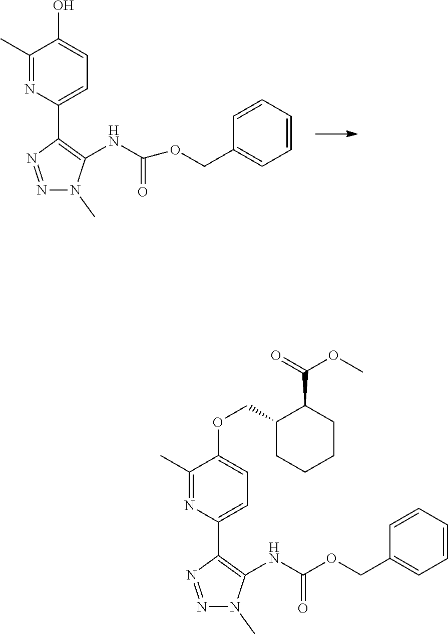 [SMILES: Cc1nc(-c2nnn(C)c2NC(=O)OCc2ccccc2)ccc1O ; COC(=O)[C@H]1CCCC[C@@H]1COc1ccc(-c2nnn(C)c2NC(=O)OCc2ccccc2)nc1C]

Intermediate W3 (157 mg, 0.318 mmol, 97.25% yield) was obtained from methyl (1S,2S)-2-(hydroxymethyl)cyclohexane-1-carboxylate (Intermediate O7, 112.66 mg, 0.650 mmol) and benzyl (4-(5-hydroxy-6-methylpyridin-2-yl)-1-methyl-1H-1,2,3-triazol-5-yl)carbamate (Intermediate W2, 111.0 mg, 0.330 mmol) using a similar method as for Intermediate O9.

LC-MS (ESI): m/z (M+1): 494.2 (Method 1)

### Step 4: methyl (1S,2S)-2-(((6-(5-amino-1-methyl-1H-1,2,3-triazol-4-yl)-2-methylpyridin-3-yl)oxy)methyl)cyclohexane-1-carboxylate (Intermediate W4)

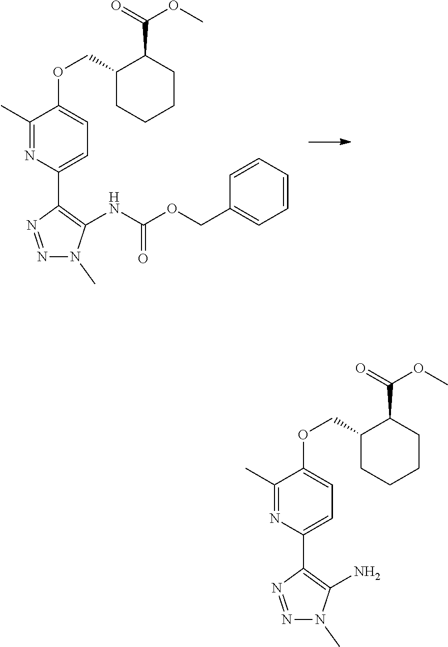

Intermediate W4 (92 mg, 0.256 mmol, 80.47% yield) was obtained as yellow oil, from methyl (1S,2S)-2-(((6-(5-(((benzyloxy)carbonyl)amino)-1-methyl-1H-1,2,3-triazol-4-yl)-2-methylpyridin-3-yl)oxy)methyl)cyclohexane-1-carboxylate (Intermediate W3, 157.0 mg, 0.320 mmol) using a similar method as for Intermediate A21.

LC-MS (ESI): m/z (M+1): 360.3 (Method 1)

### Step 5: methyl (1S,2S)-2-(((6-(5-((6-isopropoxypyrazin-2-yl)amino)-1-methyl-1H-1,2,3-triazol-4-yl)-2-methylpyridin-3-yl)oxy)methyl)cyclohexane-1-carboxylate (Intermediate W5)

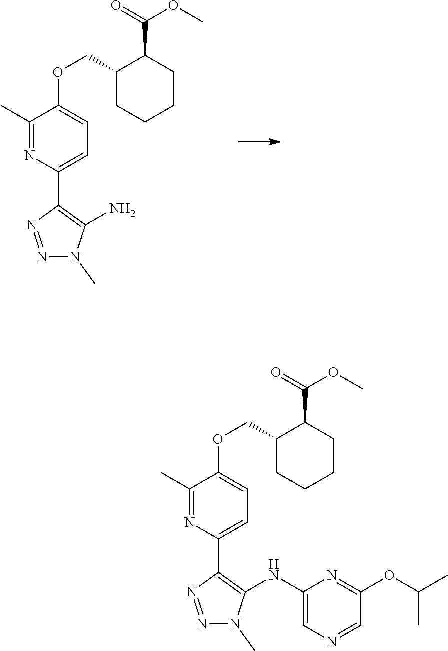

Intermediate W5 (56.8 mg, 0.115 mmol, 44.78% yield) was obtained from methyl (1S,2S)-2-(((6-(5-amino-1-methyl-1H-1,2,3-triazol-4-yl)-2-methylpyridin-3-yl)oxy)methyl)cyclohexane-1-carboxylate (Intermediate W4, 92.0 mg, 0.260 mmol) and 2-chloro-6-isopropoxypyrazine (Intermediate A23, 91.17 mg, 0.330 mmol) using a similar method as for Intermediate A25.

LC-MS (ESI): m/z (M+1): 496.3 (Method 1)

¹H NMR (400 MHz, DMSO-d6) δ ppm 9.11 (s, 1H) 7.67-7.79 (m, 2H) 7.48 (s, 1H) 7.28-7.36 (m, 1H) 4.58-4.76 (m, 1H) 3.82-3.97 (m, 5H) 2.08 (s, 3H) 1.99 (s, 3H) 1.80-1.92 (m, 3H) 1.61-1.75 (m, 3H) 1.20-1.33 (m, 4H) 1.04 (d, J=6.16 Hz, 6H)

The Intermediate in the following table were prepared from reagents reported below by using methods analogous to Intermediate W5.

| Intermediate | Structure & Name | Reagents | Analytical data |
|---|---|---|---|
| W6 | 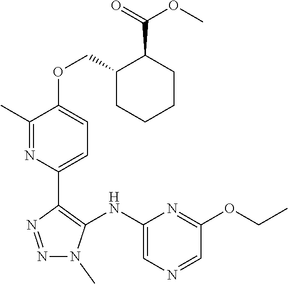 [SMILES: CCOc1cncc(Nc2c(-c3ccc(OC[C@H]4CCCC[C@@H]4C(=O)OC)c(C)n3)nnn2C)n1] | W4 and 2- chloro-6- ethoxy- pyrazine | LC-MS (ESI): m/z (M + 1): 482.3 (Method 1) 1H NMR (400 MHz, Chloroform-d) δ 8.96 (s, 1H), 7.93 (d, J = 8.53 Hz, 1H), 7.81 (s, 1H), 7.75 (s, 1H), 7.16 (d, J = 8.56 Hz, 1H), 4.23 (q, J = 7.09 Hz, 2H), 4.07 (s, 3H), 3.91 (dd, J = 9.28, 4.21 Hz, 1H), 3.81 (dd, J = 9.35, 5.82 Hz, 1H), 3.65 (s, 3H), 2.49 (s, 3H), 2.37 (td, J = 11.56, 3.68 Hz, 1H), 2.21- 1.77 (bm, 5H), 1.55- 1.07 (bm, 7H). |
| | (1S,2S)-2-(((6-(5-((6- | | |
| | ethoxypyrazin-2- | | |
| | yl)amino)-1-methyl-1H- | | |
| | 1,2,3-triazol-4-yl)-2- | | |
| | methylpyridin-3- | | |
| | yl)oxy)methyl)cyclohexane- | | |
| | 1-carboxylate | | |
| W7 | 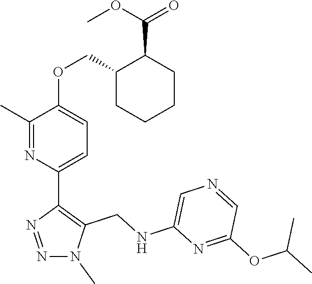 [SMILES: COC(=O)[C@H]1CCCC[C@@H]1COc1ccc(-c2nnn(C)c2CNc2cncc(OC(C)C)n2)nc1C] | T8 and A23 | LC-MS (ESI): m/z (M + 1): 510.7 (Method 4) ¹H NMR (300 MHz, DMSO-d₆) δ 7.85 (d, J = 8.6 Hz, 1H), 7.52 (d, J = 5.3 Hz, 1H), 7.50 (s, 1H), 7.42 (d, J = 8.7 Hz, 1H), 7.21 (s, 1H), 5.18-5.03 (m, 2H), 4.80 (p, J = 6.1 Hz, 1H), 4.06 (s, 3H), 3.95 (dd, J = 9.8, 4.7 Hz, 1H), 3.85 (dd, J = 9.8, 5.6 Hz, 1H), 3.57 (s, 3H), 2.42- 2.30 (m, 4H), 2.11-2.01 (m, 1H), 1.89 (d, J = 10.7 Hz, 2H), 1.73 (s, 2H), 1.48- 1.35 (m, 1H), 1.32- |
| | | | 1.22 (m, 3H), 1.06 (dd, J = |
| | (1S,2S)-2-(((6-(5-(((6- | | 6.1, 2.1 Hz, 6H) |
| | isopropoxypyrazin-2- | | |
| | yl)amino)methyl)-1- | | |
| | methyl-1H-1,2,3-triazol- | | |
| | 4-y1)-2-methylpyridin-3- | | |
| | yl)oxy)methyl)cyclohexane- | | |
| | 1-carboxylate | | |

### Step 6: (1S,2S)-2-(((6-(5-((6-isopropoxypyrazin-2-yl)amino)-1-methyl-1H-1,2,3-triazol-4-yl)-2-methylpyridin-3-yl)oxy)methyl)cyclohexane-1-carboxylic acid (Compound 71)

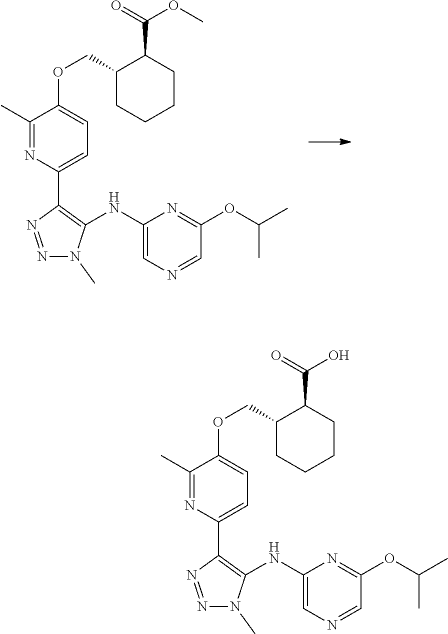

Compound 71 (11.2 mg, 0.023 mmol, 20.29% yield) was obtained as a white solid from methyl (1S,2S)-2-(((6-(5-((6-isopropoxypyrazin-2-yl)amino)-1-methyl-1H-1,2,3-triazol-4-yl)-2-methylpyridin-3-yl)oxy)methyl)cyclohexane-1-carboxylate (Intermediate V5, 56.8 mg, 0.110 mmol) using a similar method as for Intermediate B4.

LC-MS (ESI): m/z (M+1): 482.3 (Method 1)

¹H NMR (400 MHz, DMSO-d₆) δ ppm 12.10 (br s, 1H), 9.10 (s, 1H), 7.72 (d, J=8.6 Hz, 1H), 7.70 (s, 1H), 7.48 (s, 1H), 7.32 (d, J=8.6 Hz, 1H), 4.68 (spt, J=6.2 Hz, 1H), 3.92 (s, 3H), 3.86-3.91 (m, 1H), 3.77-3.85 (m, 1H), 2.18 (td, J=11.2, 3.5 Hz, 1H), 2.10 (s, 3H), 1.91-2.01 (m, 1H), 1.87-1.91 (m, 1H), 1.79-1.86 (m, 1H), 1.70 (br d, J=8.0 Hz, 2H), 1.38 (q, J=11.9 Hz, 1H), 1.13-1.30 (m, 3H), 1.04 (d, J=6.1 Hz, 6H)

The Compounds in the following table were prepared from reagents reported below by using methods analogous to Compound 71.

| Compound | Structure & Name | Reagents | Analytical data |
|---|---|---|---|
| 72 | 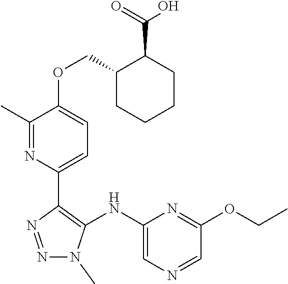 [SMILES: CCOc1cncc(Nc2c(-c3ccc(OC[C@H]4CCCC[C@@H]4C(=O)O)c(C)n3)nnn2C)n1] | W6 | LC-MS (ESI): m/z (M + 1): 468.4 (Method 1) 1H NMR (500 MHz, DMSO-d6) δ ppm 10.67- 13.38 (m, 1 H), 9.15 (s, 1 H), 7.69-7.75 (m, 2 H), 7.54 (s, 1 H), 7.34 (d, J = 8.6 Hz, 1 H), 3.85-3.99 (m, 6 H), 3.78-3.84 (m, 1 H), 2.12 (s, 3 H), 2.07- 2.24 (m, 1 H), 1.80-2.00 (m, 3 H), 1.64-1.77 (m, 2 H), 1.31-1.44 (m, 1 H), 1.15-1.31 (m, 3 H), 1.11 (t, J = 7.1 Hz, 3 H |
| | (1S,2S)-2-(((6-(5-((6- | | |
| | ethoxypyrazin-2- | | |
| | yl)amino)-1-methyl-1H- | | |
| | 1,2,3-triazol-4-y1)-2- | | |
| | methylpyridin-3- | | |
| | yl)oxy)methyl)cyclohexane- | | |
| | 1-carboxylic acid | | |
| 73 | 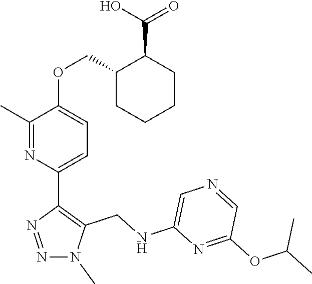 [SMILES: Cc1nc(-c2nnn(C)c2CNc2cncc(OC(C)C)n2)ccc1OC[C@H]1CCCC[C@@H]1C(=O)O] | W7 | LC-MS (ESI): m/z (M + 1): 494.3 (OO11 ¹H NMR (300 MHz, DMSO-d₆) δ 12.12 (s, 1H), 7.85 (d, J = 8.6 Hz, 1H), 7.59-7.46 (m, 2H), 7.43 (d, J = 8.6 Hz, 1H), 7.22 (s, 1H), 5.11 (d, J = 5.4 Hz, 2H), 4.87-4.73 (m, 1H), 4.06 (s, 3H), 4.00- 3.83 (m, 2H), 2.38 (s, 3H), 2.27-2.13 (m, 1H), 2.06- 1.82 (m, 3H), 1.78- 1.65 (m, 2H), 1.33-1.19 (m, 4H), 1.06 (d, J = 6.1 Hz, 6H) |
| | (1S,2S)-2-(((6-(5-( (6- | | |
| | isopropoxypyrazin-2- | | |
| | yl)amino)methyl)-1- | | |
| | methyl-1H-1,2,3-triazol- | | |
| | 4-y1)-2-methylpyridin-3- | | |
| | yl)oxy)methyl)cyclohexane- | | |
| | 1-carboxylic acid | | |

### Example 27

### Process Synthesis of Compound 74

### (1S,2S)-2-(((6-(4-(((cyclopentyl(methyl)carbamoyl)oxy)methyl)-3-methylisoxazol-5-yl)-2-methylpyridin-3-yl)oxy)methyl)cyclohexane-1-carboxylic acid

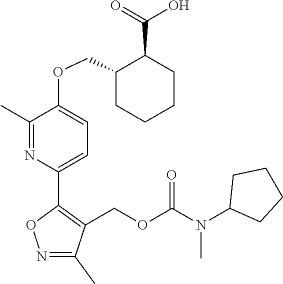 [SMILES: Cc1nc(-c2onc(C)c2COC(=O)N(C)C2CCCC2)ccc1OC[C@H]1CCCC[C@@H]1C(=O)O]

### Step 1: (5-(5-bromo-6-methylpyridin-2-yl)-3-methylisoxazol-4-yl)methyl cyclopentyl(methyl)carbamate (Intermediate X1)

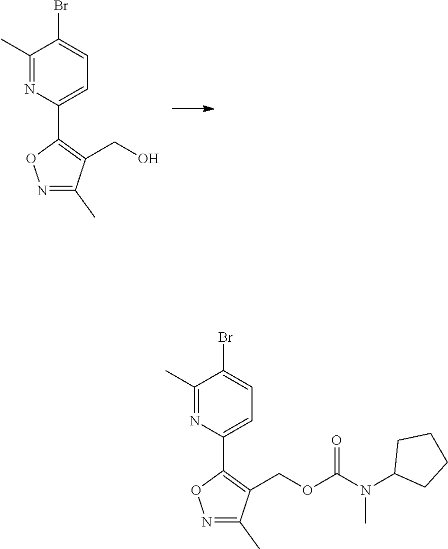 [SMILES: Cc1nc(-c2onc(C)c2CO)ccc1Br ; Cc1nc(-c2onc(C)c2COC(=O)N(C)C2CCCC2)ccc1Br]

Sodium hydride (25.49 mg, 0.640 mmol) (60% dispersion in mineral oil) was added at 0° C. to a solution of (5-(5-bromo-6-methylpyridin-2-yl)-3-methylisoxazol-4-yl)methanol (Intermediate D8, 164.0 mg, 0.580 mmol) in dry THE (3 mL). The mixture was stirred at this temperature for 10 min, then a solution of cyclopentyl(methyl)carbamic chloride (121.71 mg, 0.750 mmol) in dry THE (1 mL) was added and the mixture was stirred at 0° C. for 40 min, then it was allowed to warm up at RT. After 7 h the mixture was cooled down to 0° C. and an addition of sodium hydride (12 mg, 0.29 mmol) was made. After 10 min a solution of cyclopentyl(methyl)carbamic chloride (47 mg, 0.29 mmol) in dry THE (1 mL) was added. The mixture was then allowed to warm up at room temperature and stirred overnight. A saturated aqueous NH₄Cl solution was then added and the mixture was extracted with ethyl acetate (3 times). The mixed organic phases were dried over Na₂SO₄, filtered and concentrated in vacuo. The crude was purified by flash chromatography (gradient CyHex/EtOAc from 95/5 to 60/40) to give the title compound as a colorless oil (222 mg, 0.543 mmol, 93.9% yield).

LC-MS (ESI): m/z (M+1): 408.1 (Method 1)

The Intermediates in the following table were prepared from reagents reported below by using methods analogous to Intermediate X1.

| Intermediate | Structure & Name | Reagents | Analytical data |
|---|---|---|---|
| X2 | 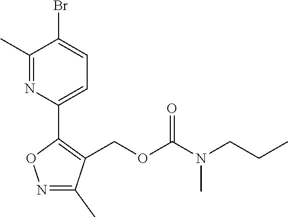 [SMILES: CCCN(C)C(=O)OCc1c(C)noc1-c1ccc(Br)c(C)n1] | D8 and N- methyl-1- propanamine | LC-MS (ESI): m/z (M + 1): 382.0 (Method 1) ¹H NMR (400 MHz, DMSO-d6) δ ppm 8.22 (d, J = 8.36 Hz, 1 H), 7.69 (d, J = 8.36 Hz, 1 H), 5.36 (br. s., 2 H), 3.00-3.22 (m, 2 H), 2.78 (br. s., 3 H), 2.65 (s, 3 H), 2.35 (s, 3 H), 1.25- 1.54 (m, 2 H), 0.58-0.87 (m, 3 H) |
| | (5-(5-bromo-6- | | |
| | methylpyridin-2-yl)-3- | | |
| | methylisoxazol-4- | | |
| | yl)methyl | | |
| | methyl(propyl)carbamate | | |
| X3 | 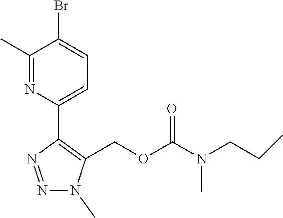 [SMILES: CCCN(C)C(=O)OCc1c(-c2ccc(Br)c(C)n2)nnn1C] | K12 and N- methyl-1- propanamine | LC-MS (ESI): m/z (M + 1): 382.0 (Method 1) ¹H NMR (400 MHz, Chloroform-d) δ 7.95- 7.82 (m, 2H), 5.77 (d, J = 8.14 Hz, 2H), 4.18 (s, 3H), 3.19 (dt, J = 56.06, 7.62 Hz, 2H), 2.88 (d, J = 37.74 Hz, 3H), 2.70 (s, 3H), 1.44 (q, J = 7.48 Hz, 1H), 0.85 (dt, J = 56.97, 7.41 Hz, 3H) |
| | (4-(5-bromo-6- | | |
| | methylpyridin-2-yl)-1- | | |
| | methyl-1H-1,2,3-triazol- | | |
| | 5-y1)methyl | | |
| | methyl(propyl)carbamate | | |

### Step 2: (3-methyl-5-(6-methyl-5-(4,4,5,5-tetramethyl-1,3,2-dioxaborolan-2-yl)pyridin-2-yl)isoxazol-4-yl)methyl cyclopentyl(methyl)carbamate (Intermediate X4)

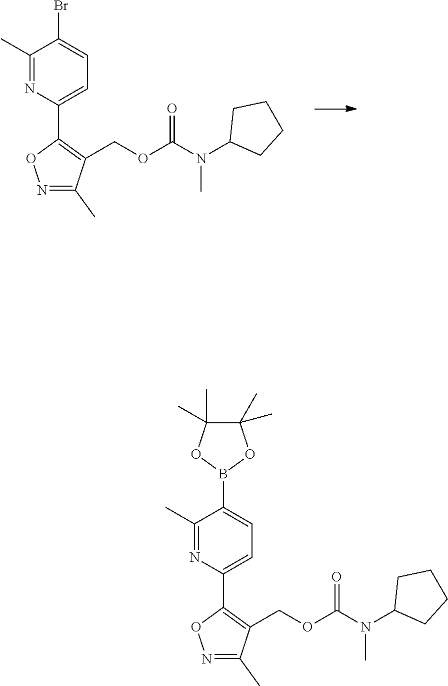 [SMILES: Cc1nc(-c2onc(C)c2COC(=O)N(C)C2CCCC2)ccc1Br ; Cc1nc(-c2onc(C)c2COC(=O)N(C)C2CCCC2)ccc1B1OC(C)(C)C(C)(C)O1]

Intermediate X4 (387 mg, crude) was obtained from (5-(5-bromo-6-methylpyridin-2-yl)-3-methylisoxazol-4-yl)methyl cyclopentyl(methyl)carbamate (Intermediate X1, 221.0 mg, 0.540 mmol) using a similar method as for Intermediate O1.

The Intermediate in the following table was prepared from reagents reported below by using methods analogous to Intermediate X4.

| Intermediate | Structure & Name | Reagents | Analytical data |
|---|---|---|---|
| X5 | 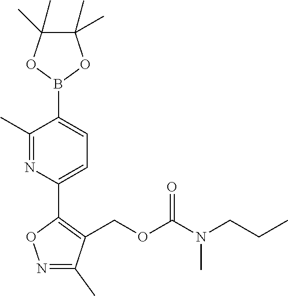 | X2 | LC-MS (ESI): m/z (M + 1): 403.2 (Method 1) |
| | (3-methyl-5-(6-methyl-5- | | |
| | (4,4,5,5-tetramethyl- | | |
| | 1,3,2-dioxaborolan-2- | | |
| | yl)pyridin-2-yl)isoxazol- | | |
| | 4-y1)methyl | | |
| | methyl(propyl)carbamate | | |
| X6 | 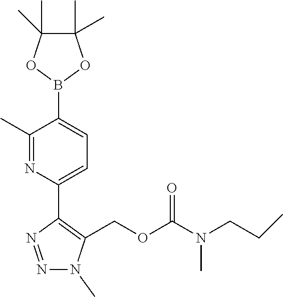 | X3 | LC-MS (ESI): m/z (M + 1): 403.2 (Method 1) |
| | (1-methyl-4-(6-methyl-5- | | |
| | (4,4,5,5-tetramethyl- | | |
| | 1,3,2-dioxaborolan-2- | | |
| | y1)pyridin-2-y1)-1H- | | |
| | 1,2,3-triazol-5-yl)methyl | | |
| | methyl(propyl)carbamate | | |

### Step 3: (5-(5-hydroxy-6-methylpyridin-2-yl)-3-methylisoxazol-4-yl)methyl cyclopentyl(methyl)carbamate (Intermediate X7)

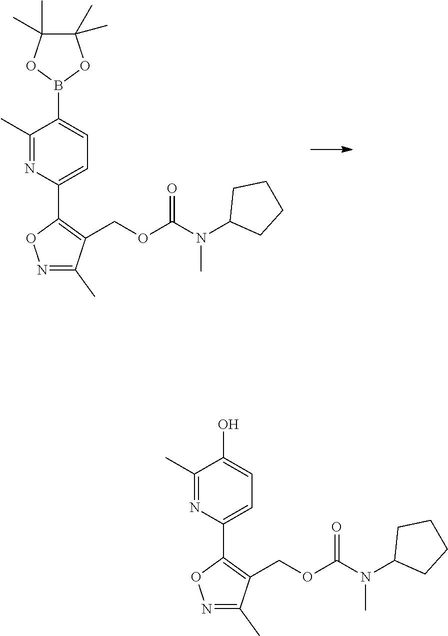 [SMILES: Cc1nc(-c2onc(C)c2COC(=O)N(C)C2CCCC2)ccc1B1OC(C)(C)C(C)(C)O1 ; Cc1nc(-c2onc(C)c2COC(=O)N(C)C2CCCC2)ccc1O]

Intermediate X7 (150 mg, 0.434 mmol, 51.1% yield) was obtained from (3-methyl-5-(6-methyl-5-(4,4,5,5-tetramethyl-1,3,2-dioxaborolan-2-yl)pyridin-2-yl)isoxazol-4-yl)methyl cyclopentyl(methyl)carbamate (Intermediate X4, 387.0 mg, 0.850 mmol) using a similar method as for Intermediate O2.

The Intermediates in the following table were prepared from reagents reported below by using methods analogous to Intermediate X7.

| Intermediate | Structure & Name | Reagents | Analytical data |
|---|---|---|---|
| X8 | 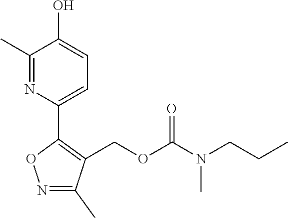 [SMILES: CCCN(C)C(=O)OCc1c(C)noc1-c1ccc(O)c(C)n1] | X5 | LC-MS (ESI): m/z (M + 1): 320.1 (Method 1) 1H NMR (400 MHz, DMSO-d6) δ ppm 10.16- 10.85 (m, 1 H), 7.61 (d, J = 8.36 Hz, 1 H), 7.25 (d, J = 8.36 Hz, 1 H), 5.35 (br. s., 2 H), 3.01-3.21 (m, 2 H), 2.71-2.83 (m, 3 H), 2.38 (s, 3 H), 2.30 (s, 3 H), 1.29-1.54 (m, 2 H), 0.62- 0.88 (m, 3 H) |
| | (5-(5-hydroxy-6- | | |
| | methylpyridin-2-y1)-3- | | |
| | methylisoxazol-4- | | |
| | yl)methyl | | |
| | methyl(propyl)carbamate | | |
| X9 | 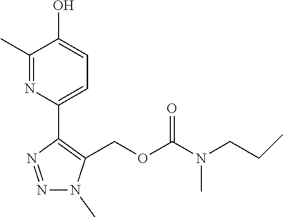 [SMILES: CCCN(C)C(=O)OCc1c(-c2ccc(O)c(C)n2)nnn1C] | X6 | LC-MS (ESI): m/z (M + 1): 320.1 (Method 1) |
| | (4-(5-hydroxy-6- | | |
| | methylpyridin-2-y1)-1- | | |
| | methyl-1H-1,2,3-triazol- | | |
| | 5-y1)methyl | | |
| | methyl(propyl)carbamate | | |

### Step 4: methyl (1S,2S)-2-(((6-(4-(((cyclopentyl(methyl)carbamoyl)oxy)methyl)-3-methylisoxazol-5-yl)-2-methylpyridin-3-yl)oxy)methyl)cyclohexane-1-carboxylate (Intermediate X1O)

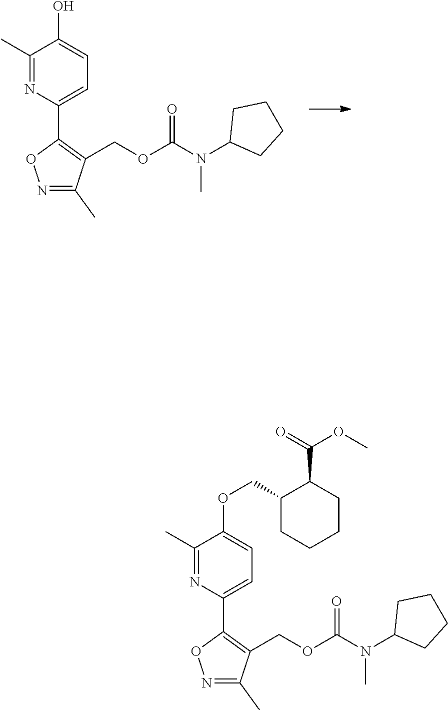 [SMILES: Cc1nc(-c2onc(C)c2COC(=O)N(C)C2CCCC2)ccc1O ; COC(=O)[C@H]1CCCC[C@@H]1COc1ccc(-c2onc(C)c2COC(=O)N(C)C2CCCC2)nc1C]

Intermediate X1O (222 mg, crude) was obtained from methyl (1S,2S)-2-(hydroxymethyl)cyclohexane-1-carboxylate (Intermediate O7, 74.79 mg, 0.430 mmol) and (5-(5-hydroxy-6-methylpyridin-2-yl)-3-methylisoxazol-4-yl)methyl cyclopentyl(methyl)carbamate (intermediate X7, 75.0 mg, 0.220 mmol using a similar method as for Intermediate O9.

LC-MS (ESI): m/z (M+1): 500.3 (Method 1)

The Intermediates in the following table were prepared from reagents reported below by using methods analogous to Intermediate X1O.

| Intermediate | Structure & Name | Reagents | Analytical data |
|---|---|---|---|
| X11 | 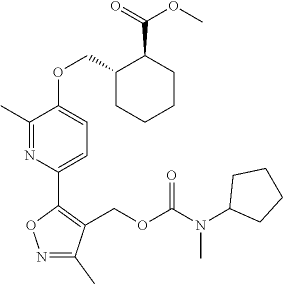 | X8 | LC-MS (ESI): m/z (M + 1): 474.3 (Method 1) |
| | methyl (1S,2S)-2-(((6-(4- | | |
| | (((cyclopentyl(methyl) | | |
| | carbamoyl)oxy)methyl)-3- | | |
| | methylisoxazol-5-yl)-2- | | |
| | methylpyridin-3- | | |
| | yl)oxy)methyl)cyclohexane- | | |
| | 1-carboxylate | | |
| X12 | 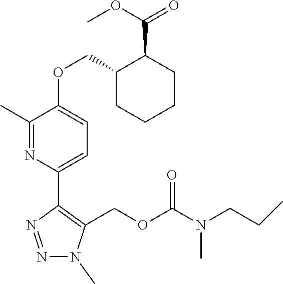 [SMILES: CCCN(C)C(=O)OCc1c(-c2ccc(OC[C@H]3CCCC[C@@H]3C(=O)OC)c(C)n2)nnn1C] | X9 | LC-MS (ESI): m/z (M + 1): 474.3 (Method 1) 1H NMR (400 MHz, DMSO-d6) δ 7.83 (d, J = 8.53 Hz, 1H), 7.40 (d, J = 8.58 Hz, 1H), 5.63 (s, 2H), 4.10 (s, 3H), 3.95 (dd, J = 9.79, 4.87 Hz, 1H), 3.85 (dd, J = 9.84, 5.60 Hz, 1H), 3.57 (s, 3H), 3.16 (d, J = 7.81 Hz, 1H), 3.05 (s, 1H), 2.77 (d, J = 16.58 Hz, 3H), 2.38 (s, 3H), 2.34 (dd, J = 11.36, 3.58 Hz, 1H), 2.10-1.98 (m, 1H), 1.90 (d, J = 12.59 Hz, 2H), 1.74 (d, J = 8.43 Hz, 2H), |
| | | | 1.55-1.17 (m, 6H), 0.73 |
| | methyl (1S,2S)-2-(((2- | | (d, J = 63.35 Hz, 3H) |
| | methyl-6-(1-methyl-5- | | |
| | (((methyl(propyl) | | |
| | carbamoyl)oxy)methyl)-1H- | | |
| | 1,2,3-triazol-4- | | |
| | yl)pyridin-3- | | |
| | yl)oxy)methyl)cyclohexane- | | |
| | 1-carboxylate | | |

### Step 5: (1S,2S)-2-(((6-(4-(((cyclopentyl(methyl)carbamoyl)oxy)methyl)-3-methylisoxazol-5-yl)-2-methylpyridin-3-yl)oxy)methyl)cyclohexane-1-carboxylic acid (Compound 74)

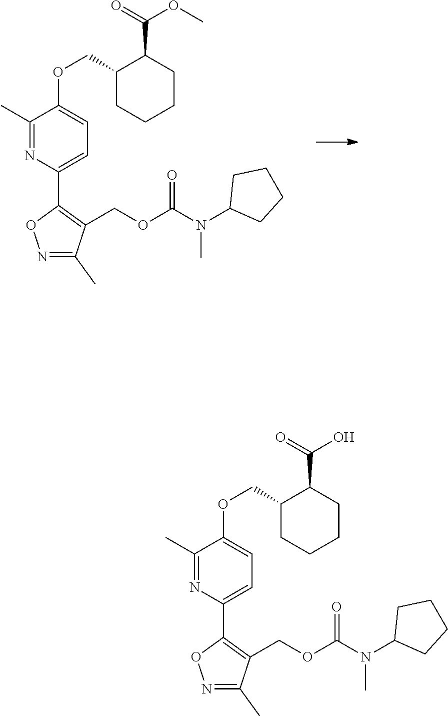 [SMILES: COC(=O)[C@H]1CCCC[C@@H]1COc1ccc(-c2onc(C)c2COC(=O)N(C)C2CCCC2)nc1C ; Cc1nc(-c2onc(C)c2COC(=O)N(C)C2CCCC2)ccc1OC[C@H]1CCCC[C@@H]1C(=O)O]

Compound 74 (54 mg, 0.111 mmol, 25.5% yield) was obtained as a white solid. from methyl (1S,2S)-2-(((6-(4-(((cyclopentyl(methyl)carbamoyl)oxy)methyl)-3-methylisoxazol-5-yl)-2-methylpyridin-3-yl)oxy)methyl)cyclohexane-1-carboxylate (Intermediate X10, 218.0 mg, 0.440 mmol) using a similar method as for Intermediate A9.

LC-MS (ESI): m/z (M+1): 486.3 (Method 1)

1H NMR (500 MHz, DMSO-d6) δ ppm 12.22 (br s, 1H), 7.70 (d, J=8.5 Hz, 1H), 7.46 (d, J=8.6 Hz, 1H), 5.35 (s, 2H), 4.06-4.65 (m, 1H), 3.96-4.05 (m, 1H), 3.85-3.95 (m, 1H), 2.65 (br s, 3H), 2.41 (s, 3H), 2.30 (s, 3H), 2.19 (td, J=11.1, 3.2 Hz, 1H), 1.96-2.05 (m, 1H), 1.81-1.95 (m, 2H), 1.72 (br d, J=9.2 Hz, 2H), 1.32-1.67 (m, 9H), 1.16-1.31 (m, 3H)

The Compounds in the following table were prepared from reagents reported below by using methods analogous to Compound 74.

| Compound | Structure & Name | Reagents | Analytical data |
|---|---|---|---|
| 75 | 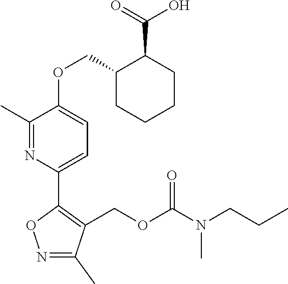 [SMILES: CCCN(C)C(=O)OCc1c(C)noc1-c1ccc(OC[C@H]2CCCC[C@@H]2C(=O)O)c(C)n1] | X11 | LC-MS (ESI): m/z (M + 1): 460.3 (Method 1) 1H NMR (500 MHz, DMSO-d6) δ ppm 12.15 (br s, 1 H), 7.70 (d, J = 8.5 Hz, 1 H), 7.46 (d, J = 8.6 Hz, 1 H), 5.22-5.46 (m, 2 H), 3.89-4.06 (m, 2 H), 2.94-3.20 (m, 2 H), 2.71- 2.84 (m, 3 H), 2.41 (s, 3 H), 2.30 (s, 3 H), 2.21 (td, J = 11.3, 3.6 Hz, 1 H), 1.96- 2.07 (m, 1 H), 1.82-1.95 (m, 2 H), 1.73 (br d, J = 9.5 Hz, 2 H), 1.13-1.52 (m, 6 H), 0.53-0.90 (m, 3 H) |
| | (1S,2S)-2-(((2-methyl-6- | | |
| | (3-methyl-4- | | |
| | (((methyl(propyl)carbamoyl) | | |
| | oxy)methyl)isoxazol- | | |
| | 5-y1)pyridin-3- | | |
| | yl)oxy)methyl)cyclohexane- | | |
| | 1-carboxylic acid | | |
| 76 | 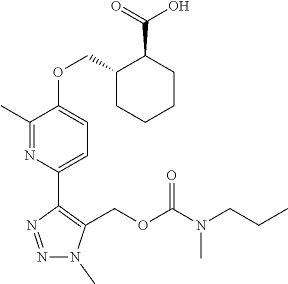 [SMILES: CCCN(C)C(=O)OCc1c(-c2ccc(OC[C@H]3CCCC[C@@H]3C(=O)O)c(C)n2)nnn1C] | X12 | LC-MS (ESI): m/z (M + 1): 460.3 (Method 1) 1H NMR (500 MHz, DMSO-d6) δ ppm 12.16 (br s, 1 H), 7.82 (br d, J = 8.4 Hz, 1 H), 7.40 (d, J = 8.5 Hz, 1 H), 5.54-5.72 (m, 2 H), 4.09 (s, 3 H), 3.93-4.01 (m, 1 H), 3.88 (dd, J = 9.6, 6.0 Hz, 1 H), 2.99-3.20 (m, 2 H), 2.69- 2.85 (m, 3 H), 2.39 (s, 3 H), 2.21 (td, J = 11.2, 3.2 Hz, 1 H), 1.96-2.05 (m, 1 H), 1.82-1.95 (m, 2 H), 1.66-1.80 (m, 2 H), 1.18- 1.53 (m, 6 H), 0.57-0.88 |
| | (1S,2S)-2-(((2-methyl-6- | | (m, 3 H) |
| | (1-methyl-5- | | |
| | (((methyl(propyl)carbamoyl) | | |
| | oxy)methyl)-1H- | | |
| | 1,2,3-triazol-4- | | |
| | yl)pyridin-3- | | |
| | yl)oxy)methyl)cyclohexane- | | |
| | 1-carboxylic acid | | |

### Example 28

### Process Synthesis of Compound 77

### (2-((4-(4-((((R)-1-(2-chlorophenyl)ethoxy)carbonyl)amino)-3-methylisoxazol-5-yl)phenoxy)methyl)cyclohexane-1-carboxylic acid trans racemate

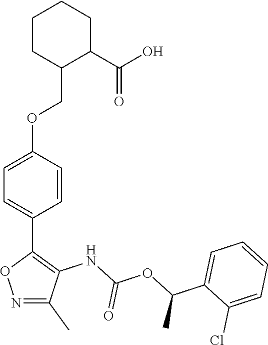 [SMILES: Cc1noc(-c2ccc(OCC3CCCCC3C(=O)O)cc2)c1NC(=O)O[C@H](C)c1ccccc1Cl]

### Step 1: 5-(4-acetoxyphenyl)-3-methylisoxazole-4-carboxylic acid (Intermediate Y1)

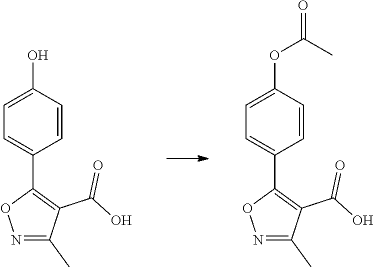 [SMILES: Cc1noc(-c2ccc(O)cc2)c1C(=O)O ; CC(=O)Oc1ccc(-c2onc(C)c2C(=O)O)cc1]

A solution of 5-(4-hydroxyphenyl)-3-methylisoxazole-4-carboxylic acid (Intermediate A13, 23.5 g, 107 mmol) in aqueous 2M KOH (107 mL, 214 mmol) was added with ice (50 g) and cooled to 0° C. with an ice bath. Acetic anhydride (13.3 mL, 139 mmol) was added dropwise and the mixture was stirred at 0° C. for 1 h. 1.2N HCl was added (pH<4) and the precipitate was collected by filtration, washed with water and dried under vacuum to obtain the title compound (26.4 g, 101 mmol, 94% yield) as an ivory solid.

LC-MS (ESI): m/z (M+1): 262.0 (Method 13)

### Step 2: (R)-4-(4-(((1-(2-chlorophenyl)ethoxy)carbonyl)amino)-3-methylisoxazol-5-yl)phenyl acetate (Intermediate Y2)

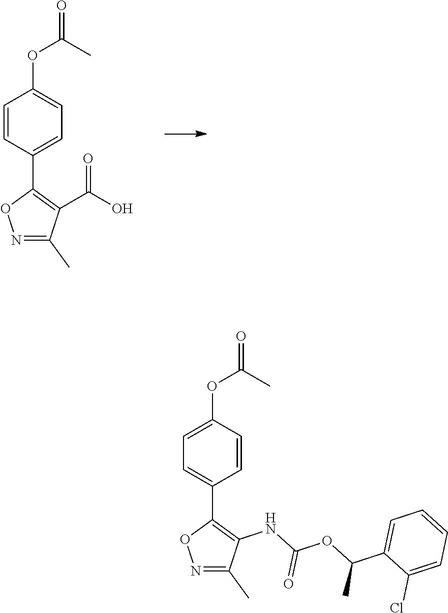 [SMILES: CC(=O)Oc1ccc(-c2onc(C)c2C(=O)O)cc1 ; CC(=O)Oc1ccc(-c2onc(C)c2NC(=O)O[C@H](C)c2ccccc2Cl)cc1]

Intermediate Y2 (1.048 g, 2.325 mmol) was obtained from 5-(4-acetoxyphenyl)-3-methylisoxazole-4-carboxylic acid (Intermediate Y1, 1.5 g, 5.1 mmol) and (R)-1-(2-chlorophenyl)ethan-1-ol (1.19 g, 7.65 mmol) using a similar method as for Intermediate A14.

LC-MS (ESI): m/z (M+1): 415.0 (Method 15)

### Step 3: (R)-4-(4-((tert-butoxycarbonyl)((1-(2-chlorophenyl)ethoxy)carbonyl)amino)-3-methylisoxazol-5-yl)phenyl acetate (Intermediate Y3)

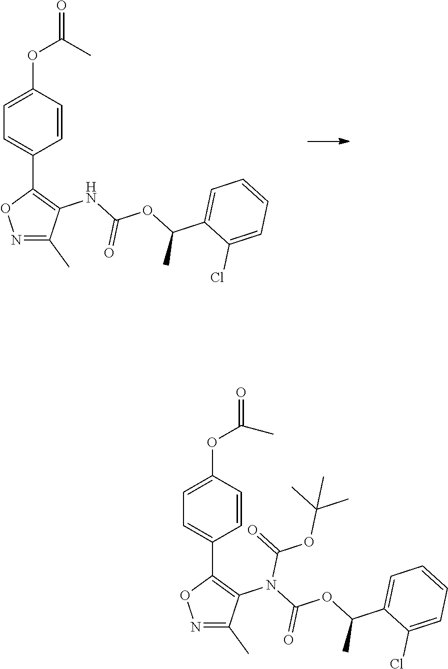 [SMILES: CC(=O)Oc1ccc(-c2onc(C)c2NC(=O)O[C@H](C)c2ccccc2Cl)cc1 ; CC(=O)Oc1ccc(-c2onc(C)c2N(C(=O)O[C@H](C)c2ccccc2Cl)C(=O)OC(C)(C)C)cc1]

To a solution of (R)-4-(4-(((1-(2-chlorophenyl)ethoxy)carbonyl)amino)-3-methylisoxazol-5-yl)phenyl acetate (Intermediate Y2, 1.048 g, 2.325 mmol) in DCM (14.5 mL), cooled with an ice bath, DIPEA (0.812 mL, 4.65 mmol) was added followed by (Boc)₂₀ (0.761 g, 3.49 mmol) and DMAP (0.028 g, 0.233 mmol) and the reaction mixture was allowed to reach RT and stirred overnight. The mixture was diluted with DCM and washed with 1.2M HCl and with sat. NaHCO₃, dried over sodium sulfate, filtered and concentrated to afford the title compound (1.064 g, 1.932 mmol, 83% yield) as an orange amorphous which was used without any additional purification.

LC-MS (ESI): m/z (M+1): 515.1 (Method 15)

The Intermediates in the following table were prepared from reagents reported below by using methods analogous to Intermediate Y3.

| Intermediate | Structure & Name | Reagents | Analytical data |
|---|---|---|---|
| Y4 | 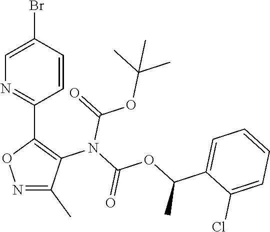 [SMILES: Cc1noc(-c2ccc(Br)cn2)c1N(C(=O)O[C@H](C)c1ccccc1Cl)C(=O)OC(C)(C)C] | A19 | LC-MS (ESI): m/z (M + 1): 538.1 (Method 14) |
| | (R)-1-(2- | | |
| | chlorophenyl)ethyl (5-(5- | | |
| | bromopyridin-2-y1)-3- | | |
| | methylisoxazol-4-yl)( tert- | | |
| | butoxycarbonyl)carbamate | | |
| Y5 | 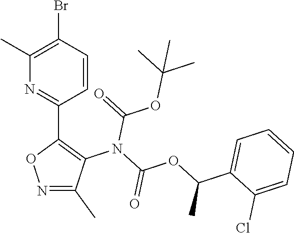 [SMILES: Cc1nc(-c2onc(C)c2N(C(=O)O[C@H](C)c2ccccc2Cl)C(=O)OC(C)(C)C)ccc1Br] | A20 | LC-MS (ESI): m/z (M + 1): 552.2 (Method 14) |
| | (R)-1-(2- | | |
| | chlorophenyl)ethyl (5-(5- | | |
| | bromo-6-methylpyridin-2- | | |
| | yl)-3-methylisoxazol-4- | | |
| | yl)(tert- | | |
| | butoxycarbonyl)carbamate | | |

### Step 4: (R)-1-(2-chlorophenyl)ethyl (tert-butoxycarbonyl)(5-(4-hydroxyphenyl)-3-methylisoxazol-4-yl)carbamate (Intermediate Y6)

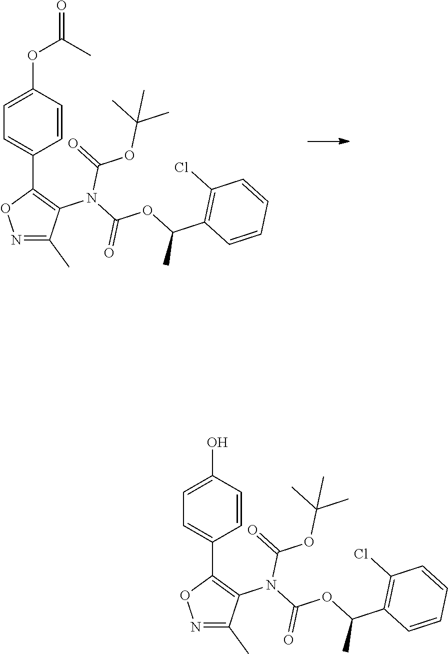 [SMILES: CC(=O)Oc1ccc(-c2onc(C)c2N(C(=O)O[C@H](C)c2ccccc2Cl)C(=O)OC(C)(C)C)cc1 ; Cc1noc(-c2ccc(O)cc2)c1N(C(=O)O[C@H](C)c1ccccc1Cl)C(=O)OC(C)(C)C]

Intermediate Y6 (0.610 g, 1.290 mmol, 470 yield) was obtained as a white powder from (R)-4-(4-((tert-butoxycarbonyl)((1-(2-chlorophenyl)ethoxy)carbonyl)amino)-3-methylisoxazol-5-yl)phenyl acetate (Intermediate Y3, 1.426 g, 2.77 mmol) using a similar method as for Compound 43.

LC-MS (ESI): m/z (M+1): 473.3 (Method 15)

The Intermediates in the following table were prepared from reagents reported below by using methods analogous to Intermediate O1 and O2.

| Intermediate | Structure & Name | Reagents | Analytical data |
|---|---|---|---|
| Y7 | 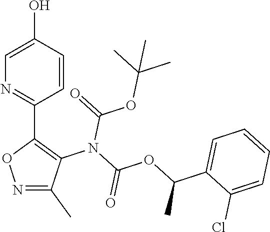 [SMILES: Cc1noc(-c2ccc(O)cn2)c1N(C(=O)O[C@H](C)c1ccccc1Cl)C(=O)OC(C)(C)C] | Y4 | LC-MS (ESI): m/z (M + 1): 474.2 (Method 14) |
| | (R)-1-(2- | | |
| | chlorophenyl)ethyl (tert- | | |
| | butoxycarbonyl)(5-(5- | | |
| | hydroxypyridin-2-y1)-3- | | |
| | methylisoxazol-4- | | |
| | yl)carbamate | | |
| Y8 | 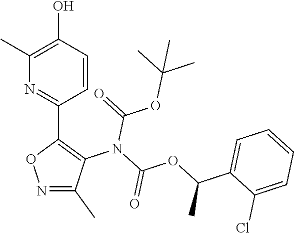 [SMILES: Cc1nc(-c2onc(C)c2N(C(=O)O[C@H](C)c2ccccc2Cl)C(=O)OC(C)(C)C)ccc1O] | Y5 | LC-MS (ESI): m/z (M + 1): 488.2 (Method 14) |
| | (R)-1-(2- | | |
| | chlorophenyl)ethyl (tert- | | |
| | butoxycarbony1)(5-(5- | | |
| | hydroxy-6- | | |
| | methylpyridin-2-y1)-3- | | |
| | methylisoxazol-4- | | |
| | yl)carbamate | | |

### Step 5: methyl 2-((4-(4-((((R)-1-(2-chlorophenyl)ethoxy)carbonyl)amino)-3-methylisoxazol-5-yl)phenoxy)methyl)cyclohexane-1-carboxylate (trans mixture) (Intermediate Y9)

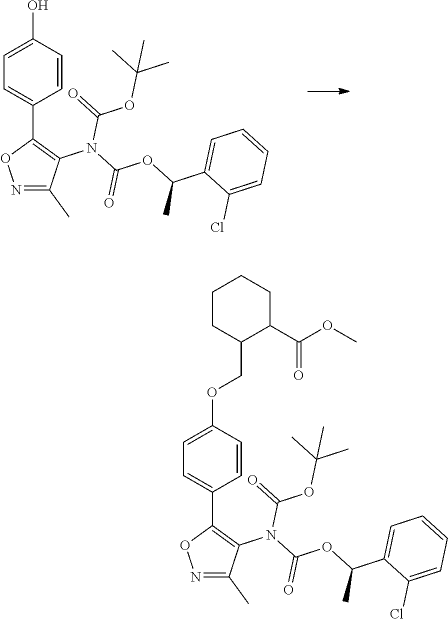 [SMILES: Cc1noc(-c2ccc(O)cc2)c1N(C(=O)O[C@H](C)c1ccccc1Cl)C(=O)OC(C)(C)C ; COC(=O)C1CCCCC1COc1ccc(-c2onc(C)c2N(C(=O)O[C@H](C)c2ccccc2Cl)C(=O)OC(C)(C)C)cc1]

Intermediate Y9 (0.148 g, 0.230 mmol, 93% yield) was obtained from methyl trans-rac 2-(hydroxymethyl)cyclohexane-1-carboxylate (0.085 g, 0.492 mmol) and (R)-1-(2-chlorophenyl)ethyl (tert-butoxycarbonyl)(5-(4-hydroxyphenyl)-3-methylisoxazol-4-yl)carbamate (Intermediate Y6, 0.120 g, 0.246 mmol) using a similar method as for Intermediate O9.

LC-MS (ESI): m/z (M+1): 527.3 (Method 15)

### Step 6: methyl 2-((4-(4-((((R)-1-(2-chlorophenyl)ethoxy)carbonyl)amino)-3-methylisoxazol-5-yl)phenoxy)methyl)cyclohexane-1-carboxylate (trans mixture) (Intermediate Y10)

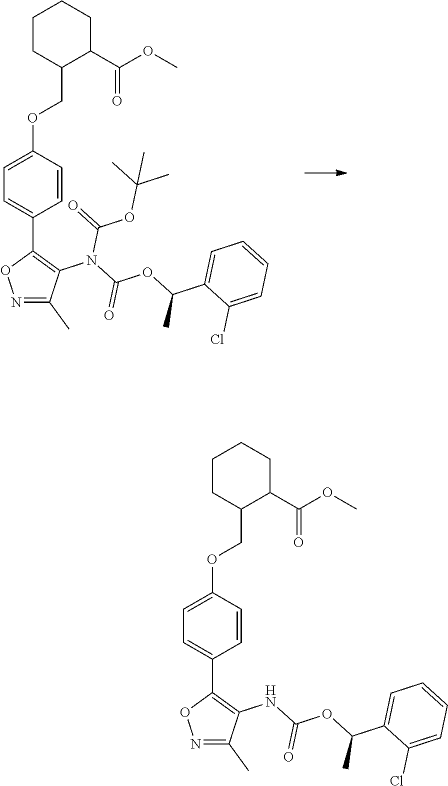 [SMILES: COC(=O)C1CCCCC1COc1ccc(-c2onc(C)c2N(C(=O)O[C@H](C)c2ccccc2Cl)C(=O)OC(C)(C)C)cc1 ; COC(=O)C1CCCCC1COc1ccc(-c2onc(C)c2NC(=O)O[C@H](C)c2ccccc2Cl)cc1]

To a solution of methyl 2-((4-(4-((((R)-1-(2-chlorophenyl)ethoxy)carbonyl)amino)-3-methylisoxazol-5-yl)phenoxy)methyl)cyclohexane-1-carboxylate trans racemate (Intermediate Y9, 0.148 g, 0.230 mmol) in DCM (5 mL) cooled to 0° C., TFA (0.178 mL, 2.305 mmol) was added and the mixture was allowed to reach RT and stirred overnight. The mixture was diluted with DCM and washed with sat. NaHCO₃ and brine. The organic phase was dried over sodium sulfate, filtered and concentrated. The crude was purified by flash chromatography (hexane:EtOAc from 90:10 to 80:20) to obtain the desired compound (0.103 g, 0.190 mmol, 82% yield) as a colorless amorphous.

LC-MS (ESI): m/z (M+1): 542.3 (Method 14)

The Intermediates in the following table were prepared from reagents reported below by using methods analogous to Intermediate Y10 (Step 7 and Step 8).

| Intermediate | Structure & Name | Reagents | Analytical data |
|---|---|---|---|
| Y11 | 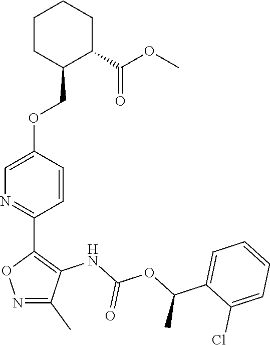 [SMILES: COC(=O)[C@H]1CCCC[C@@H]1COc1ccc(-c2onc(C)c2NC(=O)O[C@H](C)c2ccccc2Cl)nc1] | Y7 and O7 | LC-MS (ESI): m/z (M + 1): 528.4 (Method 14) |
| | methyl (1S,2S)-2-(((6-(4- | | |
| | ((((R)-1-(2- | | |
| | chlorophenyl)ethoxy) | | |
| | carbonyl)amino)-3- | | |
| | methylisoxazol-5- | | |
| | yl)pyridin-3- | | |
| | yl)oxy)methyl)cyclohexane- | | |
| | 1-carboxylate | | |
| Y12 | 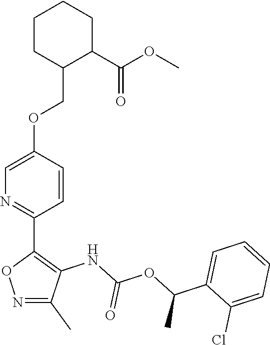 [SMILES: COC(=O)C1CCCCC1COc1ccc(-c2onc(C)c2NC(=O)O[C@H](C)c2ccccc2Cl)nc1] | Y7 and O8 | LC-MS (ESI): m/z (M + 1): 528.3 (Method 14) |
| | methyl 2-(((6-(4-((((R)- | | |
| | 1-(2- | | |
| | chlorophenyl)ethoxy) | | |
| | carbonyl)amino)-3- | | |
| | methylisoxazol-5- | | |
| | y1)pyridin-3- | | |
| | yl)oxy)methyl)cyclohexane- | | |
| | 1-carboxylate (cis | | |
| | mixture) | | |
| Y13 | 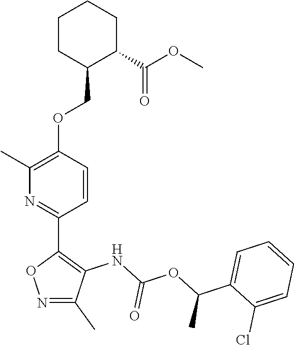 [SMILES: COC(=O)[C@H]1CCCC[C@@H]1COc1ccc(-c2onc(C)c2NC(=O)O[C@H](C)c2ccccc2Cl)nc1C] | Y8 and O7 | LC-MS (ESI): m/z (M + 1): 542.1 (Method 14) |
| | methyl (1S,2S)-2-(((6-(4- | | |
| | ((((R)-1-(2- | | |
| | chlorophenyl)ethoxy) | | |
| | carbonyl)amino)-3- | | |
| | methylisoxazol-5-y1)-2- | | |
| | methylpyridin-3- | | |
| | yl)oxy)methyl)cyclohexane- | | |
| | 1-carboxylate | | |

### Step 7: 2-((4-(4-((((R)-1-(2-chlorophenyl)ethoxy)carbonyl)amino)-3-methylisoxazol-5-yl)phenoxy)methyl)cyclohexane-1-carboxylic acid (trans mixture) (Compound 77)

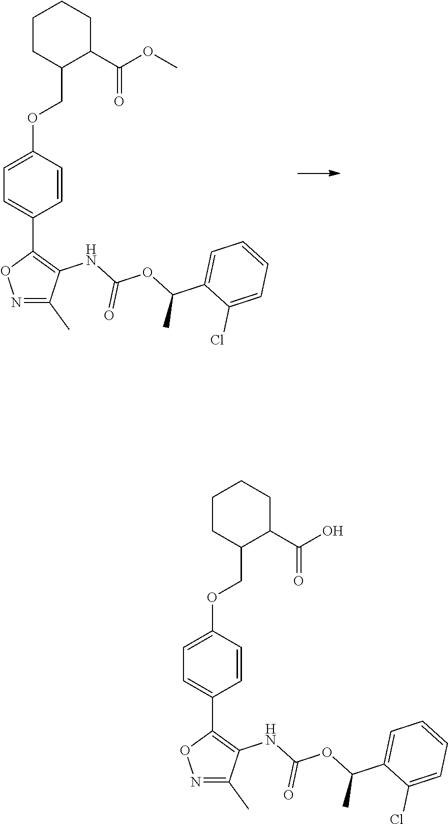 [SMILES: COC(=O)C1CCCCC1COc1ccc(-c2onc(C)c2NC(=O)O[C@H](C)c2ccccc2Cl)cc1 ; Cc1noc(-c2ccc(OCC3CCCCC3C(=O)O)cc2)c1NC(=O)O[C@H](C)c1ccccc1Cl]

Compound 77 (0.080 g, 0.152 mmol, 81% yield) was obtained as a white foam from 2-((4-(4-((((R)-1-(2-chlorophenyl)ethoxy)carbonyl)amino)-3-methylisoxazol-5-yl)phenoxy)methyl)cyclohexane-1-carboxylate (trans mixture) (Intermediate Y10, 0.102 g, 0.188 mmol) using a similar method as for Intermediate A9.

LC-MS (ESI): m/z (M+1): 528.2 (Method 14)

¹H NMR (300 MHz, DMSO-d₆) δ ppm 12.13 (br s, 1H), 8.45-9.25 (m, 1H), 7.59 (d, J=8.5 Hz, 1H), 7.05-7.70 (m, 5H), 5.87-6.05 (m, 1H), 3.84-4.02 (m, 2H), 2.38 (s, 3H), 2.17-2.30 (m, 1H), 2.15 (s, 3H), 1.80-2.09 (m, 3H), 1.67-1.79 (m, 2H), 0.93-1.65 (m, 7H)

The Compounds in the following table were prepared from reagents reported below following similar procedures as for Compound 77.

| Compound | Structure & Name | Reagents | Analytical data |
|---|---|---|---|
| 78 | 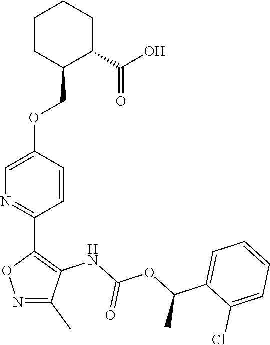 [SMILES: Cc1noc(-c2ccc(OC[C@H]3CCCC[C@@H]3C(=O)O)cn2)c1NC(=O)O[C@H](C)c1ccccc1Cl] | Y11 | LC-MS (ESI): m/z (M + 1): 514.4 (Method 14) ¹H NMR (300 MHz, DMSO-d₆) δ ppm 12.20 (br s, 1H), 8.64-9.39 (m, 1H), 8.32 (d, J = 2.7 Hz, 1H), 7.73 (d, J = 8.8 Hz, 1H), 6.83-7.70 (m, 5H), 5.86-6.06 (m, 1H), 3.86- 4.09 (m, 2H), 2.08-2.28 (m, 1H), 2.15 (s, 3H), 1.83- 2.07 (m, 3H), 1.63-1.83 (m, 2H), 1.07-1.63 (m, 7 H) |
| | (1S,2S)-2-(((6-(4-((((R)- | | |
| | 1-(2- | | |
| | chlorophenyl)ethoxy) | | |
| | carbonyl)amino)-3- | | |
| | methylisoxazol-5- | | |
| | yl)pyridin-3- | | |
| | yl)oxy)methyl)cyclohexane- | | |
| | 1-carboxylic acid | | |
| 79 | 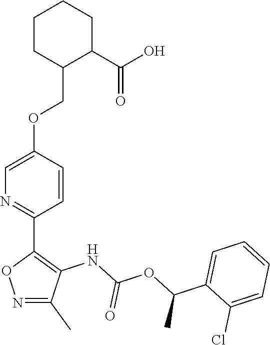 [SMILES: Cc1noc(-c2ccc(OCC3CCCCC3C(=O)O)cn2)c1NC(=O)O[C@H](C)c1ccccc1Cl] | Y12 | LC-MS (ESI): m/z (M + 1): 514.3 (Method 15) ¹H NMR (300 MHz, DMSO-d₆) δ ppm 12.05- 12.26 (m, 1H), 8.64-9.36 (m, 1H), 8.32 (d, J = 2.7 Hz, 1H), 7.73 (d, J = 8.8 Hz, 1H), 6.77-7.68 (m, 5H), 5.86-6.07 (m, 1H), 4.04-4.25 (m, 2H), 2.66- 2.78 (m, 1H), 2.20-2.36 (m, 1H), 2.15 (s, 3H), 1.71- 1.93 (m, 2H), 1.07-1.71 (m, 7 H) |
| | 2-(((6-(4-((((R)-1-(2- | | |
| | chlorophenyl)ethoxy)car | | |
| | bonyl)amino)-3- | | |
| | methylisoxazol-5- | | |
| | yl)pyridin-3- | | |
| | yl)oxy)methyl)cyclohexa | | |
| | ne-1-carboxylic acid (cis | | |
| | mixture) | | |
| 80 | 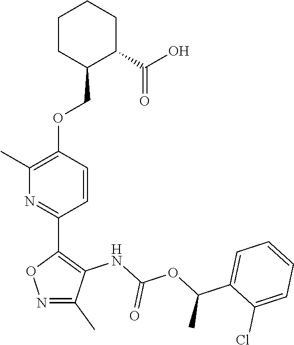 | Y13 | LC-MS (ESI): m/z (M + 1): 513.3 (Method 14) ¹H NMR (300 MHz, DMSO-d₆) δ ppm 12.17 (br s, 1H), 8.78-9.33 (m, 1H), 7.66 (d, J = 8.2 Hz, 2H), 6.85-7.78 (m, 4H), 7.03 (d, J = 8.2 Hz, 2H), 5.98 (q J = 6.3 Hz, 1H), 3.82-4.00 (m, 2H), 2.11- 2.25 (m, 1H), 2.09 (s, 3H), 1.83-2.03 (m, 3H), 1.63- 1.83 (m, 2H), 1.08-1.63 (m, 7H) |
| | (1S,2S)-2-(((6-(4-((((R)- | | |
| | 1-(2- | | |
| | chlorophenyl)ethoxy) | | |
| | carbonyl)amino)-3- | | |
| | methylisoxazol-5-yl)-2- | | |
| | methylpyridin-3- | | |
| | yl)oxy)methyl)cyclohexane- | | |
| | 1-carboxylic acid | | |

Compound 79 was submitted to chiral semipreparative chromatography.

Column Chiralpak IC (25×3.0 cm), 5μ Mobile phase n-Hexane/(2-propanol+0.1% formic acid) 85/15% v/v Flow rate (ml/min) 40 ml/min DAD detection 280 nm Loop 320 μL

### Peak 1: Compound 81

### Single Diastereomer 1 of cis-2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid

LC-MS (ESI): m/z (M+1): 514.3 (Method 1)

¹H NMR (400 MHz, DMSO-d₆) δ ppm 11.63-12.85 (m, 1H), 9.01-9.37 (m, 1H), 8.32 (d, J=2.8 Hz, 1H), 7.72 (d, J=8.6 Hz, 1H), 7.23-7.68 (m, 5H), 5.78-6.12 (m, 1H), 4.14 (d, J=7.0 Hz, 2H), 2.63-2.77 (m, 1H), 2.20-2.30 (m, 1H), 2.14 (br s, 3H), 1.30-1.89 (m, 11H)

### Peak 1: Compound 82

### Single Diastereomer 2 of cis-2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid

LC-MS (ESI): m/z (M+1): 514.3 (Method 1)

¹H NMR (400 MHz, DMSO-d₆) δ ppm 11.63-12.93 (m, 1H), 9.03-9.40 (m, 1H), 8.31 (d, J=2.9 Hz, 1H), 7.72 (d, J=8.8 Hz, 1H), 7.19-7.68 (m, 5H), 5.95 (br d, J=5.9 Hz, 1H), 4.07-4.21 (m, 2H), 2.67 (br dd, J=4.3, 2.7 Hz, 1H), 2.20-2.30 (m, 1H), 2.14 (br s, 3H), 1.29-1.87 (m, 11H)

### Pharmacological Activity of the Compounds of the Invention

### In Vitro Assays

The effectiveness of compounds of the present invention as LPA1 antagonist can be determined at the human recombinant LPA1 expressed in CHO cells, using a FLIPR assay in 384 well format.

CHO-hLPA1 cell lines are cultured in a humidified incubator at 5% C₀₂ in DMEM/F-12 (1:1) MIXTURE with 2 mM Glutamax, supplemented with 10% of Foetal Bovine Serum, 1 mM Sodium Pyruvate, 11 mM Hepes and 1× Penicillin/Streptomycin. CHO hLPA1 cells are seeded into black walled clear-bottom 384-well plates (#781091, Greiner Bio-One GmbH) at a density of 7,500 cells per well in 50 μl culture media and grown overnight in a 37° C. humidified CO2-incubator. Serial dilutions (1:3 or 1:4, 11 points CRC) of compounds are performed in 100% DMSO at 200× the final concentration. The compounds are diluted 1:50 prior to the experiment with Assay Buffer (20 mM HEPES, 145 mM NaCl, 5 mM KCl, 5.5 mM glucose, 1 mM MgCl2 and 2 mM CaCl2), pH 7.4 containing 0.01% Pluronic F-127) to obtain a solution corresponding to 5-fold the final concentration in the assay (4×, 2% DMSO). The final concentration of DMSO in the assay will be 0.5% in each well. Medium is removed by aspiration and cells are then incubated with 30 μl of a loading solution containing 5 μM of the cytoplasmic Ca2+ indicator Cal-520 AM in Assay Buffer containing 2.5 mM probenecid for 30 min at 37° C. incubator (cell loading). The loaded cell plates are transferred into the FLIPR instrument and calcium responses are monitored during the on-line addition protocols. For testing of compounds, after the cell loading, 10 μl/well of 4× antagonists' solution was added onto the cells. After 30 min pre-incubation (at 37° C.), 10 μl/well of 5× concentrated LPA EC80 was added and Ca2+ mobilization responses was followed during the on-line addition protocol. Intracellular peak fluorescence values subtracted by baseline fluorescence are exported and analysed to determine IC₅₀ values, respectively. The calcium response is expressed as percentage of the maximal inhibition of the EC80 agonist response.

The raw data obtained in unstimulated controls (DMSO, no LPA) are set as “100% inhibition”, while the raw data obtained in negative controls, i.e. in the absence of compounds and stimulating with LPA EC80, are set as “0% inhibition”.

The raw data (peak height expressed as relative fluorescence units) are normalized and transformed into “percent of inhibition”. Curve fitting and pIC₅₀ (−LogIC₅₀) estimations are carried out using a four-parameter logistic model using XLfit Software.

The results for individual compounds are provided below in Table 5 and are expressed as range of activity.

The results for individual compounds are provided below in Table 5 and are expressed as range of activity.

TABLE 5

| Compound No. | LPA1 IC5₀ |
|---|---|
| 1, 5, 9, 21, 35, 42, 56, | + |
| 3, 4, 7, 8, 12, 13, 14, 15, 17, 20, 23, | ++ |
| 24, 25, 27, 32, 33, 34, 36, 43, 45, | |
| 53, 59, 64, 72, 73, 76 | |
| 2, 6, 10, 11, 16, 18, 19, 22, 26, 28, 29, 30, 31, | +++ |
| 37, 38, 39, 40, 41, 44, 46, 47, 48, 49, 50, 51, | |
| 52, 54, 55, 57, 58, 60, 61, 62, 63, 65, 66, 67, 68, | |
| 69, 70, 71, 74, 75, 77, 78, 79, 80, 81, 82 | |

wherein the compounds are classified in term of potency with respect to their inhibitory activity on LPA1 receptors according to the following classification criterion:
+: LPA1 IC₅₀ comprised between about 600 nM and 250 nM
++: LPA1 IC₅₀ comprised between about 250 nM and 50 nM
+++: LPA1 IC₅₀ less than about 50 nM

As it can be appreciated, all the compounds of Table 5 show an antagonist activity on LPA1 receptor. In fact, it can be recognized that the symbol + indicate a good and sufficient level of activity, which can be even increased up to +++, thus confirming the high activity receptor LPA1 of the compounds of the invention.

1: A compound of formula (I) 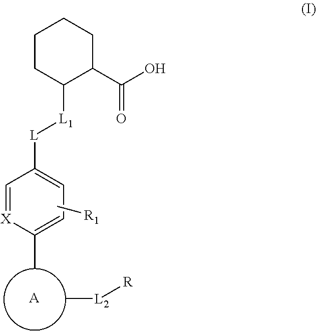
wherein X is —CH— or N;
R₁ is H or (C₁-C₄)alkyl;
L is —O— or —NH—;
L₁ is (—CH₂—)ₙ, or —C(O)—;
n is an integer between 0 and 2;
A is selected from the group consisting of 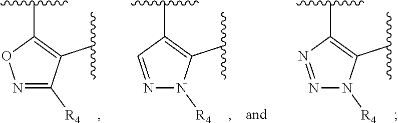
L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, —(C₁-C₄)alkyl-R₂, —NR₆R₇, —OC(O)NR₆R₇, —NHC(O)O—(C₁-C₄)alkyl-R₂, and heteroaryl, wherein any of such heteroaryl is optionally substituted by one or more groups selected from the group consisting of —OR₃, —(C₁-C₄)alkyl, —(C₁-C₄)haloalkyl, (—C₃-C₆)cycloalkyl, aryl, heteroaryl, and —NR₆R₇;
R₂ is H or is selected from the group consisting of —(C₁-C₄)alkyl, aryl, and heteroaryl, wherein any of such aryl, heteroaryl and alkyl are optionally substituted by one or more groups selected from the group consisting of —(C₁-C₄)alkyl, halo, —OR₅, and —R₅;
R₃ is H or is selected from the group consisting of —(C₁-C₄)alkyl, —(C₁-C₄)haloalkyl, and —(C₃-C₆)cycloalkyl optionally substituted by one or more halo;
R₄ is H or (C₁-C₄)alkyl;
R₅ is aryl optionally substituted by one or more groups selected from the group consisting of —(C₁-C₄)alkyl and halo; and
R₆ and R₇ are at each occurrence independently H or selected from the group consisting of —(C₁-C₆)alkyl, —(C₃-C₆)cycloalkyl, and heteroaryl, wherein any of such heteroaryl is optionally substituted by one or more groups selected from the group consisting of —OR₄, aryl, —(C₃-C₆)cycloalkyl, and heteroaryl,
with the proviso that when A is 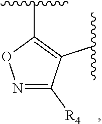
L is —NH—, L₁ is —C(O)—, X is —CH—, L₂ is —NH—, and R is not —C(O)O(C₁-C₅)alkyl-R₂.

2: A compound of formula (I) 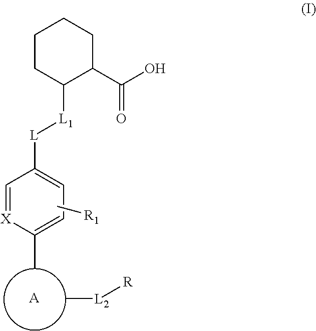
wherein L is —O— and L₁ is —CH₂—, represented by the general formula (Ia) 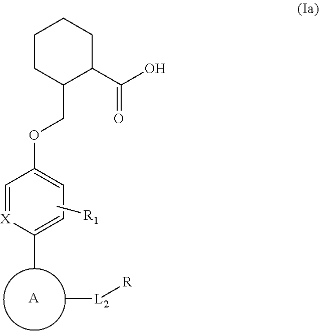
wherein
X is —CH— or N;
R₁ is H or —(C₁-C₄)alkyl;
A is selected from the group consisting of 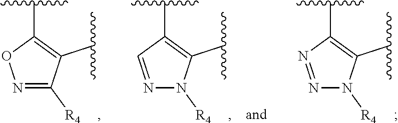
L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, heteroaryl, —(C₁-C₄)alkyl-R₂, —NR₆R₇, —OC(O)NR₆R₇, and —NH—C(O)O—(C₁-C₄)alkyl-R₂, wherein any of such heteroaryl is optionally substituted by one or more groups selected from the group consisting of —OR₃, —(C₁-C₄)alkyl, —(C₁-C₄)haloalkyl, —(C₃-C₆)cycloalkyl, aryl, heteroaryl, and —NR₆R₇;
R₂ is H or is selected from the group consisting of —(C₁-C₄)alkyl, aryl, and heteroaryl, wherein any of such aryl, heteroaryl and alkyl are optionally substituted by one or more groups selected from the group consisting of —(C₁-C₄)alkyl, halo, —OR₅, and —R₅;
R₃ is H or is selected from the group consisting of —(C₁-C₄)alkyl, —(C₁-C₄)haloalkyl, and —(C₃-C₆)cycloalkyl optionally substituted by one or more halo;
R₄ is H or (C₁-C₄)alkyl;
R₅ is aryl optionally substituted by one or more groups selected from the group consisting of —(C₁-C₄)alkyl, and halo; and
R₆ and R₇ are at each occurrence independently H or selected from the group consisting of —(C₁-C₆)alkyl, —(C₃-C₆)cycloalkyl, and heteroaryl, wherein any of such heteroaryl is optionally substituted by one or more groups selected from the group consisting of —OR₄, aryl, —(C₃-C₆)cycloalkyl, and heteroaryl.

3: A compound of formula (I) 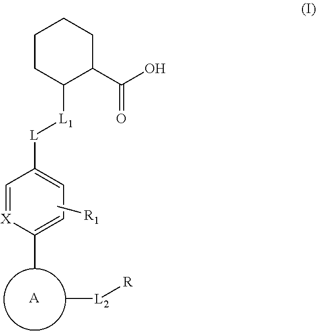
wherein X is —N, L is —NH— and L₁ is —C(O)—, represented by the general formula (Ib) 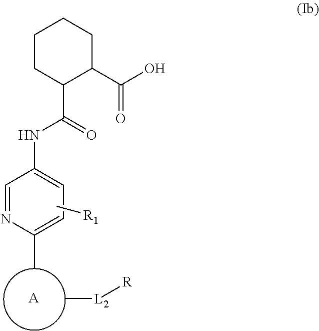
wherein
R₁ is H or (C₁-C₄)alkyl;
A is selected from the group consisting of 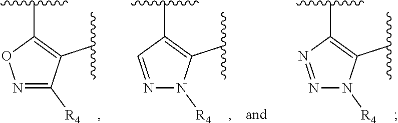
L₂ is (—CH₂—)ₙ, or —NH—;
R is selected from the group consisting of —C(O)O(C₁-C₅)alkyl-R₂, —SO₂R₂, —SO₂(C₁-C₄)alkyl-OR₅, heteroaryl, —(C₁-C₄)alkyl-R₂, —NR₆R₇, —OC(O)NR₆R₇, and —NH—C(O)O—(C₁-C₄)alkyl-R₂, wherein any of such heteroaryl is optionally substituted by one or more groups selected from the group consisting of —OR₃, —(C₁-C₄)alkyl, —(C₁-C₄)haloalkyl, —(C₃-C₆)cycloalkyl, aryl, heteroaryl, and —NR₆R₇;
R₂ is H or is selected from the group consisting of —(C₁-C₄)alkyl, aryl, and heteroaryl, wherein any of such aryl, heteroaryl and alkyl are optionally substituted by one or more groups selected from the group consisting of —(C₁-C₄)alkyl, halo, —OR₅, and —R₅;
R₃ is H or is selected from the group consisting of —(C₁-C₄)alkyl, —(C₁-C₄)haloalkyl, and —(C₃-C₆)cycloalkyl optionally substituted by one or more halo;
R₄ is H or (C₁-C₄)alkyl;
R₅ is aryl optionally substituted by one or more groups selected from the group consisting of (C₁-C₄)alkyl and halo;
R₆ and R₇ are at each occurrence independently H or selected from the group consisting of —(C₁-C₆)alkyl, —(C₃-C₆)cycloalkyl, and heteroaryl, wherein any of such heteroaryl is optionally substituted by one or more groups selected from the group consisting of —OR₄, aryl, —(C₃-C₆)cycloalkyl, and heteroaryl.

4: A compound of formula (I) 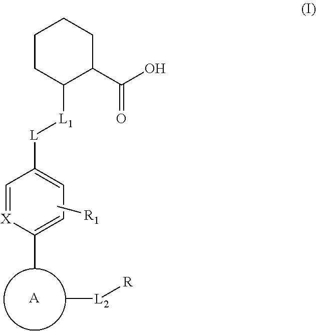
wherein X is —CH—, L is —NH—, L₁ is —C(O)—, and L₂ is —NH—, represented by the general formula (Ic) 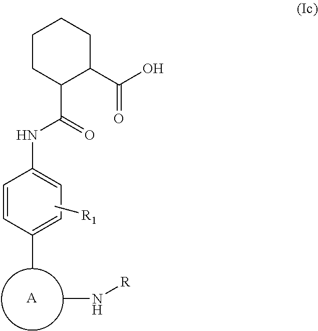
wherein
R₁ is H or —(C₁-C₄)alkyl;
A is selected from the group consisting of 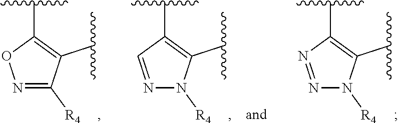
R is selected from the group consisting of —SO₂R₂, —SO₂(C₁-C₄)alkyl-OR₅, heteroaryl, —(C₁-C₄)alkyl-R₂, —NR₆R₇, —OC(O)NR₆R₇, and —NH—C(O)O—(C₁-C₄)alkyl-R₂, wherein any of such heteroaryl is optionally substituted by one or more groups selected from the group consisting of —OR₃, —(C₁-C₄)alkyl, —(C₁-C₄)haloalkyl, —(C₃-C₆)cycloalkyl, aryl, heteroaryl, and —NR₆R₇;
R₂ is H or is selected from the group consisting of —(C₁-C₄)alkyl, aryl, and heteroaryl, wherein any of such aryl, heteroaryl and alkyl are optionally substituted by one or more groups selected from the group consisting of —(C₁-C₄)alkyl, halo, —OR₅, and —R₅;
R₃ is H or is selected from the group consisting of —(C₁-C₄)alkyl, —(C₁-C₄)haloalkyl, and —(C₃-C₆)cycloalkyl optionally substituted by one or more halo;
R₄ is H or (C₁-C₄)alkyl;
R₅ is aryl optionally substituted by one or more groups selected from the group consisting of —(C₁-C₄)alkyl and halo; and
R₆ and R₇ are at each occurrence independently H or selected from the group consisting of —(C₁-C₆)alkyl, —(C₃-C₆)cycloalkyl, and heteroaryl, wherein any of such heteroaryl is optionally substituted by one or more groups selected from the group consisting of —OR₄, aryl, —(C₃-C₆)cycloalkyl, and heteroaryl.

5: The compound according to claim 1, selected from the group consisting of
(1S,2S)-2-((6-(4-((6-cyclopropoxypyrazin-2-yl)amino)-3-methylisoxazol-5-yl)-2-methylpyridin-3-yl)carbamoyl)cyclohexane-1-carboxylic acid,
(1S,2S)-2-((6-(4-((6-isopropoxypyrazin-2-yl)amino)-3-methylisoxazol-5-yl)-2-methylpyridin-3-yl)carbamoyl)cyclohexane-1-carboxylic acid,
(1S,2S)-2-((6-(4-((6-(3,3-difluorocyclobutoxy)pyrazin-2-yl)amino)-3-methylisoxazol-5-yl)-2-methylpyridin-3-yl)carbamoyl)cyclohexane-1-carboxylic acid,
(1S,2S)-2-((2-methyl-6-(3-methyl-4-((6-(trifluoromethyl)pyrazin-2-yl)amino)isoxazol-5-yl)pyridin-3-yl)carbamoyl)cyclohexane-1-carboxylic acid,
(1S,2S)-2-((6-(4-((6-(butylamino)pyrazin-2-yl)amino)-3-methylisoxazol-5-yl)-2-methylpyridin-3-yl)carbamoyl)cyclohexane-1-carboxylic acid,
(1S,2S)-2-((2-methyl-6-(3-methyl-4-((6-phenylpyrazin-2-yl)amino)isoxazol-5-yl)pyridin-3-yl)carbamoyl)cyclohexane-1-carboxylic acid,
(1S,2S)-2-((2-methyl-6-(3-methyl-4-((6-(pyridin-3-yl)pyrazin-2-yl)amino)isoxazol-5-yl)pyridin-3-yl)carbamoyl)cyclohexane-1-carboxylic acid,
(1S,2S)-2-((2-methyl-6-(3-methyl-4-((6-(pyridin-4-yl)pyrazin-2-yl)amino)isoxazol-5-yl)pyridin-3-yl)carbamoyl)cyclohexane-1-carboxylic acid,
(1S,2S)-2-((6-(4-((6-cyclopropylpyrazin-2-yl)amino)-3-methylisoxazol-5-yl)-2-methylpyridin-3-yl)carbamoyl)cyclohexane-1-carboxylic acid,
(1S,2S)-2-((6-(4-((6-(cyclohex-1-en-1-yl)pyrazin-2-yl)amino)-3-methylisoxazol-5-yl)-2-methylpyridin-3-yl)carbamoyl)cyclohexane-1-carboxylic acid,
(1S,2S)-2-((6-(4-((6-cyclopentylpyrazin-2-yl)amino)-3-methylisoxazol-5-yl)-2-methylpyridin-3-yl)carbamoyl)cyclohexane-1-carboxylic acid,
(1S,2S)-2-((6-(4-((6-isobutylpyrazin-2-yl)amino)-3-methylisoxazol-5-yl)-2-methylpyridin-3-yl)carbamoyl)cyclohexane-1-carboxylic acid,
(1S,2S)-2-((6-(4-((6-isopropylpyrazin-2-yl)amino)-3-methylisoxazol-5-yl)-2-methylpyridin-3-yl)carbamoyl)cyclohexane-1-carboxylic acid,
(1S,2S)-2-((2-methyl-6-(3-methyl-4-((6-(pyridin-2-yl)pyrazin-2-yl)amino)isoxazol-5-yl)pyridin-3-yl)carbamoyl)cyclohexane-1-carboxylic acid,
(1S,2S)-2-((6-(4-((6-(cyclopentylamino)pyrazin-2-yl)amino)-3-methylisoxazol-5-yl)-2-methylpyridin-3-yl)carbamoyl)cyclohexane-1-carboxylic acid,
2-((4-(4-((6-isopropoxypyrazin-2-yl)amino)-3-methylisoxazol-5-yl)phenyl)carbamoyl)cyclohexane-1-carboxylic acid (trans racemate),
(1S,2S)-2-((6-(5-(((4-isopropoxypyrimidin-2-yl)amino)methyl)-1-methyl-1H-pyrazol-4-yl)-2-methylpyridin-3-yl)carbamoyl)cyclohexane-1-carboxylic acid,
((1S,2S)-2-((6-(5-(((4-cyclohexylpyrimidin-2-yl)amino)methyl)-1-methyl-1H-pyrazol-4-yl)-2-methylpyridin-3-yl)carbamoyl)cyclohexane-1-carboxylic acid,
(1S,2S)-2-((2-methyl-6-(1-methyl-5-(((4-phenylpyrimidin-2-yl)amino)methyl)-1H-pyrazol-4-yl)pyridin-3-yl)carbamoyl)cyclohexane-1-carboxylic acid,
(1S,2S)-2-((4-(5-((6-isopropoxypyrazin-2-yl)amino)-1-methyl-1H-pyrazol-4-yl)phenyl)carbamoyl)cyclohexane-1-carboxylic acid,
(1S,2S)-2-((6-(5-(((isopentyl(methyl)carbamoyl)oxy)methyl)-1-methyl-1H-pyrazol-4-yl)-2-methylpyridin-3-yl)carbamoyl)cyclohexane-1-carboxylic acid,
(1S,2S)-2-((6-(4-((2-(2-chlorophenyl)ethyl)sulfonamido)-3-methylisoxazol-5-yl)-2-methylpyridin-3-yl)carbamoyl)cyclohexane-1-carboxylic acid,
(1S,2S)-2-((2-methyl-6-(3-methyl-4-((2-phenylethyl)sulfonamido)isoxazol-5-yl)pyridin-3-yl)carbamoyl)cyclohexane-1-carboxylic acid,
(1S,2S)-2-((6-(4-((2-(2-fluorophenyl)ethyl)sulfonamido)-3-methylisoxazol-5-yl)-2-methylpyridin-3-yl)carbamoyl)cyclohexane-1-carboxylic acid,
(1S,2S)-2-((6-(4-((2-(4-fluorophenoxy)ethyl)sulfonamido)-3-methylisoxazol-5-yl)-2-methylpyridin-3-yl)carbamoyl)cyclohexane-1-carboxylic acid,
(1S,2S)-2-((2-methyl-6-(3-methyl-4-((3-phenylpropyl)sulfonamido)isoxazol-5-yl)pyridin-3-yl)carbamoyl)cyclohexane-1-carboxylic acid,
(1S,2S)-2-((6-(4-((2-(4-fluorophenyl)ethyl)sulfonamido)-3-methylisoxazol-5-yl)-2-methylpyridin-3-yl)carbamoyl)cyclohexane-1-carboxylic acid,
(1S,2S)-2-((6-(4-((2-(4-chlorophenyl)ethyl)sulfonamido)-3-methylisoxazol-5-yl)-2-methylpyridin-3-yl)carbamoyl)cyclohexane-1-carboxylic acid,
(1S,2S)-2-((2-methyl-6-(3-methyl-4-((2-phenylpropyl)sulfonamido)isoxazol-5-yl)pyridin-3-yl)carbamoyl)cyclohexane-1-carboxylic acid,
Single Diastereomer 1 of (1S,2S)-2-((2-methyl-6-(3-methyl-4-((2-phenylpropyl)sulfonamido)isoxazol-5-yl)pyridin-3-yl)carbamoyl)cyclohexane-1-carboxylic acid,
Single Diastereomer 2 of (1S,2S)-2-((2-methyl-6-(3-methyl-4-((2-phenylpropyl)sulfonamido)isoxazol-5-yl)pyridin-3-yl)carbamoyl)cyclohexane-1-carboxylic acid,
(1S,2S)-2-((6-(4-((((benzyloxy)carbonyl)amino)methyl)-3-methylisoxazol-5-yl)-2-methylpyridin-3-yl)carbamoyl)cyclohexane-1-carboxylic acid,
(1S,2S)-2-((6-(4-(((((R)-1-(2-chlorophenyl)ethoxy)carbonyl)amino)methyl)-3-methylisoxazol-5-yl)-2-methylpyridin-3-yl)carbamoyl)cyclohexane-1-carboxylic acid,
(1S,2S)-2-((6-(4-(((((2-chlorobenzyl)oxy)carbonyl)amino)methyl)-3-methylisoxazol-5-yl)-2-methylpyridin-3-yl)carbamoyl)cyclohexane-1-carboxylic acid,
(1S,2S)-2-((2-methyl-6-(3-methyl-4-(((((R)-1-phenylethoxy)carbonyl)amino)methyl)isoxazol-5-yl)pyridin-3-yl)carbamoyl)cyclohexane-1-carboxylic acid,
(1S,2S)-2-((6-(4-(((4-ethoxypyrimidin-2-yl)amino)methyl)-3-methylisoxazol-5-yl)-2-methylpyridin-3-yl)carbamoyl)cyclohexane-1-carboxylic acid,
(1S,2S)-2-((2-methyl-6-(3-methyl-4-(((4-(thiophen-2-yl)pyrimidin-2-yl)amino)methyl)isoxazol-5-yl)pyridin-3-yl)carbamoyl)cyclohexane-1-carboxylic acid,
(1S,2S)-2-((2-methyl-6-(3-methyl-4-(((4-(pyridin-3-yl)pyrimidin-2-yl)amino)methyl)isoxazol-5-yl)pyridin-3-yl)carbamoyl)cyclohexane-1-carboxylic acid,
(1S,2S)-2-((2-methyl-6-(3-methyl-4-(((4-(thiophen-3-yl)pyrimidin-2-yl)amino)methyl)isoxazol-5-yl)pyridin-3-yl)carbamoyl)cyclohexane-1-carboxylic acid,
(1S,2S)-2-((2-methyl-6-(3-methyl-4-(((4-(naphthalen-1-yl)pyrimidin-2-yl)amino)methyl)isoxazol-5-yl)pyridin-3-yl)carbamoyl)cyclohexane-1-carboxylic acid,
(1S,2S)-2-((6-(4-(((4-(furan-2-yl)pyrimidin-2-yl)amino)methyl)-3-methylisoxazol-5-yl)-2-methylpyridin-3-yl)carbamoyl)cyclohexane-1-carboxylic acid,
(1S,2S)-2-((6-(5-(((((R)-1-(2-chlorophenyl)ethoxy)carbonyl)amino)methyl)-1-methyl-1H-1,2,3-triazol-4-yl)-2-methylpyridin-3-yl)carbamoyl)cyclohexane-1-carboxylic acid,
2-((4-(5-((6-isopropoxypyrazin-2-yl)amino)-1-methyl-1H-1,2,3-triazol-4-yl)phenyl)carbamoyl)cyclohexane-1-carboxylic acid (cis racemate),
(1S,2S)-2-((6-(5-(((4-cyclohexylpyrimidin-2-yl)amino)methyl)-1-methyl-1H-1,2,3-triazol-4-yl)-2-methylpyridin-3-yl)carbamoyl)cyclohexane-1-carboxylic acid,
(1S,2S)-2-((6-(4-(((cyclopentyl(methyl)carbamoyl)oxy)methyl)-3-methylisoxazol-5-yl)-2-methylpyridin-3-yl)carbamoyl)cyclohexane-1-carboxylic acid,
(1S,2S)-2-(((6-(4-((6-cyclopentylpyrazin-2-yl)amino)-3-methylisoxazol-5-yl)-2-methylpyridin-3-yl)oxy)methyl)cyclohexane-1-carboxylic acid,
(1S,2S)-2-(((6-(4-((6-ethoxypyrazin-2-yl)amino)-3-methylisoxazol-5-yl)pyridin-3-yl)oxy)methyl)cyclohexane-1-carboxylic acid,
(1S,2S)-2-(((6-(4-((6-isopropoxypyrazin-2-yl)amino)-3-methylisoxazol-5-yl)pyridin-3-yl)oxy)methyl)cyclohexane-1-carboxylic acid,
(1S,2S)-2-(((6-(3-methyl-4-((6-(2,2,2-trifluoroethoxy)pyrazin-2-yl)amino)isoxazol-5-yl)pyridin-3-yl)oxy)methyl)cyclohexane-1-carboxylic acid,
(1S,2S)-2-(((6-(4-((6-isobutoxypyrazin-2-yl)amino)-3-methylisoxazol-5-yl)pyridin-3-yl)oxy)methyl)cyclohexane-1-carboxylic acid,
(1S,2S)-2-(((6-(4-((6-ethoxypyrazin-2-yl)amino)-3-methylisoxazol-5-yl)-2-methylpyridin-3-yl)oxy)methyl)cyclohexane-1-carboxylic acid,
(1S,2S)-2-(((6-(4-((6-isopropoxypyrazin-2-yl)amino)-3-methylisoxazol-5-yl)-2-methylpyridin-3-yl)oxy)methyl)cyclohexane-1-carboxylic acid,
(1S,2S)-2-(((6-(4-((6-methoxypyrazin-2-yl)amino)-3-methylisoxazol-5-yl)-2-methylpyridin-3-yl)oxy)methyl)cyclohexane-1-carboxylic acid,
(1S,2S)-2-(((2-methyl-6-(3-methyl-4-((6-propylpyrazin-2-yl)amino)isoxazol-5-yl)pyridin-3-yl)oxy)methyl)cyclohexane-1-carboxylic acid,
(1S,2S)-2-(((6-(4-((2-(4-fluorophenoxy)ethyl)sulfonamido)-3-methylisoxazol-5-yl)-2-methylpyridin-3-yl)oxy)methyl)cyclohexane-1-carboxylic acid,
(1S,2S)-2-(((2-methyl-6-(3-methyl-4-(phenylsulfonamido)isoxazol-5-yl)pyridin-3-yl)oxy)methyl)cyclohexane-1-carboxylic acid,
(1S,2S)-2-(((2-methyl-6-(3-methyl-4-((2-phenylethyl)sulfonamido)isoxazol-5-yl)pyridin-3-yl)oxy)methyl)cyclohexane-1-carboxylic acid,
(1S,2S)-2-(((6-(5-((((R)-1-(2-chlorophenyl)ethoxy)carbonyl)amino)-1-methyl-1H-pyrazol-4-yl)-2-methylpyridin-3-yl)oxy)methyl)cyclohexane-1-carboxylic acid,
(1S,2S)-2-((4-(3-methyl-4-((((R)-1-(pyridin-3-yl)ethoxy)carbonyl)amino)isoxazol-5-yl)phenoxy)methyl)cyclohexane-1-carboxylic acid, Methyl (1S,2S)-2-(((2-methyl-6-(3-methyl-4-((((R)-1-(pyridin-3-yl)ethoxy)carbonyl)amino)isoxazol-5-yl)pyridin-3-yl)oxy)methyl)cyclohexane-1-carboxylic acid,
(1S,2S)-2-(((2-methyl-6-(1-methyl-5-(((4-phenylpyrimidin-2-yl)amino)methyl)-1H-pyrazol-4-yl)pyridin-3-yl)oxy)methyl)cyclohexane-1-carboxylic acid,
(1S,2S)-2-(((6-(5-((((benzyloxy)carbonyl)amino)methyl)-1-methyl-1H-1,2,3-triazol-4-yl)-2-methylpyridin-3-yl)oxy)methyl)cyclohexane-1-carboxylic acid,
(1S,2S)-2-(((6-(5-(((((R)-1-(2-chloropyridin-3-yl)ethoxy)carbonyl)amino)methyl)-1-methyl-1H-1,2,3-triazol-4-yl)-2-methylpyridin-3-yl)oxy)methyl)cyclohexane-1-carboxylic acid,
(1S,2S)-2-(((6-(5-(((((2-chloropyridin-3-yl)methoxy)carbonyl)amino)methyl)-1-methyl-1H-1,2,3-triazol-4-yl)-2-methylpyridin-3-yl)oxy)methyl)cyclohexane-1-carboxylic acid,
(1S,2S)-2-(((2-methyl-6-(1-methyl-5-(((4-(pyrazin-2-yl)pyrimidin-2-yl)amino)methyl)-1H-1,2,3-triazol-4-yl)pyridin-3-yl)oxy)methyl)cyclohexane-1-carboxylic acid,
(1S,2S)-2-(((6-(5-(((4-isopropoxypyrimidin-2-yl)amino)methyl)-1-methyl-1H-1,2,3-triazol-4-yl)-2-methylpyridin-3-yl)oxy)methyl)cyclohexane-1-carboxylic acid,
(1S,2S)-2-(((2-methyl-6-(3-methyl-4-(((4-(pyrazin-2-yl)pyrimidin-2-yl)amino)methyl)isoxazol-5-yl)pyridin-3-yl)oxy)methyl)cyclohexane-1-carboxylic acid,
(1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylic acid,
(1S,2S)-2-(((6-(5-((((R)-1-(2-chlorophenyl)ethoxy)carbonyl)amino)-1-methyl-1H-1,2,3-triazol-4-yl)-2-methylpyridin-3-yl)oxy)methyl)cyclohexane-1-carboxylic acid,
(1S,2S)-2-(((2-methyl-6-(1-methyl-5-(((((R)-pentan-2-yl)oxy)carbonyl)amino)-1H-1,2,3-triazol-4-yl)pyridin-3-yl)oxy)methyl)cyclohexane-1-carboxylic acid,
(1S,2S)-2-(((6-(5-((6-isopropoxypyrazin-2-yl)amino)-1-methyl-1H-1,2,3-triazol-4-yl)-2-methylpyridin-3-yl)oxy)methyl)cyclohexane-1-carboxylic acid,
(1S,2S)-2-(((6-(5-((6-ethoxypyrazin-2-yl)amino)-1-methyl-1H-1,2,3-triazol-4-yl)-2-methylpyridin-3-yl)oxy)methyl)cyclohexane-1-carboxylic acid,
(1S,2S)-2-(((6-(5-(((6-isopropoxypyrazin-2-yl)amino)methyl)-1-methyl-1H-1,2,3-triazol-4-yl)-2-methylpyridin-3-yl)oxy)methyl)cyclohexane-1-carboxylic acid,
(1S,2S)-2-(((6-(4-(((cyclopentyl(methyl)carbamoyl)oxy)methyl)-3-methylisoxazol-5-yl)-2-methylpyridin-3-yl)oxy)methyl)cyclohexane-1-carboxylic acid,
(1S,2S)-2-(((2-methyl-6-(3-methyl-4-(((methyl(propyl)carbamoyl)oxy)methyl)isoxazol-5-yl)pyridin-3-yl)oxy)methyl)cyclohexane-1-carboxylic acid,
(1S,2S)-2-(((2-methyl-6-(1-methyl-5-(((methyl(propyl)carbamoyl)oxy)methyl)-1H-1,2,3-triazol-4-yl)pyridin-3-yl)oxy)methyl)cyclohexane-1-carboxylic acid,
2-((4-(4-((((R)-1-(2-chloro phenyl)ethoxy)carbonyl)amino)-3-methylisoxazol-5-yl)phenoxy)methyl)cyclohexane-1-carboxylic acid (trans mixture),
(1S,2S)-2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid,
2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid (cis mixture),
(1S,2S)-2-(((6-(4-((((R)-1-(2-chlorophenyl)ethoxy)carbonyl)amino)-3-methylisoxazol-5-yl)-2-methylpyridin-3-yl)oxy)methyl)cyclohexane-1-carboxylic acid,
Single Diastereomer 1 of cis-2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid, and
Single Diastereomer 2 of cis-2-(((6-(4-((((R)-1-(2-chlorophenyl)ethoxy)carbonyl)amino)-3-methylisoxazol-5-yl)pyridin-3-yl)oxy)methyl)cyclohexane-1-carboxylic acid.

6: A method of making the compound of formula (I) according to claim 1, comprising reacting an intermediate compound of formula (XIV) or (XVIII) 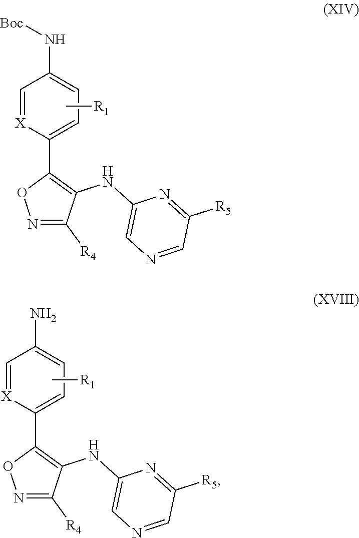
wherein X, R₁, R₄, and R₅ are as defined in claim 1.

7: A method of making the compound of formula (I) according to claim 1, comprising reacting an intermediate compound of formula (XXV): 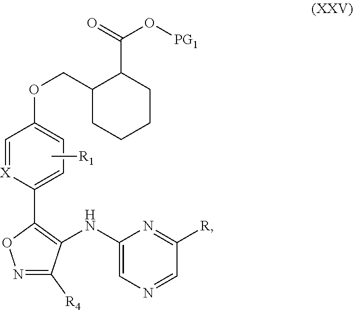
wherein:
X, R₁, and R₄ are as defined in claim 1,
PG₁ is a protecting group, and
R is aryl optionally substituted by one or more groups selected from the group consisting of (C₁-C₄)alkyl and halo.

8: A method of making the compound of formula (I) according to claim 1, comprising reacting an intermediate compound of formula (XXXV): 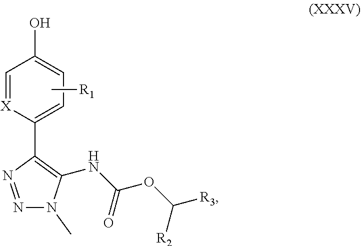
wherein X, R₁, R₂, and R₃ are as defined in claim 1.

9: A method of making the compound of formula (I) according to claim 1, comprising reacting an intermediate compound of formula (XXXVII): 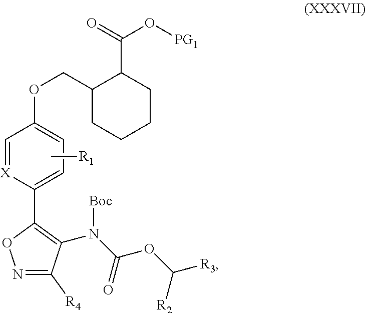
wherein:
X, R₁, R₂, R₃, and R₄ are as defined in claim 1, and
PG₁ is a protecting group.

10: A pharmaceutical composition comprising the compound according to claim 1, in admixture with one or more pharmaceutically acceptable carriers or excipients.

11: The pharmaceutical composition according to claim 10, which is suitable for oral administration.

12. (canceled)

13: A method of treating a disease, disorder, or condition associated with dysregulation of lysophosphatidic acid receptor 1 (LPA1) comprising administering the pharmaceutical composition according to claim 10 to a subject in need thereof.

14: A method of treating fibrosis and/or a disease, disorder, or condition that involves fibrosis, comprising administering the pharmaceutical composition according to claim 10 to a subject in need thereof.

15: The method according to claim 14, wherein the fibrosis comprises at least one fibrosis selected from the group consisting of pulmonary fibrosis, idiopathic pulmonary fibrosis (IPF), hepatic fibrosis, sarcoidosis, familiar pulmonary fibrosis, chronic hypersensitivity pneumonitis (CHP), kidney or renal fibrosis, ocular fibrosis, cardiac fibrosis, arterial fibrosis and systemic sclerosis.

16: The method according to claim 15, wherein the fibrosis comprises idiopathic pulmonary fibrosis (IPF).
